(19) United States
(12) Patent Application Publication
(10) Pub. No.: US 2026/0345619 A1
(43) Pub. Date: Nov. 5, 2026

(54) TREATMENT OF MODERATE-TO-SEVERE ASTHMA BY ADMINISTERING RILZABRUTINIB

(71) Applicant: Principia Biopharma Inc., Morristown, NJ (US)

(72) Inventors: Jessica Gereige, Brookline, MA (US); David Gossage, Beaufort, SC (US); Leda Mannent, Paris (FR); Renata Martincova, Prague (CZ); Vincent Mikol, Paris (FR); Benjamin Suratt, Charlestown, MA (US)

(73) Assignee: Principia Biopharma Inc., Morristown, NJ (US)

(21) Appl. No.: 19/699,349

(22) Filed: Jun. 5, 2026

Related U.S. Application Data
(63) Continuation of application No. PCT/US2024/058667, filed on Dec. 5, 2024.
(60) Provisional application No. 63/606,952, filed on Dec. 6, 2023.
(60) Provisional application No. 63/637,741, filed on Apr. 23, 2024.

(51) Int. Cl. A61K 31/519; A61P 11/06

(52) U.S. Cl. CPC A61K 31/519; A61P 11/06

(57) ABSTRACT

Methods for treating patients with moderate-to-severe asthma comprising administering a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof are disclosed.

This application is a continuation of International Application No. PCT/US2024/058667, filed Dec. 5, 2024, which claims the benefit of priority to U.S. Application No. 63/637,741 filed Apr. 23, 2024, and U.S. Application No. 63/606,952 filed Dec. 6, 2023, each of which is incorporated herein by reference in its entirety.

Disclosed herein are methods for treating moderate-to-severe asthma. BTK inhibitors and pharmaceutical compositions comprising the same are also disclosed.

Asthma is a chronic inflammatory disease of the airways characterized by airway hyperresponsiveness, acute and chronic bronchoconstriction, airway edema and mucus plugging. The inflammation component of asthma is thought to involve many cell types including epithelial cells, T lymphocytes, eosinophils, mast cells, neutrophils, innate lymphoid cells type 2 (ILC2) and their biological products. Patients with asthma most often present with symptoms of wheezing, shortness of breath, cough, and chest tightness (Global Initiative for Asthma (GINA). Global Strategy for Asthma Management and Prevention, Updated 2019, Updated 2020, Updated 2021).

Despite current treatment options, uncontrolled asthma continues to carry a significant burden, with more than 1 million moderate-to-severe asthmatic adolescent and adults in the U.S. having uncontrolled symptoms on inhaled corticosteroids (ICS) combined with a second controller or systemic corticosteroid therapy (Chung K F et al., 2014; Global Initiative for Asthma (GINA). Global Strategy for Asthma Management and Prevention, Updated 2019, Updated 2020, Updated 2021). These patients are prone to severe exacerbations and contribute disproportionately to the overall morbidity in this condition, including more hospital days, emergency room visits, and lost work/school days than matched controls with good asthma control. Additionally, they are likely to experience co-morbidities such as anxiety, depression, and insomnia (Bahadori K et al., 2009; To T et al., 2012). Furthermore, systemic corticosteroids, while clinically effective, are associated with significant side effects, such as fluid retention and swelling, glaucoma, increased blood pressure, increased risk of infections, diabetes, osteoporosis, and impaired growth in children (McCracken J L et al., 2016).

For severe uncontrolled asthma patients, there are several currently approved biologics (Pelaia C et al., 2020). In the severe, corticosteroid-refractory allergy-induced asthma population, the anti-immunoglobulin E (IgE) agent omalizumab (Xolair®) has shown efficacy. Xolair is associated with a black box warning of potential for anaphylaxis. In the severe population with an eosinophilic phenotype, the two currently approved anti-interleukin (IL)-5 agents, mepolizumab (Nucala®) and reslizumab (Cinqair®), and the anti-IL5 receptor benralizumab (Fasenra™) have shown efficacy as well. These anti-IL5 agents now provide additional therapeutic options with important limitations though. Even in the high eosinophilic group, 36% of patients on mepolizumab had no decrease in oral corticosteroid (OCS) dose or had a lack of asthma control (Bel E H et al., 2014), suggesting that targeting a single cytokine in a heterogenous disease such as asthma is not sufficient. Despite the advent of biologics in the past decade, there remains a significant unmet need for effective asthma treatments. In support of this, up to 60% of patients remain uncontrolled despite treatment with ICS/LABA.

The main objectives for new therapies are to improve asthma symptoms, lung function, and prevent exacerbations, while optimizing adherence to the treatment. As noted above, one of the reasons for the poor response to medication in some patients with severe asthma may be the heterogeneity of the disease, namely the mixed eosinophilic and neutrophilic inflammation, only partly responding to the current targeted therapies. Underlying deficiencies in innate immunity and evidence of a synergism between viral infection and allergic mechanisms in increasing risk of exacerbations are all important mechanisms to target with novel treatments (Custovic A et al., 2013). Thus, there is an important need for new therapeutic options for patients with asthma.

Compound (I) is a BTK inhibitor of the following structure:

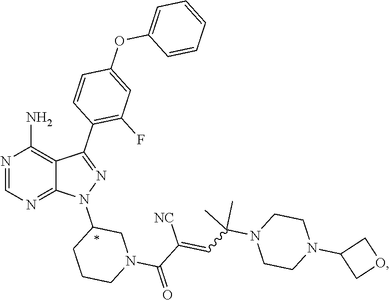 [SMILES: CC(C)(C=C(C#N)C(=O)N1CCCC(n2nc(-c3ccc(Oc4ccccc4)cc3F)c3c(N)ncnc32)C1)N1CCN(C2COC2)CC1]

wherein *C is a stereochemical center. See PCT Publication No. WO 2014/039899, which is incorporated herein by reference, e.g., Example 31.

(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, having the following structure:

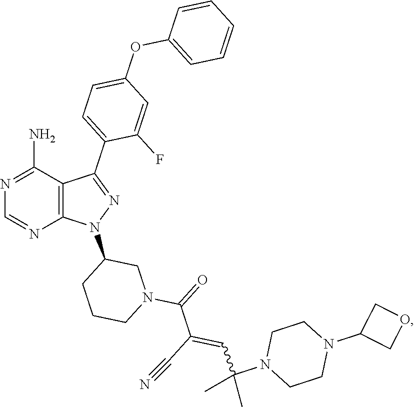 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

is also known as PRN1008 and rilzabrutinib. This compound has been disclosed in several patent publications, such as, e.g., PCT Publication Nos. WO 2014/039899, WO 2015/127310, WO 2016/100914, WO 2016/105531, WO 2018/005849, and WO 2021/150723, the contents of each of which are incorporated by reference herein.

Rilzabrutinib is a novel, highly selective, and potent small molecule inhibitor of non-T cell white blood cell signaling via B-cell receptor, FcγR, and/or FcεR signaling of the BTK pathway. In the context of ITP, rilzabrutinib has the potential to (1) inhibit B cell activation and (2) interrupt antibody-coated cell phagocytosis by FCγR in the spleen and liver (Bradshaw et al. 2021, Langrish et al. 2021, Owens et al. 2022).

Rilzabrutinib functions as a reversible covalent BTK inhibitor and is capable of both non-covalently and covalently binding to its target; in particular, its reversible cysteine binding enables high selectivity and precise BTK inhibition without a permanent modification of proteins and peptides (Langrish et al. 2021, Owens et al. 2022, Smith P F et al. 2017). Taken together, these properties allow for enhanced selectivity and extended inhibition with low systemic exposure. In comparison to first and second generation BTKi, rilzabrutinib has shown minimal cross-reactivity with other molecules and is low risk for off-target effects (Smith P F et al. 2017). Importantly, rilzabrutinib's reversible binding minimizes the likelihood of permanently modified peptides (Serafimova IM 2012). In addition, rilzabrutinib shows improved kinase selectivity relative to the covalent BTK inhibitor ibrutinib. Preclinical studies in a broad kinase enzyme inhibition panel showed that 1 μM rilzabrutinib achieved >90% inhibition of just 6 of 251 kinases sharing a common cysteine in their active site. By contrast, 1 μM ibrutinib inhibited 21 kinases. Rilzabrutinib's IC50 values were 1.3 nM for BTK, 0.8 nM for tyrosine protein kinase TEC, 1.0 nM for bone marrow tyrosine kinase on chromosome X (BMX), 1.2 nM for receptor-like kinase (RLK), 6.3 nM for B cell lymphocyte kinase (BLK), and 11 nM for ERBB4. Further preclinical assays with rilzabrutinib showed that binding to BTK persisted while that for other TEC family members decayed rapidly over time.

Rilzabrutinib has shown encouraging results for the treatment of immune-mediated diseases. In humans, rilzabrutinib is rapidly absorbed following oral administration, with a fast half-life (3-4 h) and variable pharmacokinetics (Smith P F et al., 2017).

In Phase 1 studies of rilzabrutinib with 114 healthy volunteers, target BTK occupancy levels were safely and consistently exceeded, suggesting rilzabrutinib may be highly effective in treating autoimmune diseases. Moreover, preclinical and clinical pharmacokinetic and pharmacodynamic data showed that treatment effects endured even after the compound was cleared from circulation, consistent with an extended target residence time (Hill R et al., 2015) and high target occupancy rate (>90% within four hours and high sustained occupancy over 24 h) (Smith P F et al., 2015).

Rilzabrutinib has also demonstrated a favorable safety profile in clinical studies. In contrast with non-selective, irreversible BTK inhibitors, rilzabrutinib does not alter platelet aggregation in healthy volunteers or patients with ITP and thus does not lead to bleeding problems (Langrish et al. 2021, von Hundelshausen and Seiss 2021). As an additional point of contrast with irreversible BTK inhibitors, rilzabrutinib treatment does not exert clinically relevant effects on cardiac repolarization (electrocardiogram parameters including corrected QT interval) in healthy volunteers (N=51), even when administered at supratherapeutic doses (Lipsky and Lamanna 2020). Indeed, the most commonly reported adverse events in healthy volunteers were gastrointestinal adverse events, including nausea/vomiting and diarrhea. No serious adverse events or deaths were reported, and no participants discontinued treatment due to an adverse event (Smith P F 2017).

The clinical efficacy of rilzabrutinib was further demonstrated in an open-label, Phase 1/2 study in immune thrombocytopenic purpura (ITP). In this trial, 50% of participants achieved the primary response endpoint after treatment with rilzabrutinib for at least 12 weeks. Furthermore, there were no treatment-emergent serious adverse events (TESAEs) observed. Collectively, these and other studies have shown rilzabrutinib to be a safe and effective inhibitor of BTK.

Accordingly, disclosed herein are methods of treating asthma in a human patient in need thereof, comprising administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof.

Further disclosed herein are methods of treating asthma in a human patient in need thereof, comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, wherein the human patient in need thereof has asthma that is not well controlled by treatment with an inhaled corticosteroid (ICS) and a long-acting β2 adrenergic agonist (LABA).

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof.

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof, wherein the human patient in need thereof has asthma that is not well controlled by treatment with an inhaled corticosteroid (ICS) and/or a long-acting β2 adrenergic agonist (LABA).

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof.

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA).

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof.

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof, wherein the human patient in need thereof has asthma that is not well controlled by treatment with an inhaled corticosteroid (ICS) and/or a long-acting β2 adrenergic agonist (LABA).

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof.

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA).

### BRIEF DESCRIPTION OF DRAWINGS

FIG. 1 shows a graphical representation of the study design for the 400 mg BID cohort.

FIG. 2 shows a graphical representation of the study design for the 400 mg TID cohort.

FIGS. 3A and B show a Kaplan-Meier plot of time to LOAC post-randomization for the 400 mg BID (A) and 400 mg TID (B) cohorts (mITT population).

FIGS. 4A and B show a plot of mean change from baseline in pre-bronchodilator FEV1 (L) over time for the 400 mg BID (A) and 400 mg TID (B) cohorts (on-site conventional spirometry, as observed; mITT population).

FIGS. 5A and B show a plot of LS mean change from baseline in pre-bronchodilator FEV1 over time, reduced model, for the 400 mg BID (A) and 400 mg TID (B) cohorts (mITT population).

FIGS. 6A and B show a plot of LS mean change from baseline in pre-bronchodilator percent predicted FEV1 (L) overtime, reduced model, for the 400 mg BID (A) and 400 mg TID (B) cohorts (mITT population).

FIG. 7 shows a plot of LS mean change from inhalations/day of albuterol or levalbuterol for symptom relief over time for the 400 mg BID cohort (mITT population).

FIG. 8 shows a plot of LS mean change from inhalations/day of albuterol or levalbuterol for symptom relief over time, reduced model, for the 400 mg BID cohort (mITT population).

FIGS. 9A and B show a plot of LS mean change from ACQ-5 over time for the 400 mg BID (A) and 400 mg TID (B) cohorts (mITT population).

FIG. 10 shows a plot of the proportion of participants with ACQ-5 score less than 0.75 over time for the 400 mg BID cohort (ITT population).

FIGS. 11A and B show a plot of LS mean change from baseline in ACQ-5 frequency of awakening by asthma over time for the 400 mg BID (A) and 400 mg TID (B) cohorts (mITT population).

FIGS. 12A and B show a plot of LS mean change from baseline in ACQ-5 asthma symptoms when woke up over time for the 400 mg BID (A) and 400 mg TID (B) cohorts (mITT population).

FIGS. 13A and B show a plot of LS mean change from baseline in ACQ-5 limitation of activities by asthma over time for the 400 mg BID (A) and 400 mg TID (B) cohorts (mITT population).

FIGS. 14A and B show a plot of LS mean change from baseline in ACQ-5 shortness of breath experienced over time for the 400 mg BID (A) and 400 mg TID (B) cohorts (mITT population).

FIGS. 15A and B show a plot of LS mean change from baseline in ACQ-5 wheeze over time for the 400 mg BID (A) and the 400 mg TID (B) cohorts (mITT population).

FIGS. 16A and B show a plot of LS mean change from AQLQ global score over time for the 400 mg BID (A) and 400 mg TID (B) cohorts (mITT population).

### DEFINITIONS

Unless otherwise stated, the following terms used in the specification and claims are defined for the purposes of this Application and have the following meanings. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

As used herein, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. With regard to specific values, it should be understood that specific values described herein for subject populations (e.g., the subject of the described clinical trial) represent median, mean, or statistical numbers, unless otherwise provided. Accordingly, aspects of the present disclosure requiring a particular value in a subject are supported herein by population data in which the relevant value is assessed to be a meaningful delimitation on the subject population.

As used herein, the term “active pharmaceutical ingredient” or “therapeutic agent” (“API”) refers to a biologically active compound.

As used herein, the term “approved treatment” refers to a medication that has received regulatory authorization, in any country, for its intended use.

As used herein, the terms “administer,” “administering,” or “administration” herein refer to providing, giving, dosing, and/or prescribing by either a health practitioner or an authorized agent and/or putting into, taking, or consuming by the patient or person himself or herself. For example, “administration” of an API to a patient refers to any route (e.g., oral delivery) of introducing or delivering the API to the patient. Administration includes self-administration and administration by another.

As used herein, the term “baseline” refers to a measurement obtained within four weeks prior to initiating rilzabrutinib treatment.

As used herein, “BID” and “bid” are used interchangeably to refer to twice a day. As used herein, 400 mg BID refers to a total dose of 800 mg per day.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

As used herein, an amount expressed in terms of “mg of [X]” refers to the total amount in milligrams of [X], i.e., the free base. In some embodiments, rilzabrutinib may be administered as a pharmaceutically acceptable salt of rilzabrutinib, in which case an amount expressed in terms of “mg of rilzabrutinib” refers to the total amount in milligrams of rilzabrutinib, i.e., the free base, plus the equivalent amount of one or more pharmaceutically acceptable salts of rilzabrutinib based on the weight of free base therein. For example, “400 mg of at least one compound chosen from rilzabrutinib and pharmaceutically acceptable salts thereof” includes 400 mg of rilzabrutinib and a concentration of one or more pharmaceutically acceptable salts of rilzabrutinib equivalent to 400 mg of rilzabrutinib.

As used herein, a “pharmaceutically acceptable carrier or excipient” means a carrier or an excipient that is useful in preparing a pharmaceutical composition that is generally safe, and neither biologically nor otherwise undesirable, such as, e.g., a carrier or an excipient that is acceptable for mammalian pharmaceutical use.

As used herein, the term “pharmaceutically acceptable salt” refers to a salt form, e.g., an acid addition salt, of an active pharmaceutical agent that is pharmaceutically acceptable and that possesses the desired pharmacological activity of the API of which the salt is made. Pharmaceutically acceptable salts are well known in the art and include those derived from suitable inorganic and organic acids. Such salts include, but are not limited to, salts formed with inorganic acids such as hydrochloric acid, hydrobromic acid, sulfuric acid, phosphoric acid, and the like; or formed with organic acids such as formic acid, acetic acid, propionic acid, hexanoic acid, lactic acid, malonic acid, succinic acid, malic acid, maleic acid, fumaric acid, tartaric acid, citric acid, benzoic acid, mandelic acid, methanesulfonic acid, ethanesulfonic acid, 1,2-ethanedisulfonic acid, benzenesulfonic acid, 4-toluenesulfonic acid, and the like. S. M. Berge et al. describes pharmaceutically acceptable salts in detail in J. Pharmaceutical Sciences, 1977, 66, 1-19.

As used herein, the terms “PRN1008,” “rilzabrutinib,” “(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile;” “the compound of Formula (I);” and “2-[(3R)-3-[4-amino-3-(2-fluoro-4-phenoxy-phenyl)pyrazolo[3,4-d]-pyrimidin-1-yl]piperidine-1-carbonyl]-4-methyl-4-[4-(oxetan-3-yl)piperazin-1-yl]pent-2-enenitrile” are used interchangeably to refer to a compound having the structure:

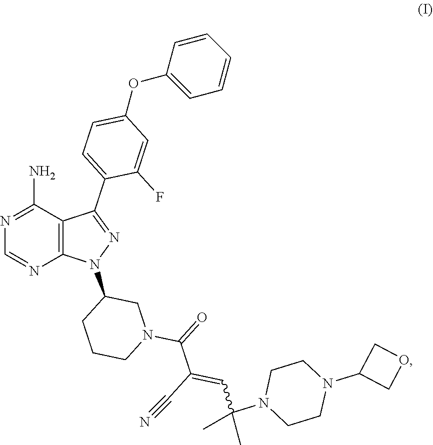 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

which is also referred to as 2-[(3R)-2-[4-amino-3-(2-fluoro-4-phenoxy-phenyl)pyrazolo[3,4-d]pyrimidin-1-yl]piperdine-1-carbonyl]-4-methyl-4-[4-(oxetan-3-yl)piperazin-1-yl]-(E and Z)-pent-2-enenitrile; (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; 1-piperidinepropanenitrile, 3-[4-amino-3-(2-fluoro-4-phenoxyphenyl)-1H-pyrazolo[3,4-d]pyrimidin-1-yl]-α-[2-methyl-2-[4-(3-oxetanyl)-1-piperazinyl]propylidene]-β-oxo-, (3R)-; (EZ)-2-[(3R)-3-[4-amino-3-(2-fluoro-4-phenoxyphenyl)pyrazolo[3,4-d]pyrimidin-1-yl]piperidine-1-carbonyl]-4-methyl-4-[4-(oxetan-3-yl)piperazin-1-yl]pent-2-enenitrile; and also by the International Nonproprietary Names for Pharmaceutical Substances (INN) as published by the World Health Organization (https://cdn.who.int/media/docs/default-source/international-nonproprietarv-names-(inn)/pl121.pdf?sfvrsn=69617906_15&download=true) having the following structure:

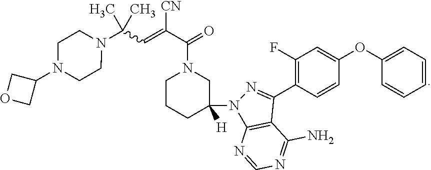 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1 ; "."]

The compound of Formula (I) includes E and Z isomers, as indicated by the wavy bond in the structure shown above. The compound of Formula (I) may be present as a salt form.

An isomer of rilzabrutinib may contain the corresponding (Z) isomer as an impurity in less than about 1% by weight; a dose of the (Z) isomer of rilzabrutinib may contain the corresponding (E) isomer as an impurity in less than about 1% by weight. When rilzabrutinib is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 1% by weight. In some embodiments, the molar ratio of (E) to (Z) isomer is 9:1. rilzabrutinib or a pharmaceutically acceptable salt thereof may also be referred to herein as a “drug,” “active agent,” “a therapeutically active agent,” or “API.”

As used herein, the term “therapeutically effective amount” refers to that of a compound that produces the desired effect for which it is administered (e.g., improvement in asthma or a symptom of asthma, or lessening the severity of asthma or a symptom of asthma). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, “TID” and “tid” are used interchangeably to refer to three times a day. As used herein, 400 mg TID refers to a total dose of 1200 mg per day.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

The present disclosure provides methods of treating asthma in a human patient in need thereof. In some embodiments, the methods comprise administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof.

In some embodiments, the human patient has mild asthma. In some embodiments, the human patient has moderate-to-severe asthma. In some embodiments, the human patient has moderate asthma. In some embodiments, the human patient has severe asthma. In some embodiments, the human patient has asthma that is not well controlled. In some embodiments, the human patient has asthma that is not well controlled for one or more symptoms. In some embodiments, the one or more symptoms is selected from frequency of awakening by asthma, asthma symptoms when woke up, limitation of activities by asthma, shortness of breath, and wheeze. In some embodiments, the one or more symptom is frequency of awakening by asthma. In some embodiments, the one or more symptom is asthma symptoms when woke up. In some embodiments, the one or more symptom is limitation of activities by asthma. In some embodiments, the one or more symptom is shortness of breath. In some embodiments, the one or more symptom is wheeze. In some embodiments, the human patient is receiving treatment with step 3, 4, or 5 of the Global Initiative for Asthma (GINA). In some embodiments, the human patient is receiving treatment with step 3 of GINA. In some embodiments, the human patient is receiving treatment with step 4 of GINA. In some embodiments, the human patient is receiving treatment with step 5 of GINA. In some embodiments, the human patient has asthma that is not well controlled by treatment with an inhaled corticosteroid (ICS) and/or a long-acting β2 adrenergic agonist (LABA). In some embodiments, the human patient has asthma that is not well controlled by treatment with an ICS. In some embodiments, the human patient has asthma that is not well controlled by treatment with an LABA.

In some embodiments, the human patient is withdrawn from ICS/LABA therapy. In some embodiments, the human patient is not withdrawn from ICS/LABA therapy.

In some embodiments, the human patient does not experience a ≥30% reduction from baseline in morning PEF.

In some embodiments, the human patient does not require ≥6 additional reliever puffs of salbutamol/albuterol or levosalbutamol/levalbuterol within a 24-hour period for two consecutive 24-hour periods. In some embodiments, the human patient does not require ≥6 additional reliever puffs of salbutamol/albuterol or levosalbutamol/levalbuterol within a 24-hour period. In some embodiments, the human patient does not require ≥5 additional reliever puffs of salbutamol/albuterol or levosalbutamol/levalbuterol within a 24-hour period. In some embodiments, the human patient does not require ≥4 additional reliever puffs of salbutamol/albuterol or levosalbutamol/levalbuterol within a 24-hour period. In some embodiments, the human patient does not require ≥3 additional reliever puffs of salbutamol/albuterol or levosalbutamol/levalbuterol within a 24-hour period. In some embodiments, the human patient does not require ≥2 additional reliever puffs of salbutamol/albuterol or levosalbutamol/levalbuterol within a 24-hour period.

In some embodiments, the human patient does not require an increase in ICS≥4 times the last prescribed ICS dose. In some embodiments, the human patient does not require an increase in ICS≥3 times the last prescribed ICS dose. In some embodiments, the human patient does not require an increase in ICS≥2 times the last prescribed ICS dose. In some embodiments, the human patient does not require an increase in ICS≥50% of the last prescribed ICS dose. In some embodiments, the human patient does not require an increase in ICS dose. In some embodiments, the human patient achieves a decrease in ICS dose.

In some embodiments, the human patient does not require the use of systemic steroid treatment, optionally wherein the systemic steroid treatment comprises oral or parenteral treatment.

In some embodiments, the human patient does not require hospitalization or an emergency room visit for asthma exacerbation.

In some embodiments, the human patient achieves an increase in pre-bronchodilator FEV1 relative to baseline pre-bronchodilator FEV1. In some embodiments, the human patient achieves an increase in the percent predicted pre-bronchodilator FEV1 prior to baseline pre-bronchodilator FEV1.

In some embodiments, the human patient achieves a reduction in Asthma Control Questionnaire-5 (ACQ-5) score relative to a baseline ACQ-5 score. In some embodiments, the human patient achieves a reduction in ACQ-5 of ≥0.5 relative to baseline ACQ-5. In some embodiments, the human patient achieves an ACQ-5 score of ≤0.75.

In some embodiments, the human patient achieves an increase in Asthma Quality of Life Questionnaire with Standardized Activities (AQLQ(S)) relative to baseline AQLQ(S).

In some embodiments, the human patient achieves a reduction in asthma daytime symptom diary (ADSD) relative to baseline ADSD. In some embodiments, the human patient achieves a reduction in asthma nighttime symptom diary (ANSD) relative to baseline ANSD. In some embodiments, wherein the human patient achieves a reduction in ADSD relative to baseline ADSD and/or a reduction in ANSD relative to baseline ANSD, the patient receives concomitant treatment with ICS and/or LABA.

In some embodiments, the human patient achieves a reduction in inhalations/day of albuterol or levalbuterol for symptom relief relative to baseline inhalations/day. In some embodiments, wherein the human patient achieves a reduction in inhalations/day of albuterol or levalbuterol for symptom relief relative to baseline inhalations/day, the patient receives concomitant treatment with ICS/LABA.

In some embodiments, the human patient achieves a reduction in Patient Global Impression of Severity (PGIS) relative to baseline PGIS. In some embodiments, the human patient achieves an improvement in Patient Global Impression of Change (PGIC).

In some embodiments, the human patient has had asthma for at least 12 months.

In some embodiments, the human patient is being treated with a moderate to high dose of ICS therapy in combination with LABA. In some embodiments, the human patient is being treated with a moderate dose of ICS therapy in combination with LABA. In some embodiments, the human patient is being treated with a high dose of ICS therapy in combination with LABA. In some embodiments, the ICS is fluticasone propionate and the human patient is being treated with ≥250 μg of fluticasone propionate BID or comparable ICS daily dosage to a maximum of 2000 μg/day of fluticasone propionate or clinically comparable ICS. In some embodiments, the human patient has received treatment with the ICS for at least 3 months. In some embodiments, the human patient has received treatment with a stable dose of the ICS for at least 1 month.

In some embodiments, the human patient has a baseline reversibility of at least 12% and 200 mL in FEV1 15 to 30 minutes after administration of 200-400 μg of albuterol/salbutamol or levalbuterol/levosalbutamol. In some embodiments, the human patient has a history reversibility of at least 12% and 200 mL in FEV1 15 to 30 minutes after administration of 200-400 μg of albuterol/salbutamol or levalbuterol/levosalbutamol within the 5 years prior to initiation rilzabrutinib treatment. In some embodiments, the human patient has a history of positive response to methacholine challenge within the 5 years prior to initiation rilzabrutinib treatment. In some embodiments, the human patient has a history of one or more of the following within the 2 years prior to initiation of rilzabrutinib treatment: (a) treatment with a systemic steroid for worsening asthma, optionally wherein the systemic steroid treatment comprises oral or parenteral treatment; and (b) hospitalization or an emergency room visit for asthma exacerbation.

In some embodiments, the treatment period is at least 12 weeks.

In some embodiments, the at least one compound consists of at least one compound chosen from the (E) isomer of rilzabrutinib and pharmaceutically acceptable salts thereof. In some embodiments, the at least one compound consists of at least one compound chosen from the (Z) isomer of rilzabrutinib and pharmaceutically acceptable salts thereof. In some embodiments, the at least one compound consists of a mixture of (E) and (Z) isomers of rilzabrutinib or a pharmaceutically acceptable salt of the foregoing.

In some embodiments, the methods comprise administering rilzabrutinib to the human patient twice a day. In some embodiments, the methods comprise administering to the human patient 400 mg of rilzabrutinib twice a day. In some embodiments, the methods comprise administering rilzabrutinib to the human patient three times a day. In some embodiments, the methods comprise administering to the human patient 400 mg of rilzabrutinib three times a day.

In some embodiments, the at least one compound is the (E) isomer of rilzabrutinib. In some embodiments, the at least one compound is the (Z) isomer of rilzabrutinib. In some embodiments, the at least one compound consists of a mixture of (E) and (Z) isomers of rilzabrutinib.

In some embodiments, the at least one compound is orally administered to the human patient. In some embodiments, the at least one compound is administered to the human patient in the form of at least one tablet. In some embodiments, the at least one compound is administered with water.

In some embodiments, the at least one compound is rilzabrutinib.

In some embodiments, the methods comprise administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, wherein the human patient in need thereof has asthma that is not well controlled by treatment with an inhaled corticosteroid (ICS) and/or a long-acting β2 adrenergic agonist (LABA)

The present disclosure provides for the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof.

The present disclosure provides for the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof, wherein the human patient in need thereof has asthma that is not well controlled by treatment with an inhaled corticosteroid (ICS) and/or a long-acting β2 adrenergic agonist (LABA).

The present disclosure provides for the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof.

The present disclosure provides for the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA).

The present disclosure provides for a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof.

The present disclosure provides for a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof, wherein the human patient in need thereof has asthma that is not well controlled by treatment with an inhaled corticosteroid (ICS) and/or a long-acting β2 adrenergic agonist (LABA).

The present disclosure provides for a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof.

The present disclosure provides for a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA).

### Pharmaceutical Compositions:

In some embodiments of the present disclosure, rilzabrutinib is administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet.

In some embodiments of the present disclosure, rilzabrutinib is orally administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet.

In some embodiments, rilzabrutinib is administered in the form of a film-coated tablet.

In some embodiments of the present disclosure, rilzabrutinib is administered in the form of at least one tablet comprising: at least one compound chosen from rilzabrutinib and pharmaceutically acceptable salts thereof; and at least one pharmaceutically acceptable excipient. In some embodiments, rilzabrutinib is administered in the form of at least one tablet comprising: at least one compound chosen from rilzabrutinib and pharmaceutically acceptable salts thereof; at least one filler; at least one disintegrant; at least one lubricant; and at least one film coating.

In some embodiments, rilzabrutinib is administered with a glass of water.

The proportion and nature of any pharmaceutically acceptable excipient may be determined by the chosen route of administration and standard pharmaceutical practice. Except insofar as any conventional pharmaceutically acceptable excipient is incompatible with rilzabrutinib, such as by producing any undesirable biological effect or otherwise interacting in a deleterious manner with any other component(s) of the pharmaceutically composition, its use is contemplated to be within the scope of this disclosure.

Some non-limiting examples of materials which may serve as pharmaceutically acceptable excipients include: (1) sugars, such as, e.g., lactose, glucose, and sucrose; (2) starches, such as, e.g., corn starch and potato starch; (3) cellulose and its derivatives, such as, e.g., sodium carboxymethyl cellulose, ethyl cellulose, and cellulose acetate; (4) powdered tragacanth; (5) malt; (6) gelatin; (7) talc; (8) excipients, such as, e.g., cocoa butter and suppository waxes; (9) oils, such as, e.g., peanut oil, cottonseed oil, safflower oil, sesame oil, olive oil, corn oil, and soybean oil; (10) glycols, such as, e.g., propylene glycol; (11) polyols, such as, e.g., glycerin, sorbitol, mannitol, and polyethylene glycol; (12) esters, such as, e.g., ethyl oleate and ethyl laurate; (13) agar; (14) buffering agents, such as, e.g., magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

One skilled in the art can readily select the proper form and route of administration depending upon the disorder or condition to be treated, the stage of the disorder or condition, and other relevant circumstances.

### EXAMPLES

The following example is intended to be illustrative and is not meant in any way to limit the scope of the disclosure.

### ABBREVIATIONS

ACQ-5 Asthma Control Questionnaire-5
AD Atopic dermatitis
ADSD Asthma daytime symptom diary
AE Adverse event
AESI Adverse event of special interest
ANSD Asthma nighttime symptom diary
AQLQ(S) Asthma Quality of Life Questionnaire with Standardized Activities
ATC Anatomical therapeutic chemical
BD Bronchodilator
BDR Bronchodilator response
BID Twice daily
BTK Bruton's tyrosine kinase
CI Confidence interval
CS Corticosteroid
CSICF Core study informed consent form
CYP Cytochrome P450
DPI Dry powder inhaler
ECG Electrocardiogram
EOT End of treatment
ETD Early treatment discontinuation
FcγR Fc-gamma receptor
FcεR Fc-epsilon receptor
FEF Forced expiratory flow
FVC Forced vital capacity
HIV Human immunodeficiency virus
HR Hazard ratio
HRT Hormonal replacement therapy
ICS Inhaled corticosteroid
IgG4-RD Immunoglobin G4-related disease
IL Interleukin
ILC2 Innate lymphoid cells type 2
IMP Investigational medicinal product
IRT Interactive response technology
ITP Immune thrombocytopenia
ITT Intent-to-treat
LABA Long-acting β2 adrenergic agonist
LAR Legally authorized representative
LOAC Loss of asthma control
LOCF Last observation carried forward
LS Least squared
MCID Minimal clinically important difference
MDI Metered dose inhaler
μg micrograms
mg milligrams
OCS Oral corticosteroid
PBMC Peripheral blood mononuclear cell
PCSA Potentially clinically significant abnormality
PD Pharmacodynamics
PEF Peak exploratory flow
PGIC Patient global impression of change
PGIS Patient global impression of severity
PK Pharmacokinetics
PO Orally
RD Risk difference
SAE Serious adverse event
SUSAR Suspected unexpected serious adverse reaction
TB Tuberculosis
TBI Tuberculosis infection
TEAE Treatment-emergent adverse event
TID Three times a day
V Visit
wAIHA Warm autoimmune hemolytic anemia
Wk Week
WOCBP Woman of childbearing potential
WONCBP Woman of non-childbearing potential

### Example 1: A Randomized, Double-Blind, Placebo-Controlled, Parallel-Group, 12-Week Proof-of-Concept (PoC) Study to Assess the Efficacy, Safety, and Tolerability of Rilzabrutinib in Patients with Moderate-to-Severe Asthma Who are not Well Controlled on Inhaled Corticosteroid (ICS) Plus Long-Acting β2 Adrenergic Agonist (LABA) Therapy Study Design

This is a global, multicenter, Phase 2, double-blind, 2 arm, parallel treatment, placebo-controlled randomized, proof-of-concept (PoC) study with 2 staggered cohorts (2 arms in each cohort) evaluating the efficacy and safety of rilzabrutinib in adult participants (aged 18-70 years, inclusive) with moderate-to-severe asthma who are not well controlled on inhaled corticosteroid (ICS) plus long-acting β2 adrenergic agonist (LABA) therapy.

Approximately 192 adult participants meeting the inclusion/exclusion criteria were to be stratified by IgE levels at screening (IgE<100 IU/mL versus IgE≥100 IU/mL) and by region and were to be randomized in a 1:1 allocation ratio to either rilzabrutinib or matching placebo. An enrollment cap was to ensure that at least 60% of the participants have IgE≥100 IU/mL at screening.

Participants were to be enrolled into 2 staggered dose regimen cohorts: a 400 mg BID cohort and a 400 mg TID cohort, starting with the 400 mg BID cohort. These 2 dosing regimen cohorts were to be evaluated with the same double-blind fashion and planned assessments in this study. Participants could only be enrolled in one cohort and were not to be allowed to participate in the 400 mg TID cohort if they had already enrolled in the 400 mg BID cohort.

Study treatment was to include IMP (rilzabrutinib or placebo) added-on to a background therapy of ICS/LABA (fluticasone/salmeterol (non-investigational medicinal product), standardized at screening). Background therapy of ICS/LABA was to be withdrawn during the 12-week randomized treatment period and resumed at the end of the IMP treatment period, as outlined below:
Screening period (4 weeks (D-28-D-1))
Randomized IMP treatment period (12 weeks±3 days)
   Background therapy stabilization phase (4 weeks)
   Background therapy withdrawal phase (4-5 weeks)
   No background therapy phase (3-4 weeks)
Post IMP treatment safety follow-up period (4 weeks±3 days)

At screening, participants must have been using at least moderate to high doses of ICS therapy (≥250 μg of fluticasone propionate BID or comparable ICS daily dosage (1) to a maximum of 2000 μg/day of fluticasone propionate or clinically comparable) in combination with a LABA for at least 3 months with a stable dose ≥1 month prior to Screening Visit 1.

During the Screening period, participants were to be:
Trained on using electronic questionnaires (e-diary) at the Screening visit (Visit 1) and will record ANSD (Asthma Nighttime Symptom Diary) and ADSD (Asthma Daytime Symptom Diary), compliance with background treatment starting after the screening visit and continuing through the study period using this e-diary. Clinical site staff were to check patient data collected on the e-diary at each visit.
Trained on the use of the electronic home PEF meter/spirometry device. Clinical site staff were to check patient data collected on the home PEF meter/spirometry device at each visit.
Queried on personal history of comorbid atopic conditions as well as history of allergy (including aeroallergens, dust mites, and molds). Results of historical skin testing specifying the technique (prick/puncture (percutaneous) or intradermal (intracutaneous) or RAST) was to be collected.

After completion of screening procedures, all eligible participants were to be switched to clinically comparable doses of the study-specific ICS/LABA combination therapy with fluticasone/salmeterol, as approved by local Health Authority:
Fluticasone/salmeterol—DPI:
   1 inhalation of 250/50 μg BID or
   1 inhalation of 500/50 μg BID
OR
Fluticasone/salmeterol—MDI:
   2 inhalations of 115/21 μg (230/42 μg) BID or
   2 inhalations of 230/21 μg (460/42 μg) BID
OR
Fluticasone/salmeterol—MDI:
   2 inhalations of 125/25 μg (250/50 μg) BID or
   2 inhalations of 250/25 μg (500/50 μg) BID

Participants who satisfied the inclusion and exclusion criteria were to be randomized (1:1 ratio) to either rilzabrutinib (400 mg) or matching placebo, administered orally BID for 12 weeks.

### Background Therapy (ICS/LABA) Withdrawal Phase

At Week 4 (Visit 6), the LABA component (salmeterol) was to be withdrawn, and participants were to be switched from their BID fluticasone/salmeterol combination therapy to a clinically comparable ICS dose of fluticasone BID monotherapy, as approved by local Health Authority:
Fluticasone (DPI formulation):
   1 inhalation of 250 μg BID or
   2 inhalations of 250 μg (500 μg) BID,
OR
Fluticasone (MDI formulation):
   2 inhalations of 110 μg (220 μg) BID or
   2 inhalations of 220 μg (440 μg) BID
OR
Fluticasone (MDI formulation):
   2 inhalations of 125 μg (250 μg) BID or
   2 inhalations of 250 μg (500 μg) BID

The ICS component (fluticasone) was to be withdrawn by a stepwise dose reduction starting at Week 6 (Visit 8) and was to continue at each Week 7 (Visit 9), Week 8 (Visit 10), and, in those being tapered from high dose ICS, Week 9 (Visit 11), provided that participants did not experience an LOAC event (Table 1).

TABLE 1
Withdrawal of fluticasone - downward titration doses (administered twice a day).

| | Wk 4/V 6 | Wk 6/V8 | Wk 7/V 9 | Wk 8/V 10 | Wk 9/V 11 |
|---|---|---|---|---|---|
| Fluticasone | 250 | 100 | 50 | 0 | 0 |
| DPI Dose (μg) | 500 | 250 | 100 | 50 | 0 |
| administered | | | | | |
| BID | | | | | |
| Fluticasone | 220 | 110 | 44 | 0 | 0 |
| MDI Dose | 440 | 220 | 110 | 44 | 0 |
| (μg) | 250 | 125 | 50 | 0 | 0 |
| administered | 500 | 250 | 125 | 50 | 0 |
| BID | | | | | |

Participants were to use the same inhaler type (either DPI or MDI) throughout the study.

Participants that met the criteria for a LOAC, at any time during the randomized IMP treatment phase, were to (be):
Discontinue early from IMP treatment,
Evaluated and receive treatment by the Investigator with standard of care according to standard medical practice. If practicable and safe, the participant was to undergo EOT Visit (Visit 14) assessment prior to the administration of rescue medications.
Resume their individual prescreening ICS/LABA background therapy and,
Followed for safety during a 4-week Post IMP Treatment Period.

### Early Treatment Discontinuation (ETD) Follow-Up

Participants who discontinued IMP treatment prior to completing the 12-week IMP treatment (due to LOAC or due to early treatment discontinuation), were to be evaluated as soon as possible at the individual participant's EOT Visit and using procedures as planned for the EOT Visit at Week 12 (Visit 14). At their EOT visit, participants were to resume their prescreening ICS/LABA background therapy or appropriate therapy as needed per treating physician were to be resumed after the EOT study assessments had been performed and entered the 4-week safety follow-up period/Post IMP Treatment Period, concluding with the EOS Visit (Visit 16).

### Post IMP Treatment Follow-Up

Participants who completed the 12-week randomized IMP treatment period at Week 12 (Visit 14) EOT visit, as per protocol, were to resume their prescreening ICS/LABA therapy and enter the 4-week safety follow-up period/Post IMP Treatment Period concluding with the EOS Visit (Visit 16).

At EOT visit, if a participant's asthma could be adequately controlled by the prescreening ICS/LABA therapy, additional controller therapies may also have been prescribed based on the Investigator's clinical judgement.

### Objectives and Endpoints

Objectives and endpoints for this study are described in Table 2 below.

TABLE 2
Objectives and endpoints.

| | |
|---|---|
| Objectives | Endpoints |

Primary

| | |
|---|---|
| Evaluate the effects of rilzabrutinib compared | Proportion of participants with an LOAC event |
| to placebo on reducing the incidence of “loss | during the treatment period, defined as any one |
| of asthma control” (LOAC) events | of the following: (1) a 30% or greater reduction |
| | from baseline in morning peak expiratory flow |
| | (PEF) on 2 consecutive days; (2) ≥6 additional |
| | reliever puffs of salbutamol/albuterol or |
| | levosalbutamol/levalbuterol in a 24-hour period |
| | (compared to baseline) on 2 consecutive days; |
| | (3) increase in ICS ≥4 times the last prescribed |
| | ICS dose (or ≥50% of the prescribed ICS dose at |
| | V2 if background therapy withdrawal |
| | completed); (4) requiring use of systemic (oral |
| | and/or parenteral) steroid treatment; (5) |
| | requiring hospitalization or emergency room |
| | visit for asthma exacerbation |

Secondary

| | |
|---|---|
| Evaluate the effects of rilzabrutinib compared | Pre-bronchodilator FEV1 change from baseline |
| to placebo on lung function as measured by | at EOT (key secondary endpoint) |
| FEV1 | Post-bronchodilator FEV1 change from baseline |
| | at EOT |
| | The absolute change in the percent predicted |
| | FEV1 from baseline to EOT (pre- and post- |
| | bronchodilator) |
| Assess the effects of rilzabrutinib on other | Change from baseline in pre- and post- |
| elements of spirometric lung function | bronchodilator FEV1 and other lung function |
| | measurements (PEF, forced vital capacity |
| | (FVC), and forced expiratory flow (FEV) 25- |
| | 75%) at each spirometry endpoint |
| Assess the effect of rilzabrutinib on patient | Asthma Control Questionnaire-5 (ACQ-5) score |
| reported outcomes | change from baseline at EOT and at each |
| | assessment time point |
| | Asthma Quality of Life Questionnaire with |
| | Standardized Activities (AQLQ(S) Self- |
| | administered score change from baseline at EOT |
| | and at each assessment time point |
| Assess the effects of rilzabrutinib on asthma | Change from baseline at EOT and change from |
| symptoms | baseline at each week for Asthma Daytime |
| | Symptom Diary (ADSD) and Asthma Nighttime |
| | Symptom Diary (ANSD) scores. |
| Assess the plasma pharmacokinetics (PK) of | Plasma PK concentrations of rilzabrutinib in |
| rilzabrutinib in participants with asthma | participants with asthma |
| Assess the safety of rilzabrutinib in | Adverse events (AEs), vital signs, labs |
| participants with asthma | |
| Assess the use of rilzabrutinib on | Change from baseline and at EOT and change |
| bronchodilator therapy | from baseline at each week in numbers of |
| | inhalations/day of albuterol or levalbuterol |
| | for symptom relief |

Exploratory

| | |
|---|---|
| Assess the effect of rilzabrutinib based on | Primary and secondary outcomes in subgroups |
| baseline blood IgE levels | of participants with baseline blood IgE levels |
| | of ≥100 or <100 IU/uL |
| Assess the effects of rilzabrutinib on fraction | Change in FENO from baseline to EOT |
| of exhaled nitric oxide (FENO) | |
| Assess the effects of rilzabrutinib on allergic | Skin prick test (SPT) to be administered in |
| phenotype | participants with documented previously |
| | positive allergy testing at baseline and EOT |
| Assess the effects of rilzabrutinib on | Change from baseline in forced oscillatory |
| oscillatory lung mechanics | technique (FOT) |
| Assess the effects of rilzabrutinib on | Patient Global Impression of Severity (PGIS) |
| biomarkers in participants with asthma | change from baseline to EOT and at each |
| | assessment time point |
| | Patient Global Impression of Change (PGIC) |
| | from baseline to EOT and at each assessment |
| | time point |

### Appropriateness of Measurements

The assessments used in this study are all standard endpoints in evaluation of disease activity and response to therapy in asthma.

The general construct of this 12-week Loss of Asthma Control (LOAC) study consisted of a 4-week standardized ICS/LABA background therapy stabilization phase, a 4or 5-week background therapy withdrawal phase and a 3- or 4-week no background therapy phase, followed by a 4-week post IMP treatment safety follow-up period at the beginning of which, pre-screening asthma controller medications are restarted. Participants had access to short-acting β-agonist (SABA) reliever medication at all times during the protocol. The primary endpoint of this study is defined as the proportion of participants with an LOAC event during the treatment period, which consists of any of the following: 1) A 30% or greater reduction from baseline in morning PEF on 2 consecutive days; 2) 6 or more additional reliever puffs of salbutamol/albuterol or levosalbutamol/levalbuterol in a 24-hour period (compared to baseline) on 2 consecutive days; 3) Increase in ICS≥4 times the last prescribed ICS dose (or ≥50% of the prescribed ICS dose at V2 if background therapy withdrawal completed); 4) Requiring use of systemic (oral and/or parenteral) steroid treatment; or 5) Requiring hospitalization or emergency room visit for asthma exacerbation.

This approach allowed for a relatively brief study capable of rapidly identifying the loss of asthma control and minimizing the duration of patient exposure to loss of controller therapy.

Spirometry is a standard test to measure patients' lung function. It was to be performed in accordance with the American Thoracic Society (ATS)/European Respiratory Society (ERS) guidelines (Graham B L et al., 2019).

The ACQ-5 was designed to measure both the adequacy of asthma control and change in asthma control which may have occurred either spontaneously or as a result of treatment. Measurement properties such as reliability and ability to detect change have been documented in the literature (Juniper et al. 2005).

The AQLQ(S) is a self-administered patient reported outcome to measure the functional impairments that are most troublesome to adolescents and adults ≥12 years of age as a result of their asthma. The instrument has been used in many clinical trials, and it has been shown to be reliable, valid (patient interviews), and sensitive to change. The MCID for AQLQ(S) is 0.5 (Juniper et al. 1994).

ADSD and ANSD are PRO measures developed by the PRO Consortium′ Asthma Working Group. Both instruments have been designed to measure asthma symptoms in adult and adolescent (12 years of age and older) patients diagnosed with mild to severe asthma. ADSD and ANSD assess asthma severity based on patient self-report of asthma core symptoms. They have demonstrated adequate evidence of content validity and cross-sectional measurement properties (i.e., internal consistency reliability, test-retest reliability, convergent validity, and known-groups validity) to measure symptoms of asthma (Gater et al. 2016).

In terms of safety, TFAFs, SAFs, adverse events of special interest (AESIs), vital signs, and laboratory analyses were to be reported.

### Study Population

Inclusion criteria are summarized in Table 3. Participants were eligible to be included in the study only if all of the criteria applied.

TABLE 3
Inclusion Criteria.

| | |
|---|---|
| Age | Participants must have been from 18 to 70 years |
| | of age at the time of signing the informed |
| | consent |
| Type of participant and disease | A physician diagnosis of asthma for at least 12 |
| characteristics | months based on the Global Initiative for |
| | Asthma (GINA) 2019, 202, or 2021 Guidelines |
| | (Global Initiative for Asthma (GINA). Global |
| | Strategy for Asthma Management and |
| | Prevention, Updated 2019, Updated 2020, |
| | Updated 2021) |
| | Participants with existing treatment with at least |
| | moderate to high doses of ICS therapy (≥250 μg |
| | of fluticasone propionate BID or comparable |
| | ICS daily dosage (1) to a maximum of 2000 |
| | μg/day of fluticasone propionate or clinically |
| | comparable) in combination with a LABA as a |
| | second controller for at least 3 months with a |
| | stable dose ≥1 month prior to V 1. The lower |
| | limit of allowed ICS dose (500 μg of fluticasone |
| | propionate daily) corresponds to upper limit of |
| | medium dose defined by GINA guidelines. |
| | Patients receiving medium ICS dose <500 μg |
| | daily were not eligible. |
| | Participants with prebronchodilator FEV >40% |
| | of predicted normal at V 1 screening. |
| | Prebronchodilator FEV1 ≥50% of predicted |
| | normal at V 2/baseline. |
| | Participants with reversibility of at least 12% |
| | and 200 mL in FEV1 15 to 30 minutes after |
| | administration of 2 to 4 puffs (200-400 μg) of |
| | albuterol/salbutamol or levalbuterol/levosalbutamol |
| | during screening or documented history of a |
| | reversibility test that met this criterion within |
| | 5 years prior to V 1 or documented positive |
| | response to methacholine challenge (a decrease |
| | in FEV1 by 20% (PC₂₀) of <8 mg/mL) within 5 years |
| | prior to V 1/screening were considered acceptable |
| | to meet this inclusion criterion. |
| | Participants must have experienced, within 2 |
| | years prior to V 1, any of the following asthma |
| | exacerbation events at least once: (1) treatment |
| | with a systemic steroid (oral or parenteral) for |
| | worsening asthma; and (2) hospitalization or |
| | emergency medical care visit for worsening |
| | asthma. |
| Weight | Body mass index (BMI) ≥17.5 and ≤40 kg/m² |
| Sex, contraceptive/barrier method, and | All contraceptive use by men and women should |
| pregnancy testing requirements | have been consistent with local regulations |
| | regarding the methods of contraception for those |
| | participating in clinical studies |
| | Male participants were eligible to participate if |
| | they agreed to the following during the study |
| | intervention period and for at least 13 weeks |
| | after the last administration of study |
| | intervention: |
| | Refrain from donating or cryopreserving |
| | sperm; AND |
| | (1) Abstain from heterosexual intercourse |
| | as their preferred and usual lifestyle |
| | (i.e., be abstinent on a long term and |
| | persistent basis) and agree to remain |
| | abstinent; OR (2) agree to use contraception/ |
| | barrier¹ |
| | Female participants were eligible to participate |
| | if they were not pregnant or breastfeeding, and |
| | one of the following conditions applied: |
| | She was a woman of non-childbearing |
| | potential (WONCBP); OR |
| | She was a woman of childbearing potential |
| | (WOCBP)² and agreed to use a contraceptive |
| | method that is highly effective (with a |
| | failure rate of <1%), preferably with |
| | low user dependency, during the study |
| | intervention period (to be effective |
| | before starting the intervention) and |
| | for at least 4 weeks after the last |
| | administration of study intervention and |
| | agreed not to donate or cryopreserve eggs |
| | (ova, oocytes) for the purpose of |
| | reproduction during this period. |
| Informed consent | Capable of giving signed informed consent, |
| | which included compliance with the |
| | requirements listed in the informed consent form |
| | (ICF) and in this protocol. In countries where |
| | legal age of majority is above 18 years, a |
| | specific ICF must also have been signed by the |
| | participant's legally authorized representative |
| | (LAR). |
| COVID-19 vaccination | Participants must have completed COVID-19 |
| | vaccination per current regional health authority |
| | recommendations prior to screening. |

¹A male condom and an additional highly effective contraceptive method when having sexual intercourse with a woman of childbearing potential who is not currently pregnant; or a male condom when engaging in any activity that allows for passage of ejaculate to another person.
²A WOCBP must have had a negative highly sensitive pregnancy test ((urine or serum) as required by local regulations) within 28 days before the first administration of study intervention. If a urine test could not be confirmed as negative (e.g., an ambiguous result), a serum pregnancy test was to be required. In such cases, the participant must have been excluded from participation if the serum pregnancy result was positive.

Exclusion criteria are summarized in Table 4. Participants were excluded from the study if any of the criteria applied.

TABLE 4
Exclusion criteria.

| | |
|---|---|
| Medical conditions | History of serious infections requiring |
| | intravenous therapy with the potential for |
| | recurrence (as judged by the Site Investigator, |
| | with less than 4 weeks' interval between |
| | resolution of serious infection and first dose of |
| | study drug, or currently active moderate-to-sever |
| | infection at screening (Grade 2 or higher). |
| | Chronic lung disease (for example, chronic |
| | obstructive pulmonary disease (COPD), or |
| | idiopathic pulmonary fibrosis (IPF)) which may |
| | impair lung function, or another diagnosed |
| | pulmonary or systemic disease associated with |
| | elevated peripheral eosinophil counts, for e.g., |
| | eosinophilic granulomatosis with polyangiitis. |
| | History of life-threatening asthma (i.e., severe |
| | exacerbation that requires intubation). |
| | Participants with any of the following events |
| | within the 4 weeks prior to their screening V 1 |
| | or during the screening period: (1) treatment |
| | with 1 or more systemic (oral and/or parenteral) |
| | steroid bursts for worsening asthma; and (2) |
| | hospitalization or emergency medical care visit |
| | for worsening asthma. |
| | Asthma Control Questionnaire 5-question |
| | version (ACQ-5) score <1.25 or >3.0 at V |
| | 2/randomization. During the screening period, |
| | an ACQ-5 of up to ≤4 was acceptable. |
| | Current smoker or cessation of smoking within |
| | the 6 months prior to V 1. |
| | Previous smoker with a smoking history of >10 |
| | pack-years. |
| | Current or chronic history of liver disease. This |
| | included but was not limited to hepatitis virus |
| | infections, drug- or alcohol-related liver disease, |
| | non-alcoholic steatohepatitis, autoimmune |
| | hepatitis, hemochromatosis, Wilson's disease, |
| | α-1 antitrypsin deficiency, primary biliary |
| | cholangitis, primary sclerosing cholangitis, or |
| | any other liver disease considered clinically |
| | significant by the Investigator. |
| | Symptomatic herpes zoster within 3 months |
| | prior to screening. |
| | Refractory nausea and vomiting, malabsorption, |
| | external biliary shunt, or significant bowel |
| | resection that would preclude adequate |
| | rilzabrutinib/placebo absorption. |
| | Conditions that may have predisposed the |
| | participant to excessive bleeding: |
| | A bleeding disorder or known platelet |
| | dysfunction at any time prior to the |
| | screening visit. |
| | The participant had major surgery |
| | within 4 weeks prior to the screening |
| | visit, which could affect the |
| | participant's safety or affect immune |
| | response (as judged by the Investigator) |
| | or had planned any elective major |
| | surgery during the study. |
| | A history of significant bleeding event |
| | within 6 months prior to screening, |
| | according to the Investigator's judgment |
| | such as, but not limited to, cerebral or |
| | gastrointestinal bleeding. |
| | History of solid organ transplant |
| | A history of malignancy of any type within 5 |
| | years before D 1, other than surgically excised |
| | non-melanoma skin cancers or in situ cervical |
| | cancers |
| | Was not up-to-date with recommended |
| | vaccinations per local guidelines. |
| Prior/concomitant therapy | Anti-immunoglobulin E (IgE) therapy (e.g., |
| | omalizumab (Xolair ®) within 130 days prior to |
| | V 1 or any other biologic therapy (including |
| | anti-IL4/4R or IL-5/5R monoclonal antibodies |
| | (mAb) or systemic immunosuppressant (e.g., |
| | methotrexate) to treat inflammatory disease or |
| | autoimmune disease (e.g., rheumatoid arthritis, |
| | inflammatory bowel disease, primary biliary |
| | cirrhosis, systemic lupus erythematosus, |
| | multiple sclerosis) and other diseases, within 2 |
| | months or 5 half-lives prior to V 1, whichever is |
| | longer. |
| | Use of inhalers other than ICSs, LABAs, and |
| | short-acting beta agonists (no LAMAs or |
| | mucolytics) and leukotriene receptor antagonists |
| | (montelukast, zafirkulast) during the study |
| | period. |
| | Participants who had received bronchial |
| | thermoplasty within 2 years prior to V 1 or plan |
| | to begin therapy during the screening period or |
| | the randomized treatment period. |
| | Use of proton pump inhibitor drugs such as |
| | omeprazole and esomeprazole within 3 days of |
| | D 1 (it was acceptable to change patient to H2 |
| | receptor blocking drugs prior to D 1) |
| | Use of known systemic strong-to-moderate |
| | inducers³ or inhibitors⁴ of CYP3A within 14 |
| | days or 5 half-lives (whichever is longer) of |
| | study D 1 and until the end of the active |
| | treatment period. |
| | Live vaccine except Bacille Calmette Guerin |
| | vaccination within 28 days prior to D 1 or plans |
| | to receive one during the trial; Calmette Guerin |
| | vaccination within 12 months prior to screening. |
| | COVID-19 vaccine within 14 days prior to study |
| | D 1 |
| Prior/concurrent clinical | Previous use of a BTK inhibitor |
| study experience | Had received any investigational drug (or is |
| | currently using an investigational device) within |
| | the 30 days before D 1, or at least 5 times the |
| | respective elimination half-life time (whichever |
| | is longer). |
| Diagnostic assessments | Any of the following laboratory abnormalities at |
| | the screening visit (identified by the central |
| | laboratory): |
| | Absolute neutrophil count <1.5 × 10⁹/L |
| | Aspartate aminotransferase (AST) or |
| | alanine aminotransferase (ALT) >1.5 |
| | upper limit of normal (ULN) |
| | Total bilirubin >1.5 ULN (isolated |
| | bilirubin >1.5 × ULN is acceptable if |
| | total bilirubin is fractionated and direct |
| | bilirubin <35%) |
| | IgG <500 mg/dL |
| | Abnormal international normalized ratio |
| | (INR) test and activated partial |
| | thromboplastin time (aPTT) judged by |
| | the Investigator to be clinically |
| | significant |
| | A platelet count <150,000/μL⁵ |
| | Positive for human immunodeficiency virus |
| | (HIV) at screening |
| | Presence of hepatitis B surface antigen (HBsAg) |
| | or hepatitis B core antibody (HBcAb) with |
| | positive DNA test result at screening or within 3 |
| | months prior to first administration of study |
| | intervention. |
| | Positive hepatitis C antibody test result at |
| | screening or within 3 months prior to starting |
| | study intervention⁶. |
| | Exclusion related to tuberculosis infection |
| | (TBI): |
| | Active TBI or a history of incompletely |
| | treated TBI regardless of screening |
| | Quantiferon (QFT) result. |
| | QFT positive patients (no active |
| | disease) are excluded from the study |
| | unless the following conditions were |
| | met: (1) patients with a history of prior |
| | documented completed |
| | chemoprophylaxis for latent TBI (e.g., |
| | acceptable treatments would be 9 |
| | months of isoniazid 300 mg oral daily or |
| | equivalent proven regimen per local |
| | guidelines) or treatment of active TBI |
| | who had obtained consultation with a |
| | specialist to rule out active TBI or treat |
| | active TBI; (2) consultation with and |
| | prior approval from the Sponsor were |
| | required in either of the aforementioned |
| | scenarios. Otherwise, a documented |
| | negative screening for TB via a negative |
| | QFT within 3 months prior to screening |
| | (and if required per local standard of |
| | care, a chest X-ray), was sufficient and |
| | no further screening with QFT was |
| | required; if clinically indicated, the test |
| | may have been repeated based on |
| | clinical judgment, borderline results, or |
| | clinical suspicion of TBI. |
| | Clinically significant abnormality |
| | consistent with prior/active TBI based |
| | upon chest radiograph with at least |
| | posterior-anterior view (radiograph must |
| | have been taken within 12 weeks prior |
| | to screening visit or during the screening |
| | period). Additional lateral view was |
| | recommended but not required. |
| | Suspected extrapulmonary TBI |
| | regardless of screening QFT result. |
| | Patients at high risk of contracting TB, |
| | such as close contact with individuals |
| | with active or latent TBI. |
| | Electrocardiogram (ECG) findings of QT |
| | corrected for heart rate (QTc) >450 msec |
| | (males) or >470 msec (females), poorly |
| | controlled atrial fibrillation (i.e., symptomatic |
| | patients or a ventricular rate above 100 |
| | beats/min on ECG), or other clinically |
| | significant cardiovascular abnormalities. |
| | Active COVID-19 infection as documented by a |
| | positive COVID-19 molecular test |

³Strong CYP3A inducers include apalutamide, carbamazepine, enzalutamide, mitotane, phenytoin, rifampin, and St. John's wort. Moderate CYP3A inducers include Bosentan, efavirenz, etravirine, phenobarbital, and primidone. Source: Drug Development and Drug Interactions: Table of Substrates, Inhibitors and Inducers, U.S. Food and Drug Administration..
⁴Strong CYP3A inhibitors include boceprevir, clarithromycin, cobicistat, danoprevir/ritonavir, elvitegravir/ritonavir, grapefruit juice, idelalisib, indinavir/ritonavir, itraconazole, ketoconazole, lopinavir/ritonavir, nefazodone, nelfinavir, paritaprevir/ritonavir and ombitasvir and/or dasabuvir, posaconazole, ritonavir, saquinavir/ritonavir, telaprevir, telithromycin, tipranavir/ritonavir, troleandomycin, and voriconazole. Moderate CYP3A inhibitors include aprepitant, ciprofloxacin, conivaptan, crizotinib, cyclosporine, dronedarone, erythromycin, fluconazole, fluvoxamine, imatinib, tofisopam, and verapamil. Source: Drug Development and Drug Interactions: Table of Substrates, Inhibitors and Inducers, U.S. Food and Drug Administration.
⁵A one-time retest value at screening may have been performed by the Investigator if abnormal laboratory test values are found.
⁶Participants with positive hepatitis C antibody due to prior resolved disease could be enrolled, only if a confirmatory negative Hepatitis C RNA test was obtained.

### Lifestyle Considerations

Participants were to refrain from consumption of grapefruit or grapefruit juice, from 7 days before the start of study intervention until the completion of the final dose.

No restriction of caffeine or activity was required. Smoking was not permitted during the trial and current smokers were excluded from participation.

### Retesting: Laboratory Inclusion/Exclusion Criteria

If a participant did not meet certain laboratory inclusion/exclusion criteria at Screening due to the following laboratory results, the Investigator may have repeated one or more of these tests once during the screening period:
ALT or AST >1.5×ULN (may have been repeated if 1.5 to 3×ULN)
Total bilirubin >1.5×ULN (may have been repeated if 1.5 to 3×ULN)
Absolute neutrophils count <1.5×10⁹/L (may have been repeated if 1.2 to 1.5×10⁹/L)
A platelet count <150,000/μL (may have been repeated if 120,000/μL to 150,000/μL)
Positive result for SARS-CoV-2 infection

If the participant met the laboratory eligibility criteria on the second assessment, he or she was to be permitted to enter the study. It was not to be considered a retesting if blood samples had to be redrawn because of sample handling problems, breakage, sample integrity, or laboratory error.

If a hematology/chemistry lab value fell outside the normal range and was judged by the Investigator to be clinically significant and was NOT one of explicit exclusionary laboratory testing, it may have been repeated once and if the repeat value was no longer considered clinically significant then that specific laboratory testing result should not have disqualified a participant from the study.

### Study Intervention(s) and Concomitant Therapy

Study intervention is defined as any investigational intervention(s), marketed product(s), placebo, or medical device(s) intended to be administered to a study participant according to the study protocol (Tables 5-6).

Rilzabrutinib and placebo tablets were supplied ready for use; no preparation was needed. Participants were to use study medication directly from the dispensed bottles.

Participants were to administer rilzabrutinib or placebo by mouth twice daily (400 mg BID cohort) or three times daily (400 mg TID cohort). Consecutive doses should ideally have been administered approximately 12 hours apart (and not less than 8 hours apart) for 400 mg BID dosing, and ideally approximately 6 hours apart (and not less than 4 hours apart) for 400 mg TID dosing. Tablets should not have been broken or crushed.

Study intervention was to be dispensed at the study visits. The study intervention to be taken by a participant was to be allocated using an IRT. The site was to contact the IRT prior to the start of study intervention administration for each participant. The site was to record the intervention assignment on the applicable case report form, if required.

TABLE 5
Overview of study interventions administered.

| | | |
|---|---|---|
| Intervention label | Rilzabrutinib | Placebo |
| Intervention name | Rilzabrutinib | Placebo |
| Type | Drug | Drug |
| Dose formulation | Tablets (modified-capsule | Tablets (modified-capsule |
| | shaped tablets) | shaped tablets) |
| Unit dose strength(s) | 400 mg | 0 mg |
| Dosage level(s) | 400 mg (1 tablet) BID or TID | 1 tablet BID or TID |
| Route of administration | Oral | Oral |
| Use | Experimental | Placebo |
| IMP or NIMP | IMP | IMP |
| Packaging and labeling | Rilzabrutinib tablets were to | Placebo tablets were to be |
| | be supplied in white plastic | supplied in white plastic |
| | bottles with child-resistant | bottles with child-resistant |
| | induction-sealed caps. The | induction-sealed caps. The |
| | bottles were to be labeled as | bottles were to be labeled as |
| | required per country requirement | required per country requirement |
| Current/former name(s) | Rilzabrutinib | Not applicable |
| or alias(es) | | |

TABLE 6
Arms and associated interventions.

| | Rilzabrutinib | | Rilzabrutinib | |
|---|---|---|---|---|
| Arm name | 400 mg BID | Placebo BID | 400 mg TID | Placebo TID |
| Associated | Rilzabrutinib and | Placebo and | Rilzabrutinib and | Placebo and |
| interventions | ICS/LABA | ICS/LABA | ICS/LABA | ICS/LABA |
| (intervention | | | | |
| label(s)) | | | | |

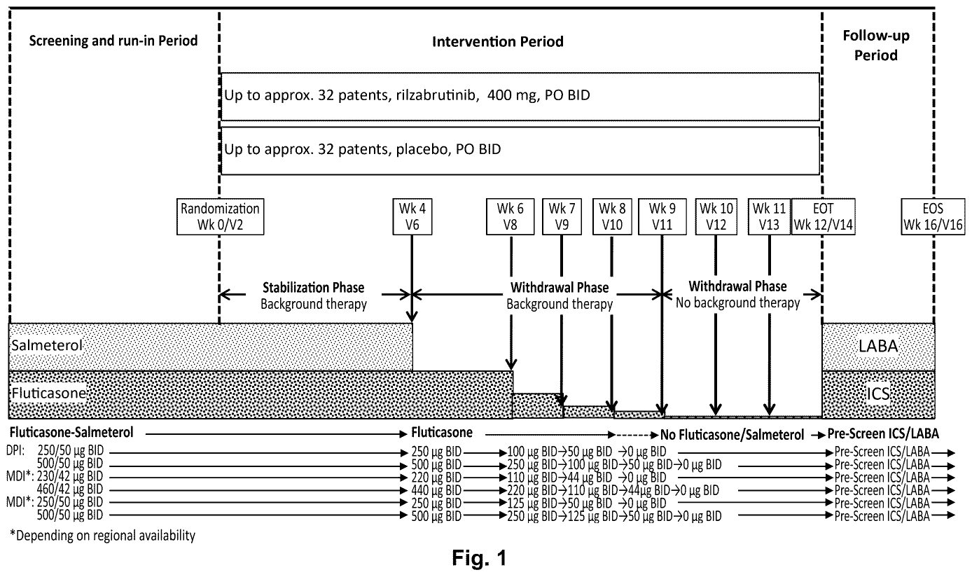

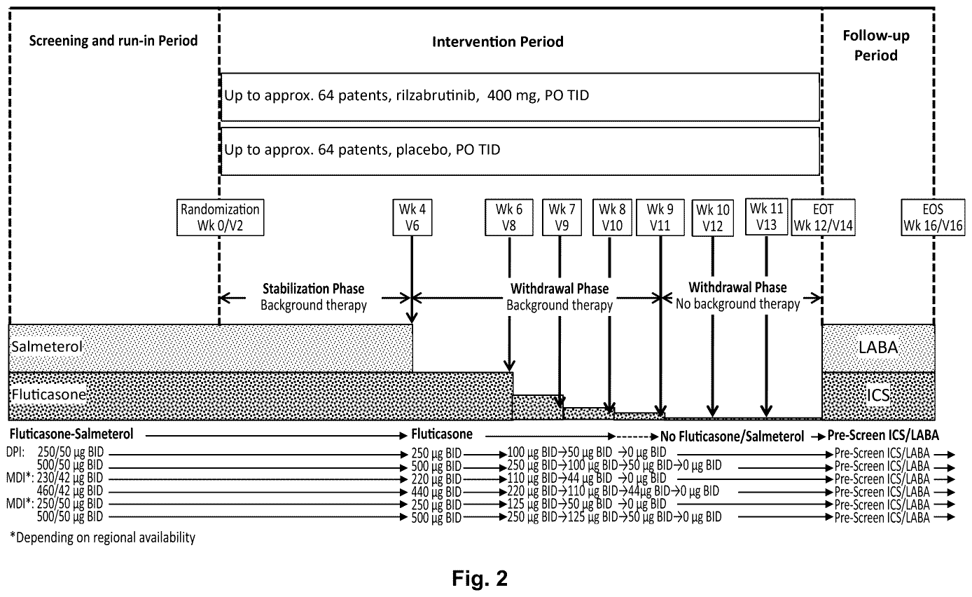

Participants were to continue their background therapy of ICS/LABA (fluticasone/salmeterol (non-investigational medicinal product), standardized at screening). Background therapy of ICS/LABA was to be withdrawn during the 12-week randomized treatment period and resumed at the end of the IMP treatment period (FIGS. 1-2 and Table 7).

TABLE 7
Background therapy.

| Intervention label | ICS/LABA stabilization phase | ICS/LABA withdrawal phase |
|---|---|---|
| | V2 Week 0 to V6 Week 4 | V6 Week 4 to V10 Week 8 |
| | | (V11 Week 9 in those tapering |
| | | from high dose ICS) |
| Intervention name | Fluticasone/Salmeterol | Fluticasone monotherapy |
| | combination therapy | |
| Type | Drug | |
| Dose formulation | Dry powder inhaler (DPI) or metered dose inhaler (MDI) | |
| Unit dose strength(s) | DPI: 1 inhalation of 250/50 μg BID | DPI: 1 inhalation of 250 μg BID |
| Dosage level(s) | or 1 inhalation of 500/50 μg BID | or 2 inhalations of 250 μg BID |
| | OR | OR |
| | MDI: 2 inhalations of 115/21 μg BID | MDI: 2 inhalations of 110 μg BID |
| | or 2 inhalations 230/21 μg BID | or 2 inhalations of 220 μg BID |
| | OR | OR |
| | MDI: 2 inhalations of 125/25 μg BID | MDI: 2 inhalations of 125 μg BID |
| | or 2 inhalations of 250/25 μg BID | or 2 inhalations of 250 μg BID |

| | |
|---|---|
| Route of administration | Inhalation |
| Use | Background therapy |
| IMP or NIMP | NIMP |
| Packaging and labelling | Not applicable |
| Current/former name(s) | Not applicable |
| or alias(es) | |

Participants were to use the same inhaler type (either DPI or MDI) throughout the study.

### Dose Modification

Rilzabrutinib dose modification was not allowed.

### Concomitant Therapy

Any medication or vaccine (including over-the-counter or prescription medicines, vitamins, and/or herbal supplements) that the participant was receiving at the time of enrollment or received during the study must have been recorded along with:
Reason for use
Dates of administration including start and end dates
Dosage information including dose and

The Medical Monitor should have been contacted if there were any questions regarding concomitant or prior therapy.

Participants were to have abstained from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator, the medication would not interfere with the study.

Medications that were not listed as exclusion criteria were permitted for use during the trial.

These include the following:
Oral corticosteroid rinses (mouth washes) were allowed, as are ocular, intranasal, and topical corticosteroids.
Clinically relevant drugs that are substrates of cytochrome P450 (CYP)3A, including those considered to be sensitive CYP3A substrates⁷ were permitted. Appropriate caution was to be used when co-administering sensitive CYP3A substrates with rilzabrutinib and a benefit-risk assessment was to be conducted for each medication. Consideration should also have been given to avoidance of high doses, dose reduction, or replacement of sensitive and narrow therapeutic index CYP3A substrate drugs. Systemic moderate or strong inhibitors (including foods such as grapefruit juice) and inducers of CYP3A should have been avoided. ⁷ Sensitive CYP3A substrates include Alfentanil, avanafil, budesonide, buspirone, conivaptan, darifenacin, darunavir, dasatinib, dronedarone, ebastine, eletriptan, eplerenone, everolimus, felodipine, ibrutinib, indinavir, lomitapide, lovastatin, lurasidone, maraviroc, midazolam, naloxegol, nisoldipine, quetiapine, saquinavir, sildenafil, simvastatin, sirolimus, tacrolimus, ticagrelor, tolvaptan, tipranavir, triazolam, and vardenafil. Source: Drug Development and Drug Interactions: Table of Substrates, Inhibitors and Inducers, U.S. Food and Drug Administration. This list is not intended to be exhaustive and may be out of date. For updated information, please refer to https://www.fda.gov/drugs/drug-interactionslabeling/drug-development-and-drug-interactions-table-substrates-inhibitors-and-inducers or product labeling.
Histamine 2 (H2) receptor blocking drugs were permitted provided they could have been given 2-3 hours after administration of rilzabrutinib or placebo. For patients receiving the IMP three times a day, H2-receptor blockers must have been taken once daily only, 2-3 hours after the evening dose of rilzabrutinib or placebo.
Antacids were permitted provided they were given 2 hours or more apart from rilzabrutinib or placebo.
Previously initiated immunotherapy was to be allowed to continue during the study.

The following concomitant treatments were not permitted during the screening or treatment phases:
Any inhaled steroid other than the standardized fluticasone/salmeterol or fluticasone
background therapy administered as per protocol
Systemic steroids (except systemic steroids to treat asthma exacerbations)
LABA other than the salmeterol component of the fixed dose combination administered per protocol
Ipratropium bromide or other inhaled anti-cholinergic agents (tiotropium)*
Methylxanthines (theophylline, aminophyllines)*
Inhaled mucolytic*
Leukotriene receptor antagonists or leukotriene synthesis inhibitors* *The third controller was not allowed to be used for an additional 2 weeks prior to screening visit (V1).
Lipoxygenase inhibitors
Cromones
Anti-IL4R mAb (eg, dupilumab (Dupixent®))
Anti-IL5 or IL-5R mAb (eg, benralizumab (Fasenra®), mepolizumab (Nucala®), or reslizumab (Cinqair®))
Anti-IgE mAb (eg, omalizumab (Xolair®))
Anti-TSLP mAb (eg, tezepelumab)
Systemic immunosuppressant (e.g., methotrexate, any anti-TNF mAbs, B and/or T cell targeted immunosuppressive therapies)
Bronchial thermoplasty
Intravenous Ig (IVIG) therapy
Live Attenuated Vaccines⁸ ⁸ Live (attenuated) vaccines include Chickenpox (Varicella), Intranasal influenza, Measles (Rubeola), Measles-mumps-rubella (MMR) combination, Mumps, Oral polio (Sabin), Oral typhoid, Rubella, Smallpox (Vaccinia), Shingles (Herpes zoster), Bacille Calmette-Guerin, Yellow fever. Note: Shingrix is allowed for Shingles as a non-live Varicella vaccine.
Beta-adrenergic receptor blockers (except for a selective beta-1 adrenergic receptor blocker used with stable dose at least 1 month prior to Visit 1)
Asthma relievers other than salbutamol/albuterol or levosalbutamol/levalbuterol: their use was not recommended during the study period. In case of use in exceptional circumstances (e.g., prescribed by a physician not participating in the study), their use was to be documented in the patient's file and reported in the eCRF.
Initiation of immunotherapy
Other investigational drugs
Use of proton-pump inhibitor drugs such as omeprazole and esomeprazole (allowed during screening provided they were stopped within 3 days of Day 1).
Use of known systemic strong-to-moderate inducers or inhibitors of CYP3A (allowed during screening provided they were stopped days within 14 days or 5 half-lives (whichever is longer) of Day 1).

### Rescue Medicine

If medically necessary, participants may have received rescue therapy for asthma at the discretion of the Investigator as an LOAC event. If practicable and safe, the participant should have undergone EOT Visit (Visit 14) assessment prior to the administration of rescue medications. Participants who discontinued IMP treatment prior to completing the 12-week IMP treatment (due to LOAC or due to other early treatment discontinuation), were to be evaluated as soon as possible at the individual participants' EOT Visit, using procedures as planned for the EOT Visit at Week 12 (Visit 14). At their EOT visit, participants were to resume their prescreening ICS/LABA background therapy and enter the 4-week safety follow-up period/Post IMP Treatment Period. If a participant's asthma could not be consistently controlled on his/her original ICS/LABA therapy, and there was a safety concern, additional controller therapies may have been prescribed based on the Investigator's clinical judgement.

### Efficacy Assessments

The following efficacy parameters were to be analyzed for assessing the primary and secondary endpoints.

The primary endpoint was the proportion of patients with LOAC. The key secondary endpoint for the study was change in pre-bronchodilator FEV1 from baseline at EOT.

Other efficacy endpoints included:
Change from baseline in pre- and post-bronchodilator FEV1 and other lung function measurement (peak expiratory flow (PEF), forced vital capacity (FVC), forced expiratory flow (FEF) 25-75%) at each spirometry endpoint.
Asthma Control Questionnaire-5 (ACQ-5) score change from baseline at EOT and at each assessment time point.
Asthma Quality of Life Questionnaire with Standardized Activities (AQLQ(S)) Self-Administered score change from baseline at EOT and at each assessment time point.
Change from baseline at EOT and change from baseline at each week for asthma symptom scores in the morning and evening (ADSD and ANSD). Questionnaire was to be administered daily and score averaged weekly for analysis.
Change from baseline at EOT and change from baseline at each week in number of inhalations/day of albuterol or levalbuterol for symptom relief.

### Disease-Specific Efficacy Measures

### Spirometry

Spirometry was to be performed in accordance with the American Thoracic Society (ATS)/European Respiratory Society (ERS) guidelines (Graham et al. 2019) and prior to administration of IMP at the planned time point. For prebronchodilator measured parameters, including FEV1, PEF, FVC and FEF 25%-75%, spirometry was to be performed after a wash out period of bronchodilators according to their action duration, for example, withholding the last dose of salbutamol/albuterol or levosalbutamol/levalbuterol for at least 6 hours and withholding the last dose of LABA for at least 12 hours.

Reversibility is defined as an increase of the absolute FEV1 after administration of bronchodilator and is measured by spirometry as postbronchodilator increase in FEV1 in percent of the prebronchodilator FEV1. After spirometry for measuring prebronchodilator FEV1, participants were to receive 2-4 puffs of albuterol/salbutamol or levalbuterol/levosalbutamol from a primed MDI. The postbronchodilator spirometry may have been repeated several times within 30 minutes after administration of bronchodilator. Alternatively, and only if it was consistent with usual office practice (to be documented), reversibility may have been performed using inhalation of nebulized albuterol/salbutamol or levalbuterol/levosalbutamol.

### ACQ-5 (Asthma Control Questionnaire, 5-Question Version)

The ACQ-5 is a questionnaire that measures the adequacy of asthma control and any changes in asthma control that may occur spontaneously or as a result of treatment. The ACQ-5 has five questions on the asthma symptoms and patients are asked to recall how their asthma has been during the previous week and to respond on a 7-point scale for each question (0=no impairment, 6=maximum impairment). The ACQ-5 score is the mean of the 5 questions and, therefore, between 0 (totally controlled) and 6 (severely uncontrolled). A high score indicates low asthma control. Participants with a score below 1.0 reflected adequately controlled asthma and participants with scores above 1.0 reflected inadequately controlled asthma. On the 7-point scale of the ACQ-5, a change or difference in score of 0.5 is the smallest change that can be considered clinically important, corresponding to the Minimal Clinically Important Difference (MCID) defined by the developer.

Measurement properties such as reliability and ability to detect change have been documented in the literature (Juniper et al. 2005). Participants were to complete the questionnaire in an electronic diary during onsite visits.

Asthma Quality of Life Questionnaire with Standardized Activities (AQLQ(S)) self-administered (≥12 years)

The AQLQ(S) was designed as a self-administered patient reported outcome to measure the functional impairments that are most troublesome to adolescents and adults ≥12 years of age as a result of their asthma. The instrument is comprised of 32 items, each rated on a 7-point Likert scales from 1 to 7. The AQLQ(S) has 4 domains. The domains and the number of items in each domain are as follows:
Symptoms (12 items)
Activity limitation (11 items)
Emotional function (5 items)
Environmental stimuli (4 items)

A global score is calculated ranging from 1 to 7 and a score by domain. Higher scores indicate better quality of life. The instrument has been used in many clinical trials, and it has been shown to be reliable, valid (patient interviews), and sensitive to change. The MCID for AQLQ(S) is 0.5 (Juniper et al. 1994). Patients were to complete the questionnaire in an electronic diary during on-site visits.

### Asthma Daytime Symptom Diary (ADSD) and Asthma Nighttime Symptom Diary (ANSD)

The ADSD and ANSD are PRO measures developed by the PRO Consortium′ Asthma Working Group. Both instruments have been designed to measure asthma symptoms in adult and adolescent (12 years of age and older) patients diagnosed with mild to severe asthma. ADSD and ANSD assess asthma severity based on patient self-report of asthma core symptoms, i.e., difficulty of breathing; wheezing; shortness of breath; chest tightness; chest pain; and cough. Patients were asked to complete the ADSD every night before they went to bed, thinking about their asthma symptoms on that day, from when they got up this morning until then; the ANSD when getting up, thinking about their asthma symptoms from the previous night from when they went to bed until then. They have demonstrated adequate evidence of content validity and cross-sectional measurement properties (i.e., internal consistency reliability, test-retest reliability, convergent validity, and known-groups validity) to measure symptoms of asthma.

Both the ADSD and ANSD are composed of 6 items rated using an 11-point NRS that ranges from 0=None to 10=As bad as you can imagine (Gater et al. 2016). The participants were to record their daytime and nighttime asthma symptoms in an electronic diary, once in the evening and once in the morning, respectively. The participants were to complete the ADSD and the ANSD.

### Patient Global Impression of Severity (PGIS) and Patient Global Impression of Change (PGIC)

The Patient Global Impression of Severity (PGIS) is a 1-item questionnaire that asks participants to provide a self-assessment of their overall asthma severity for the past week on a 4-point scale. Response choices are: “None”, “Mild”, “Moderate” and “Severe”.

The Patient Global Impression of Change (PGIC) is a 1-item questionnaire that asks the participant to provide a self-assessment of overall change in their asthma since the participant started taking the study medication on a 7-point scale. The response options range from “Very Much Better” to “Very Much Worse.”

Participants were to complete the 2 items in an electronic diary during on-site visits.

### Electronic Diary PEF Meter

The electronic diary/PEF meter was to be dispensed at Visit 1 and recorded information was to be downloaded from this device on the other indicated days. The electronic diary/PEF meter was to be used for daily recording of salbutamol/albuterol or levosalbutamol/levalbuterol use, asthma controller drug use, ADSD/ANSD, and AM and PM PEF.

On a daily basis throughout the study, the participant was to use an electronic diary/PEF meter to:
Measure morning and evening PEF.
Respond to the morning and evening asthma symptom score ADSD/ANSD questions.
Indicate the number of inhalations/day of salbutamol/albuterol or levosalbutamol/levalbuterol for symptom relief.
Record the number of inhalations/day of ICS/LABA or ICS background therapy.

At screening (Visit 1), participants were to be issued an electronic diary and PEF meter. Participants were to be instructed on the use of the devices, and written instructions on the use of the electronic PEF meter were to be provided to the participants. In addition, the Investigator was to instruct the participants on how to record the following variables in the electronic PEF meter:
AM PEF performed within 15 minutes after arising (between 5:30 AM and 12 PM).
PM PEF performed in the evening (between 5:30 PM and 12 AM).
Participants were to try to withhold albuterol or levalbuterol for at least 6 hours prior to measuring their PEF.
Three PEF efforts were to be performed by the participants; all 3 values were to be recorded by the electronic PEF meter, and the highest value was to be used for evaluation.

Baseline AM PEF was to be the mean AM measurement recorded for the 7 days prior to the first dose of IMP, and baseline PM PEF was to be the mean PM measurement recorded for the 7 days prior to the first dose of IMP. Period stability limit is defined as the respective mean AM or PM PEF obtained over the last 7 days prior to Day 1. There should have been at least 4 days of measurements for setting up the stability limit, and the first dosing visit may have been rescheduled until data for 4 days are available.

The following efficacy parameters (described in more detail below) were to be analyzed for assessing exploratory endpoints: fractional exhaled nitric oxide (FeNo) and oscillometry.

### Fractional Exhaled Nitric Oxide (FeNO)

FeNO levels (ppb) were to be collected on site with a dedicated medical device such as a commercially available hand worn device (NIOX VERO®). Participants were to inhale to total lung capacity through the device and then exhale for 10 seconds at 50 mL/sec.

### Oscillometry

Oscillometry is a complementary technique to spirometry determining the physiological behavior of the lung in asthmatics. Oscillometry was to be conducted during tidal breathing using the Tremoflo® device (Thorasys, Montreal, Canada) according to ERS recommended guidelines (Oostveen et al. 2003). A minimum of 3 recordings that achieve coefficient of variation of ≤15% were required for quality control (Peters et al. 2016). Only these data were to be used for the data analysis. The individual measurements could have been reviewed manually to confirm that a quality measurement was indeed performed.

### Safety Assessments

Safety assessments included physical examinations, measurements of vital signs, electrocardiograms, clinical safety laboratory assessments, and pregnancy testing.

### Adverse Events (AEs), Serious Adverse Events (SAEs), and Other Safety Reporting

### AE Definition

An AE is any untoward medical occurrence in a patient or clinical study participant, temporally associated with the use of study intervention, whether or not considered related to the study intervention. An AE could therefore have been any unfavorable and unintended sign (including an abnormal laboratory finding), symptom, or disease (new or exacerbated) temporally associated with the use of study intervention.

### SAE Definition

An SAE is defined as any adverse event that, at any dose:
Resulted in death
Was life-threatening
Required inpatient hospitalization or prolongation of existing hospitalization.
Resulted in persistent or significant disability/incapacity.
Was a congenital anomaly/birth defect.
Other situations such as significant medical events that may have jeopardized the participant or may have required medical or surgical intervention to prevent one of the other outcomes listed in the above definition (e.g., invasive or malignant cancers, intensive treatment in an emergency room or at home for allergic bronchospasm, blood dyscrasias or convulsions that did not result in hospitalization, or development of drug dependency or drug abuse).

### AESI Definition

An adverse event of special interest (AESI) is an AE (serious or nonserious) of scientific and medical concern specific to the Sponsor's product or program, for which ongoing monitoring and immediate notification by the Investigator to the Sponsor was required. Such events may have required further investigation in order to characterize and understand them. Adverse events of special interest may have been added, modified or removed during a study by protocol amendment. AESIs include the following:
Pregnancy of a female participant entered in a study as well as pregnancy occurring in a female partner of a male participant entered in a study with IMP.
Symptomatic overdose (serious or nonserious) with IMP.
Other project-specific AESI(s), including: severe infections including opportunistic infections; tuberculosis or initiation of medications for suspected tuberculosis; active COVID-19 infection as documented by a positive COVID-19 molecular test; major hemorrhagic events, including symptomatic bleeding in a critical area or organ such as the CNS, or intraocular bleeding, resulting in an SAE; serious adverse events (or non-serious adverse events with a severity level consistent with Grade 3 CTCAE of cytopenia; and atrial fibrillation.

### Populations for Analyses

The populations for analyses are defined in Table 8.

TABLE 8
Populations for Analyses.

| Population | Description |
|---|---|
| Screened | All participants who signed the ICF. |
| Randomized | All participants from screened population who were |
| | allocated to a randomized intervention by IRT |
| | regardless of whether the intervention was received. |
| Intent-to-treat (ITT) | All randomized participants. |
| Modified ITT (mITT) | All participants from ITT population who received at |
| | least one dose of investigational product analyzed |
| | according to the treatment group allocated by |
| | randomization. |
| | Randomized participants for whom it was unclear whether |
| | they took the study medication were included in the |
| | mITT population. |
| Safety | All randomized participants who took at least 1 dose of |
| | study intervention. Participants were analyzed according |
| | to the intervention they actually received. |
| Pharmacokinetic (PK) | All randomized and treated participants (safety population) |
| | with at least one post-baseline PK sample with adequate |
| | documentation of dosing and sampling dates and times. |
| | Participants were analyzed according to the intervention |
| | they actually received. |

Participants exposed to study intervention before or without being randomized were not to be considered randomized and were not to be included in any analysis population. The safety experience of these participants was to be reported separately.

Randomized participants for whom it was unclear whether they took the study intervention were not to be considered as exposed and were to be included in the safety population as randomized.

For any participant randomized more than once, only the data associated with the first randomization was to be used in any analysis population. The safety experience associated with any later randomization was to be reported separately.

### Statistical Analyses

This section is a summary of the planned statistical analyses of the most important endpoints including primary and key secondary endpoints.

### General Considerations

The baseline value is defined as the last available value before the first dose of double-blind IMP. For participants randomized but not treated, the baseline value is defined as the last available value before randomization.

Unless otherwise specified, analyses were to be performed by intervention group (and overall for baseline and demographics characteristics).

Continuous data were to be summarized using the number of available data, mean, standard deviation (SD), median, Q1, Q3, minimum and maximum for each intervention group. Categorical and ordinal data were to be summarized using the number and percentage of patients in each intervention group.

The observation period was to be divided into 3 segments:
The pre-treatment period is defined as the period up to the first IMP administration.
The on-treatment period is defined as the period from the first IMP administration to the last IMP administration+1 day.
The post-treatment period is defined as the period from the end of the on-treatment period up to the follow-up visit.

The treatment-emergent period consisted of on-treatment period and post-treatment period.

### Primary Endpoint(s)

The primary endpoint was proportion of participants with LOAC during the 12-week treatment period.

The incidence of LOAC was to be analyzed between rilzabrutinib and placebo. The primary analysis was to be performed using a logistic regression model with treatment (Cramer 2002), baseline IgE strata, region (pooled country), and number of asthma exacerbation events (as defined in I 06) within 2 years prior to screening as the covariates. Note that if the number of participants in some regions was too low, these regions may have been pooled with other regions. This strategy also applied to all the rest of the analyses where region was one of the covariates.

The following comparisons were to be tested without control for type I error:
Rilzabrutinib 400 mg TID versus its matching placebo.
Rilzabrutinib 400 mg BID versus its matching placebo.

Comparison may also have been done on the pooled 400 mg BID and 400 mg TID dose level groups of rilzabrutinib versus placebo.

The odds ratio, 95% confidence interval (CI) and nominal p-value for each comparison was to be estimated from this model.

### Sensitivity Analysis

Sensitivity analyses using all observed values were also to be conducted.

### Supportive Analyses

Time to LOAC post-randomization was to be analyzed using a Cox regression model with treatment (Cox 1972), baseline IgE strata, and region (pooled country) as covariates. The Kaplan-Meier (KM) method was to be used to estimate the probabilities of the event at specific time points for each treatment group (Kaplan and Meier 1958). Nominal p-value from log-rank test stratified by baseline IgE strata and region was also to be provided.

### Subgroup Analysis

To assess the consistency in treatment effects across different subgroup levels, subgroup analyses were to be performed for the primary efficacy endpoint with respect to age group, gender, region, and other factors.

### Secondary Endpoint(s)

A summary of the primary estimand for key secondary endpoints is shown in Table 9 below.

TABLE 9
Summary of primary estimand for key secondary endpoints.

| | Estimands | | | |
|---|---|---|---|---|
| Endpoint | | | | Population-level |
| category | | | Intercurrent event(s) | summary (analysis and |
| (estimand) | Endpoint | Population | handling strategy | missing data handling) |

Secondary objective: To evaluate the effects of rilzabrutinib
compared to placebo on lung function as measured by pre-BD FEV1.

| | | | | |
|---|---|---|---|---|
| Key | Pre- | miTT | Occurrence of LOAC. | The missing values at |
| secondary | bronchodilator | | For LOAC with rescue | after discontinuation of |
| endpoint | FEV1 change | | medication, data | study intervention due to |
| | from baseline | | collected after LOAC | other reasons or after |
| | at EOT | | was to be set to missing, | occurrence of LOAC |
| | | | and missing data was to | without rescue |
| | | | be imputed with LOCF | medication were to be |
| | | | (hypothetical policy). | imputed using multiple |
| | | | For LOAC without | imputation (MI). |
| | | | rescue medication, all | Mean difference of pre- |
| | | | observed values after | BD FEV1 at EOT |
| | | | LOAC were to be | between intervention |
| | | | included (treatment | groups was to be |
| | | | policy). If study | estimated form analysis |
| | | | intervention was | of covariance |
| | | | discontinued due to lack | (ANCOVA). Statistical |
| | | | of efficacy or AE related | inference obtained from |
| | | | to asthma worsening, | each imputed data was to |
| | | | data were to be imputed | be combined using |
| | | | with LOCF (hypothetical | Rubin's rule. Least |
| | | | policy). If study | squared (LS) means, |
| | | | intervention was | differences in LS |
| | | | discontinued for other | means, the |
| | | | reasons, all observed | corresponding 95% CI |
| | | | data after discontinuation | and p-value was to be |
| | | | of study intervention | presented. |
| | | | were to be included | |
| | | | (treatment policy). If | |
| | | | OCS were used without | |
| | | | LOAC, data collected | |
| | | | after start of OCS were | |
| | | | to be set to missing and | |
| | | | missing data were to be | |
| | | | imputed with LOCF | |
| | | | (hypothetical policy). | |

### Secondary Efficacy Endpoints

Secondary efficacy endpoints included:
Post-bronchodilator FEVT1 change from baseline at FOT.
The absolute change in the percent predicted FEVT1 from baseline to FOT (pre- and postbronchodilator).
Change from baseline in pre- and post-bronchodilator FEVT1 and in other lung function measurement (peak expiratory flow (PEF), forced vital capacity (FVC), forced expiratory flow (FEF) 25-75%) at each spirometry endpoint.
Asthma Control Questionnaire-5 (ACQ-5) score change from baseline at FOT and at each assessment time point.
Asthma Quality of Life Questionnaire with Standardized Activities (AQLQ(S)) Self-administered score change from baseline at FOT and at each assessment time point.
Change from baseline at EOT and change from baseline at each week for asthma daytime symptom diary and asthma nighttime symptom diary scores.
Change from baseline at EOT and change from baseline at each week in numbers of inhalations/day of albuterol or levalbuterol for symptom relief.

Other secondary efficacy endpoints (e.g., FEV1, FVC, PEF, ACQ-5 score, AQLQ, asthma symptom scores) were to be analyzed using the same approach as for the key secondary endpoint, except for that age, gender and height were to be only included in the models for spirometry variables as response variable.

### Multiplicity Considerations

No adjustments for multiplicity across secondary efficacy endpoints were planned for this Phase 2 study. Nominal p-values were to be reported.

### Other Safety Analysis

The safety variables, including AEs, vital signs, physical examination, and laboratory values were to be summarized using descriptive statistics in each treatment group in the safety population.

The summary of safety results were to be presented by treatment group. All safety analyses were to be performed on the safety population. The baseline value is defined generally as the last available value before the first dose of IMP.

### Adverse Events

In general, AEs were to be analyzed in the following 3 categories:
Pre-treatment AEs: AEs that developed, worsened, or became serious during the pre-treatment period.
TEAEs: AEs that developed, worsened, or became serious during the treatment-emergent period.
Post-treatment AEs: AEs that developed, worsened, or became serious during the post-treatment period.

### Example 2: Rilzabrutinib in Asthma

A study was initiated to evaluate the effect of rilzabrutinib treatment in N=196 patients with moderate-to-severe asthma uncontrolled by ICS/LABA. Over the course of this 12-week PoC withdrawal study, N=64 patients were administered 400 mg BID rilzabrutinib or a placebo, while N=132 patients were administered 400 mg TID rilzabrutinib or placebo.

The primary endpoint for this study was the proportion of participants with an LOAC event during the treatment period. Secondary endpoints included pre-bronchodilator FEV1 change from baseline at EOT, ACQ-5 score change from baseline, and AQLQ score change from baseline. Other secondary and exploratory endpoints are described in Table 2.

Compared to patients who received the placebo, patients who received 400 mg BID or 400 mg TID rilzabrutinib exhibited a marked and meaningful improvement in symptoms despite ICS/LABA withdrawal (Table 10). Rilzabrutinib-treated patients experienced a numerical reduction in LOAC events, a significant and meaningful improvement in asthma symptoms (ACQ-5), and an improvement in quality of life as measured by AQLQ. Furthermore, patients did not exhibit any change in FEV1 following ICS/LABA withdrawal.

Additionally, rilzabrutinib was generally safe, with no evidence of new safety concerns.

TABLE 10
Summary of the primary and key secondary endpoints (mITT population).

| | | | Difference | | | | Difference | |
|---|---|---|---|---|---|---|---|---|
| | Placebo | Rilzabrutinib | vs. | | Placebo | Rilzabrutinib | vs. | |
| | BID | 400 mg BID | placebo | | TID | 400 mg TID | placebo | |
| Parameter | (N = 32) | (N = 32) | (95% CI) | P-value | (N = 68) | (N = 64) | (95% CI) | P-value |
| Primary | | | | | | | | |
| endpoint | | | | | | | | |
| Odds ratio | 16 | 12 (37.5%) | 0.570 | 0.2880 | 20 | 12 (18.8%) | 0.584 | 0.2083 |
| incidence of | (50.0%) | | (0.202, 1.608) | | (29.4%) | | (0.253, | |
| LOAC | | | | | | | 1.349) | |
| Key | | | | | | | | |
| secondary | | | | | | | | |
| endpoint | | | | | | | | |
| LS Mean | −0.08 | −0.07 (0.08) | 0.01 | 0.9466 | −0.04 | −0.13 (0.05) | −0.09 | 0.1523 |
| change from | (0.08) | | (−0.17, 0.19) | | (0.05) | | (−0.21, 0.03) | |
| baseline in | | | | | | | | |
| pre- | | | | | | | | |
| bronchodilator | | | | | | | | |
| FEV1 (L) at | | | | | | | | |
| Week 12 | | | | | | | | |

### Patient Demographics

The 132 and 64 patients enrolled in the 400 mg TID and 400 mg BID cohorts, respectively, were characterized by the demographic information provided in Tables 11-19. The median age of enrolled patients in the 400 mg TID cohort was 50 years, while the median age of enrolled patients in the 400 mg BID cohort was 52 years. For all patients in the 400 mg TID cohort, the median time since first diagnosis of asthma was 17.96 years. For all patients in the 400 mg BID cohort, the median time since first diagnosis of asthma was 20.79 years.

TABLE 11
Analysis population.

| | 400 mg BID cohort | | | 400 mg TID cohort | | |
|---|---|---|---|---|---|---|
| | Placebo | Rilzabrutinib | | Placebo | Rilzabrutinib | |
| n (%) | BID | 400 mg BID | All BID | TID | 400 mg TID | All TID |
| Randomized population | 32 (100) | 32 (100) | 64 (100) | 68 (100) | 64 (100) | 132 |
| | | | | | | (100) |
| Efficacy population | | | | | | |
| Modified intent-to-treat | 32 (100) | 32 (100) | 64 (100) | 68 (100) | 64 (100) | 132 |
| (mITT) | | | | | | (100) |
| Safety population | 32 | 32 | 64 | 68 | 64 | 132 |
| Pharmacokinetic (PK) | 0 | 31 (96.9) | 31 (48.4) | 0 | 64 (100) | 64 (48.5) |
| population | | | | | | |

TABLE 12
Participant disposition.

| | 400 mg BID cohort | | | 400 mg TID cohort | | |
|---|---|---|---|---|---|---|
| | Placebo | Rilzabrutinib | | Placebo | Rilzabrutinib | |
| | BID | 400 mg BID | All | TID | 400 mg TID | All |
| n (%) | (N = 32) | (N = 32) | (N = 64) | (N = 68) | (N = 64) | (N = 132) |
| Exposed but not | 0 | 0 | 0 | 0 | 0 | 0 |
| randomized | | | | | | |
| Randomized and | 0 | 0 | 0 | 0 | 0 | 0 |
| not exposed | | | | | | |
| Randomized and | 32 (100) | 32 (100) | 64 (100) | 68 (100) | 64 (100) | 132 (100) |
| exposed | | | | | | |
| Still on study | 0 | 0 | 0 | 0 | 0 | 0 |
| intervention or | | | | | | |
| missing EOT | | | | | | |
| page | | | | | | |
| Completed the | 18 (56.3) | 18 (56.3) | 36 (56.3) | 45 (66.2) | 44 (68.8) | 89 (67.4) |
| study | | | | | | |
| intervention | | | | | | |
| period | | | | | | |
| Did not | 14 (43.8) | 14 (43.8) | 28 (43.8) | 23 (33.8) | 20 (31.3) | 43 (32.6) |
| complete the | | | | | | |
| study | | | | | | |
| intervention | | | | | | |
| period | | | | | | |
| Reason for | | | | | | |
| permanent study | | | | | | |
| intervention | | | | | | |
| discontinuation | | | | | | |
| Adverse event | 2 (6.3) | 2 (6.3) | 4 (6.3) | 2 (2.9) | 5 (7.8) | 7 (5.3) |
| Related to | 2 (6.3) | 0 | 2 (3.1) | 1 (1.5) | 3 (4.7) | 4 (3.0) |
| COVID-19 | | | | | | |
| Not related to | 0 | 2 (6.3) | 2 (3.1) | 1 (1.5) | 2 (3.1) | 3 (2.3) |
| COVID-19 | | | | | | |
| Lack of | 0 | 0 | 0 | 1 (1.5) | 1 (1.6) | 2 (1.5) |
| efficacy | | | | | | |
| Poor | 1 (3.1) | 1 (3.1) | 2 (3.1) | 0 | 1 (1.6) | 1 (0.8) |
| compliance to | | | | | | |
| protocol | | | | | | |
| Withdrawal by | 0 | 1 (3.1) | 1 (1.6) | 1 (1.5) | 3 (4.7) | 4 (3.0) |
| subject | | | | | | |
| Loss of asthma | 10 (31.3) | 8 (25.0) | 18 (28.1) | 18 (26.5) | 10 (15.6) | 28 (21.2) |
| control | | | | | | |
| (LOAC) | | | | | | |
| Other | 1 (3.1) | 2 (6.3) | 3 (4.7) | 1 (1.5) | 0 | 1 (0.8) |
| Related to | 0 | 0 | 0 | 0 | 0 | 0 |
| COVID-19 | | | | | | |
| Not related to | 1 (3.1) | 2 (6.3) | 3 (4.7) | 1 (1.5) | 0 | 1 (0.8) |
| COVID-19 | | | | | | |
| Reason for study | | | | | | |
| intervention | | | | | | |
| withdrawal by | | | | | | |
| subject | | | | | | |
| Adverse event | 0 | 1 (3.1) | 1 (1.6) | 0 | 2 (3.1) | 2 (1.5) |
| Study | 0 | 0 | 0 | 0 | 0 | 0 |
| procedure | | | | | | |
| Inconvenience | 0 | 0 | 0 | 0 | 0 | 0 |
| of device use | | | | | | |
| Other | 0 | 0 | 0 | 0 | 0 | 0 |
| Related to | 0 | 0 | 0 | 0 | 0 | 0 |
| COVID-19 | | | | | | |
| Not related to | 0 | 0 | 0 | 0 | 0 | 0 |
| COVID-19 | | | | | | |
| Completed the | 31 (96.9) | 30 (93.8) | 61 (95.3) | 67 (98.5) | 60 (93.8) | 127 (96.2) |
| study period | | | | | | |
| Did not complete | 1 (3.1) | 2 (6.3) | 3 (4.7) | 1 (1.5) | 4 (6.3) | 5 (3.8) |
| the study period | | | | | | |
| Reason for study | | | | | | |
| discontinuation | | | | | | |
| Adverse event | 0 | 1 (3.1) | 1 (1.6) | 0 | 0 | 0 |
| Related to | 0 | 0 | 0 | 0 | 0 | 0 |
| COVID-19 | | | | | | |
| Not related to | 0 | 1 (3.1) | 1 (1.6) | 0 | 0 | 0 |
| COVID-19 | | | | | | |
| Poor | 1 (3.1) | 0 | 1 (1.6) | 0 | 0 | 0 |
| compliance to | | | | | | |
| protocol | | | | | | |
| Withdrawal by | 0 | 0 | 0 | 1 (1.5) | 4 (6.3) | 5 (3.8) |
| subject | | | | | | |
| Site terminated | 0 | 0 | 0 | 0 | 0 | 0 |
| by sponsor | | | | | | |
| Study | 0 | 1 (3.1) | 1 (1.6) | 0 | 0 | 0 |
| terminated by | | | | | | |
| sponsor | | | | | | |
| Other | 0 | 0 | 0 | 0 | 0 | 0 |
| Related to | 0 | 0) | 0 | 0 | 0 | 0 |
| COVID-19 | | | | | | |
| Not related to | 0 | 0 | 0 | 0 | 0 | 0 |
| COVID-19 | | | | | | |
| Status at last | 32 (100) | 31 (96.9) | 63 (98.4) | 68 (100) | 64 (100) | 132 (100) |
| contact | | | | | | |
| Alive | 32 (100) | 31 (96.9) | 63 (98.4) | 68 (100) | 64 (100) | 132 (100) |
| Dead | 0 | 0 | 0 | 0 | 0 | 0 |

TABLE 13
Demographic and participant characteristics at baseline (randomized population).

| | 400 mg BID cohort | | | 400 mg TID cohort | | |
|---|---|---|---|---|---|---|
| | Placebo | Rilzabrutinib | | Placebo | Rilzabrutinib | |
| | BID | 400 mg BID | All | TID | 400 mg TID | All |
| | (N = 32) | (N = 32) | (N = 64) | (N = 68) | (N = 64) | (N = 132) |
| Age (years) | | | | | | |
| Number | 32 | 32 | 64 | 68 | 64 | 132 |
| Mean (SD) | 50.8 (12.3) | 48.0 (13.4) | 49.4 (12.8) | 48.6 (13.7) | 48.8 (13.3) | 48.7 (13.5) |
| Median | 53.5 | 51.5 | 52.0 | 48.5 | 50.0 | 50.0 |
| Min; Max | 28; 70 | 21; 70 | 21; 70 | 18; 70 | 18; 68 | 18; 70 |
| Age group 1 (years) | | | | | | |
| [n (%)] | | | | | | |
| Number | 32 | 32 | 64 | 68 | 64 | 132 |
| <45 | 10 (31.3) | 12 (37.5) | 22 (34.4) | 29 (42.6) | 25 (39.1) | 54 (40.9) |
| ≥45 | 22 (68.8) | 20 (62.5) | 42 (65.6) | 39 (57.4) | 39 (60.9) | 78 (59.1) |
| Age group 2 (years) | | | | | | |
| [n (%)] | | | | | | |
| Number | 32 | 32 | 64 | 68 | 64 | 132 |
| <65 | 28 (87.5) | 29 (90.6) | 57 (89.1) | 57 (83.8) | 56 (87.5) | 113 (85.6) |
| ≥65 | 4 (12.5) | 3 (9.4) | 7 (10.9) | 11 (16.2) | 8 (12.5) | 19 (14.4) |
| Sex [n (%)] | | | | | | |
| Number | 32 | 32 | 64 | 68 | 64 | 132 |
| Male | 8 (25.0) | 14 (43.8) | 22 (34.4) | 27 (39.7) | 25 (39.1) | 52 (39.4) |
| Female | 24 (75.0) | 18 (56.3) | 42 (65.6) | 41 (60.3) | 39 (60.9) | 80 (60.6) |
| Height (cm) | | | | | | |
| Number | 32 | 32 | 64 | 68 | 64 | 132 |
| Mean (SD) | 164.9 | 166.0 (8.8) | 165.4 (9.5) | 167.9 | 169.5 (11.9) | 168.6 (11.8) |
| | (10.2) | | | (11.8) | | |
| Median | 165.0 | 164.5 | 165.0 | 169.5 | 169.0 | 169.0 |
| Min; Max | 148; 185 | 150; 184 | 148; 185 | 140; 194 | 146; 194 | 140; 194 |
| Weight (kg) | | | | | | |
| Number | 32 | 32 | 64 | 68 | 64 | 132 |
| Mean (SD) | 77.0 (13.1) | 78.0 (15.2) | 77.5 (14.0) | 77.2 (14.1) | 83.2 (12.4) | 80.1 (13.6) |
| Median | 76.5 | 75.5 | 76.0 | 78.0 | 83.7 | 81.9 |
| Min; Max | 50; 117 | 54; 126 | 50; 126 | 48; 104 | 54; 112 | 48; 112 |
| Weight by category | | | | | | |
| (kg) [n (%)] | | | | | | |
| Number | 32 | 32 | 64 | 68 | 64 | 132 |
| <60 | 4 (12.5) | 3 (9.4) | 7 (10.9) | 12 (17.6) | 2 (3.1) | 14 (10.6) |
| ≥60-<90 | 25 (78.1) | 23 (71.9) | 48 (75.0) | 40 (58.8) | 41 (64.1) | 81 (61.4) |
| ≥90 | 3 (9.4) | 6 (18.8) | 9 (14.1) | 16 (23.5) | 21 (32.8) | 37 (28.0) |
| BMI (kg/m²) | | | | | | |
| Number | 32 | 32 | 64 | 68 | 64 | 132 |
| Mean (SD) | 28.39 | 28.18 (4.01) | 28.29 | 27.46 | 29.08 (4.29) | 28.24 (4.59) |
| | (4.79) | | (4.38) | (4.75) | | |
| Median | 27.60 | 27.75 | 27.65 | 26.75 | 28.90 | 27.55 |
| Min; Max | 20.5; 40.1 | 21.8; 38.2 | 20.5; 40.1 | 19.6; 38.3 | 21.6; 38.7 | 19.6; 38.7 |
| BMI by category | | | | | | |
| (kg/m²) [n (%)] | | | | | | |
| Number | 32 | 32 | 64 | 68 | 64 | 132 |
| <25 | 6 (18.8) | 8 (25.0) | 14 (21.9) | 22 (32.4) | 12 (18.8) | 34 (25.8) |
| ≥25-<30 | 17 (53.1) | 16 (50.0) | 33 (51.6) | 27 (39.7) | 29 (45.3) | 56 (42.4) |
| ≥30 | 9 (28.1) | 8 (25.0) | 17 (26.6) | 19 (27.9) | 23 (35.9) | 42 (31.8) |
| Region [n (%)] | | | | | | |
| Number | 32 | 32 | 64 | 68 | 64 | 132 |
| Eastern Europe | 4 (12.5) | 3 (9.4) | 7 (10.9) | 45 (66.2) | 43 (67.2) | 88 (66.7) |
| Latin America | 25 (78.1) | 25 (78.1) | 50 (78.1) | 19 (27.9) | 19 (29.7) | 38 (28.8) |
| Western Countries | 1 (3.1) | 2 (6.3) | 3 (4.7) | 2 (2.9) | 2 (3.1) | 4 (3.0) |
| Asia and Pacific | 2 (6.3) | 2 (6.3) | 4 (6.3) | 2 (2.9) | 0 | 2 (1.5) |
| Region | | | | | | |

TABLE 14
Asthma history characteristics at baseline (randomized population).

| | 400 mg BID cohort | | | 400 mg TID cohort | | |
|---|---|---|---|---|---|---|
| | Placebo | Rilzabrutinib | | | Rilzabrutinib | |
| | BID | 400 mg BID | All | Placebo TID | 400 mg TID | All |
| | (N = 32) | (N = 32) | (N = 64) | (N = 68) | (N = 64) | (N = 132) |
| Age of onset of | | | | | | |
| asthma (years) | | | | | | |
| Number | 32 | 32 | 64 | 68 | 64 | 132 |
| Mean (SD) | 28.1 (16.6) | 24.2 (16.4) | 26.1 (16.5) | 28.5 (18.0) | 28.7 (16.5) | 28.6 (17.2) |
| Median | 29.5 | 26.5 | 27.5 | 28.0 | 30.0 | 29.5 |
| Q1; Q3 | 15.0; 40.0 | 9.5; 36.0 | 11.5; 37.0 | 13.0; 44.5 | 13.5; 40.5 | 13.0; 41.5 |
| Min; Max | 0; 65 | 0; 60 | 0; 65 | 0; 63 | 1; 61 | 0; 63 |
| Age of onset of | | | | | | |
| asthma group 1 | | | | | | |
| (years) [n (%)] | | | | | | |
| Number | 32 | 32 | 64 | 68 | 64 | 132 |
| <12 | 7 (21.9) | 9 (28.1) | 16 (25.0) | 15 (22.1) | 14 (21.9) | 29 (22.0) |
| ≥12-<18 | 2 (6.3) | 5 (15.6) | 7 (10.9) | 5 (7.4) | 5 (7.8) | 10 (7.6) |
| ≥18-<40 | 15 (46.9) | 14 (43.8) | 29 (45.3) | 28 (41.2) | 27 (42.2) | 55 (41.7) |
| ≥40 | 8 (25.0) | 4 (12.5) | 12 (18.8) | 20 (29.4) | 18 (28.1) | 38 (28.8) |
| Age of onset | | | | | | |
| of asthma | | | | | | |
| group 2 | | | | | | |
| (years) [n (%)] | | | | | | |
| Number | 32 | 32 | 64 | 68 | 64 | 132 |
| <18 | 9 (28.1) | 14 (43.8) | 23 (35.9) | 20 (29.4) | 19 (29.7) | 39 (29.5) |
| ≥18 | 23 (71.9) | 18 (56.3) | 41 (64.1) | 48 (70.6) | 45 (70.3) | 93 (70.5) |
| Time since first | | | | | | |
| diagnosis of | | | | | | |
| asthma (years) | | | | | | |
| Number | 32 | 32 | 64 | 68 | 64 | 132 |
| Mean (SD) | 23.47 | 24.89 (14.90) | 24.18 | 20.93 (14.00) | 21.02 (13.28) | 20.97 (13.61) |
| | (13.87) | | (14.30) | | | |
| Median | 21.58 | 20.50 | 20.79 | 17.29 | 20.04 | 17.96 |
| Q1; Q3 | 12.63; | 14.96; 34.92 | 13.71; | 11.08; 32.21 | 9.67; 29.08 | 10.38; 30.00 |
| | 33.54 | | 33.54 | | | |
| Min; Max | 1.3; 54.0 | 3.8; 56.5 | 1.3; 56.5 | 1.1; 55.8 | 1.8; 62.2 | 1.1; 62.2 |
| With history of | | | | | | |
| atopic medical | | | | | | |
| conditions [n (%)] | | | | | | |
| Number | 32 | 32 | 64 | 68 | 64 | 132 |
| Yes | 17 (53.1) | 20 (62.5) | 37 (57.8) | 24 (35.3) | 21 (32.8) | 45 (34.1) |
| Ongoing | 16 (50.0) | 19 (59.4) | 35 (54.7) | 24 (35.3) | 21 (32.8) | 45 (34.1) |
| Smoking history | | | | | | |
| [n (%)] | | | | | | |
| Number | 32 | 32 | 64 | 68 | 64 | 132 |
| Former | 8 (25.0) | 4 (12.5) | 12 (18.8) | 6 (8.8) | 6 (9.4) | 12 (9.1) |
| Never | 24 (75.0) | 28 (87.5) | 52 (81.3) | 62 (91.2) | 58 (90.6) | 120 (90.9) |
| Time since | | | | | | |
| cessation | | | | | | |
| (years) | | | | | | |
| Number | 8 | 4 | 12 | 6 | 6 | 12 |
| Mean (SD) | 19.74 | 19.46 (10.13) | 19.65 | 21.82 (17.53) | 20.63 (11.86) | 21.22 (14.28) |
| | (15.64) | | (13.55) | | | |
| Median | 21.21 | 20.67 | 21.21 | 17.25 | 20.63 | 20.63 |
| Q1; Q3 | 3.92; 32.17 | 12.29; 26.63 | 6.33; 31.21 | 9.33; 41.17 | 15.58; 25.42 | 9.33; 32.00 |
| Min; Max | 0.8; 42.6 | 6.2; 30.3 | 0.8; 42.6 | 2.5; 43.4 | 2.9; 38.6 | 2.5; 43.4 |
| Pack-year | | | | | | |
| Number | 8 | 4 | 12 | 6 | 6 | 12 |
| Mean (SD) | 1.402 | 4.500 (1.291) | 2.434 | 5.183 (2.669) | 3.092 (2.288) | 4.138 (2.610) |
| | (1.139) | | (1.899) | | | |
| Median | 1.300 | 4.500 | 2.400 | 4.500 | 3.000 | 4.000 |
| Q1; Q3 | 0.400; | 3.500; 5.500 | 0.900; | 4.000; 7.500 | 1.000; 4.650 | 2.050; 5.750 |
| | 2.400 | | 3.500 | | | |
| Min; Max | 0.01; 3.00 | 3.00; 6.00 | 0.01; 6.00 | 1.60; 9.00 | 0.40; 6.50 | 0.40; 9.00 |
| E-cigarette | | | | | | |
| history [n (%)] | | | | | | |
| Number | 32 | 32 | 64 | 68 | 64 | 132 |
| Former | 0 | 0 | 0 | 0 | 0 | 0 |
| Never | 32 (100) | 32 (100) | 64 (100) | 68 (100) | 64 (100) | 132 (100) |

TABLE 15
Asthma severity and control at baseline (randomized population).

| | 400 mg BID cohort | | | 400 mg TID cohort | | |
|---|---|---|---|---|---|---|
| | Placebo | Rilzabrutinib | | Placebo | Rilzabrutinib | |
| | BID | 400 mg BID | All | TID | 400 mg TID | All |
| | (N = 32) | (N = 32) | (N = 64) | (N = 68) | (N = 64) | (N = 132) |
| ICS/LABA dose | | | | | | |
| level [n (%)] | | | | | | |
| Number | 32 | 32 | 64 | 68 | 64 | 132 |
| High | 29 (90.6) | 27 (84.4) | 56 (87.5) | 33 (48.5) | 35 (54.7) | 68 (51.5) |
| Medium | 3 (9.4) | 5 (15.6) | 8 (12.5) | 35 (51.5) | 29 (45.3) | 64 (48.5) |
| ICS/LABA total | | | | | | |
| dose (ug) | | | | | | |
| Number | 32 | 32 | 64 | 68 | 64 | 132 |
| Mean (SD) | 953.1 | 953.1 (265.2) | 953.1 | 742.6 | 773.4 (250.9) | 757.6 |
| | (148.1) | | (213.0) | (251.7) | | (250.8) |
| Median | 1000.0 | 1000.0 | 1000.0 | 500.0 | 1000.0 | 1000.0 |
| Q1; Q3 | 1000.0; | 1000.0; | 1000.0; | 500.0; | 500.0; 1000.0 | 500.0; |
| | 1000.0 | 1000.0 | 1000.0 | 1000.0 | | 1000.0 |
| Min; Max | 500; | 500; 2000 | 500; | 500; | 500; 1000 | 500; |
| | 1000 | | 2000 | 1000 | | 1000 |
| LABA total daily dose | | | | | | |
| (ug) | | | | | | |
| Number | 32 | 32 | 64 | 68 | 64 | 132 |
| Mean (SD) | 98.4 (8.8) | 100.0 (22.0) | 99.2 | 97.8 | 96.1 (13.5) | 97.0 |
| | | | (16.6) | (18.2) | | (16.1) |
| Median | 100.0 | 100.0 | 100.0 | 100.0 | 100.0 | 100.0 |
| Q1; Q3 | 100.0; | 100.0; 100.0 | 100.0; | 100.0; | 100.0; 100.0 | 100.0; |
| | 100.0 | | 100.0 | 100.0 | | 100.0 |
| Min; Max | 50; 100 | 50; 200 | 50; 200 | 50; 200 | 50; 100 | 50; 200 |
| Number of prescribed | | | | | | |
| salbutamol/albuterol or | | | | | | |
| levosalbutamol/levalbuterol | | | | | | |
| (puffs) | | | | | | |
| Number | 19 | 23 | 42 | 53 | 47 | 100 |
| Mean (SD) | 1.5 (0.5) | 2.4 (2.3) | 2.0 (1.8) | 2.3 (2.9) | 2.0 (1.8) | 2.2 (2.4) |
| Median | 2.0 | 2.0 | 2.0 | 1.0 | 1.0 | 1.0 |
| Q1; Q3 | 1.0; 2.0 | 1.0; 2.0 | 1.0; 2.0 | 1.0; 2.0 | 1.0; 2.0 | 1.0; 2.0 |
| Min; Max | 1; 2 | 0; 8 | 0; 8 | 1; 20 | 1; 8 | 1; 20 |
| Time since last asthma | | | | | | |
| exacerbation (months) | | | | | | |
| Number | 32 | 32 | 64 | 68 | 64 | 132 |
| Mean (SD) | 6.94 | 7.69 (4.83) | 7.31 | 8.51 | 9.64 (4.90) | 9.06 |
| | (3.73) | | (4.30) | (4.55) | | (4.74) |
| Median | 6.00 | 7.00 | 6.00 | 8.00 | 9.00 | 8.00 |
| Q1; Q3 | 4.00; | 4.00; 10.00 | 4.00; | 6.00; | 6.00; 12.50 | 6.00; |
| | 8.50 | | 9.00 | 11.00 | | 11.50 |
| Min; Max | 2.0; 15.0 | 2.0; 23.0 | 2.0; 23.0 | 1.0; 24.0 | 2.0; 24.0 | 1.0; 24.0 |
| Number of asthma | | | | | | |
| exacerbations experienced 2 | | | | | | |
| years before the screening | | | | | | |
| visit | | | | | | |
| Number | 32 | 32 | 64 | 68 | 64 | 132 |
| Mean (SD) | 2.3 (1.5) | 1.9 (1.0) | 2.1 (1.2) | 1.3 (0.7) | 1.5 (0.8) | 1.4 (0.7) |
| Median | 2.0 | 2.0 | 2.0 | 1.0 | 1.0 | 1.0 |
| Q1; Q3 | 1.0; 2.5 | 1.0; 2.5 | 1.0; 2.5 | 1.0; 1.5 | 1.0; 2.0 | 1.0; 2.0 |
| Min; Max | 1; 8 | 1; 5 | 1; 8 | 1; 6 | 1; 5 | 1; 6 |
| Number of asthma | | | | | | |
| exacerbations experienced 2 | | | | | | |
| years before the screening | | | | | | |
| visit [n (%)] | | | | | | |
| Number | 32 | 32 | 64 | 68 | 64 | 132 |
| 1 | 9 (28.1) | 13 (40.6) | 22 (34.4) | 51 (75.0) | 36 (56.3) | 87 (65.9) |
| 2 | 15 (46.9) | 11 (34.4) | 26 (40.6) | 16 (23.5) | 23 (35.9) | 39 (29.5) |
| 3 | 3 (9.4) | 7 (21.9) | 10 (15.6) | 0 | 4 (6.3) | 4 (3.0) |
| ≥4 | 5 (15.6) | 1 (3.1) | 6 (9.4) | 1 (1.5) | 1 (1.6) | 2 (1.5) |
| Number of asthma | | | | | | |
| exacerbations experienced 1 | | | | | | |
| year before the screening | | | | | | |
| visit | | | | | | |
| Number | 32 | 32 | 64 | 68 | 64 | 132 |
| Mean (SD) | 1.6 (0.9) | 1.4 (0.8) | 1.5 (0.8) | 0.8 (0.6) | 0.8 (0.8) | 0.8 (0.7) |
| Median | 2.0 | 1.0 | 1.0 | 1.0 | 1.0 | 1.0 |
| Q1; Q3 | 1.0; 2.0 | 1.0; 2.0 | 1.0; 2.0 | 0.0; 1.0 | 0.0; 1.0 | 0.0; 1.0 |
| Min; Max | 0; 4 | 0; 3 | 0; 4 | 0; 3 | 0; 4 | 0; 4 |
| Number of asthma | | | | | | |
| exacerbations experienced 1 | | | | | | |
| year before the screening | | | | | | |
| visit [n (%)] | | | | | | |
| Number | 32 | 32 | 64 | 68 | 64 | 132 |
| 0 | 2 (6.3) | 2 (6.3) | 4 (6.3) | 19 (27.9) | 23 (35.9) | 42 (31.8) |
| 1 | 13 (40.6) | 19 (59.4) | 32 (50.0) | 43 (63.2) | 31 (48.4) | 74 (56.1) |
| 2 | 13 (40.6) | 8 (25.0) | 21 (32.8) | 5 (7.4) | 9 (14.1) | 14 (10.6) |
| 3 | 3 (9.4) | 3 (9.4) | 6 (9.4) | 1 (1.5) | 0 | 1 (0.8) |
| ≥4 | 1 (3.1) | 0 | 1 (1.6) | 0 | 1 (1.6) | 1 (0.8) |
| Number of asthma | | | | | | |
| exacerbations experienced | | | | | | |
| 1 year before the | | | | | | |
| screening visit group 2 [n | | | | | | |
| (%)] | | | | | | |
| Number | 32 | 32 | 64 | 68 | 64 | 132 |
| 0 | 2 (6.3) | 2 (6.3) | 4 (6.3) | 19 (27.9) | 23 (35.9) | 42 (31.8) |
| ≥1 | 30 (93.8) | 30 (93.8) | 60 (93.8) | 49 (72.1) | 41 (64.1) | 90 (68.2) |
| Number of asthma | | | | | | |
| exacerbations required | | | | | | |
| hospitalization or emergency | | | | | | |
| medical care within 2 years | | | | | | |
| before the screening visit | | | | | | |
| Number | 32 | 32 | 64 | 68 | 64 | 132 |
| Mean (SD) | 0.5 (1.3) | 0.1 (0.3) | 0.3 (1.0) | 0.3 (0.6) | 0.2 (0.5) | 0.2 (0.5) |
| Median | 0.0 | 0.0 | 0.0 | 0.0 | 0.0 | 0.0 |
| Q1; Q3 | 0.0; 0.5 | 0.0; 0.0 | 0.0; 0.0 | 0.0; 0.0 | 0.0; 0.0 | 0.0; 0.0 |
| Min; Max | 0; 7 | 0; 1 | 0; 7 | 0; 2 | 0; 2 | 0; 2 |
| Number of asthma | | | | | | |
| exacerbations required | | | | | | |
| hospitalization or emergency | | | | | | |
| medical care within 2 years | | | | | | |
| before the screening visit | | | | | | |
| group [n (%)] | | | | | | |
| Number | 32 | 32 | 64 | 68 | 64 | 132 |
| 0 | 24 (75.0) | 29 (90.6) | 53 (82.8) | 54 (79.4) | 55 (85.9) | 109 (82.6) |
| 1 | 4 (12.5) | 3 (9.4) | 7 (10.9) | 10 (14.7) | 6 (9.4) | 16 (12.1) |
| 2 | 3 (9.4) | 0 | 3 (4.7) | 4 (5.9) | 3 (4.7) | 7 (5.3) |
| 3 | 0 | 0 | 0 | 0 | 0 | 0 |
| ≥4 | 1 (3.1) | 0 | 1 (1.6) | 0 | 0 | 0 |
| Number of asthma | | | | | | |
| exacerbations required | | | | | | |
| hospitalization or emergency | | | | | | |
| medical care within 1 year | | | | | | |
| before the screening visit | | | | | | |
| Number | 32 | 32 | 64 | 68 | 64 | 132 |
| Mean (SD) | 0.3 (0.6) | 0.1 (0.3) | 0.2 (0.5) | 0.2 (0.4) | 0.1 (0.4) | 0.2 (0.4) |
| Median | 0.0 | 0.0 | 0.0 | 0.0 | 0.0 | 0.0 |
| Q1; Q3 | 0.0; 0.0 | 0.0; 0.0 | 0.0; 0.0 | 0.0; 0.0 | 0.0; 0.0 | 0.0; 0.0 |
| Min; Max | 0; 3 | 0; 1 | 0; 3 | 0; 1 | 0; 2 | 0; 2 |
| Number of asthma | | | | | | |
| exacerbations required | | | | | | |
| hospitalization or emergency | | | | | | |
| medical care within 1 year | | | | | | |
| before the screening visit | | | | | | |
| group [n (%)] | | | | | | |
| Number | 32 | 32 | 64 | 68 | 64 | 132 |
| 0 | 26 (81.3) | 29 (90.6) | 55 (85.9) | 55 (80.9) | 58 (90.6) | 113 (85.6) |
| 1 | 5 (15.6) | 3 (9.4) | 8 (12.5) | 13 (19.1) | 5 (7.8) | 18 (13.6) |
| 2 | 0 | 0 | 0 | 0 | 1 (1.6) | 1 (0.8) |
| 3 | 1 (3.1) | 0 | 1 (1.6) | 0 | 0 | 0 |
| ≥4 | 0 | 0 | 0 | 0 | 0 | 0 |
| Pre-bronchodilator | | | | | | |
| FEV1 (L) | | | | | | |
| Number | 32 | 31 | 63 | 67 | 63 | 130 |
| Mean (SD) | 2.16 | 2.11 (0.67) | 2.13 | 2.42 | 2.50 (0.95) | 2.46 |
| | (0.77) | | (0.72) | (0.90) | | (0.92) |
| Median | 2.10 | 1.94 | 2.02 | 2.15 | 2.36 | 2.20 |
| Q1; Q3 | 1.47; | 1.59; 2.52 | 1.59 : | 1.77; | 1.73; 3.10 | 1.75; |
| | 2.50 | | 2.52 | 2.98 | | 3.03 |
| Min; Max | 1.2; 3.9 | 1.0; 3.8 | 1.0; 3.9 | 1.0; 5.0 | 1.1; 5.3 | 1.0; 5.3 |
| Pre-bronchodilator FEV1 | | | | | | |
| percent predicted (%) | | | | | | |
| Number | 32 | 31 | 63 | 68 | 63 | 131 |
| Mean (SD) | 71.7 | 66.4 (10.4) | 69.1 | 76.0 | 75.7 (16.2) | 75.9 |
| | (12.7) | | (11.8) | (16.7) | | (16.4) |
| Median | 72.5 | 64.0 | 68.0 | 74.5 | 78.0 | 77.0 |
| Q1; Q3 | 61.5; | 58.0; 75.0 | 59.0; | 62.5; | 65.0; 85.0 | 64.0; |
| | 79.5 | | 78.0 | 87.0 | | 86.0 |
| Min; Max | 54; 115 | 50; 87 | 50; 115 | 50; 115 | 35; 118 | 35; 118 |
| Pre-bronchodilator FEV1 | | | | | | |
| percent predicted group | | | | | | |
| (%) [n (%)] | | | | | | |
| Number | 32 | 31 | 63 | 68 | 63 | 131 |
| <80 | 24 (75.0) | 26 (83.9) | 50 (79.4) | 41 (60.3) | 37 (58.7) | 78 (59.5) |
| ≥80 | 8 (25.0) | 5 (16.1) | 13 (20.6) | 27 (39.7) | 26 (41.3) | 53 (40.5) |
| Post-bronchodilator FEV1 | | | | | | |
| (L) | | | | | | |
| Number | 32 | 32 | 64 | 63 | 59 | 122 |
| Mean (SD) | 2.39 | 2.40 (0.75) | 2.40 | 2.60 | 2.66 (0.98) | 2.63 |
| | (0.77) | | (0.76) | (0.91) | | (0.94) |
| Median | 2.30 | 2.21 | 2.26 | 2.35 | 2.51 | 2.42 |
| Q1; Q3 | 1.77; | 1.96; 2.79 | 1.88; | 1.94; | 1.83; 3.27 | 1.91; |
| | 2.86 | | 2.85 | 3.03 | | 3.08 |
| Min; Max | 1.3; 4.3 | 1.2; 4.3 | 1.2; 4.3 | 1.1; 5.1 | 1.3; 5.3 | 1.1; 5.3 |
| Post-bronchodilator FEV1 | | | | | | |
| percent predicted (%) | | | | | | |
| Number | 32 | 32 | 64 | 67 | 62 | 129 |
| Mean (SD) | 79.3 | 75.7 (14.9) | 77.5 | 82.0 | 81.9 (14.9) | 82.0 |
| | (12.9) | | (13.9) | (16.2) | | (15.5) |
| Median | 80.0 | 75.0 | 77.0 | 82.0 | 81.0 | 81.0 |
| Q1; Q3 | 70.5; | 64.0; 85.5 | 67.5; | 68.0; | 71.0; 91.0 | 70.0; |
| | 87.0 | | 86.5 | 93.0 | | 91.0 |
| Min; Max | 53; 111 | 50; 126 | 50; 126 | 50; 117 | 51; 122 | 50; 122 |
| FEV1 reversibility at | | | | | | |
| screening (%) | | | | | | |
| Number | 30 | 32 | 62 | 67 | 64 | 131 |
| Mean (SD) | 22.901 | 25.238 | 24.107 | 21.265 | 23.026 | 22.125 |
| | (10.238) | (12.189) | (11.257) | (14.469) | (10.145) | (12.527) |
| Median | 20.000 | 21.600 | 20.000 | 18.000 | 19.750 | 19.000 |
| Q1; Q3 | 14.000; | 15.500; | 15.000; | 14.000; | 15.700; | 15.000; |
| | 29.000 | 35.500 | 30.600 | 26.000 | 28.825 | 27.000 |
| Min; Max | 12.00; | 10.00; 55.00 | 10.00; | −1.00; | 12.20; 55.00 | −1.00; |
| | 46.00 | | 55.00 | 105.00 | | 105.00 |
| FEV1 reversibility (%) | | | | | | |
| Number | 30 | 29 | 59 | 60 | 56 | 116 |
| Mean (SD) | 11.364 | 12.927 (9.681) | 12.132 | 7.142 | 7.932 | 7.523 |
| | (13.206) | | (11.535) | (8.179) | (10.140) | (9.146) |
| Median | 8.329 | 13.632 | 9.396 | 4.708 | 5.539 | 5.239 |
| Q1; Q3 | 4.079; | 5.722; 18.199 | 4.977; | 2.062; | 1.105; 11.469 | 1.634; |
| | 13.434 | | 16.887 | 10.190 | | 10.658 |
| Min; Max | −2.84; | −1.82; 33.42 | −2.84; | −4.56; | −6.78; 53.68 | −6.78; |
| | 67.78 | | 67.78 | 33.53 | | 53.68 |
| Baseline FEV1 | | | | | | |
| reversibility group | | | | | | |
| (%) [n (%)] | | | | | | |
| Number | 30 | 29 | 59 | 60 | 56 | 116 |
| <12 | 21 (70.0) | 14 (48.3) | 35 (59.3) | 48 (80.0) | 43 (76.8) | 91 (78.4) |
| ≥12 | 9 (30.0) | 15 (51.7) | 24 (40.7) | 12 (20.0) | 13 (23.2) | 25 (21.6) |
| PEF (L/min) | | | | | | |
| Number | 32 | 32 | 64 | 68 | 63 | 131 |
| Mean (SD) | 319.66 | 307.34 | 313.50 | 304.13 | 323.69 | 313.54 |
| | (130.75) | (123.27) | (126.21) | (121.76) | (107.13) | (114.94) |
| Median | 297.19 | 305.07 | 302.07 | 277.79 | 307.86 | 289.71 |
| Q1; Q3 | 214.29; | 211.46; | 211.46; | 212.10; | 245.14; | 227.43; |
| | 391.83 | 352.59 | 383.18 | 371.07 | 387.43 | 382.43 |
| Min; Max | 148.6; | 116.0; 611.1 | 116.0; | 96.5; | 144.8; 567.3 | 96.5; |
| | 632.4 | | 632.4 | 631.3 | | 631.3 |
| PM PEF (L/min) | | | | | | |
| Number | 32 | 32 | 64 | 67 | 62 | 129 |
| Mean (SD) | 322.39 | 320.15 | 321.27 | 308.20 | 330.75 | 319.04 |
| | (127.71) | (117.16) | (121.57) | (118.90) | (104.19) | (112.21) |
| Median | 298.29 | 317.78 | 306.91 | 283.43 | 319.36 | 306.29 |
| Q1; Q3 | 221.95; | 220.92; | 220.92; | 218.33; | 260.43; | 234.29; |
| | 387.43 | 378.00 | 378.93 | 380.57 | 409.43 | 383.71 |
| Min; Max | 153.4; | 135.6; 622.6 | 135.6; | 99.7; | 146.0; 557.6 | 99.7; |
| | 651.6 | | 651.6 | 596.2 | | 596.2 |
| ACQ-5 score | | | | | | |
| Number | 32 | 32 | 64 | 68 | 64 | 132 |
| Mean (SD) | 2.19 | 2.04 (0.37) | 2.12 | 2.18 | 2.24 (0.48) | 2.21 |
| | (0.40) | | (0.38) | (0.40) | | (0.44) |
| Median | 2.20 | 2.00 | 2.00 | 2.20 | 2.20 | 2.20 |
| Q1; Q3 | 2.00; | 1.80; 2.20 | 1.80; | 1.80; | 2.00; 2.60 | 2.00; |
| | 2.40 | | 2.30 | 2.40 | | 2.60 |
| Min; Max | 1.4; 3.2 | 1.4; 2.8 | 1.4; 3.2 | 1.2; 3.2 | 1.4; 4.0 | 1.2; 4.0 |
| ACQ-5 score group [n | | | | | | |
| (%)] | | | | | | |
| Number | 32 | 32 | 64 | 68 | 64 | 132 |
| <2 | 15 (46.9) | 19 (59.4) | 34 (53.1) | 29 (42.6) | 24 (37.5) | 53 (40.2) |
| >2 | 17 (53.1) | 13 (40.6) | 30 (46.9) | 39 (57.4) | 40 (62.5) | 79 (59.8) |
| AQLQ global score | | | | | | |
| Number | 31 | 32 | 63 | 67 | 62 | 129 |
| Mean (SD) | 4.67 | 4.91 (1.04) | 4.79 | 4.68 | 4.96 (0.71) | 4.82 |
| | (1.07) | | (1.06) | (0.83) | | (0.79) |
| Median | 4.69 | 5.19 | 4.88 | 4.56 | 4.94 | 4.81 |
| Q1; Q3 | 4.13; | 4.31; 5.64 | 4.13; | 4.00; | 4.38; 5.44 | 4.22; |
| | 5.59 | | 5.59 | 5.41 | | 5.41 |
| Min; Max | 2.1; 6.2 | 2.4; 6.4 | 2.1; 6.4 | 2.8; 6.3 | 3.7; 6.6 | 2.8; 6.6 |
| PGIS score | | | | | | |
| Number | 31 | 32 | 63 | 67 | 62 | 129 |
| Mean (SD) | 1.6 (0.5) | 1.3 (0.6) | 1.5 (0.6) | 1.7 (0.6) | 1.6 (0.6) | 1.7 (0.6) |
| Median | 2.0 | 1.0 | 1.0 | 2.0 | 2.0 | 2.0 |
| Q1; Q3 | 1.0; 2.0 | 1.0; 2.0 | 1.0; 2.0 | 1.0; 2.0 | 1.0; 2.0 | 1.0; 2.0 |
| Min; Max | 1; 2 | 0; 3 | 0; 3 | 0; 3 | 0; 2 | 0; 3 |
| Number of inhalations of | | | | | | |
| salbutamol/albuterol or | | | | | | |
| levosalbutamol/levalbuterol/ | | | | | | |
| 24 hours (puffs) | | | | | | |
| Number | 32 | 32 | 64 | 67 | 62 | 129 |
| Mean (SD) | 2.52 | 1.57 (1.98) | 2.04 | 0.94 | 0.82 (1.50) | 0.88 |
| | (3.02) | | (2.58) | (2.13) | | (1.85) |
| Median | 1.29 | 0.62 | 0.85 | 0.00 | 0.00 | 0.00 |
| Q1; Q3 | 0.00; | 0.00; 2.93 | 0.00; | 0.00; | 0.00; 1.00 | 0.00; |
| | 4.71 | | 4.00 | 1.14 | | 1.00 |
| Min; Max | 0.0; 12.3 | 0.0; 7.4 | 0.0; 12.3 | 0.0; 12.5 | 0.0; 7.3 | 0.0; 12.5 |

TABLE 16
Summary of baseline biomarkers (randomized population).

| | 400 mg BID cohort | | | 400 mg TID cohort | | |
|---|---|---|---|---|---|---|
| | Placebo | Rilzabrutinib | | Placebo | Rilzabrutinib | |
| | BID | 400 mg BID | All | TID | 400 mg TID | All |
| | (N = 32) | (N = 32) | (N = 64) | (N = 68) | (N = 64) | (N = 132) |
| Blood eosinophils | | | | | | |
| (10⁹/L) | | | | | | |
| Number | 32 | 32 | 64 | 68 | 64 | 132 |
| Mean (SD) | 0.326 | 0.381 (0.334) | 0.354 | 0.267 | 0.248 (0.215) | 0.257 |
| | (0.242) | | (0.290) | (0.290) | | (0.256) |
| Median | 0.285 | 0.275 | 0.280 | 0.170 | 0.165 | 0.170 |
| Q1; Q3 | 0.160; | 0.140; 0.495 | 0.150; | 0.100; | 0.100; 0.325 | 0.100; |
| | 0.420 | | 0.470 | 0.315 | | 0.320 |
| Min; Max | 0.02; 1.18 | 0.03; 1.59 | 0.02; 1.59 | 0.02; 1.51 | 0.04; 1.06 | 0.02; 1.51 |
| Blood eosinophils | | | | | | |
| group [n (%)] | | | | | | |
| Number | 32 | 31 | 64 | 68 | 64 | 132 |
| <0.15 | 6 (18.8) | 8 (25.8) | 14 (21.9) | 29 (42.6) | 28 (43.8) | 57 (43.2) |
| ≥0.15-<0.3 | 13 (40.6) | 10 (31.3) | 23 (35.9) | 20 (29.4) | 18 (28.1) | 38 (28.8) |
| ≥0.3 | 13 (40.6) | 14 (43.8) | 27 (42.2) | 19 (27.9) | 18 (28.1) | 37 (28.0) |
| FeNO (ppb) | | | | | | |
| Number | 31 | 32 | 63 | 64 | 62 | 126 |
| Mean (SD) | 36.5 (34.4) | 37.4 (39.3) | 37.0 (36.7) | 24.4 (22.2) | 23.0 (17.5) | 23.7 (19.9) |
| Median | 26.0 | 27.0 | 26.0 | 17.5 | 17.0 | 17.5 |
| Q1; Q3 | 13.0; 46.0 | 18.5; 39.5 | 15.0; 42.0 | 11.5; 31.0 | 11.0; 32.0 | 11.0; 32.0 |
| Min; Max | 8; 161 | 11; 231 | 8; 231 | 5; 147 | 5; 85 | 5; 147 |
| FeNO group 1 [n (%)] | | | | | | |
| Number | 31 | 32 | 63 | 64 | 62 | 126 |
| <25 | 15 (48.4) | 12 (37.5) | 27 (42.9) | 42 (65.6) | 42 (67.7) | 84 (66.7) |
| ≥25-<50 | 10 (32.3) | 15 (46.9) | 25 (39.7) | 18 (28.1) | 16 (25.8) | 34 (27.0) |
| ≥50 | 6 (19.4) | 5 (15.6) | 11 (17.5) | 4 (6.3) | 4 (6.5) | 8 (6.3) |
| FeNO group | | | | | | |
| 2 [n (%)] | | | | | | |
| Number | 31 | 32 | 63 | 64 | 62 | 126 |
| <35 | 19 (61.3) | 21 (65.6) | 40 (63.5) | 50 (78.1) | 47 (75.8) | 97 (77.0) |
| ≥35 | 12 (38.7) | 11 (34.4) | 23 (36.5) | 14 (21.9) | 15 (24.2) | 29 (23.0) |
| Total IgE (IU/mL) | | | | | | |
| Number | 32 | 32 | 64 | 68 | 64 | 132 |
| Mean (SD) | 526.43 | 509.28 | 517.85 | 386.70 | 517.94 | 450.33 |
| | (630.53) | (815.43) | (723.11) | (724.05) | (924.94) | (826.98) |
| Median | 281.05 | 178.40 | 210.80 | 119.65 | 132.65 | 121.85 |
| Q1; Q3 | 88.70; | 95.55; 682.20 | 93.30; | 64.35; | 40.00; 466.70 | 60.85; |
| | 718.35 | | 708.25 | 366.40 | | 410.25 |
| Min; Max | 10.9; | 7.9; 4030.0 | 7.9; | 2.9; 3630.0 | 0.2; 4015.0 | 0.2; 4015.0 |
| | 2413.0 | | 4030.0 | | | |
| Total IgE level | | | | | | |
| (IU/mL) [n (%)] | | | | | | |
| Number | 32 | 32 | 64 | 68 | 64 | 132 |
| <100 | 11 (34.4) | 8 (25.0) | 19 (29.7) | 27 (39.7) | 23 (35.9) | 50 (37.9) |
| ≥100 | 21 (65.6) | 24 (75.0) | 45 (70.3) | 41 (60.3) | 41 (64.1) | 82 (62.1) |
| Total IgG (IU/mL) | | | | | | |
| Number | 32 | 32 | 64 | 68 | 64 | 132 |
| Mean (SD) | 12.325 | 11.941 (2.805) | 12.133 | 11.546 | 11.643 | 11.593 |
| | (2.582) | | (2.681) | (2.590) | (2.601) | (2.586) |
| Median | 12.210 | 11.645 | 12.020 | 10.985 | 11.385 | 11.205 |
| Q1; Q3 | 10.640; | 9.705; 14.055 | 10.160; | 9.630; | 9.830; 13.120 | 9.650; |
| | 13.490 | | 13.615 | 12.920 | | 13.100 |
| Min; Max | 6.90; | 7.59; 17.90 | 6.90; | 6.63; 19.75 | 6.28; 19.80 | 6.28; 19.80 |
| | 20.46 | | 20.46 | | | |
| Total IgA (IU/mL) | | | | | | |
| Number | 32 | 32 | 64 | 68 | 64 | 132 |
| Mean (SD) | 2543.4 | 2785.9 | 2664.7 | 2510.1 | 2540.5 | 2524.8 |
| | (1226.2) | (1299.6) | (1259.3) | (1108.5) | (1349.0) | (1226.3) |
| Median | 2665.0 | 2440.0 | 2600.0 | 2190.0 | 2175.0 | 2190.0 |
| Q1; Q3 | 1570.0; | 1785.0; | 1695.0; | 1585.0; | 1650.0; | 1640.0; |
| | 3295.0 | 3525.0 | 3470.0 | 3340.0 | 2990.0 | 3240.0 |
| Min; Max | 360; 5640 | 890; 6690 | 360; 6690 | 730; 5100 | 930; 8950 | 730; 8950 |
| Total IgM (IU/mL) | | | | | | |
| Number | 32 | 32 | 64 | | | |
| Mean (SD) | 1.030 | 1.075 (0.579) | 1.053 | 1.189 | 1.121 (0.718) | 1.156 |
| | (0.525) | | (0.549) | (0.683) | | (0.698) |
| Median | 0.955 | 1.025 | 0.990 | 1.005 | 0.895 | 0.950 |
| Q1; Q3 | 0.645; | 0.610; 1.365 | 0.610; | 0.800; | 0.670; 1.360 | 0.730; |
| | 1.285 | | 1.330 | 1.310 | | 1.330 |
| Min; Max | 0.29; 2.40 | 0.26; 2.93 | 0.26; 2.93 | 0.25; 3.66 | 0.30; 3.88 | 0.25; 3.88 |

TABLE 17
Atopic comorbidity history (randomized population).

| | 400 mg BID cohort | | | 400 mg TID cohort | | |
|---|---|---|---|---|---|---|
| | Placebo | Rilzabrutinib | | Placebo | Rilzabrutinib | |
| | BID | 400 mg BID | All | TID | 400 mg TID | All |
| n (%) | (N = 32) | (N = 32) | (N = 64) | (N = 68) | (N = 64) | (N = 132) |
| Any comorbidity | | | | | | |
| history | | | | | | |
| Number | 32 | 32 | 64 | 68 | 64 | 132 |
| Yes | 29 (90.6) | 29 (90.6) | 58 (90.6) | 48 (70.6) | 44 (68.8) | 92 (69.7) |
| Ongoing | 29 (90.6) | 28 (87.5) | 57 (89.1) | 48 (70.6) | 44 (68.8) | 92 (69.7) |
| condition | | | | | | |
| Atopic diseases | | | | | | |
| Number | 32 | 32 | 64 | 68 | 64 | 132 |
| Yes | 17 (53.1) | 20 (62.5) | 37 (57.8) | 24 (35.3) | 21 (32.8) | 45 (34.1) |
| Ongoing | 16 (50.0) | 19 (59.4) | 35 (54.7) | 24 (35.3) | 21 (32.8) | 45 (34.1) |
| condition | | | | | | |
| Atopy and type 2 | | | | | | |
| diseases | | | | | | |
| Number | 32 | 32 | 64 | 68 | 64 | 132 |
| Yes | 29 (90.6) | 29 (90.6) | 58 (90.6) | 48 (70.6) | 44 (68.8) | 92 (69.7) |
| Ongoing | 29 (90.6) | 28 (87.5) | 57 (89.1) | 48 (70.6) | 44 (68.8) | 92 (69.7) |
| condition | | | | | | |
| Atopic dermatitis | | | | | | |
| history | | | | | | |
| Number | 32 | 32 | 64 | 68 | 64 | 132 |
| Yes | 2 (6.3) | 2 (6.3) | 4 (6.3) | 2 (2.9) | 0 | 2 (1.5) |
| Ongoing | 2 (6.3) | 2 (6.3) | 4 (6.3) | 2 (2.9) | 0 | 2 (1.5) |
| condition | | | | | | |
| Allergic | | | | | | |
| conjunctivitis and | | | | | | |
| allergic rhinitis | | | | | | |
| history | | | | | | |
| Number | 32 | 32 | 64 | 68 | 64 | 132 |
| Yes | 4 (12.5) | 3 (9.4) | 7 (10.9) | 2 (2.9) | 2 (3.1) | 4 (3.0) |
| Ongoing | 4 (12.5) | 3 (9.4) | 7 (10.9) | 2 (2.9) | 2 (3.1) | 4 (3.0) |
| condition | | | | | | |
| Allergic | | | | | | |
| conjunctivitis | | | | | | |
| history | | | | | | |
| Number | 32 | 32 | 64 | 68 | 64 | 132 |
| Yes | 4 (12.5) | 3 (9.4) | 7 (10.9) | 2 (2.9) | 2 (3.1) | 4 (3.0) |
| Ongoing | 4 (12.5) | 3 (9.4) | 7 (10.9) | 2 (2.9) | 2 (3.1) | 4 (3.0) |
| condition | | | | | | |
| Allergic rhinitis | | | | | | |
| history | | | | | | |
| Number | 32 | 32 | 64 | 68 | 64 | 132 |
| Yes | 16 (50.0) | 18 (56.3) | 34 (53.1) | 21 (30.9) | 20 (31.3) | 41 (31.1) |
| Ongoing | 15 (46.9) | 17 (53.1) | 32 (50.0) | 21 (30.9) | 20 (31.3) | 41 (31.1) |
| condition | | | | | | |
| Chronic | | | | | | |
| rhinosinusitis | | | | | | |
| history | | | | | | |
| Number | 32 | 32 | 64 | 68 | 64 | 132 |
| Yes | 6 (18.8) | 5 (15.6) | 11 (17.2) | 3 (4.4) | 4 (6.3) | 7 (5.3) |
| Ongoing | 6 (18.8) | 5 (15.6) | 11 (17.2) | 3 (4.4) | 4 (6.3) | 7 (5.3) |
| condition | | | | | | |
| Nasal polyposis | | | | | | |
| history | | | | | | |
| Number | 32 | 32 | 64 | 68 | 64 | 132 |
| Yes | 1 (3.1) | 2 (6.3) | 3 (4.7) | 0 | 1 (1.6) | 1 (0.8) |
| Ongoing | 1 (3.1) | 2 (6.3) | 3 (4.7) | 0 | 1 (1.6) | 1 (0.8) |
| condition | | | | | | |
| Hypersensitivity to | | | | | | |
| aspirin or other | | | | | | |
| NSAID | | | | | | |
| Number | 32 | 32 | 64 | 68 | 64 | 132 |
| Yes | 2 (6.3) | 1 (3.1) | 3 (4.7) | 0 | 2 (3.1) | 2 (1.5) |
| Ongoing | 2 (6.3) | 1 (3.1) | 3 (4.7) | 0 | 2 (3.1) | 2 (1.5) |
| condition | | | | | | |
| Eosinophilic | | | | | | |
| esophagitis history | | | | | | |
| Number | 32 | 32 | 64 | 68 | 64 | 132 |
| Yes | 0 | 0 | 0 | 0 | 0 | 0 |
| Ongoing | 0 | 0 | 0 | 0 | 0 | 0 |
| condition | | | | | | |
| Food allergy | | | | | | |
| history | | | | | | |
| Number | 32 | 32 | 64 | 68 | 64 | 132 |
| Yes | 0 | 0 | 0 | 2 (2.9) | 1 (1.6) | 3 (2.3) |
| Ongoing | 0 | 0 | 0 | 2 (2.9) | 1 (1.6) | 3 (2.3) |
| condition | | | | | | |
| Hives history | | | | | | |
| Number | 32 | 32 | 64 | 68 | 64 | 132 |
| Yes | 1 (3.1) | 2 (6.3) | 3 (4.7) | 0 | 1 (1.6) | 1 (0.8) |
| Ongoing | 1 (3.1) | 2 (6.3) | 3 (4.7) | 0 | 0 | 0 |
| condition | | | | | | |
| Rubber sensitivity | | | | | | |
| Number | 32 | 32 | 64 | 68 | 64 | 132 |
| Yes | 1 (3.1) | 0 | 1 (1.6) | 0 | 0 | 0 |
| Ongoing | 1 (3.1) | 0 | 1 (1.6) | 0 | 0 | 0 |
| condition | | | | | | |

TABLE 18
Prior medications for asthma-number of participants by
predefined categories and standardized medication name (randomized population)

| | 400 mg BID cohort | | | 400 mg TID cohort | | |
|---|---|---|---|---|---|---|
| Predefined categories | Placebo | Rilzabrutinib | | Placebo | Rilzabrutinib | |
| Standardized | BID | 400 mg BID | All | TID | 400 mg TID | All |
| Medication Name n (%) | (N = 32) | (N = 32) | (N = 64) | (N = 68) | (N = 64) | (N = 132) |
| Any prior medications | 32 | 32 (100) | 64 (100) | 68 (100) | 64 (100) | 132 (100) |
| | (100) | | | | | |
| Pre-screening background | 32 | 32 (100) | 64 (100) | 68 (100) | 64 (100) | 132 (100) |
| therapy | (100) | | | | | |
| Salmeterol | 15 | 9 (28.1) | 24 (37.5) | 10 (14.7) | 8 (12.5) | 18 (13.6) |
| | (46.9) | | | | | |
| Fluticasone | 15 | 6 (18.8) | 21 (32.8) | 8 (11.8) | 4 (6.3) | 12 (9.1) |
| | (46.9) | | | | | |
| Fluticasone | 5 (15.6) | 11 (34.4) | 16 (25.0) | 26 (38.2) | 30 (46.9) | 56 (42.4) |
| propionate; salmeterol | | | | | | |
| xinafoate | | | | | | |
| Fluticasone; salmeterol | 4 (12.5) | 6 (18.8) | 10 (15.6) | 1 (1.5) | 3 (4.7) | 4 (3.0) |
| Budesonide; formoterol | 4 (12.5) | 3 (9.4) | 7 (10.9) | 8 (11.8) | 8 (12.5) | 16 (12.1) |
| fumarate | | | | | | |
| Beclometasone | 2 (6.3) | 2 (6.3) | 4 (6.3) | 14 (20.6) | 7 (10.9) | 21 (15.9) |
| dipropionate; formoterol | | | | | | |
| fumarate | | | | | | |
| Budesonide | 2 (6.3) | 2 (6.3) | 4 (6.3) | 3 (4.4) | 2 (3.1) | 5 (3.8) |
| Fluticasone propionate | 1 (3.1) | 3 (9.4) | 4 (6.3) | 3 (4.4) | 4 (6.3) | 7 (5.3) |
| Formoterol | 2 (6.3) | 1 (3.1) | 3 (4.7) | 3 (4.4) | 1 (1.6) | 4 (3.0) |
| Fluticasone | 1 (3.1) | 1 (3.1) | 2 (3.1) | 1 (1.5) | 3 (4.7) | 4 (3.0) |
| furoate; vilanterol | | | | | | |
| trifenatate | | | | | | |
| Beclometasone | 0 | 0 | 0 | 2 (2.9) | 0 | 2 (1.5) |
| dipropionate | | | | | | |
| Beclometasone; formoterol | 0 | 0 | 0 | 2 (2.9) | 2 (3.1) | 4 (3.0) |
| Budesonide; formoterol | 0 | 0 | 0 | 2 (2.9) | 2 (3.1) | 4 (3.0) |
| Formoterol fumarate | 0 | 0 | 0 | 2 (2.9) | 0 | 2 (1.5) |
| Glycopyrronium | 0 | 0 | 0 | 1 (1.5) | 0 | 1 (0.8) |
| bromide; indacaterol | | | | | | |
| acetate; mometasone | | | | | | |
| furoate | | | | | | |
| Systemic steroids | 3 (9.4) | 1 (3.1) | 4 (6.3) | 3 (4.4) | 2 (3.1) | 5 (3.8) |
| Meprednisone | 1 (3.1) | 1 (3.1) | 2 (3.1) | 1 (1.5) | 1 (1.6) | 2 (1.5) |
| Prednisone | 2 (6.3) | 0 | 2 (3.1) | 2 (2.9) | 0 | 2 (1.5) |
| Methylprednisolone | 0 | 0 | 0 | 0 | 1 (1.6) | 1 (0.8) |
| Asthma reliever | 29 | 31 (96.9) | 60 (93.8) | 66 (97.1) | 61 (95.3) | 127 (96.2) |
| | (90.6) | | | | | |
| Salbutamol | 29 | 31 (96.9) | 60 (93.8) | 66 (97.1) | 60 (93.8) | 126 (95.5) |
| | (90.6) | | | | | |
| Salbutamol sulfate | 0 | 0 | 0 | 0 | 1 (1.6) | 1 (0.8) |
| Antihistamines | 3 (9.4) | 2 (6.3) | 5 (7.8) | 1 (1.5) | 0 | 1 (0.8) |
| Fexofenadine | 1 (3.1) | 1 (3.1) | 2 (3.1) | 0 | 0 | 0 |
| hydrochloride | | | | | | |
| Levocetirizine | 2 (6.3) | 0 | 2 (3.1) | 0 | 0 | 0 |
| Azelastine | 0 | 1 (3.1) | 1 (1.6) | 0 | 0 | 0 |
| Desloratadine | 0 | 1 (3.1) | 1 (1.6) | 0 | 0 | 0 |
| Levocetirizine | 0 | 0 | 0 | 1 (1.5) | 0 | 1 (0.8) |
| dihydrochloride | | | | | | |
| Mucolytics | 0 | 2 (6.3) | 2 (3.1) | 0 | 0 | 0 |
| Carbocisteine | 0 | 1 (3.1) | 1 (1.6) | 0 | 0 | 0 |
| Erdosteine | 0 | 1 (3.1) | 1 (1.6) | 0 | 0 | 0 |
| Leukotriene receptor | 1 (3.1) | 1 (3.1) | 2 (3.1) | 0 | 2 (3.1) | 2 (1.5) |
| antagonists or leukotriene | | | | | | |
| synthesis inhibitors | | | | | | |
| Montelukast sodium | 1 (3.1) | 1 (3.1) | 2 (3.1) | 0 | 0 | 0 |
| Azelastine | 0 | 1 (3.1) | 1 (1.6) | 0 | 0 | 0 |
| Montelukast | 0 | 0 | 0 | 0 | 2 (3.1) | 2 (1.5) |
| Anticholinergic | 0 | 0 | 0 | 1 (1.5) | 0 | 1 (0.8) |
| bronchodilators | | | | | | |
| Ipratropium bromide | 0 | 0 | 0 | 1 (1.5) | 0 | 1 (0.8) |
| Other | 1 (3.1) | 0 | 1 (1.6) | 0 | 0 | 0 |
| Doxofylline | 1 (3.1) | 0 | 1 (1.6) | 0 | 0 | 0 |
| Hedera helix extract | 1 (3.1) | 0 | 1 (1.6) | 0 | 0 | 0 |

TABLE 19
Concomitant medications for asthma - number of participants by predefined
categories and standardized medication name (randomized population).

| | 400 mg BID cohort | | 400 mg TID cohort | |
|---|---|---|---|---|
| Predefined categories | | Rilzabrutinib | | Rilzabrutinib |
| Standardized | Placebo BID | 400 mg BID | Placebo TID | 400 mg TID |
| Medication Name n (%) | (N = 32) | (N = 32) | (N = 68) | (N = 64) |
| Any concomitant | 32 | 32 | 68 | 64 |
| medications | (100) | (100) | (100) | (100) |
| Controller | 32 | 32 | 68 | 64 |
| | (100) | (100) | (100) | (100) |
| Fluticasone | 32 | 32 | 68 | 64 |
| | (100) | (100) | (100) | (100) |
| Salmeterol | 32 | 32 | 68 | 64 |
| | (100) | (100) | (100) | (100) |
| Formoterol | 0 | 0 | 1 | 0 |
| | | | (1.5) | |
| Antibiotics | 0 | 3 | 0 | 0 |
| | | (9.4) | | |
| Azithromycin | 0 | 3 | 0 | 0 |
| | | (9.4) | | |
| Leukotriene receptor | 0 | 0 | 0 | 0 |
| antagonists or leukotriene | | | | |
| synthesis inhibitors | | | | |
| Anticholinergic | 0 | 0 | 1 | 0 |
| bronchodilators | | | (1.5) | |
| Ipratropium bromide | 0 | 0 | 1 | 0 |
| | | | (1.5) | |
| Antihistamines and | 3 | 1 | 0 | 0 |
| antiallergics | (9.4) | (3.1) | | |
| Fexofenadine | 1 | 1 | 0 | 0 |
| hydrochloride | (3.1) | (3.1) | | |
| Levocetirizine | 2 | 0 | 0 | 0 |
| | (6.3) | | | |
| Systemic steroid | 4 | 5 | 2 | 1 |
| | (12.5) | (15.6) | (2.9) | (1.6) |
| Prednisone | 1 | 3 | 0 | 0 |
| | (3.1) | (9.4) | | |
| Betamethasone | 0 | 1 | 0 | 0 |
| dipropionate | | (3.1) | | |
| Meprednisone | 2 | 1 | 1 | 1 |
| | (6.3) | (3.1) | (1.5) | (1.6) |
| Methylprednisolone | 1 | 1 | 1 | 0 |
| | (3.1) | (3.1) | (1.5) | |
| Asthma reliever | 29 | 31 | 65 | 60 |
| | (90.6) | (96.9) | (95.6) | (93.8) |
| Salbutamol | 29 | 31 | 65 | 59 |
| | (90.6) | (96.9) | (95.6) | (92.2) |
| Salbutamol sulfate | 0 | 0 | 0 | 1 |
| | | | | (1.6) |
| Mucolytics | 0 | 2 | 0 | 0 |
| | | (6.3) | | |
| Carbocisteine | 0 | 1 | 0 | 0 |
| | | (3.1) | | |
| Erdosteine | 0 | 1 | 0 | 0 |
| | | (3.1) | | |
| Xanthines | 0 | 0 | 1 | 0 |
| | | | (1.5) | |
| Theophylline | 0 | 0 | 1 | 0 |
| | | | (1.5) | |
| PPI | 0 | 0 | 0 | 0 |

n (%) = number and percentage of participants with at least one concomitant medication

### LOAC

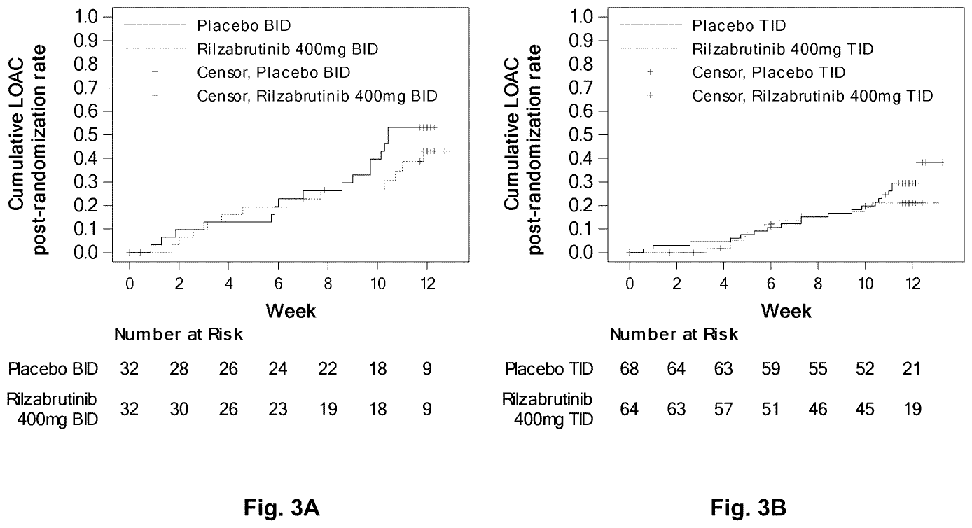

Treatment with rilzabrutinib yielded a numerical reduction in LOAC events relative to treatment with the placebo for patients in both the 400 mg BID and 400 mg TID cohorts (FIG. 3 and Tables 20-28). Although 50% of placebo-treated patients in the 400 mg BID cohort experienced LOAC during the treatment period, only 37.5% of rilzabrutinib-treated patients in the 400 mg BID cohort experienced LOAC (Table 20). Similar results were observed for the 400 mg TID cohort, in which 29.4% of placebo-treated patients and 18.8% of rilzabrutinib-treated patients experienced LOAC (Table 20). This represents a relative risk reduction of 25% and 36%, for the 400 mg BID and 400 mg TID cohorts, respectively.

Subgroup analysis further supports the efficacy of rilzabrutinib treatment in both cohorts. In general, patients receiving 400 mg BID or 400 mg TID rilzabrutinib exhibited a numerical improvement in the incidence of LOAC events regardless of baseline demographic characteristics, disease characteristics, ICS/LABA dose level, biomarker levels, eosinophil count, and FeNO (ppb) (Tables 24-28).

TABLE 20
Primary analysis: incidence of LOAC (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| | (N = 32) | (N = 32) | (N = 68) | (N = 64) |
| Number of | | | | |
| participants | | | | |
| with LOAC | | | | |
| Number | 32 | 32 | 68 | 64 |
| No | 16 | 20 | 48 | 52 |
| | (50.0) | (62.5) | (70.6) | (81.3) |
| Yes | 16 | 12 | 20 | 12 |
| | (50.0) | (37.5) | (29.4) | (18.8) |
| Crude RRR vs. | | 25.0 | | 36.1 |
| placebo | | | | |
| OR vs. placebo | | 0.570 | | 0.584 |
| (95% CI) | | (0.202, 1.608) | | (0.253, 1.349) |
| P-value vs. | | 0.2880 | | 0.2083 |
| placebo | | | | |
| RD vs. Placebo | | −0.123 | | −0.088 |
| (95% CI) | | (−0.361, 0.115) | | (−0.238, 0.061) |
| RR vs. placebo | | 0.756 | | 0.653 |
| (95% CI) | | (0.433, 1.319) | | (0.348, 1.226) |
| RRR vs. placebo | | 24.4 | | 34.7 |
| (%) | | | | |

LOAC: Loss of asthma control, OR: Odds ratio, RD: Risk difference, LATAM: Latin America, ROW: Rest of the world

TABLE 21
Critical or major protocol deviations potentially impact
on the primary endpoint (randomized population).

| | 400 mg BID cohort | | 400 mg TID cohort | |
|---|---|---|---|---|
| Deviation category | | Rilzabrutinib | | Rilzabrutinib |
| Deviation term | Placebo BID | 400 mg BID | Placebo TID | 400 mg TID |
| n (%) | (N = 32) | (N = 32) | (N = 68) | (N = 64) |
| Any critical or major protocol | 2 | 1 | 2 | 3 |
| deviation with potential impact | (6.3) | (3.1) | (2.9) | (4.7) |
| on the primary endpoint | | | | |
| Inclusion/exclusion criteria | 0 | 0 | 1 | 1 |
| | | | (1.5) | (1.6) |
| Asthma Control Questionnaire | 0 | 0 | 1 | 0 |
| 5-question version (ACQ-5) | | | (1.5) | |
| score of >4 during the screening | | | | |
| period or <1.25 or >3.0 at V2. | | | | |
| Participants with | 0 | 0 | 0 | 1 |
| prebronchodilator FEV1 > 40% | | | | (1.6) |
| of predicted normal at V1. | | | | |
| Prebronchodilator FEV1 >= | | | | |
| 50% but <= 85% of predicted | | | | |
| normal at V2. | | | | |
| Concomitant medications/ | 2 | 1 | 1 | 2 |
| therapy | (6.3) | (3.1) | (1.5) | (3.1) |
| NIMP administered but not as | 2 | 1 | 1 | 2 |
| per protocol | (6.3) | (3.1) | (1.5) | (3.1) |

TABLE 22
Summary of LOAC events (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| | (N = 32) | (N = 32) | (N = 68) | (N = 64) |
| Number of participants | | | | |
| with LOAC | | | | |
| Number | 32 | 32 | 68 | 64 |
| No | 16 | 20 | 48 | 52 |
| | (50.0) | (62.5) | (70.6) | (81.3) |
| Yes | 16 | 12 | 20 | 12 |
| | (50.0) | (37.5) | (29.4) | (18.8) |
| Triggered by use of | 8 | 6 | 9 | 1 |
| rescue medication | (25.0) | (18.8) | (13.2) | (1.6) |
| Number of participants | 4 | 2 | 7 | 0 |
| with ≥6 additional | (12.5) | (6.3) | (10.3) | |
| reliever puffs of | | | | |
| salbutamol/albuterol | | | | |
| or levosalbutamol/ | | | | |
| levalbuterol in a | | | | |
| 24 hour period | | | | |
| (compared with | | | | |
| baseline) on 2 | | | | |
| consecutive days | | | | |
| Number of participants | 0 | 1 | 0 | 0 |
| with increase in ICS ≥4 | | (3.1) | | |
| times the last prescribed | | | | |
| ICS dose (or ≥50% of | | | | |
| the prescribed ICS dose | | | | |
| at baseline if background | | | | |
| therapy withdrawal | | | | |
| completed) | | | | |
| Number of participants | 4 | 4 | 2 | 1 |
| requiring use of systemic | (12.5) | (12.5) | (2.9) | (1.6) |
| (oral and/or parenteral) | | | | |
| steroid treatment | | | | |
| Not triggered by use of | 8 | 6 | 10 | 10 |
| rescue medication | (25.0) | (18.8) | (14.7) | (15.6) |
| Number of participants | 8 | 6 | 10 | 10 |
| with ≥30% reduction | (25.0) | (18.8) | (14.7) | (15.6) |
| from baseline in AM PEF | | | | |
| on 2 consecutive days | | | | |
| Number of participants | 0 | 0 | 0 | 0 |
| requiring hospitalization | | | | |
| or emergency room visit | | | | |
| Intervention discontinuation | 0 | 0 | 1 | 1 |
| due to lack of efficacy or | | | (1.5) | (1.6) |
| an AE related to asthma | | | | |
| worsening | | | | |

TABLE 23
Supportive analysis: time to LOAC post-randomization (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| | (N = 32) | (N = 32) | (N = 68) | (N = 64) |
| Number of | 16 | 12 | 20 | 12 |
| participants with | (50.0) | (37.5) | (29.4) | (18.8) |
| LOAC | | | | |
| Number of | 16 | 20 | 48 | 52 |
| participants | (50.0) | (62.5) | (70.6) | (81.3) |
| censored | | | | |
| Q1 of time to LOAC | 50.0 | 55.0 | 78.0 | NC |
| (days) (95% CI) | (14.0, 72.0) | (19.0, 84.0) | (52.0, NC) | (44.0, NC) |
| Median time to | 74.0 | NC | NC | NC |
| LOAC (days) (95% | (64.0, NC) | (73.0, NC) | (87.0, NC) | (NC, NC) |
| CI) | | | | |
| Q3 of time to LOAC | NC | NC | NC | NC |
| (days) (95% CI) | (NC, NC) | (NC, NC) | (NC, NC) | (NC, NC) |
| Probability of | | | | |
| LOAC (95% CI) at | | | | |
| Week 4 | 0.129 | 0.161 | 0.045 | 0.017 |
| | (0.041, 0.270) | (0.059, 0.309) | (0.012, 0.115) | (0.001, 0.080) |
| Week 9 | 0.330 | 0.265 | 0.168 | 0.156 |
| | (0.173, 0.496) | (0.125, 0.429) | (0.089, 0.267) | (0.077, 0.261) |
| Week 12 | 0.531 | 0.432 | 0.295 | 0.211 |
| | (0.341, 0.689) | (0.243, 0.607) | (0.189, 0.408) | (0.117, 0.325) |
| HR vs. Placebo | | 0.750 | | 0.679 |
| (95% CI) | | (0.352, 1.595) | | (0.327, 1.408) |
| P-value vs. placebo | | 0.4543 | | 0.2980 |
| P-value vs. placebo | | 0.3948 | | 0.2794 |

NE: Not able to estimate, LATAM: Latin America, ROW: Rest of the world

TABLE 24
Subgroup analysis: incidence of LOAC by
demographics subgroups (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| | (N = 32) | (N = 32) | (N = 68) | (N = 64) |
| Age group 1 (years) | | | | |
| <45 | | | | |
| Number | 10 | 12 | 29 | 25 |
| LOAC | 3 | 6 | 11 | 2 |
| | (30.0) | (50.0) | (37.9) | (8.0) |
| OR vs. placebo | | 2.489 | | 0.142 |
| (95% CI) | | (0.342, 18.129) | | (0.028, 0.725) |
| P-value vs. placebo | | 0.3680 | | 0.0189 |
| RD vs. Placebo | | 0.184 | | −0.299 |
| (95% CI) | | (−0.266, 0.634) | | (−0.505, −0.093) |
| ≥45 | | | | |
| Number | 22 | 20 | 39 | 39 |
| LOAC | 13 | 6 | 9 | 10 |
| | (59.1) | (30.0) | (23.1) | (25.6) |
| OR vs. placebo | | 0.266 | | 1.149 |
| (95% CI) | | (0.069, 1.023) | | (0.408, 3.236) |
| P-value vs. placebo | | 0.0540 | | 0.7920 |
| RD vs. Placebo | | −0.295 | | 0.026 |
| (95% CI) | | (−0.583, −0.007) | | (−0.165, 0.216) |
| Overall p-value for | | 0.0738 | | 0.0298 |
| interaction | | | | |
| Gender | | | | |
| Male | | | | |
| Number | 8 | 14 | 27 | 25 |
| LOAC | 2 | 5 | 7 | 4 |
| | (25.0) | (35.7) | (25.9) | (16.0) |
| OR vs. placebo | | 2.062 | | 0.423 |
| (95% CI) | | (0.173, 24.590) | | (0.091, 1.966) |
| P-value vs. placebo | | 0.5671 | | 0.2721 |
| RD vs. Placebo | | 0.256 | | −0.111 |
| (95% CI) | | (−0.420, 0.931) | | (−0.461, 0.238) |
| Female | | | | |
| Number | 24 | 18 | 41 | 39 |
| LOAC | 14 | 7 | 13 | 8 |
| | (58.3) | (38.9) | (31.7) | (20.5) |
| OR vs. placebo | | 0.311 | | 0.640 |
| (95% CI) | | (0.076, 1.263) | | (0.224, 1.828) |
| P-value vs. placebo | | 0.1023 | | 0.4042 |
| RD vs. Placebo | | −0.258 | | −0.084 |
| (95% CI) | | (−0.560, 0.045) | | (−0.291, 0.123) |
| Overall p-value for | | 0.1040 | | 0.9230 |
| interaction | | | | |
| Baseline weight | | | | |
| group (kg) | | | | |
| <60 | | | | |
| Number | 4 | 3 | 12 | 2 |
| LOAC | 4 | 0 | 3 | 1 |
| | (100) | | (25.0) | (50.0) |
| OR vs. placebo | | NC | | 3.000 |
| (95% CI) | | (NC, NC) | | (0.140, 64.262) |
| P-value vs. placebo | | NC | | 0.4823 |
| RD vs. Placebo | | NC | | 0.250 |
| (95% CI) | | (NC, NC) | | (−0.485, 0.985) |
| ≥60-<90 | | | | |
| Number | 25 | 23 | 40 | 41 |
| LOAC | 10 | 10 | 13 | 8 |
| | (40.0) | (43.5) | (32.5) | (19.5) |
| OR vs. placebo | | 1.154 | | 0.503 |
| (95% CI) | | (0.366, 3.640) | | (0.182, 1.392) |
| P-value vs. placebo | | 0.8071 | | 0.1860 |
| RD vs. Placebo | | 0.035 | | −0.130 |
| (95% CI) | | (−0.244, 0.314) | | (−0.319, 0.059) |
| ≥90 | | | | |
| Number | 3 | 6 | 16 | 21 |
| LOAC | 2 | 2 | 4 | 3 |
| | (66.7) | (33.3) | (25.0) | (14.3) |
| OR vs. placebo | | 0.250 | | 0.500 |
| (95% CI) | | (0.013, 4.729) | | (0.095, 2.645) |
| P-value vs. placebo | | 0.3554 | | 0.4147 |
| RD vs. Placebo | | −0.333 | | −0.107 |
| (95% CI) | | (−0.987, 0.320) | | (−0.367, 0.153) |
| Overall p-value for | | 0.0116 | | 0.4929 |
| interaction | | | | |
| Baseline BMI group | | | | |
| (kg/m²) | | | | |
| <25 | | | | |
| Number | 6 | 8 | 22 | 12 |
| LOAC | 4 | 3 | 8 | 3 |
| | (66.7) | (37.5) | (36.4) | (25.0) |
| OR vs. placebo | | 0.300 | | 0.583 |
| (95% CI) | | (0.033, 2.763) | | (0.121, 2.801) |
| P-value vs. placebo | | 0.2879 | | 0.5008 |
| RD vs. Placebo | | −0.292 | | −0.114 |
| (95% CI) | | (−0.796, 0.213) | | (−0.431, 0.203) |
| ≥25-<30 | | | | |
| Number | 17 | 16 | 27 | 29 |
| LOAC | 8 | 6 | 8 | 3 |
| | (47.1) | (37.5) | (29.6) | (10.3) |
| OR vs. placebo | | 0.675 | | 0.274 |
| (95% CI) | | (0.168, 2.709) | | (0.064, 1.172) |
| P-value vs. placebo | | 0.5794 | | 0.0807 |
| RD vs. Placebo | | −0.096 | | −0.193 |
| (95% CI) | | (−0.431, 0.240) | | (−0.398, 0.012) |
| ≥30 | | | | |
| Number | 9 | 8 | 19 | 23 |
| LOAC | 4 | 3 | 4 | 6 |
| | (44.4) | (37.5) | (21.1) | (26.1) |
| OR vs. placebo | | 0.750 | | 1.324 |
| (95% CI) | | (0.107, 5.238) | | (0.313, 5.604) |
| P-value vs. placebo | | 0.7717 | | 0.7034 |
| RD vs. Placebo | | −0.069 | | 0.050 |
| (95% CI) | | (−0.536, 0.397) | | (−0.206, 0.307) |
| P-value vs. placebo | | 0.7706 | | 0.2506 |
| Region | | | | |
| Eastern Europe | | | | |
| Number | 4 | 3 | 45 | 43 |
| LOAC | 2 | 1 | 14 | 8 |
| | (50.0) | (33.3) | (31.1) | (18.6) |
| OR vs. placebo | | 0.500 | | 0.506 |
| (95% CI) | | (0.023, 11.088) | | (0.187, 1.368) |
| P-value vs. placebo | | 0.6611 | | 0.1794 |
| RD vs. Placebo | | −0.167 | | −0.125 |
| (95% CI) | | (−0.891, 0.558) | | (−0.303, 0.053) |
| Latin America | | | | |
| Number | 25 | 25 | 19 | 19 |
| LOAC | 11 | 9 | 5 | 4 |
| | (44.0) | (36.0) | (26.3) | (21.1) |
| OR vs. placebo | | 0.716 | | 0.747 |
| (95% CI) | | (0.230, 2.230) | | (0.166, 3.357) |
| P-value vs. placebo | | 0.5642 | | 0.7032 |
| RD vs. Placebo | | −0.080 | | −0.053 |
| (95% CI) | | (−0.351, 0.191) | | (−0.322, 0.217) |
| Western Countries | | | | |
| Number | 1 | 2 | 2 | 2 |
| LOAC | 1 | 0 | 1 | 0 |
| | (100) | | (50.0) | |
| OR vs. placebo | | NC | | NC |
| (95% CI) | | (NC, NC) | | (NC, NC) |
| P-value vs. placebo | | NC | | NC |
| RD vs. Placebo | | NC | | NC |
| (95% CI) | | (NC, NC) | | (NC, NC) |
| APAC | | | | |
| Number | 2 | 2 | 2 | 0 |
| LOAC | 2 | 2 | 0 | 0 |
| | (100) | (100) | | |
| OR vs. placebo | | NC | | NC |
| (95% CI) | | (NC, NC) | | (NC, NC) |
| P-value vs. placebo | | NC | | NC |
| RD vs. Placebo | | NC | | NC |
| (95% CI) | | (NC, NC) | | (NC, NC) |
| Overall p-value for | | NC | | NC |
| interaction | | | | |

LATAM: Latin America, ROW: Rest of the world

TABLE 25
Subgroup analysis: incidence of LOAC by disease and other characteristics subgroups (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| | BID | | 400 mg BID | | TID | | 400 mg TID | |
| | (N = 32) | | (N = 32) | | (N = 68) | | (N = 64) | |
| Age at onset of asthma (years) | | | | | | | | |
| <12 | | | | | | | | |
| Number | 7 | | 9 | | 15 | | 14 | |
| LOAC | 2 | (28.6) | 4 | (44.4) | 6 | (40.0) | 2 | (14.3) |
| OR vs. placebo (95% CI) | | | 2.000 | (0.244, 16.362) | | | 0.250 | (0.041, 1.541) |
| P-value vs. placebo | | | 0.5180 | | | | 0.1352 | |
| RD vs. Placebo (95% CI) | | | 0.159 | (−0.308, 0.625) | | | −0.257 | (−0.565, 0.051) |
| ≥12-<18 | | | | | | | | |
| Number | 2 | | 5 | | 5 | | 5 | |
| LOAC | 1 | (50.0) | 1 | (20.0) | 1 | (20.0) | 0 | |
| OR vs. placebo (95% CI) | | | 0.250 | (0.007, 8.560) | | | NC | (NC, NC) |
| P-value vs. placebo | | | 0.4419 | | | | NC | |
| RD vs. Placebo (95% CI) | | | −0.300 | (−1.077, 0.477) | | | NC | (NC, NC) |
| ≥18-<40 | | | | | | | | |
| Number | 15 | | 14 | | 28 | | 27 | |
| LOAC | 9 | (60.0) | 4 | (28.6) | 10 | (35.7) | 6 | (22.2) |
| OR vs. placebo (95% CI) | | | 0.267 | (0.056, 1.260) | | | 0.514 | (0.156, 1.694) |
| P-value vs. placebo | | | 0.0953 | | | | 0.2742 | |
| RD vs. Placebo (95% CI) | | | −0.314 | (−0.657, 0.028) | | | −0.135 | (−0.372, 0.102) |
| ≥40 | | | | | | | | |
| Number | 8 | | 4 | | 20 | | 18 | |
| LOAC | 4 | (50.0) | 3 | (75.0) | 3 | (15.0) | 4 | (22.2) |
| OR vs. placebo (95% CI) | | | 3.000 | (0.211, 42.624) | | | 1.619 | (0.309, 8.478) |
| P-value vs. placebo | | | 0.4171 | | | | 0.5684 | |
| RD vs. Placebo (95% CI) | | | 0.250 | (−0.298, 0.798) | | | 0.072 | (−0.176, 0.320) |
| Overall P-value for interaction | | | 0.1351 | | | | 0.3189 | |
| Age at onset of asthma (years) | | | | | | | | |
| <18 | | | | | | | | |
| Number | 9 | | 14 | | 20 | | 19 | |
| LOAC | 3 | (33.3) | 5 | (35.7) | 7 | (35.0) | 2 | (10.5) |
| OR vs. placebo (95% CI) | | | 1.402 | (0.206, 9.530) | | | 0.218 | (0.039, 1.232) |
| P-value vs. placebo | | | 0.7295 | | | | 0.0847 | |
| RD vs. Placebo (95% CI) | | | 0.126 | (−0.346, 0.597) | | | −0.245 | (−0.495, 0.006) |
| ≥18 | | | | | | | | |
| Number | 23 | | 18 | | 48 | | 45 | |
| LOAC | 13 | (56.5) | 7 | (38.9) | 13 | (27.1) | 10 | (22.2) |
| OR vs. placebo (95% CI) | | | 0.323 | (0.079, 1.322) | | | 0.769 | (0.298, 1.986) |
| P-value vs. placebo | | | 0.1160 | | | | 0.5876 | |
| RD vs. Placebo (95% CI) | | | −0.258 | (−0.560, 0.045) | | | −0.049 | (−0.223, 0.126) |
| Overall P-value for interaction | | | 0.2794 | | | | 0.1729 | |
| Number of asthma | | | | | | | | |
| exacerbations within | | | | | | | | |
| 2 years before | | | | | | | | |
| screening visit | | | | | | | | |
| 1 | | | | | | | | |
| Number | 9 | | 13 | | 51 | | 36 | |
| LOAC | 3 | (33.3) | 5 | (38.5) | 17 | (33.3) | 5 | (13.9) |
| OR vs. placebo (95% CI) | | | 1.218 | (0.176, 8.423) | | | 0.323 | (0.106, 0.978) |
| P-value vs. placebo | | | 0.8416 | | | | 0.0457 | |
| RD vs. Placebo (95% CI) | | | 0.059 | (−0.390, 0.508) | | | −0.194 | (−0.366, −0.023) |
| 2 | | | | | | | | |
| Number | 15 | | 11 | | 16 | | 23 | |
| LOAC | 9 | (60.0) | 4 | (36.4) | 3 | (18.8) | 7 | (30.4) |
| OR vs. placebo (95% CI) | | | 0.471 | (0.084, 2.634) | | | 1.896 | (0.407, 8.824) |
| P-value vs. placebo | | | 0.3917 | | | | 0.4149 | |
| RD vs. Placebo (95% CI) | | | −0.199 | (−0.617, 0.219) | | | 0.117 | (−0.151, 0.385) |
| >2 | | | | | | | | |
| Number | 8 | | 8 | | 1 | | 5 | |
| LOAC | 4 | (50.0) | 3 | (37.5) | 0 | | 0 | |
| OR vs. placebo (95% CI) | | | 0.508 | (0.044, 5.914) | | | NC | (NC, NC) |
| P-value vs. placebo | | | 0.5890 | | | | NC | |
| RD vs. Placebo (95% CI) | | | −0.115 | (−0.780, 0.551) | | | NC | (NC, NC) |
| Overall P-value for interaction | | | 0.8727 | | | | NC | |
| Atopic medical conditions | | | | | | | | |
| Yes | | | | | | | | |
| Number | 17 | | 20 | | 24 | | 21 | |
| LOAC | 8 | (47.1) | 8 | (40.0) | 6 | (25.0) | 4 | (19.0) |
| OR vs. placebo (95% CI) | | | 0.681 | (0.174, 2.666) | | | 0.821 | (0.175, 3.846) |
| P-value vs. placebo | | | 0.5815 | | | | 0.8026 | |
| RD vs. Placebo (95% CI) | | | −0.069 | (−0.384, 0.246) | | | −0.031 | (−0.320, 0.257) |
| No | | | | | | | | |
| Number | 15 | | 12 | | 44 | | 43 | |
| LOAC | 8 | (53.3) | 4 | (33.3) | 14 | (31.8) | 8 | (18.6) |
| OR vs. placebo (95% CI) | | | 0.380 | (0.065, 2.206) | | | 0.493 | (0.180, 1.353) |
| P-value vs. placebo | | | 0.2809 | | | | 0.1700 | |
| RD vs. Placebo (95% CI) | | | −0.204 | (−0.557, 0.149) | | | −0.130 | (−0.311, 0.051) |
| Overall P-value for interaction | | | 0.6695 | | | | 0.6159 | |
| Background ICS dose | | | | | | | | |
| level at randomization | | | | | | | | |
| (400 mg TID only) | | | | | | | | |
| Medium | | | | | | | | |
| Number | | | | | 35 | | 29 | |
| LOAC | | | | | 14 | (40.0) | 6 | (20.7) |
| OR vs. placebo (95% CI) | | | | | | | 0.421 | (0.125, 1.414) |
| P-value vs. placebo | | | | | | | 0.1615 | |
| RD vs. placebo (95% CI) | | | | | | | −0.173 | (−0.407, 0.061) |
| High | | | | | | | | |
| Number | | | | | 33 | | 35 | |
| LOAC | | | | | 6 | (18.2) | 6 | (17.1) |
| OR vs. placebo (95% CI) | | | | | | | 0.911 | (0.253, 3.276) |
| P-value vs. placebo | | | | | | | 0.8867 | |
| RD vs. placebo (95% CI) | | | | | | | −0.011 | (−0.218, 0.195) |
| Overall P-value for interaction | | | | | | | 0.3913 | |
| Smoking history | | | | | | | | |
| Former | | | | | | | | |
| Number | 8 | | 4 | | 6 | | 6 | |
| LOAC | 4 | (50.0) | 3 | (75.0) | 3 | (50.0) | 2 | (33.3) |
| OR vs. placebo (95% CI) | | | 3.000 | (0.211, 42.624) | | | 0.500 | (0.049, 5.154) |
| P-value vs. placebo | | | 0.4171 | | | | 0.5603 | |
| RD vs. Placebo (95% CI) | | | 0.250 | (−0.298, 0.798) | | | −0.167 | (−0.717, 0.383) |
| Never | | | | | | | | |
| Number | 24 | | 28 | | 62 | | 58 | |
| LOAC | 12 | (50.0) | 9 | (32.1) | 17 | (27.4) | 10 | (17.2) |
| OR vs. placebo (95% CI) | | | 0.474 | (0.154, 1.461) | | | 0.551 | (0.229, 1.330) |
| P-value vs. placebo | | | 0.1936 | | | | 0.1853 | |
| RD vs. Placebo (95% CI) | | | −0.179 | (−0.443, 0.086) | | | −0.102 | (−0.249, 0.046) |
| Overall P-value for interaction | | | 0.2175 | | | | 0.7814 | |
| Baseline pre-bronchodilator | | | | | | | | |
| FEV1 (L) (400 mg BID only) | | | | | | | | |
| <Median (2.022) | | | | | | | | |
| Number | 14 | | 17 | | | | | |
| LOAC | 7 | (50.0) | 7 | (41.2) | | | | |
| OR vs. placebo (95% CI) | | | 0.700 | (0.168, 2.910) | | | | |
| P-value vs. placebo | | | 0.6237 | | | | | |
| RD vs. Placebo (95% CI) | | | −0.088 | (−0.439, 0.263) | | | | |
| P-value vs. placebo | | | 0.6224 | | | | | |
| ≥Median (2.022) | | | | | | | | |
| Number | 18 | | 14 | | | | | |
| LOAC | 9 | (50.0) | 5 | (35.7) | | | | |
| OR vs. placebo (95% CI) | | | 0.556 | (0.133, 2.325) | | | | |
| P-value vs. placebo | | | 0.4209 | | | | | |
| RD vs. Placebo (95% CI) | | | −0.143 | (−0.484, 0.198) | | | | |
| P-value vs. placebo | | | 0.4117 | | | | | |
| Overall P-value for interaction | | | 0.7743 | | | | | |
| Baseline pre-bronchodilator | | | | | | | | |
| percent predicted FEV1 (%) | | | | | | | | |
| (400 mg BID only) | | | | | | | | |
| <Median (68) | | | | | | | | |
| Number | 12 | | 17 | | | | | |
| LOAC | 6 | (50.0) | 7 | (41.2) | | | | |
| OR vs. placebo (95% CI) | | | 0.700 | (0.158, 3.099) | | | | |
| P-value vs. placebo | | | 0.6384 | | | | | |
| RD vs. Placebo (95% CI) | | | −0.088 | (−0.455, 0.279) | | | | |
| P-value vs. placebo | | | 0.6376 | | | | | |
| ≥ Median (68) | | | | | | | | |
| Number | 20 | | 14 | | | | | |
| LOAC | 10 | (50.0) | 5 | (35.7) | | | | |
| OR vs. placebo (95% CI) | | | 0.556 | (0.137, 2.256) | | | | |
| P-value vs. placebo | | | 0.4110 | | | | | |
| RD vs. Placebo (95% CI) | | | −0.143 | (−0.476, 0.190) | | | | |
| P-value vs. placebo | | | 0.4007 | | | | | |
| Overall P-value for interaction | | | 0.5557 | | | | | |
| Baseline pre-bronchodilator | | | | | | | | |
| FEV1 (L) (400 mg TID only) | | | | | | | | |
| <Median (2.1955) | | | | | | | | |
| Number | | | | | 36 | | 29 | |
| LOAC | | | | | 10 | (27.8) | 7 | (24.1) |
| OR vs. placebo (95% CI) | | | | | | | 0.827 | (0.270, 2.536) |
| P-value vs. placebo | | | | | | | 0.7401 | |
| RD vs. placebo (95% CI) | | | | | | | −0.036 | (−0.250, 0.177) |
| ≥Median (2.1955) | | | | | | | | |
| Number | | | | | 31 | | 34 | |
| LOAC | | | | | 10 | (32.3) | 5 | (14.7) |
| OR vs. placebo (95% CI) | | | | | | | 0.362 | (0.108, 1.216) |
| P-value vs. placebo | | | | | | | 0.1003 | |
| RD vs. placebo (95% CI) | | | | | | | −0.176 | (−0.379, 0.028) |
| Overall P-value for interaction | | | | | | | 0.3196 | |
| Baseline pre-bronchodilator | | | | | | | | |
| percent predicted | | | | | | | | |
| FEV1 group 1 (%) | | | | | | | | |
| (400 mg TID only) | | | | | | | | |
| <Median (77) | | | | | | | | |
| Number | | | | | 36 | | 29 | |
| LOAC | | | | | 9 | (25.0) | 4 | (13.8) |
| OR vs. placebo (95% CI) | | | | | | | 0.452 | (0.118, 1.730) |
| P-value vs. placebo | | | | | | | 0.2464 | |
| RD vs. placebo (95% CI) | | | | | | | −0.129 | (−0.394, 0.135) |
| ≥Median (77) | | | | | | | | |
| Number | | | | | 32 | | 34 | |
| LOAC | | | | | 11 | (34.4) | 8 | (23.5) |
| OR vs. placebo (95% CI) | | | | | | | 0.599 | (0.185, 1.937) |
| P-value vs. placebo | | | | | | | 0.3916 | |
| RD vs. placebo (95% CI) | | | | | | | −0.078 | (−0.320, 0.165) |
| Overall P-value for interaction | | | | | | | 0.6831 | |
| Baseline pre-bronchodilator | | | | | | | | |
| percent predicted | | | | | | | | |
| FEV1 group 2 (%) | | | | | | | | |
| (400 mg TID only) | | | | | | | | |
| <80 | | | | | | | | |
| Number | | | | | 41 | | 37 | |
| LOAC | | | | | 10 | (24.4) | 7 | (18.9) |
| OR vs. placebo (95% CI) | | | | | | | 0.736 | (0.243, 2.229) |
| P-value vs. placebo | | | | | | | 0.5884 | |
| RD vs. placebo (95% CI) | | | | | | | −0.047 | (−0.244, 0.149) |
| ≥80 | | | | | | | | |
| Number | | | | | 27 | | 26 | |
| LOAC | | | | | 10 | (37.0) | 5 | (19.2) |
| OR vs. placebo (95% CI) | | | | | | | 0.429 | (0.112, 1.648) |
| P-value vs. placebo | | | | | | | 0.2176 | |
| RD vs. placebo (95% CI) | | | | | | | −0.139 | (−0.406, 0.128) |
| Overall P-value for interaction | | | | | | | 0.5675 | |
| Baseline FEV1 | | | | | | | | |
| reversibility (%) | | | | | | | | |
| <12 | | | | | | | | |
| Number | 21 | | 14 | | 48 | | 43 | |
| LOAC | 10 | (47.6) | 5 | (35.7) | 14 | (29.2) | 9 | (20.9) |
| OR vs. placebo (95% CI) | | | 0.611 | (0.152, 2.450) | | | 0.643 | (0.245, 1.684) |
| P-value vs. placebo | | | 0.4870 | | | | 0.3685 | |
| RD vs. Placebo (95% CI) | | | −0.119 | (−0.449, 0.211) | | | −0.082 | (−0.259, 0.095) |
| ≥12 | | | | | | | | |
| Number | 9 | | 15 | | 12 | | 13 | |
| LOAC | 5 | (55.6) | 6 | (40.0) | 2 | (16.7) | 1 | (7.7) |
| OR vs. placebo (95% CI) | | | 0.533 | (0.100, 2.839) | | | 0.417 | (0.033, 5.299) |
| P-value vs. placebo | | | 0.4612 | | | | 0.4998 | |
| RD vs. Placebo (95% CI) | | | −0.156 | (−0.564, 0.253) | | | −0.090 | (−0.346, 0.166) |
| Overall P-value for interaction | | | 0.8792 | | | | 0.7503 | |
| Baseline ACQ-5 score | | | | | | | | |
| ≤2 | | | | | | | | |
| Number | 15 | | 19 | | 29 | | 24 | |
| LOAC | 7 | (46.7) | 7 | (36.8) | 7 | (24.1) | 6 | (25.0) |
| OR vs. placebo (95% CI) | | | 0.651 | (0.153, 2.769) | | | 1.162 | (0.297, 4.543) |
| P-value vs. placebo | | | 0.5608 | | | | 0.8289 | |
| RD vs. Placebo (95% CI) | | | −0.098 | (−0.429, 0.233) | | | 0.025 | (−0.209, 0.259) |
| >2 | | | | | | | | |
| Number | 17 | | 13 | | 39 | | 40 | |
| LOAC | 9 | (52.9) | 5 | (38.5) | 13 | (33.3) | 6 | (15.0) |
| OR vs. placebo (95% CI) | | | 0.475 | (0.090, 2.502) | | | 0.346 | (0.113, 1.060) |
| P-value vs. placebo | | | 0.3800 | | | | 0.0632 | |
| RD vs. Placebo (95% CI) | | | −0.125 | (−0.473, 0.224) | | | −0.181 | (−0.379, 0.018) |

| | | |
|---|---|---|
| Overall P-value for interaction | 0.7235 | 0.1501 |

LATAM: Latin America,
ROW: Rest of the world

TABLE 26
Subgroup analysis: incidence of LOAC by baseline
ICS/LABA dose level subgroups (mITT population).

| | Placebo | | Rilzabrutinib | |
|---|---|---|---|---|
| | BID | | 400 mg BID | |
| | (N = 32) | | (N = 32) | |
| Background ICS/LABA | | | | |
| dose level at | | | | |
| randomization | | | | |
| Medium | | | | |
| Number | 3 | | 5 | |
| LOAC | 2 | (66.7) | 1 | (20.0) |
| OR vs. placebo (95% CI) | | | 0.125 | (0.005, 3.225) |
| P-value vs. placebo | | | 0.2099 | |
| RD vs. Placebo (95% CI) | | | −0.467 | (−1.105, 0.172) |
| P-value vs. placebo | | | 0.1519 | |
| High | | | | |
| Number | 29 | | 27 | |
| LOAC | 14 | (48.3) | 11 | (40.7) |
| OR vs. placebo (95% CI) | | | 0.737 | (0.256, 2.122) |
| P-value vs. placebo | | | 0.5713 | |
| RD vs. Placebo (95% CI) | | | −0.075 | (−0.335, 0.184) |

| | |
|---|---|
| P-value vs. placebo | 0.5695 |
| Overall P-value for interaction | 0.3273 |

LATAM: Latin America,
ROW: Rest of the world

TABLE 27
Subgroup analysis: incidence of LOAC by baseline biomarker subgroups (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| | BID | | 400 mg BID | | TID | | 400 mg TID | |
| | (N = 32) | | (N = 32) | | (N = 68) | | (N = 64) | |
| Baseline IgE level (IU/mL) | | | | | | | | |
| <100 | | | | | | | | |
| Number | 11 | | 8 | | 27 | | 23 | |
| LOAC | 5 | (45.5) | 2 | (25.0) | 7 | (25.9) | 4 | (17.4) |
| OR vs. placebo (95% CI) | | | 0.412 | (0.053, 3.217) | | | 0.602 | (0.151, 2.390) |
| P-value vs. placebo | | | 0.3977 | | | | 0.4702 | |
| RD vs. Placebo (95% CI) | | | −0.220 | (−0.676, 0.236) | | | −0.085 | (−0.312, 0.141) |
| ≥100 | | | | | | | | |
| Number | 21 | | 24 | | 41 | | 41 | |
| LOAC | 11 | (52.4) | 10 | (41.7) | 13 | (31.7) | 8 | (19.5) |
| OR vs. placebo (95% CI) | | | 0.652 | (0.192, 2.222) | | | 0.522 | (0.189, 1.440) |
| P-value vs. placebo | | | 0.4946 | | | | 0.2093 | |
| RD vs. Placebo (95% CI) | | | −0.092 | (−0.376, 0.192) | | | −0.122 | (−0.309, 0.065) |
| Overall P-value for interaction | | | 0.6260 | | | | 0.9272 | |
| Baseline median IgE level | | | | | | | | |
| (IU/mL) (400 mg BID only) | | | | | | | | |
| <Median (210.8) | | | | | | | | |
| Number | 13 | | 19 | | | | | |
| LOAC | 5 | (38.5) | 6 | (31.6) | | | | |
| OR vs. placebo (95% CI) | | | 0.744 | (0.161, 3.434) | | | | |
| P-value vs. placebo | | | 0.7042 | | | | | |
| RD vs. Placebo (95% CI) | | | −0.053 | (−0.393, 0.286) | | | | |
| P-value vs. placebo | | | 0.7585 | | | | | |
| ≥Median (210.8) | | | | | | | | |
| Number | 19 | | 13 | | | | | |
| LOAC | 11 | (57.9) | 6 | (46.2) | | | | |
| OR vs. placebo (95% CI) | | | 0.604 | (0.136, 2.679) | | | | |
| P-value vs. placebo | | | 0.5067 | | | | | |
| RD vs. Placebo (95% CI) | | | −0.124 | (−0.482, 0.233) | | | | |
| P-value vs. placebo | | | 0.4960 | | | | | |
| Overall P-value for interaction | | | 0.9289 | | | | | |
| Baseline median IgA level | | | | | | | | |
| (IU/mL) (400 mg BID only) | | | | | | | | |
| <Median (2600) | | | | | | | | |
| Number | 15 | | 17 | | | | | |
| LOAC | 9 | (60.0) | 4 | (23.5) | | | | |
| OR vs. placebo (95% CI) | | | 0.205 | (0.045, 0.942) | | | | |
| P-value vs. placebo | | | 0.0416 | | | | | |
| RD vs. Placebo (95% CI) | | | −0.365 | (−0.684, −0.045) | | | | |
| P-value vs. placebo | | | 0.0253 | | | | | |
| ≥Median (2600) | | | | | | | | |
| Number | 17 | | 15 | | | | | |
| LOAC | 7 | (41.2) | 8 | (53.3) | | | | |
| OR vs. placebo (95% CI) | | | 1.633 | (0.402, 6.625) | | | | |
| P-value vs. placebo | | | 0.4927 | | | | | |
| RD vs. Placebo (95% CI) | | | 0.122 | (−0.223, 0.466) | | | | |
| P-value vs. placebo | | | 0.4888 | | | | | |
| Overall P-value for interaction | | | 0.0553 | | | | | |
| Baseline median IgE level | | | | | | | | |
| (IU/mL) (400 mg TID only) | | | | | | | | |
| <Median (121.85) | | | | | | | | |
| Number | | | | | 34 | | 32 | |
| LOAC | | | | | 9 | (26.5) | 6 | (18.8) |
| OR vs. placebo (95% CI) | | | | | | | 0.681 | (0.209, 2.219) |
| P-value vs. placebo | | | | | | | 0.5236 | |
| RD vs. placebo (95% CI) | | | | | | | −0.070 | (−0.274, 0.135) |
| ≥Median (121.85) | | | | | | | | |
| Number | | | | | 34 | | 32 | |
| LOAC | | | | | 11 | (32.4) | 6 | (18.8) |
| OR vs. placebo (95% CI) | | | | | | | 0.510 | (0.156, 1.672) |
| P-value vs. placebo | | | | | | | 0.2663 | |
| RD vs. placebo (95% CI) | | | | | | | −0.122 | (−0.356, 0.112) |
| Overall P-value for interaction | | | | | | | 0.7981 | |
| Baseline median IgA level | | | | | | | | |
| (mg/L) (400 mg TID only) | | | | | | | | |
| <Median (2190) | | | | | | | | |
| Number | | | | | 34 | | 32 | |
| LOAC | | | | | 9 | (26.5) | 6 | (18.8) |
| OR vs. placebo (95% CI) | | | | | | | 0.774 | (0.217, 2.765) |
| P-value vs. placebo | | | | | | | 0.6933 | |
| RD vs. placebo (95% CI) | | | | | | | −0.011 | (−0.239, 0.218) |
| ≥Median (2190) | | | | | | | | |
| Number | | | | | 34 | | 32 | |
| LOAC | | | | | 11 | (32.4) | 6 | (18.8) |
| OR vs. placebo (95% CI) | | | | | | | 0.514 | (0.160, 1.646) |
| P-value vs. placebo | | | | | | | 0.2622 | |
| RD vs. placebo (95% CI) | | | | | | | −0.120 | (−0.347, 0.107) |
| Overall P-value for interaction | | | | | | | 0.6801 | |
| Baseline blood eosinophil | | | | | | | | |
| level 1 (10\hat{ }9/L) | | | | | | | | |
| <0.15 | | | | | | | | |
| Number | 6 | | 8 | | 29 | | 28 | |
| LOAC | 3 | (50.0) | 2 | (25.0) | 8 | (27.6) | 6 | (21.4) |
| OR vs. placebo (95% CI) | | | 0.527 | (0.017, 16.019) | | | 0.716 | (0.212, 2.415) |
| P-value vs. placebo | | | 0.7133 | | | | 0.5900 | |
| RD vs. Placebo (95% CI) | | | −0.135 | (−0.945, 0.675) | | | −0.062 | (−0.284, 0.161) |
| P-value vs. placebo | | | 0.7433 | | | | | |
| ≥0.15 (400 mg BID only) | | | | | | | | |
| Number | 26 | | 23 | | | | | |
| LOAC | 13 | (50.0) | 10 | (43.5) | | | | |
| OR vs. placebo (95% CI) | | | 0.737 | (0.221, 2.453) | | | | |
| P-value vs. placebo | | | 0.6186 | | | | | |
| RD vs. Placebo (95% CI) | | | −0.077 | (−0.361, 0.207) | | | | |
| P-value vs. placebo | | | 0.5938 | | | | | |
| Overall P-value for interaction | | | 0.4802 | | | | | |
| ≥0.15-<0.3 (400 | | | | | | | | |
| mg TID only) | | | | | | | | |
| Number | | | | | 20 | | 18 | |
| LOAC | | | | | 3 | (15.0) | 4 | (22.2) |
| OR vs. placebo (95% CI) | | | | | | | 1.619 | (0.309, 8.478) |
| P-value vs. placebo | | | | | | | 0.5684 | |
| RD vs. placebo (95% CI) | | | | | | | 0.072 | (−0.176, 0.320) |
| ≥0.3 (400 mg TID only) | | | | | | | | |
| Number | | | | | 19 | | 18 | |
| LOAC | | | | | 9 | (47.4) | 2 | (11.1) |
| OR vs. placebo (95% CI) | | | | | | | 0.139 | (0.025, 0.779) |
| P-value vs. placebo | | | | | | | 0.0248 | |
| RD vs. placebo (95% CI) | | | | | | | −0.363 | (−0.630, −0.095) |
| Overall P-value for interaction | | | | | | | 0.1249 | |
| Baseline blood eosinophil | | | | | | | | |
| level 2 (10\hat{ }9/L) | | | | | | | | |
| <0.3 | | | | | | | | |
| Number | 19 | | 17 | | 49 | | 46 | |
| LOAC | 10 | (52.6) | 5 | (29.4) | 11 | (22.4) | 10 | (21.7) |
| OR vs. placebo (95% CI) | | | 0.231 | (0.042, 1.283) | | | 1.019 | (0.376, 2.763) |
| P-value vs. placebo | | | 0.0939 | | | | 0.9711 | |
| RD vs. Placebo (95% CI) | | | −0.197 | (−0.567, 0.174) | | | 0.047 | (−0.153, 0.247) |
| ≥0.3 | | | | | | | | |
| Number | 13 | | 14 | | 19 | | 18 | |
| LOAC | 6 | (46.2) | 7 | (50.0) | 9 | (47.4) | 2 | (11.1) |
| OR vs. placebo (95% CI) | | | 0.951 | (0.192, 4.710) | | | 0.146 | (0.025, 0.844) |
| P-value vs. placebo | | | 0.9511 | | | | 0.0316 | |
| RD vs. Placebo (95% CI) | | | −0.013 | (−0.398, 0.372) | | | −0.350 | (−0.653, −0.047) |
| Overall P-value for interaction | | | 0.2298 | | | | 0.0574 | |
| Baseline FeNO level 1 (ppb) | | | | | | | | |
| <25 | | | | | | | | |
| Number | 15 | | 12 | | 42 | | 42 | |
| LOAC | 8 | (53.3) | 3 | (25.0) | 14 | (33.3) | 9 | (21.4) |
| OR vs. placebo (95% CI) | | | 0.261 | (0.044, 1.567) | | | 0.567 | (0.208, 1.548) |
| P-value vs. placebo | | | 0.1420 | | | | 0.2682 | |
| RD vs. Placebo (95% CI) | | | −0.227 | (−0.621, 0.166) | | | −0.065 | (−0.275, 0.145) |
| ≥25 | | | | | | | | |
| Number | 16 | | 20 | | 22 | | 20 | |
| LOAC | 8 | (50.0) | 9 | (45.0) | 4 | (18.2) | 3 | (15.0) |
| OR vs. placebo (95% CI) | | | 0.946 | (0.233, 3.843) | | | 0.798 | (0.151, 4.218) |
| P-value vs. placebo | | | 0.9383 | | | | 0.7903 | |
| RD vs. Placebo (95% CI) | | | −0.007 | (−0.333, 0.320) | | | −0.053 | (−0.313, 0.207) |
| Overall P-value for interaction | | | 0.2742 | | | | 0.6308 | |
| Baseline FeNO | | | | | | | | |
| level 2 (ppb) | | | | | | | | |
| <35 | | | | | | | | |
| Number | 19 | | 21 | | 50 | | 47 | |
| LOAC | 11 | (57.9) | 8 | (38.1) | 14 | (28.0) | 9 | (19.1) |
| OR vs. placebo (95% CI) | | | 0.448 | (0.126, 1.589) | | | 0.655 | (0.246, 1.743) |
| P-value vs. placebo | | | 0.2136 | | | | 0.3965 | |
| RD vs. Placebo (95% CI) | | | −0.198 | (−0.502, 0.106) | | | −0.029 | (−0.221, 0.162) |
| ≥35 | | | | | | | | |
| Number | 12 | | 11 | | 14 | | 15 | |
| LOAC | 5 | (41.7) | 4 | (36.4) | 4 | (28.6) | 3 | (20.0) |
| OR vs. placebo (95% CI) | | | 0.800 | (0.149, 4.297) | | | 0.628 | (0.108, 3.647) |
| P-value vs. placebo | | | 0.7947 | | | | 0.6038 | |
| RD vs. Placebo (95% CI) | | | −0.053 | (−0.451, 0.345) | | | −0.094 | (−0.398, 0.210) |

| | | |
|---|---|---|
| Overall P-value for interaction | 0.1981 | 0.9120 |

LATAM: Latin America,
ROW: Rest of the world

TABLE 28
Subgroup analysis: incidence of LOAC in the baseline
low EOS and low FeNO subgroup (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| | BID | | 400 mg BID | | TID | | 400 mg TID | |
| | (N = 32) | | (N = 32) | | (N = 68) | | (N = 64) | |
| Baseline blood eosinophil | | | | | | | | |
| (10\hat{ }9/L) and FeNO (ppb) | | | | | | | | |
| Low EOS (<0.15) and low | | | | | | | | |
| FeNO (<25) | | | | | | | | |
| Number | 4 | | 3 | | 23 | | 21 | |
| LOAC | 3 | (75.0) | 0 | | 8 | (34.8) | 4 | (19.0) |
| OR vs. placebo (95% CI) | | | NC | (NC, NC) | | | 0.441 | (0.110, 1.765) |
| P-value vs. placebo | | | NC | | | | 0.2474 | |
| RD vs. Placebo (95% CI) | | | NC | (NC, NC) | | | −0.157 | (−0.414, 0.100) |
| All others | | | | | | | | |
| Number | 27 | | 28 | | 41 | | 41 | |
| LOAC | 13 | (48.1) | 12 | (42.9) | 10 | (24.4) | 8 | (19.5) |
| OR vs. placebo (95% CI) | | | 0.808 | (0.279, 2.338) | | | 0.752 | (0.263, 2.150) |
| P-value vs. placebo | | | 0.6938 | | | | 0.5942 | |
| RD vs. Placebo (95% CI) | | | −0.053 | (−0.316, 0.210) | | | −0.049 | (−0.228, 0.130) |

| | | |
|---|---|---|
| P-value vs. placebo | 0.6933 | 0.4970 |

LATAM: Latin America,
ROW: Rest of the world

### FEV1

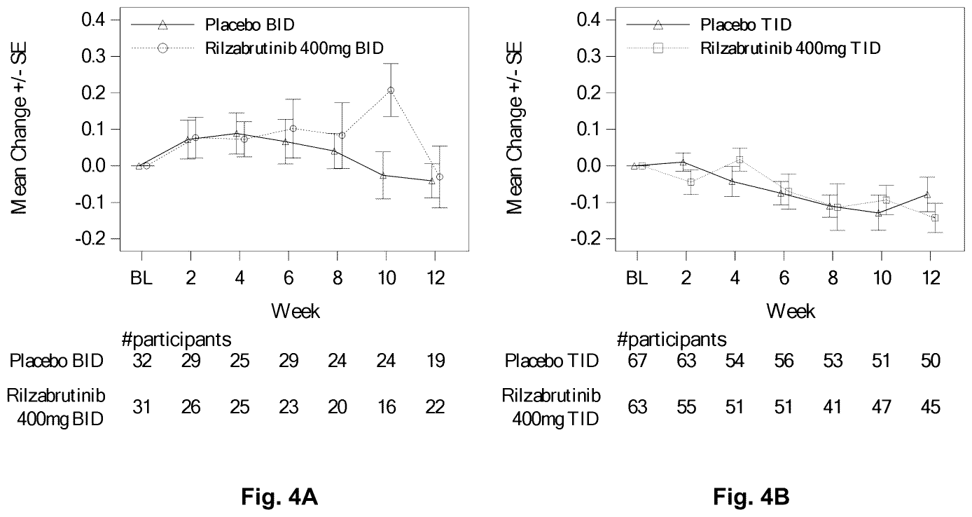

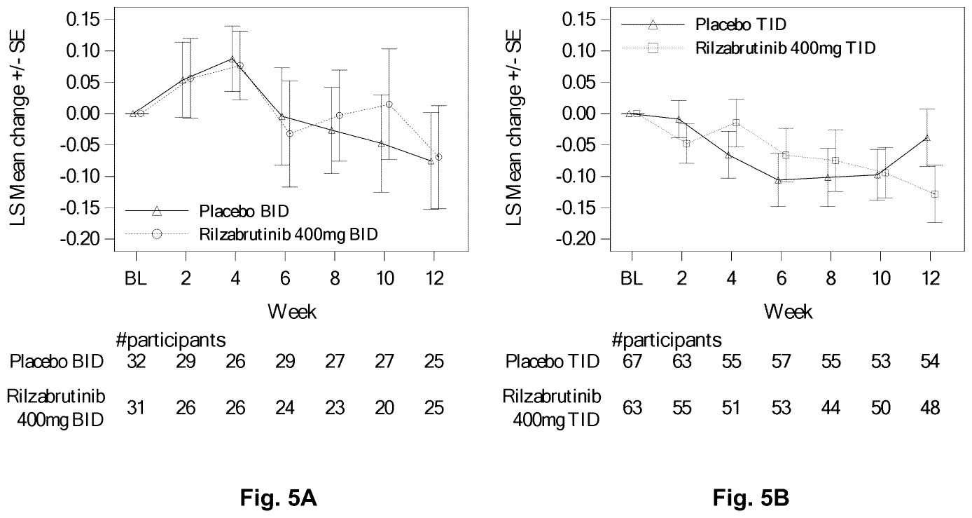

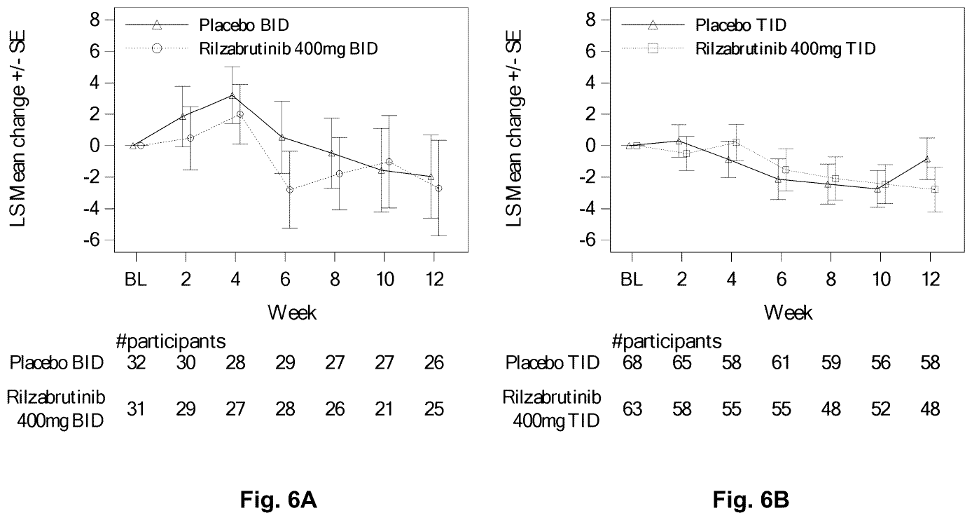

Rilzabrutinib treatment was not associated with an improvement in FEV1 values relative to treatment with the placebo (FIGS. 4-6 and Tables 29-38).

TABLE 29
Key secondary analysis: change from baseline in pre-bronchodilator
FEV1 (L) at Week 12, reduced model (mITT population).

| | 400 mg BID cohort | | | | 400 mg TID cohort | | | |
|---|---|---|---|---|---|---|---|---|
| | Placebo | | Rilzabrutinib 400 | | Placebo | | Rilzabrutinib 400 | |
| Pre-bronchodilator | BID | | mg BID | | TID | | mg TID | |
| FEV1 (L) | (N = 32) | | (N = 32) | | (N = 68) | | (N = 64) | |
| Baseline | | | | | | | | |
| Number | 32 | | 31 | | 67 | | 63 | |
| Mean (SD) | 2.16 | (0.77) | 2.11 | (0.67) | 2.42 | (0.90) | 2.50 | (0.95) |
| Median | 2.10 | | 1.94 | | 2.15 | | 2.36 | |
| Q1; Q3 | 1.47; | 2.50 | 1.59; | 2.52 | 1.77; | 2.98 | 1.73; | 3.10 |
| Min; Max | 1.2; | 3.9 | 1.0; | 3.8 | 1.0; | 5.0 | 1.1; | 5.3 |
| Week 12 | | | | | | | | |
| Number | 25 | (16/9) | 26 | (21/5) | 55 | (45/10) | 49 | (46/3) |
| (observed/LOCF | | | | | | | | |
| imputed) | | | | | | | | |
| Mean (SD) | 2.00 | (0.72) | 2.02 | (0.77) | 2.47 | (0.90) | 2.43 | (0.83) |
| Median | 1.82 | | 1.88 | | 2.39 | | 2.39 | |
| Q1; Q3 | 1.44; | 2.33 | 1.57; | 2.57 | 1.76; | 3.10 | 1.86; | 2.80 |
| Min; Max | 1.0; | 3.6 | 0.6; | 3.8 | 0.9; | 5.0 | 1.1; | 4.5 |
| Change from | | | | | | | | |
| baseline | | | | | | | | |
| Number | 25 | (16/9) | 25 | (20/5) | 54 | (44/10) | 48 | (45/3) |
| (observed/LOCF | | | | | | | | |
| imputed) | | | | | | | | |
| Mean (SD) | −0.06 | (0.22) | −0.04 | (0.39) | −0.06 | (0.31) | −0.15 | (0.27) |
| Median | −0.06 | | −0.02 | | −0.07 | | −0.12 | |
| Q1; Q3 | −0.18; | 0.10 | −0.21; | 0.12 | −0.21; | 0.06 | −0.28; | 0.05 |
| Min; Max | −0.6; | 0.3 | −1.0; | 0.7 | −0.7; | 1.2 | −1.1; | 0.4 |
| LS Mean (SE) | −0.08 | (0.08) | −0.07 | (0.08) | −0.04 | (0.05) | −0.13 | (0.05) |
| LS Mean Diff vs. | | | 0.01 | (−0.17, 0.19) | | | | |
| placebo (95% CI) | | | | | | | | |
| P-value vs. | | | 0.9466 | | | | −0.09 | (−0.21, 0.03) |
| placebo | | | | | | | | |

LATAM: Latin America,
ROW: Rest of the world

TABLE 30
Supplementary analysis (MMRM): change from baseline in pre-
bronchodilator FEV1 (L) at EOT (Week 12; mITT population).

| | 400 mg BID cohort | | | | 400 mg TID cohort | | | |
|---|---|---|---|---|---|---|---|---|
| | Placebo | | Rilzabrutinib 400 | | Placebo | | Rilzabrutinib 400 | |
| Pre-bronchodilator | BID | | mg BID | | TID | | mg TID | |
| FEV1 (L) | (N = 32) | | (N = 32) | | (N = 68) | | (N = 64) | |
| Baseline | | | | | | | | |
| Number | 32 | | 31 | | 67 | | 63 | |
| Mean (SD) | 2.16 | (0.77) | 2.11 | (0.67) | 2.42 | (0.90) | 2.50 | (0.95) |
| Median | 2.10 | | 1.94 | | 2.15 | | 2.36 | |
| Q1; Q3 | 1.47; | 2.50 | 1.59; | 2.52 | 1.77; | 2.98 | 1.73; | 3.10 |
| Min; Max | 1.2; | 3.9 | 1.0; | 3.8 | 1.0; | 5.0 | 1.1; | 5.3 |
| Week 12 | | | | | | | | |
| Number | 16 | | 21 | | 45 | | 46 | |
| Mean (SD) | 2.03 | (0.69) | 2.01 | (0.83) | 2.37 | (0.83) | 2.46 | (0.81) |
| Median | 1.78 | | 1.88 | | 2.22 | | 2.40 | |
| Q1; Q3 | 1.61; | 2.40 | 1.57; | 2.30 | 1.76; | 2.82 | 1.89; | 2.80 |
| Min; Max | 1.0; | 3.3 | 0.6; | 3.8 | 0.9; | 4.1 | 1.1; | 4.5 |
| Change from | | | | | | | | |
| baseline | | | | | | | | |
| Number | 16 | | 20 | | 44 | | 45 | |
| Mean (SD) | −0.03 | (0.21) | −0.02 | (0.41) | −0.07 | (0.34) | −0.14 | (0.27) |
| Median | −0.06 | | −0.04 | | −0.06 | | −0.09 | |
| Q1; Q3 | −0.16; | 0.17 | −0.22; | 0.32 | −0.27; | 0.06 | −0.28; | 0.05 |
| Min; Max | −0.4; | 0.3 | −1.0; | 0.7 | −0.7; | 1.2 | −1.1; | 0.4 |
| LS Mean (SE) | 0.00 | (0.08) | −0.01 | (0.08) | −0.08 | (0.05) | −0.16 | (0.05) |
| LS Mean diff | | | 0.00 | (−0.21, 0.21) | | | −0.08 | (−0.21, 0.05) |
| vs. placebo | | | | | | | | |
| (95% CI) | | | | | | | | |
| P-value vs. | | | 0.9747 | | | | 0.2225 | |
| placebo | | | | | | | | |

LATAM: Latin America,
ROW: Rest of the world

TABLE 31
Summary of pre-bronchodilator FEV1 (L) over time
(on-site conventional spirometry before LOAC
and OCS use as observed; mITT population).

| | Placebo | | Rilzabrutinib | |
|---|---|---|---|---|
| Pre-bronchodilator | BID | | 400 mg BID | |
| FEV1 (L) | (N = 32) | | (N = 32) | |
| Baseline | | | | |
| Number | 32 | | 31 | |
| Mean (SD) | 2.16 | (0.77) | 2.11 | (0.67) |
| Median | 2.10 | | 1.94 | |
| Q1; Q3 | 1.47; | 2.50 | 1.59; | 2.52 |
| Min; Max | 1.2; | 3.9 | 1.0; | 3.8 |
| Week 2 | | | | |
| Number | 28 | | 27 | |
| Mean (SD) | 2.23 | (0.77) | 2.20 | (0.68) |
| Median | 2.15 | | 2.08 | |
| Q1; Q3 | 1.68; | 2.63 | 1.79; | 2.64 |
| Min; Max | 1.2; | 4.0 | 1.0; | 4.0 |
| Change from baseline | | | | |
| Number | 28 | | 26 | |
| Mean (SD) | 0.07 | (0.29) | 0.08 | (0.29) |
| Median | 0.05 | | 0.04 | |
| Q1; Q3 | −0.05; | 0.18 | −0.08; | 0.22 |
| Min; Max | −0.7; | 0.8 | −0.6; | 0.6 |
| Percent change | | | | |
| from baseline | | | | |
| Number | 28 | | 26 | |
| Mean (SD) | 4.24 | (15.23) | 4.47 | (12.91) |
| Median | 1.71 | | 2.02 | |
| Q1; Q3 | −2.49; | 9.97 | −2.56; | 11.40 |
| Min; Max | −29.8; | 45.4 | −19.9; | 38.3 |
| Week 4 | | | | |
| Number | 24 | | 25 | |
| Mean (SD) | 2.24 | (0.75) | 2.05 | (0.62) |
| Median | 2.11 | | 1.96 | |
| Q1; Q3 | 1.73; | 2.73 | 1.70; | 2.36 |
| Min; Max | 1.0; | 3.9 | 1.0; | 3.3 |
| Change from baseline | | | | |
| Number | 24 | | 25 | |
| Mean (SD) | 0.08 | (0.29) | 0.07 | (0.24) |
| Median | 0.02 | | 0.02 | |
| Q1; Q3 | −0.08; | 0.19 | −0.07; | 0.17 |
| Min; Max | −0.4; | 0.9 | −0.3; | 0.6 |
| Percent change | | | | |
| from baseline | | | | |
| Number | 24 | | 25 | |
| Mean (SD) | 4.31 | (16.19) | 3.72 | (12.19) |
| Median | 0.51 | | 0.55 | |
| Q1; Q3 | −5.00; | 8.08 | −4.02; | 11.68 |
| Min; Max | −17.9; | 54.0 | −16.7; | 30.7 |
| Week 6 | | | | |
| Number | 27 | | 23 | |
| Mean (SD) | 2.08 | (0.67) | 2.24 | (0.79) |
| Median | 2.19 | | 2.01 | |
| Q1; Q3 | 1.46; | 2.53 | 1.56; | 2.71 |
| Min; Max | 1.0; | 3.5 | 1.0; | 3.9 |
| Change from baseline | | | | |
| Number | 27 | | 22 | |
| Mean (SD) | 0.05 | (0.33) | 0.09 | (0.39) |
| Median | 0.07 | | 0.07 | |
| Q1; Q3 | −0.13; | 0.18 | −0.11; | 0.25 |
| Min; Max | −0.7; | 0.9 | −0.7; | 1.2 |
| Percent change | | | | |
| from baseline | | | | |
| Number | 27 | | 22 | |
| Mean (SD) | 3.38 | (17.82) | 3.99 | (17.73) |
| Median | 2.56 | | 3.66 | |
| Q1; Q3 | −5.05; | 12.12 | −4.84; | 11.59 |
| Min; Max | −35.5; | 53.4 | −27.7; | 44.3 |
| Week 8 | | | | |
| Number | 24 | | 20 | |
| Mean (SD) | 2.05 | (0.68) | 2.10 | (0.85) |
| Median | 2.03 | | 1.83 | |
| Q1; Q3 | 1.49; | 2.45 | 1.51; | 2.51 |
| Min; Max | 0.9; | 3.6 | 0.9; | 3.9 |
| Change from baseline | | | | |
| Number | 24 | | 19 | |
| Mean (SD) | 0.04 | (0.24) | 0.08 | (0.41) |
| Median | −0.01 | | 0.11 | |
| Q1; Q3 | −0.12; | 0.19 | −0.07; | 0.28 |
| Min; Max | −0.3; | 0.7 | −1.1; | 0.7 |
| Percent change | | | | |
| from baseline | | | | |
| Number | 24 | | 19 | |
| Mean (SD) | 2.52 | (14.71) | 3.28 | (18.63) |
| Median | −0.52 | | 3.73 | |
| Q1; Q3 | −5.85; | 7.52 | −4.40; | 17.61 |
| Min; Max | −25.8; | 41.1 | −42.2; | 27.5 |
| Week 10 | | | | |
| Number | 21 | | 17 | |
| Mean (SD) | 2.03 | (0.75) | 2.29 | (0.76) |
| Median | 1.99 | | 1.93 | |
| Q1; Q3 | 1.57; | 2.40 | 1.78; | 2.57 |
| Min; Max | 0.8; | 3.8 | 1.6; | 4.2 |
| Change from baseline | | | | |
| Number | 21 | | 16 | |
| Mean (SD) | 0.03 | (0.29) | 0.21 | (0.29) |
| Median | −0.05 | | 0.12 | |
| Q1; Q3 | −0.17; | 0.23 | −0.01; | 0.42 |
| Min; Max | −0.4; | 0.8 | −0.2; | 0.7 |
| Percent change | | | | |
| from baseline | | | | |
| Number | 21 | | 16 | |
| Mean (SD) | 1.05 | (15.30) | 10.71 | (16.43) |
| Median | −1.77 | | 4.52 | |
| Q1; Q3 | −8.09; | 13.55 | −0.45; | 24.37 |
| Min; Max | −28.7; | 28.2 | −11.9; | 45.6 |
| Week 12 | | | | |
| Number | 16 | | 21 | |
| Mean (SD) | 2.03 | (0.69) | 2.01 | (0.83) |
| Median | 1.78 | | 1.88 | |
| Q1; Q3 | 1.61; | 2.40 | 1.57; | 2.30 |
| Min; Max | 1.0; | 3.3 | 0.6; | 3.8 |
| Change from baseline | | | | |
| Number | 16 | | 20 | |
| Mean (SD) | −0.03 | (0.21) | −0.02 | (0.41) |
| Median | −0.06 | | −0.04 | |
| Q1; Q3 | −0.16; | 0.17 | −0.22; | 0.32 |
| Min; Max | −0.4; | 0.3 | −1.0; | 0.7 |
| Percent change | | | | |
| from baseline | | | | |
| Number | 16 | | 20 | |
| Mean (SD) | −0.55 | (11.79) | −2.63 | (21.27) |
| Median | −2.60 | | −2.04 | |
| Q1; Q3 | −10.10; | 9.07 | −11.09; | 12.57 |
| Min; Max | −18.0; | 23.4 | −54.6; | 32.7 |
| Week 16 | | | | |
| Number | 19 | | 19 | |
| Mean (SD) | 2.21 | (0.80) | 2.30 | (0.84) |
| Median | 2.09 | | 2.01 | |
| Q1; Q3 | 1.48; | 2.66 | 1.80; | 2.67 |
| Min; Max | 1.0; | 3.8 | 0.9; | 4.2 |
| Change from baseline | | | | |
| Number | 19 | | 18 | |
| Mean (SD) | 0.06 | (0.29) | 0.29 | (0.37) |
| Median | 0.12 | | 0.28 | |
| Q1; Q3 | −0.12; | 0.28 | −0.03; | 0.44 |
| Min; Max | −0.7; | 0.6 | −0.3; | 1.1 |
| Percent change | | | | |
| from baseline | | | | |
| Number | 19 | | 18 | |
| Mean (SD) | 2.89 | (15.39) | 13.96 | (17.98) |
| Median | 4.48 | | 15.23 | |
| Q1; Q3 | −5.52; | 9.15 | −1.39; | 22.70 |
| Min; Max | −33.5; | 34.5 | −12.4; | 57.3 |

TABLE 32
Summary of pre-bronchodilator FEV1 (L) over time (on-site
conventional spirometry as observed; mITT population).

| | Placebo | | Rilzabrutinib | |
|---|---|---|---|---|
| Pre-bronchodilator | BID | | 400 mg BID | |
| FEV1 (L) | (N = 32) | | (N = 32) | |
| Baseline | | | | |
| Number | 32 | | 31 | |
| Mean (SD) | 2.16 | (0.77) | 2.11 | (0.67) |
| Median | 2.10 | | 1.94 | |
| Q1; Q3 | 1.47; | 2.50 | 1.59; | 2.52 |
| Min; Max | 1.2; | 3.9 | 1.0; | 3.8 |
| Week 2 | | | | |
| Number | 29 | | 27 | |
| Mean (SD) | 2.20 | (0.77) | 2.20 | (0.68) |
| Median | 2.13 | | 2.08 | |
| Q1; Q3 | 1.66; | 2.55 | 1.79; | 2.64 |
| Min; Max | 1.2; | 4.0 | 1.0; | 4.0 |
| Change from baseline | | | | |
| Number | 29 | | 26 | |
| Mean (SD) | 0.07 | (0.29) | 0.08 | (0.29) |
| Median | 0.03 | | 0.04 | |
| Q1; Q3 | −0.04; | 0.17 | −0.08; | 0.22 |
| Min; Max | −0.7; | 0.8 | −0.6; | 0.6 |
| Percent change | | | | |
| from baseline | | | | |
| Number | 29 | | 26 | |
| Mean (SD) | 4.12 | (14.97) | 4.47 | (12.91) |
| Median | 1.16 | | 2.02 | |
| Q1; Q3 | −2.46; | 8.38 | −2.56; | 11.40 |
| Min; Max | −29.8; | 45.4 | −19.9; | 38.3 |
| Week 4 | | | | |
| Number | 25 | | 25 | |
| Mean (SD) | 2.29 | (0.79) | 2.05 | (0.62) |
| Median | 2.12 | | 1.96 | |
| Q1; Q3 | 1.79; | 2.75 | 1.70; | 2.36 |
| Min; Max | 1.0; | 3.9 | 1.0; | 3.3 |
| Change from baseline | | | | |
| Number | 25 | | 25 | |
| Mean (SD) | 0.09 | (0.28) | 0.07 | (0.24) |
| Median | 0.03 | | 0.02 | |
| Q1; Q3 | −0.08; | 0.19 | −0.07; | 0.17 |
| Min; Max | −0.4; | 0.9 | −0.3; | 0.6 |
| Percent change | | | | |
| from baseline | | | | |
| Number | 25 | | 25 | |
| Mean (SD) | 4.36 | (15.85) | 3.72 | (12.19) |
| Median | 0.66 | | 0.55 | |
| Q1; Q3 | −4.25; | 7.82 | −4.02; | 11.68 |
| Min; Max | −17.9; | 54.0 | −16.7; | 30.7 |
| Week 6 | | | | |
| Number | 29 | | 24 | |
| Mean (SD) | 2.11 | (0.70) | 2.24 | (0.78) |
| Median | 2.19 | | 2.06 | |
| Q1; Q3 | 1.51; | 2.53 | 1.63; | 2.64 |
| Min; Max | 1.0; | 3.5 | 1.0; | 3.9 |
| Change from baseline | | | | |
| Number | 29 | | 23 | |
| Mean (SD) | 0.07 | (0.33) | 0.10 | (0.39) |
| Median | 0.07 | | 0.07 | |
| Q1; Q3 | −0.06; | 0.18 | −0.11; | 0.33 |
| Min; Max | −0.7; | 0.9 | −0.7; | 1.2 |
| Percent change | | | | |
| from baseline | | | | |
| Number | 29 | | 23 | |
| Mean (SD) | 4.36 | (18.18) | 4.61 | (17.58) |
| Median | 2.56 | | 5.14 | |
| Q1; Q3 | −4.60; | 12.12 | −4.84; | 13.93 |
| Min; Max | −35.5; | 53.4 | −27.7; | 44.3 |
| Week 8 | | | | |
| Number | 24 | | 21 | |
| Mean (SD) | 2.05 | (0.68) | 2.08 | (0.83) |
| Median | 2.03 | | 1.86 | |
| Q1; Q3 | 1.49; | 2.45 | 1.52; | 2.45 |
| Min; Max | 0.9; | 3.6 | 0.9; | 3.9 |
| Change from baseline | | | | |
| Number | 24 | | 20 | |
| Mean (SD) | 0.04 | (0.24) | 0.08 | (0.40) |
| Median | −0.01 | | 0.09 | |
| Q1; Q3 | −0.12; | 0.19 | −0.06; | 0.25 |
| Min; Max | −0.3; | 0.7 | −1.1; | 0.7 |
| Percent change | | | | |
| from baseline | | | | |
| Number | 24 | | 20 | |
| Mean (SD) | 2.52 | (14.71) | 3.30 | (18.14) |
| Median | −0.52 | | 3.64 | |
| Q1; Q3 | −5.85; | 7.52 | −4.06; | 14.97 |
| Min; Max | −25.8; | 41.1 | −42.2; | 27.5 |
| Week 10 | | | | |
| Number | 24 | | 17 | |
| Mean (SD) | 1.96 | (0.74) | 2.29 | (0.76) |
| Median | 1.97 | | 1.93 | |
| Q1; Q3 | 1.40; | 2.33 | 1.78; | 2.57 |
| Min; Max | 0.8; | 3.8 | 1.6; | 4.2 |
| Change from baseline | | | | |
| Number | 24 | | 16 | |
| Mean (SD) | −0.02 | (0.30) | 0.21 | (0.29) |
| Median | −0.10 | | 0.12 | |
| Q1; Q3 | −0.18; | 0.20 | −0.01; | 0.42 |
| Min; Max | −0.5; | 0.8 | −0.2; | 0.7 |
| Percent change | | | | |
| from baseline | | | | |
| Number | 24 | | 16 | |
| Mean (SD) | −1.48 | (16.21) | 10.71 | (16.43) |
| Median | −5.47 | | 4.52 | |
| Q1; Q3 | −9.15; | 13.20 | −0.45; | 24.37 |
| Min; Max | −30.1; | 28.2 | −11.9; | 45.6 |
| Week 12 | | | | |
| Number | 19 | | 23 | |
| Mean (SD) | 2.13 | (0.71) | 1.97 | (0.81) |
| Median | 2.04 | | 1.82 | |
| Q1; Q3 | 1.67; | 2.47 | 1.43; | 2.30 |
| Min; Max | 1.0; | 3.5 | 0.6; | 3.8 |
| Change from baseline | | | | |
| Number | 19 | | 22 | |
| Mean (SD) | −0.04 | (0.20) | −0.03 | (0.40) |
| Median | −0.08 | | −0.05 | |
| Q1; Q3 | −0.19; | 0.14 | −0.23; | 0.31 |
| Min; Max | −0.4; | 0.3 | −1.0; | 0.7 |
| Percent change | | | | |
| from baseline | | | | |
| Number | 19 | | 22 | |
| Mean (SD) | −1.25 | (11.01) | −3.27 | (20.46) |
| Median | −2.79 | | −2.70 | |
| Q1; Q3 | −8.91; | 8.26 | −11.12; | 9.91 |
| Min; Max | −18.0; | 23.4 | −54.6; | 32.7 |
| Week 16 | | | | |
| Number | 19 | | 19 | |
| Mean (SD) | 2.21 | (0.80) | 2.30 | (0.84) |
| Median | 2.09 | | 2.01 | |
| Q1; Q3 | 1.48; | 2.66 | 1.80; | 2.67 |
| Min; Max | 1.0; | 3.8 | 0.9; | 4.2 |
| Change from baseline | | | | |
| Number | 19 | | 18 | |
| Mean (SD) | 0.06 | (0.29) | 0.29 | (0.37) |
| Median | 0.12 | | 0.28 | |
| Q1; Q3 | −0.12; | 0.28 | −0.03; | 0.44 |
| Min; Max | −0.7; | 0.6 | −0.3; | 1.1 |
| Percent change | | | | |
| from baseline | | | | |
| Number | 19 | | 18 | |
| Mean (SD) | 2.89 | (15.39) | 13.96 | (17.98) |
| Median | 4.48 | | 15.23 | |
| Q1; Q3 | −5.52; | 9.15 | −1.39; | 22.70 |
| Min; Max | −33.5; | 34.5 | −12.4; | 57.3 |

All data were summarized as observed.

TABLE 33
Change from baseline in pre-bronchodilator FEV1 (L) over time (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| Pre-bronchodilator | BID | | 400 mg BID | | TID | | 400 mg TID | |
| FEV1 (L) | (N = 32) | | (N = 32) | | (N = 68) | | (N = 64) | |
| Baseline | | | | | | | | |
| Number | 32 | | 31 | | 67 | | 63 | |
| Mean (SD) | 2.16 | (0.77) | 2.11 | (0.67) | 2.42 | (0.90) | 2.50 | (0.95) |
| Median | 2.10 | | 1.94 | | 2.15 | | 2.36 | |
| Q1; Q3 | 1.47; | 2.50 | 1.59; | 2.52 | 1.77; | 2.98 | 1.73; | 3.10 |
| Min; Max | 1.2; | 3.9 | 1.0; | 3.8 | 1.0; | 5.0 | 1.1; | 5.3 |
| Week 2 | | | | | | | | |
| Number | 29 | (28/1) | 27 | (27/0) | 64 | (63/1) | 56 | (56/0) |
| (observed/LOCF | | | | | | | | |
| imputed) | | | | | | | | |
| Mean (SD) | 2.20 | (0.77) | 2.20 | (0.68) | 2.48 | (0.94) | 2.47 | (0.80) |
| Median | 2.13 | | 2.08 | | 2.25 | | 2.37 | |
| Q1; Q3 | 1.66; | 2.55 | 1.79; | 2.64 | 1.75; | 3.07 | 1.76; | 3.06 |
| Min; Max | 1.2; | 4.0 | 1.0; | 4.0 | 1.0; | 5.2 | 1.1; | 4.1 |
| Change from | | | | | | | | |
| baseline | | | | | | | | |
| Number | 29 | (28/1) | 26 | (26/0) | 63 | (62/1) | 55 | (55/0) |
| (observed/LOCF | | | | | | | | |
| imputed) | | | | | | | | |
| Mean (SD) | 0.07 | (0.29) | 0.08 | (0.29) | 0.01 | (0.20) | −0.04 | (0.25) |
| Median | 0.03 | | 0.04 | | 0.02 | | −0.02 | |
| Q1; Q3 | −0.04; | 0.17 | −0.08; | 0.22 | −0.12; | 0.15 | −0.17; | 0.10 |
| Min; Max | −0.7; | 0.8 | −0.6; | 0.6 | −0.6; | 0.6 | −0.7; | 0.7 |
| LS Mean (SE) | 0.05 | (0.06) | 0.06 | (0.06) | −0.01 | (0.03) | −0.05 | (0.03) |
| LS Mean Diff vs. | | | 0.00 | (−0.15, 0.15) | | | −0.04 | (−0.12, 0.04) |
| placebo (95% CI) | | | | | | | | |
| P-value vs. | | | 0.9752 | | | | 0.3401 | |
| placebo | | | | | | | | |
| Week 4 | | | | | | | | |
| Number | 26 | (24/2) | 26 | (25/1) | 56 | (55/1) | 51 | (51/0) |
| (observed/LOCF | | | | | | | | |
| imputed) | | | | | | | | |
| Mean (SD) | 2.25 | (0.79) | 2.07 | (0.62) | 2.47 | (0.92) | 2.58 | (0.86) |
| Median | 2.11 | | 1.96 | | 2.30 | | 2.50 | |
| Q1; Q3 | 1.66; | 2.75 | 1.70; | 2.56 | 1.75; | 3.12 | 1.82; | 3.14 |
| Min; Max | 1.0; | 3.9 | 1.0; | 3.3 | 1.0; | 5.3 | 1.2; | 4.8 |
| Change from | | | | | | | | |
| baseline | | | | | | | | |
| Number | 26 | (24/2) | 26 | (25/1) | 55 | (54/1) | 51 | (51/0) |
| (observed/LOCF | | | | | | | | |
| imputed) | | | | | | | | |
| Mean (SD) | 0.08 | (0.28) | 0.07 | (0.24) | −0.04 | (0.30) | 0.02 | (0.22) |
| Median | 0.02 | | 0.02 | | −0.05 | | −0.01 | |
| Q1; Q3 | −0.08; | 0.18 | −0.07; | 0.17 | −0.17; | 0.13 | −0.13; | 0.13 |
| Min; Max | −0.4; | 0.9 | −0.3; | 0.6 | −0.9; | 0.8 | −0.5; | 0.6 |
| LS Mean (SE) | 0.09 | (0.05) | 0.08 | (0.05) | −0.07 | (0.04) | −0.01 | (0.04) |
| LS Mean Diff vs. | | | −0.01 | (−0.14, 0.12) | | | 0.05 | (−0.05, 0.15) |
| placebo (95% CI) | | | | | | | | |
| P-value vs. | | | 0.8701 | | | | 0.3010 | |
| placebo | | | | | | | | |
| Week 6 | | | | | | | | |
| Number | 29 | (27/2) | 25 | (23/2) | 58 | (56/2) | 54 | (52/2) |
| (observed/LOCF | | | | | | | | |
| imputed) | | | | | | | | |
| Mean (SD) | 2.10 | (0.72) | 2.24 | (0.77) | 2.35 | (0.88) | 2.52 | (0.87) |
| Median | 2.19 | | 2.01 | | 2.23 | | 2.37 | |
| Q1; Q3 | 1.46; | 2.53 | 1.70; | 2.64 | 1.65; | 2.97 | 1.84; | 3.17 |
| Min; Max | 1.0; | 3.6 | 1.0; | 3.9 | 1.0; | 5.0 | 1.0; | 4.8 |
| Change from | | | | | | | | |
| baseline | | | | | | | | |
| Number | 29 | (27/2) | 24 | (22/2) | 57 | (55/2) | 53 | (51/2) |
| (observed/LOCF | | | | | | | | |
| imputed) | | | | | | | | |
| Mean (SD) | 0.05 | (0.32) | 0.09 | (0.38) | −0.08 | (0.24) | −0.07 | (0.34) |
| Median | 0.07 | | 0.07 | | −0.05 | | −0.09 | |
| Q1; Q3 | −0.06; | 0.18 | −0.10; | 0.22 | −0.22; | 0.08 | −0.23; | 0.08 |
| Min; Max | −0.7; | 0.9 | −0.7; | 1.2 | −0.6; | 0.4 | −1.3; | 0.9 |
| LS Mean (SE) | −0.00 | (0.08) | −0.03 | (0.08) | −0.11 | (0.04) | −0.07 | (0.04) |
| LS Mean Diff vs. | | | −0.03 | (−0.22, 0.17) | | | 0.04 | (−0.07, 0.15) |
| placebo (95% CI) | | | | | | | | |
| P-value vs. | | | 0.7797 | | | | 0.4831 | |
| placebo | | | | | | | | |
| Week 8 | | | | | | | | |
| Number | 27 | (24/3) | 24 | (20/4) | 56 | (53/3) | 45 | (42/3) |
| (observed/LOCF | | | | | | | | |
| imputed) | | | | | | | | |
| Mean (SD) | 2.05 | (0.74) | 2.09 | (0.81) | 2.36 | (0.89) | 2.41 | (0.82) |
| Median | 2.00 | | 1.83 | | 2.23 | | 2.38 | |
| Q1; Q3 | 1.44; | 2.48 | 1.51; | 2.58 | 1.60; | 3.03 | 1.81; | 2.96 |
| Min; Max | 0.9; | 3.6 | 0.9; | 3.9 | 0.7; | 5.0 | 1.1; | 4.3 |
| Change from | | | | | | | | |
| baseline | | | | | | | | |
| Number | 27 | (24/3) | 23 | (19/4) | 55 | (52/3) | 44 | (41/3) |
| (observed/LOCF | | | | | | | | |
| imputed) | | | | | | | | |
| Mean (SD) | 0.02 | (0.26) | 0.04 | (0.41) | −0.11 | (0.22) | −0.12 | (0.40) |
| Median | 0.00 | | 0.05 | | −0.08 | | −0.10 | |
| Q1; Q3 | −0.12; | 0.13 | −0.09; | 0.23 | −0.26; | 0.04 | −0.21; | 0.08 |
| Min; Max | −0.6; | 0.7 | −1.1; | 0.7 | −0.6; | 0.4 | −2.3; | 0.6 |
| LS Mean (SE) | −0.03 | (0.07) | −0.00 | (0.07) | −0.10 | (0.05) | −0.07 | (0.05) |
| LS Mean Diff vs. | | | 0.02 | (−0.14, 0.19) | | | 0.03 | (−0.10, 0.15) |
| placebo (95% CI) | | | | | | | | |
| P-value vs. | | | 0.7849 | | | | 0.6679 | |
| placebo | | | | | | | | |
| Week 10 | | | | | | | | |
| Number | 27 | (21/6) | 21 | (17/4) | 54 | (50/4) | 50 | (47/3) |
| (observed/LOCF | | | | | | | | |
| imputed) | | | | | | | | |
| Mean (SD) | 2.02 | (0.77) | 2.24 | (0.73) | 2.32 | (0.88) | 2.53 | (0.88) |
| Median | 1.99 | | 1.93 | | 2.15 | | 2.50 | |
| Q1; Q3 | 1.38; | 2.40 | 1.76; | 2.57 | 1.62; | 2.98 | 1.78; | 3.15 |
| Min; Max | 0.8; | 3.8 | 1.3; | 4.2 | 0.9; | 5.0 | 1.1; | 4.4 |
| Change from | | | | | | | | |
| baseline | | | | | | | | |
| Number | 27 | (21/6) | 20 | (16/4) | 53 | (49/4) | 50 | (47/3) |
| (observed/LOCF | | | | | | | | |
| imputed) | | | | | | | | |
| Mean (SD) | 0.00 | (0.28) | 0.13 | (0.33) | −0.10 | (0.28) | −0.10 | (0.27) |
| Median | −0.05 | | 0.09 | | −0.07 | | −0.09 | |
| Q1; Q3 | −0.17; | 0.18 | −0.05; | 0.36 | −0.25; | 0.05 | −0.26; | 0.09 |
| Min; Max | −0.6; | 0.8 | −0.7; | 0.7 | −1.0; | 0.6 | −1.0; | 0.4 |
| LS Mean (SE) | −0.05 | (0.08) | 0.02 | (0.09) | −0.10 | (0.04) | −0.09 | (0.04) |
| LS Mean Diff vs. | | | 0.06 | (−0.13, 0.26) | | | 0.00 | (−0.10, 0.11) |
| placebo (95% CI) | | | | | | | | |
| P-value vs. | | | 0.5282 | | | | 0.9509 | |
| placebo | | | | | | | | |
| Week 12 | | | | | | | | |
| Number | 25 | (16/9) | 26 | (21/5) | 55 | (45/10) | 49 | (46/3) |
| (observed/LOCF | | | | | | | | |
| imputed) | | | | | | | | |
| Mean (SD) | 2.00 | (0.72) | 2.02 | (0.77) | 2.47 | (0.90) | 2.43 | (0.83) |
| Median | 1.82 | | 1.88 | | 2.39 | | 2.39 | |
| Q1; Q3 | 1.44; | 2.33 | 1.57; | 2.57 | 1.76; | 3.10 | 1.86; | 2.80 |
| Min; Max | 1.0; | 3.6 | 0.6; | 3.8 | 0.9; | 5.0 | 1.1; | 4.5 |
| Change from | | | | | | | | |
| baseline | | | | | | | | |
| Number | 25 | (16/9) | 25 | (20/5) | 54 | (44/10) | 48 | (45/3) |
| (observed/LOCF | | | | | | | | |
| imputed) | | | | | | | | |
| Mean (SD) | −0.06 | (0.22) | −0.04 | (0.39) | −0.06 | (0.31) | −0.15 | (0.27) |
| Median | −0.06 | | −0.02 | | −0.07 | | −0.12 | |
| Q1; Q3 | −0.18; | 0.10 | −0.21; | 0.12 | −0.21; | 0.06 | −0.28; | 0.05 |
| Min; Max | −0.6; | 0.3 | −1.0; | 0.7 | −0.7; | 1.2 | −1.1; | 0.4 |
| LS Mean (SE) | −0.08 | (0.08) | −0.07 | (0.08) | −0.04 | (0.05) | −0.13 | (0.05) |
| LS Mean Diff vs. | | | 0.01 | (−0.17, 0.19) | | | −0.09 | (−0.21, 0.03) |
| placebo (95% CI) | | | | | | | | |
| P-value vs. | | | 0.9466 | | | | 0.1523 | |
| placebo | | | | | | | | |

LATAM: Latin America,
ROW: Rest of the world

TABLE 34
Subgroup analysis: change from baseline in pre-bronchodilator FEV1
(L) at EOT (Week 12) by demographics subgroups (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| Pre-bronchodilator | BID | | 400 mg BID | | TID | | 400 mg TID | |
| FEV1 (L) | (N = 32) | | (N = 32) | | (N = 68) | | (N = 64) | |
| Age group 1 (years) | | | | | | | | |
| <45 | | | | | | | | |
| Number | 8 | | 9 | | 26 | | 22 | |
| Mean (SD) | −0.06 | (0.21) | −0.05 | (0.52) | −0.07 | (0.24) | −0.24 | (0.34) |
| LS Mean (SE) | −0.01 | (0.19) | −0.13 | (0.19) | −0.06 | (0.08) | −0.24 | (0.08) |
| LS Mean Diff vs. | | | −0.13 | (−0.60, 0.35) | | | −0.18 | (−0.38, 0.02) |
| placebo (95% CI) | | | | | | | | |
| P-value vs. placebo | | | 0.6056 | | | | 0.0756 | |
| ≥45 | | | | | | | | |
| Number | 17 | | 16 | | 28 | | 26 | |
| Mean (SD) | −0.06 | (0.23) | −0.03 | (0.32) | −0.06 | (0.38) | −0.08 | (0.17) |
| LS Mean (SE) | −0.16 | (0.10) | −0.14 | (0.10) | −0.03 | (0.05) | −0.04 | (0.06) |
| LS Mean Diff vs. | | | 0.02 | (−0.19, 0.23) | | | −0.01 | (−0.17, 0.14) |
| placebo (95% CI) | | | | | | | | |
| P-value vs. placebo | | | 0.8611 | | | | 0.8854 | |
| Overall p-value | | | 0.9627 | | | | 0.1988 | |
| for interaction | | | | | | | | |
| Gender | | | | | | | | |
| Male | | | | | | | | |
| Number | 6 | | 11 | | 22 | | 21 | |
| Mean (SD) | −0.12 | (0.31) | −0.08 | (0.53) | −0.07 | (0.36) | −0.16 | (0.29) |
| LS Mean (SE) | −0.07 | (0.19) | −0.23 | (0.19) | −0.05 | (0.08) | −0.15 | (0.08) |
| LS Mean Diff vs. | | | −0.16 | (−0.72, 0.41) | | | −0.09 | (−0.31, 0.12) |
| placebo (95% CI) | | | | | | | | |
| P-value vs. placebo | | | 0.5836 | | | | 0.3923 | |
| Female | | | | | | | | |
| Number | 19 | | 14 | | 32 | | 27 | |
| Mean (SD) | −0.04 | (0.18) | −0.01 | (0.26) | −0.06 | (0.28) | −0.14 | (0.25) |
| LS Mean (SE) | −0.03 | (0.10) | 0.00 | (0.09) | −0.02 | (0.05) | −0.11 | (0.06) |
| LS Mean Diff vs. | | | 0.04 | (−0.17, 0.24) | | | −0.09 | (−0.24, 0.05) |
| placebo (95% CI) | | | | | | | | |
| P-value vs. placebo | | | 0.7294 | | | | 0.2138 | |
| Overall p-value | | | 0.7567 | | | | 0.9813 | |
| for interaction | | | | | | | | |
| Baseline weight group (kg) | | | | | | | | |
| <60 | | | | | | | | |
| Number | 4 | | 3 | | 3 | | 0 | |
| Mean (SD) | −0.10 | (0.08) | −0.02 | (0.04) | 0.11 | (0.31) | NC | (NC) |
| LS Mean (SE) | NC | (NC) | NC | (NC) | NC | (NC) | NC | (NC) |
| LS Mean Diff vs. | | | NC | (NC, NC) | | | NC | (NC, NC) |
| placebo (95% CI) | | | | | | | | |
| P-value vs. placebo | | | NC | | | | NC | |
| ≥60-<90 | | | | | | | | |
| Number | 18 | | 18 | | 29 | | 28 | |
| Mean (SD) | −0.06 | (0.25) | −0.01 | (0.43) | −0.08 | (0.37) | −0.15 | (0.31) |
| LS Mean (SE) | −0.12 | (0.12) | −0.09 | (0.11) | −0.05 | (0.07) | −0.13 | (0.07) |
| LS Mean Diff vs. | | | 0.03 | (−0.23, 0.28) | | | −0.07 | (−0.25, 0.10) |
| placebo (95% CI) | | | | | | | | |
| P-value vs. placebo | | | 0.8258 | | | | 0.4062 | |
| ≥90 | | | | | | | | |
| Number | 3 | | 4 | | 15 | | 18 | |
| Mean (SD) | −0.00 | (0.10) | −0.17 | (0.42) | −0.15 | (0.21) | −0.17 | (0.20) |
| LS Mean (SE) | 0.12 | (0.23) | −0.25 | (0.37) | −0.10 | (0.06) | −0.15 | (0.06) |
| LS Mean Diff vs. | | | −0.37 | (−1.14, 0.40) | | | −0.05 | (−0.21, 0.11) |
| placebo (95% CI) | | | | | | | | |
| P-value vs. placebo | | | 0.3471 | | | | 0.5184 | |
| Overall p-value | | | 0.8383 | | | | 0.9293 | |
| for interaction | | | | | | | | |
| Baseline BMI group (kg/m²) | | | | | | | | |
| <25 | | | | | | | | |
| Number | 5 | | 7 | | 19 | | 8 | |
| Mean (SD) | −0.10 | (0.07) | 0.02 | (0.51) | −0.04 | (0.41) | −0.21 | (0.45) |
| LS Mean (SE) | −0.24 | (0.33) | −0.09 | (0.32) | −0.03 | (0.10) | −0.19 | (0.15) |
| LS Mean Diff vs. | | | 0.15 | (−0.48, 0.78) | | | −0.16 | (−0.48, 0.16) |
| placebo (95% CI) | | | | | | | | |
| P-value vs. placebo | | | 0.6436 | | | | 0.3337 | |
| ≥25-<30 | | | | | | | | |
| Number | 14 | | 12 | | 20 | | 25 | |
| Mean (SD) | −0.08 | (0.25) | −0.00 | (0.36) | −0.06 | (0.27) | −0.14 | (0.23) |
| LS Mean (SE) | −0.12 | (0.12) | −0.06 | (0.12) | −0.06 | (0.06) | −0.11 | (0.06) |
| LS Mean Diff vs. | | | 0.06 | (−0.25, 0.37) | | | −0.05 | (−0.22, 0.11) |
| placebo (95% CI) | | | | | | | | |
| P-value vs. placebo | | | 0.7084 | | | | 0.5147 | |
| ≥30 | | | | | | | | |
| Number | 6 | | 6 | | 15 | | 15 | |
| Mean (SD) | 0.03 | (0.23) | −0.18 | (0.33) | −0.09 | (0.25) | −0.14 | (0.22) |
| LS Mean (SE) | 0.09 | (0.27) | −0.04 | (0.25) | −0.04 | (0.07) | −0.11 | (0.07) |
| LS Mean Diff vs. | | | −0.13 | (−0.63, 0.37) | | | −0.07 | (−0.27, 0.13) |
| placebo (95% CI) | | | | | | | | |
| P-value vs. placebo | | | 0.6158 | | | | 0.5078 | |
| Overall p-value | | | 0.7816 | | | | 0.6728 | |
| for interaction | | | | | | | | |
| Region | | | | | | | | |
| Eastern Europe | | | | | | | | |
| Number | 4 | | 1 | | 38 | | 32 | |
| Mean (SD) | −0.24 | (0.31) | −0.21 | (NC) | −0.10 | (0.26) | −0.18 | (0.28) |
| LS Mean (SE) | NC | (NC) | NC | (NC) | −0.10 | (0.05) | −0.18 | (0.05) |
| LS Mean Diff vs. | | | NC | (NC, NC) | | | −0.08 | (−0.22, 0.06) |
| placebo (95% CI) | | | | | | | | |
| P-value vs. placebo | | | NC | | | | 0.2618 | |
| Latin America | | | | | | | | |
| Number | 19 | | 21 | | 14 | | 14 | |
| Mean (SD) | −0.02 | (0.19) | −0.05 | (0.39) | 0.03 | (0.44) | −0.08 | (0.27) |
| LS Mean (SE) | −0.06 | (0.10) | −0.07 | (0.08) | 0.04 | (0.12) | −0.08 | (0.11) |
| LS Mean Diff vs. | | | −0.01 | (−0.25, 0.22) | | | −0.12 | (−0.37, 0.12) |
| placebo (95% CI) | | | | | | | | |
| P-value vs. placebo | | | 0.9056 | | | | 0.3256 | |
| Western Countries | | | | | | | | |
| Number | 1 | | 2 | | 1 | | 2 | |
| Mean (SD) | −0.05 | (NC) | 0.24 | (0.67) | −0.14 | (NC) | −0.18 | (0.06) |
| LS Mean (SE) | NC | (NC) | NC | (NC) | NC | (NC) | NC | (NC) |
| LS Mean Diff vs. | | | NC | (NC, NC) | | | NC | (NC, NC) |
| placebo (95% CI) | | | | | | | | |
| P-value vs. placebo | | | NC | | | | NC | |
| APAC | | | | | | | | |
| Number | 1 | | 1 | | 1 | | 0 | |
| Mean (SD) | −0.07 | (NC) | −0.09 | (NC) | 0.02 | (NC) | NC | (NC) |
| LS Mean (SE) | NC | (NC) | NC | (NC) | NC | (NC) | NC | (NC) |
| LS Mean Diff vs. | | | NC | (NC, NC) | | | NC | (NC, NC) |
| placebo (95% CI) | | | | | | | | |

| | | |
|---|---|---|
| P-value vs. placebo | NC | NC |
| Overall p-value | 0.8887 | 0.8187 |
| for interaction | | |

LATAM: Latin America,
ROW: Rest of the world

TABLE 35
Subgroup analysis: change from baseline in pre-bronchodilator FEV1 (L) at EOT
(Week 12) by disease and other characteristics subgroups (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| Pre-bronchodilator | BID | | 400 mg BID | | TID | | 400 mg TID | |
| FEV1 (L) | (N = 32) | | (N = 32) | | (N = 68) | | (N = 64) | |
| Age at onset of | | | | | | | | |
| asthma (years) | | | | | | | | |
| <12 | | | | | | | | |
| Number | 7 | | 8 | | 12 | | 11 | |
| Mean (SD) | −0.08 | (0.21) | 0.00 | (0.48) | −0.07 | (0.25) | −0.25 | (0.38) |
| LS Mean (SE) | −0.06 | (0.25) | 0.12 | (0.32) | −0.05 | (0.10) | −0.34 | (0.11) |
| LS Mean Diff vs. | | | 0.18 | (−0.43, 0.80) | | | −0.28 | (−0.58, 0.01) |
| placebo (95% CI) | | | | | | | | |
| P-value vs. placebo | | | 0.5568 | | | | 0.0576 | |
| ≥12-<18 | | | | | | | | |
| Number | 1 | | 4 | | 5 | | 4 | |
| Mean (SD) | −0.01 | (NC) | 0.12 | (0.29) | 0.31 | (0.56) | −0.01 | (0.38) |
| LS Mean (SE) | NC | (NC) | NC | (NC) | 0.45 | (0.42) | 0.05 | (0.41) |
| LS Mean Diff vs. | | | NC | (NC, NC) | | | −0.40 | (−1.36, 0.57) |
| placebo (95% CI) | | | | | | | | |
| P-value vs. placebo | | | NC | | | | 0.4199 | |
| ≥18-<40 | | | | | | | | |
| Number | 11 | | 11 | | 23 | | 21 | |
| Mean (SD) | −0.06 | (0.28) | −0.08 | (0.36) | −0.13 | (0.30) | −0.17 | (0.23) |
| LS Mean (SE) | −0.14 | (0.13) | −0.14 | (0.13) | −0.10 | (0.06) | −0.18 | (0.07) |
| LS Mean Diff vs. | | | 0.00 | (−0.29, 0.29) | | | −0.08 | (−0.24, 0.09) |
| placebo (95% CI) | | | | | | | | |
| P-value vs. placebo | | | 0.9979 | | | | 0.3506 | |
| ≥40 | | | | | | | | |
| Number | 6 | | 2 | | 14 | | 12 | |
| Mean (SD) | −0.03 | (0.12) | −0.31 | (0.55) | −0.09 | (0.19) | −0.07 | (0.14) |
| LS Mean (SE) | −0.07 | (0.27) | 0.00 | (0.63) | −0.05 | (0.07) | 0.01 | (0.08) |
| LS Mean Diff vs. | | | 0.07 | (−0.91, 1.05) | | | 0.06 | (−0.13, 0.25) |
| placebo (95% CI) | | | | | | | | |
| P-value vs. placebo | | | 0.8887 | | | | 0.5330 | |
| Overall p-value | | | 0.9178 | | | | 0.2462 | |
| for interaction | | | | | | | | |
| Age at onset of | | | | | | | | |
| asthma (years) | | | | | | | | |
| <18 | | | | | | | | |
| Number | 8 | | 12 | | 17 | | 15 | |
| Mean (SD) | −0.07 | (0.19) | 0.04 | (0.41) | 0.04 | (0.39) | −0.18 | (0.38) |
| LS Mean (SE) | −0.09 | (0.18) | −0.00 | (0.18) | 0.02 | (0.10) | −0.25 | (0.11) |
| LS Mean Diff vs. | | | 0.08 | (−0.30, 0.47) | | | −0.27 | (−0.55, −0.00) |
| placebo (95% CI) | | | | | | | | |
| P-value vs. placebo | | | 0.6639 | | | | 0.0498 | |
| ≥18 | | | | | | | | |
| Number | 17 | | 13 | | 37 | | 33 | |
| Mean (SD) | −0.05 | (0.23) | −0.11 | (0.37) | −0.11 | (0.26) | −0.14 | (0.20) |
| LS Mean (SE) | −0.13 | (0.11) | −0.17 | (0.11) | −0.07 | (0.05) | −0.10 | (0.05) |
| LS Mean Diff vs. | | | −0.03 | (−0.27, 0.21) | | | −0.02 | (−0.16, 0.11) |
| placebo (95% CI) | | | | | | | | |
| P-value vs. placebo | | | 0.7986 | | | | 0.7110 | |
| Overall p-value | | | 0.4111 | | | | 0.0777 | |
| for interaction | | | | | | | | |
| Number of asthma | | | | | | | | |
| exacerbations | | | | | | | | |
| within 2 years before | | | | | | | | |
| screening visit | | | | | | | | |
| 1 | | | | | | | | |
| Number | 5 | | 10 | | 40 | | 31 | |
| Mean (SD) | −0.02 | (0.19) | 0.05 | (0.51) | −0.03 | (0.30) | −0.16 | (0.29) |
| LS Mean (SE) | −0.03 | (0.22) | −0.08 | (0.17) | −0.01 | (0.05) | −0.14 | (0.06) |
| LS Mean Diff vs. | | | −0.05 | (−0.57, 0.47) | | | −0.13 | (−0.27, 0.01) |
| placebo (95% CI) | | | | | | | | |
| P-value vs. placebo | | | 0.8488 | | | | 0.0625 | |
| 2 | | | | | | | | |
| Number | 14 | | 9 | | 14 | | 14 | |
| Mean (SD) | −0.07 | (0.27) | −0.06 | (0.28) | −0.17 | (0.34) | −0.16 | (0.24) |
| LS Mean (SE) | −0.11 | (0.12) | −0.01 | (0.16) | −0.14 | (0.09) | −0.08 | (0.09) |
| LS Mean Diff vs. | | | 0.10 | (−0.22, 0.41) | | | 0.06 | (−0.18, 0.30) |
| placebo (95% CI) | | | | | | | | |
| P-value vs. placebo | | | 0.5439 | | | | 0.6174 | |
| >2 | | | | | | | | |
| Number | 6 | | 6 | | 0 | | 3 | |
| Mean (SD) | −0.05 | (0.10) | −0.15 | (0.34) | NC | (NC) | 0.01 | (0.06) |
| LS Mean (SE) | −0.20 | (0.18) | −0.36 | (0.14) | NC | (NC) | NC | (NC) |
| LS Mean Diff vs. | | | −0.16 | (−0.52, 0.20) | | | NC | (NC, NC) |
| placebo (95% CI) | | | | | | | | |
| P-value vs. placebo | | | 0.3826 | | | | NC | |
| Overall p-value | | | 0.7653 | | | | 0.4094 | |
| for interaction | | | | | | | | |
| Atopic medical conditions | | | | | | | | |
| Yes | | | | | | | | |
| Number | 15 | | 16 | | 16 | | 16 | |
| Mean (SD) | 0.02 | (0.18) | 0.03 | (0.25) | −0.07 | (0.25) | −0.09 | (0.25) |
| LS Mean (SE) | 0.05 | (0.09) | 0.02 | (0.08) | −0.07 | (0.07) | −0.12 | (0.07) |
| LS Mean Diff vs. | | | −0.03 | (−0.19, 0.12) | | | −0.04 | (−0.23, 0.14) |
| placebo (95% CI) | | | | | | | | |
| P-value vs. placebo | | | 0.6793 | | | | 0.6505 | |
| No | | | | | | | | |
| Number | 10 | | 9 | | 38 | | 32 | |
| Mean (SD) | −0.17 | (0.23) | −0.16 | (0.56) | −0.06 | (0.34) | −0.18 | (0.28) |
| LS Mean (SE) | −0.25 | (0.16) | −0.15 | (0.19) | −0.04 | (0.07) | −0.15 | (0.07) |
| LS Mean Diff vs. | | | 0.10 | (−0.39, 0.60) | | | −0.11 | (−0.26, 0.04) |
| placebo (95% CI) | | | | | | | | |
| P-value vs. placebo | | | 0.6862 | | | | 0.1594 | |
| Overall p-value | | | 0.8948 | | | | 0.6133 | |
| for interaction | | | | | | | | |
| Background ICS dose level | | | | | | | | |
| at randomization (400 | | | | | | | | |
| mg TID only) | | | | | | | | |
| Medium | | | | | | | | |
| Number | | | | | 29 | | 21 | |
| Mean (SD) | | | | | −0.06 | (0.26) | −0.18 | (0.28) |
| LS Mean (SE) | | | | | −0.05 | (0.06) | −0.15 | (0.07) |
| LS Mean Diff vs. | | | | | | | −0.10 | (−0.25, 0.05) |
| placebo (95% CI) | | | | | | | | |
| P-value vs. placebo | | | | | | | 0.1837 | |
| High | | | | | | | | |
| Number | | | | | 25 | | 27 | |
| Mean (SD) | | | | | −0.07 | (0.37) | −0.13 | (0.27) |
| LS Mean (SE) | | | | | −0.07 | (0.07) | −0.13 | (0.07) |
| LS Mean Diff vs. | | | | | | | −0.07 | (−0.25, 0.11) |
| placebo (95% CI) | | | | | | | | |
| P-value vs. placebo | | | | | | | 0.4593 | |
| Overall p-value | | | | | | | 0.7498 | |
| for interaction | | | | | | | | |
| Smoking history | | | | | | | | |
| Former | | | | | | | | |
| Number | 7 | | 4 | | 3 | | 3 | |
| Mean (SD) | −0.07 | (0.28) | −0.36 | (0.39) | −0.06 | (0.09) | −0.21 | (0.28) |
| LS Mean (SE) | −0.36 | (0.22) | −0.57 | (0.43) | −0.07 | (0.16) | −0.12 | (0.16) |
| LS Mean Diff vs. | | | −0.21 | (−0.96, 0.55) | | | −0.05 | (−0.46, 0.37) |
| placebo (95% CI) | | | | | | | | |
| P-value vs. placebo | | | 0.5908 | | | | 0.8328 | |
| Never | | | | | | | | |
| Number | 18 | | 21 | | 51 | | 45 | |
| Mean (SD) | −0.05 | (0.19) | 0.02 | (0.37) | −0.06 | (0.32) | −0.15 | (0.27) |
| LS Mean (SE) | −0.08 | (0.11) | −0.05 | (0.10) | −0.04 | (0.05) | −0.13 | (0.05) |
| LS Mean Diff vs. | | | 0.03 | (−0.18, 0.24) | | | −0.09 | (−0.21, 0.04) |
| placebo (95% CI) | | | | | | | | |
| P-value vs. placebo | | | 0.7800 | | | | 0.1745 | |
| Overall p-value | | | 0.1306 | | | | 0.9511 | |
| for interaction | | | | | | | | |
| Baseline pre-bronchodilator | | | | | | | | |
| FEV1 (L) (400 mg BID only) | | | | | | | | |
| <Median (2.022) | | | | | | | | |
| Number | 12 | | 13 | | | | | |
| Mean (SD) | −0.08 | (0.25) | −0.06 | (0.27) | | | | |
| LS Mean (SE) | −0.12 | (0.13) | −0.08 | (0.14) | | | | |
| LS Mean Diff vs. | | | 0.04 | (−0.19, 0.27) | | | | |
| placebo (95% CI) | | | | | | | | |
| P-value vs. placebo | | | 0.7330 | | | | | |
| ≥Median (2.022) | | | | | | | | |
| Number | 13 | | 12 | | | | | |
| Mean (SD) | −0.04 | (0.19) | −0.02 | (0.50) | | | | |
| LS Mean (SE) | −0.07 | (0.13) | −0.09 | (0.14) | | | | |
| LS Mean Diff vs. | | | −0.03 | (−0.38, 0.32) | | | | |
| placebo (95% CI) | | | | | | | | |
| P-value vs. placebo | | | 0.8705 | | | | | |
| Overall p-value | | | 0.8540 | | | | | |
| for interaction | | | | | | | | |
| Baseline pre-bronchodilator | | | | | | | | |
| percent predicted FEV1 (%) | | | | | | | | |
| (400 mg BID only) | | | | | | | | |
| <Median (68) | | | | | | | | |
| Number | 11 | | 16 | | | | | |
| Mean (SD) | −0.08 | (0.26) | −0.05 | (0.41) | | | | |
| LS Mean (SE) | −0.24 | (0.16) | −0.21 | (0.16) | | | | |
| LS Mean Diff vs. | | | 0.03 | (−0.27, 0.34) | | | | |
| placebo (95% CI) | | | | | | | | |
| P-value vs. placebo | | | 0.8283 | | | | | |
| ≥Median (68) | | | | | | | | |
| Number | 14 | | 9 | | | | | |
| Mean (SD) | −0.04 | (0.18) | −0.02 | (0.39) | | | | |
| LS Mean (SE) | −0.06 | (0.13) | −0.07 | (0.13) | | | | |
| LS Mean Diff vs. | | | −0.01 | (−0.28, 0.26) | | | | |
| placebo (95% CI) | | | | | | | | |
| P-value vs. placebo | | | 0.9404 | | | | | |
| Overall p-value | | | 0.7813 | | | | | |
| for interaction | | | | | | | | |
| Baseline pre-bronchodilator | | | | | | | | |
| FEV1 (L) (400 mg TID only) | | | | | | | | |
| <Median (2.1955) | | | | | | | | |
| Number | | | | | 24 | | 18 | |
| Mean (SD) | | | | | 0.05 | (0.35) | −0.05 | (0.23) |
| LS Mean (SE) | | | | | 0.05 | (0.07) | −0.03 | (0.08) |
| LS Mean Diff vs. | | | | | | | −0.09 | (−0.28, 0.10) |
| placebo (95% CI) | | | | | | | | |
| P-value vs. placebo | | | | | | | 0.3726 | |
| ≥Median (2.1955) | | | | | | | | |
| Number | | | | | 30 | | 30 | |
| Mean (SD) | | | | | −0.15 | (0.26) | −0.21 | (0.28) |
| LS Mean (SE) | | | | | −0.13 | (0.06) | −0.22 | (0.06) |
| LS Mean Diff vs. | | | | | | | −0.09 | (−0.24, 0.07) |
| placebo (95% CI) | | | | | | | | |
| P-value vs. placebo | | | | | | | 0.2881 | |
| Overall p-value | | | | | | | 0.9725 | |
| for interaction | | | | | | | | |
| Baseline pre-bronchodilator | | | | | | | | |
| percent predicted FEV1 | | | | | | | | |
| group 1 (%) (400 mg | | | | | | | | |
| TID only) | | | | | | | | |
| <Median (77) | | | | | | | | |
| Number | | | | | 28 | | 21 | |
| Mean (SD) | | | | | −0.01 | (0.37) | −0.10 | (0.25) |
| LS Mean (SE) | | | | | 0.01 | (0.07) | −0.03 | (0.08) |
| LS Mean Diff vs. | | | | | | | −0.04 | (−0.24, 0.15) |
| placebo (95% CI) | | | | | | | | |
| P-value vs. placebo | | | | | | | 0.6453 | |
| ≥Median (77) | | | | | | | | |
| Number | | | | | 26 | | 27 | |
| Mean (SD) | | | | | −0.12 | (0.24) | −0.19 | (0.28) |
| LS Mean (SE) | | | | | −0.09 | (0.06) | −0.21 | (0.06) |
| LS Mean Diff vs. | | | | | | | −0.13 | (−0.28, 0.03) |
| placebo (95% CI) | | | | | | | | |
| P-value vs. placebo | | | | | | | 0.1033 | |
| Overall p-value | | | | | | | 0.4854 | |
| for interaction | | | | | | | | |
| Baseline pre-bronchodilator | | | | | | | | |
| percent predicted FEV1 | | | | | | | | |
| group 2 (%) (400 | | | | | | | | |
| mg TID only) | | | | | | | | |
| <80 | | | | | | | | |
| Number | | | | | 33 | | 26 | |
| Mean (SD) | | | | | −0.03 | (0.35) | −0.13 | (0.30) |
| LS Mean (SE) | | | | | −0.01 | (0.06) | −0.09 | (0.07) |
| LS Mean Diff vs. | | | | | | | −0.08 | (−0.25, 0.08) |
| placebo (95% CI) | | | | | | | | |
| P-value vs. placebo | | | | | | | 0.3269 | |
| ≥80 | | | | | | | | |
| Number | | | | | 21 | | 22 | |
| Mean (SD) | | | | | −0.12 | (0.25) | −0.17 | (0.24) |
| LS Mean (SE) | | | | | −0.09 | (0.06) | −0.20 | (0.06) |
| LS Mean Diff vs. | | | | | | | −0.11 | (−0.28, 0.06) |
| placebo (95% CI) | | | | | | | | |
| P-value vs. placebo | | | | | | | 0.2034 | |
| Overall p-value | | | | | | | 0.8177 | |
| for interaction | | | | | | | | |
| Baseline FEV1 | | | | | | | | |
| reversibility (%) | | | | | | | | |
| <12 | | | | | | | | |
| Number | 14 | | 10 | | 40 | | 35 | |
| Mean (SD) | −0.16 | (0.20) | −0.24 | (0.47) | −0.06 | (0.33) | −0.18 | (0.25) |
| LS Mean (SE) | −0.23 | (0.10) | −0.23 | (0.12) | −0.04 | (0.05) | −0.16 | (0.05) |
| LS Mean Diff vs. | | | −0.00 | (−0.26, 0.26) | | | −0.12 | (−0.25, 0.02) |
| placebo (95% CI) | | | | | | | | |
| P-value vs. placebo | | | 0.9928 | | | | 0.0828 | |
| ≥12 | | | | | | | | |
| Number | 9 | | 14 | | 9 | | 10 | |
| Mean (SD) | 0.04 | (0.15) | 0.08 | (0.27) | 0.01 | (0.30) | 0.00 | (0.27) |
| LS Mean (SE) | 0.12 | (0.12) | 0.07 | (0.11) | 0.02 | (0.13) | −0.01 | (0.12) |
| LS Mean Diff vs. | | | −0.05 | (−0.26, 0.16) | | | −0.02 | (−0.34, 0.29) |
| placebo (95% CI) | | | | | | | | |
| P-value vs. placebo | | | 0.6636 | | | | 0.8822 | |
| Overall p-value | | | 0.4867 | | | | 0.4422 | |
| for interaction | | | | | | | | |
| Baseline ACQ-5 score | | | | | | | | |
| ≤2 | | | | | | | | |
| Number | 12 | | 14 | | 20 | | 12 | |
| Mean (SD) | 0.00 | (0.20) | 0.00 | (0.47) | −0.20 | (0.24) | −0.10 | (0.19) |
| LS Mean (SE) | −0.15 | (0.16) | −0.12 | (0.13) | −0.11 | (0.06) | −0.12 | (0.08) |
| LS Mean Diff vs. | | | 0.04 | (−0.28, 0.35) | | | −0.01 | (−0.20, 0.19) |
| placebo (95% CI) | | | | | | | | |
| P-value vs. placebo | | | 0.8203 | | | | 0.9587 | |
| >2 | | | | | | | | |
| Number | 13 | | 11 | | 34 | | 36 | |
| Mean (SD) | −0.11 | (0.22) | −0.09 | (0.29) | 0.01 | (0.33) | −0.17 | (0.29) |
| LS Mean (SE) | −0.08 | (0.10) | −0.12 | (0.12) | 0.00 | (0.06) | −0.15 | (0.06) |
| LS Mean Diff vs. | | | −0.04 | (−0.29, 0.20) | | | −0.16 | (−0.31, 0.00) |
| placebo (95% CI) | | | | | | | | |

| | | |
|---|---|---|
| P-value vs. placebo | 0.7321 | 0.0511 |
| Overall p-value | 0.9187 | 0.2088 |
| for interaction | | |

LATAM: Latin America,
ROW: Rest of the world

TABLE 36
Subgroup analysis: change from baseline in pre-
bronchodilator FEV1 (L) at EOT (Week 12) by baseline
ICS/LABA dose level (mITT population).

| | Placebo | | Rilzabrutinib | |
|---|---|---|---|---|
| Pre-bronchodilator | BID | | 400 mg BID | |
| FEV1 (L) | (N = 32) | | (N = 32) | |
| Background ICS/LABA dose | | | | |
| level at randomization | | | | |
| Medium | | | | |
| Number | 2 | | 2 | |
| Mean (SD) | −0.07 | (0.00) | 0.04 | (0.47) |
| LS Mean (SE) | NC | (NC) | NC | (NC) |
| LS Mean Diff vs. | | | NC | (NC, NC) |
| placebo (95% CI) | | | | |
| P-value vs. placebo | | | NC | |
| High | | | | |
| Number | 23 | | 23 | |
| Mean (SD) | −0.06 | (0.23) | −0.05 | (0.40) |
| LS Mean (SE) | −0.12 | (0.09) | −0.11 | (0.09) |
| LS Mean Diff vs. | | | 0.02 | (−0.19, 0.23) |
| placebo (95% CI) | | | | |

| | |
|---|---|
| P-value vs. placebo | 0.8815 |
| Overall p-value | 0.8062 |
| for interaction | |

LATAM: Latin America,
ROW: Rest of the world

TABLE 37
Subgroup analysis: change from baseline in pre-bronchodilator FEV1 (L)
at EOT (Week 12) by baseline biomarker subgroups (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| Pre-bronchodilator | BID | | 400 mg BID | | TID | | 400 mg TID | |
| FEV1 (L) | (N = 32) | | (N = 32) | | (N = 68) | | (N = 64) | |
| Baseline IgE level (IU/mL) | | | | | | | | |
| <100 | | | | | | | | |
| Number | 7 | | 7 | | 22 | | 18 | |
| Mean (SD) | −0.10 | (0.30) | −0.09 | (0.52) | −0.08 | (0.21) | −0.20 | (0.31) |
| LS Mean (SE) | −0.07 | (0.27) | −0.11 | (0.26) | 0.02 | (0.12) | −0.10 | (0.13) |
| LS Mean Diff vs. | | | −0.03 | (−0.56, 0.50) | | | −0.11 | (−0.27, 0.05) |
| placebo (95% CI) | | | | | | | | |
| P-value vs. placebo | | | 0.8986 | | | | 0.1765 | |
| ≥100 | | | | | | | | |
| Number | 18 | | 18 | | 32 | | 30 | |
| Mean (SD) | −0.04 | (0.19) | −0.02 | (0.35) | −0.05 | (0.37) | −0.12 | (0.24) |
| LS Mean (SE) | −0.06 | (0.20) | −0.04 | (0.18) | −0.06 | (0.09) | −0.13 | (0.09) |
| LS Mean Diff vs. | | | 0.02 | (−0.20, 0.23) | | | −0.07 | (−0.24, 0.10) |
| placebo (95% CI) | | | | | | | | |
| P-value vs. placebo | | | 0.8821 | | | | 0.4002 | |
| Overall p-value | | | 0.5502 | | | | 0.6890 | |
| for interaction | | | | | | | | |
| Baseline median IgE | | | | | | | | |
| level (IU/mL) | | | | | | | | |
| (400 mg BID only) | | | | | | | | |
| <Median (210.8) | | | | | | | | |
| Number | 8 | | 17 | | | | | |
| Mean (SD) | −0.11 | (0.28) | −0.02 | (0.37) | | | | |
| LS Mean (SE) | −0.13 | (0.15) | −0.11 | (0.13) | | | | |
| LS Mean Diff vs. | | | 0.01 | (−0.34, 0.37) | | | | |
| placebo (95% CI) | | | | | | | | |
| P-value vs. placebo | | | 0.9444 | | | | | |
| ≥Median (210.8) | | | | | | | | |
| Number | 17 | | 8 | | | | | |
| Mean (SD) | −0.03 | (0.19) | −0.08 | (0.45) | | | | |
| LS Mean (SE | −0.04 | (0.11) | −0.02 | (0.14) | | | | |
| LS Mean Diff vs. | | | 0.01 | (−0.29, 0.31) | | | | |
| placebo (95% CI) | | | | | | | | |
| P-value vs. placebo | | | 0.9284 | | | | | |
| Overall p-value | | | 0.8938 | | | | | |
| for interaction | | | | | | | | |
| Baseline median IgE level | | | | | | | | |
| (IU/mL) (400 mg TID only) | | | | | | | | |
| <Median (121.85) | | | | | | | | |
| Number | | | | | 28 | | 24 | |
| Mean (SD) | | | | | −0.10 | (0.23) | −0.17 | (0.29) |
| LS Mean (SE) | | | | | −0.08 | (0.07) | −0.16 | (0.07) |
| LS Mean Diff vs. | | | | | | | −0.08 | (−0.23, 0.07) |
| placebo (95% CI) | | | | | | | | |
| P-value vs. placebo | | | | | | | 0.3135 | |
| ≥Median (121.85) | | | | | | | | |
| Number | | | | | 26 | | 24 | |
| Mean (SD) | | | | | −0.03 | (0.39) | −0.13 | (0.25) |
| LS Mean (SE) | | | | | −0.22 | (0.22) | −0.33 | (0.23) |
| LS Mean Diff vs. | | | | | | | −0.11 | (−0.30, 0.07) |
| placebo (95% CI) | | | | | | | | |
| P-value vs. placebo | | | | | | | 0.2365 | |
| Overall p-value | | | | | | | 0.8457 | |
| for interaction | | | | | | | | |
| Baseline median IgA level | | | | | | | | |
| (mg/L) (400 mg TID only) | | | | | | | | |
| <Median (2190) | | | | | | | | |
| Number | | | | | 29 | | 21 | |
| Mean (SD) | | | | | −0.04 | (0.23) | −0.12 | (0.26) |
| LS Mean (SE) | | | | | −0.03 | (0.06) | −0.12 | (0.07) |
| LS Mean Diff vs. | | | | | | | −0.09 | (−0.25, 0.07) |
| placebo (95% CI) | | | | | | | | |
| P-value vs. placebo | | | | | | | 0.2572 | |
| ≥Median (2190) | | | | | | | | |
| Number | | | | | 25 | | 27 | |
| Mean (SD) | | | | | −0.09 | (0.39) | −0.17 | (0.28) |
| LS Mean (SE) | | | | | −0.06 | (0.07) | −0.15 | (0.07) |
| LS Mean Diff vs. | | | | | | | −0.09 | (−0.27, 0.09) |
| placebo (95% CI) | | | | | | | | |
| P-value vs. placebo | | | | | | | 0.3176 | |
| Overall p-value | | | | | | | 0.9795 | |
| for interaction | | | | | | | | |
| Baseline blood eosinophil | | | | | | | | |
| level 1 (10\hat{ }9/L) | | | | | | | | |
| <0.15 | | | | | | | | |
| Number | 5 | | 8 | | 22 | | 22 | |
| Mean (SD) | 0.03 | (0.18) | −0.10 | (0.49) | −0.15 | (0.27) | −0.17 | (0.32) |
| LS Mean (SE) | −0.04 | (0.26) | −0.08 | (0.16) | −0.13 | (0.07) | −0.17 | (0.07) |
| LS Mean Diff vs. | | | −0.04 | (−0.65, 0.57) | | | −0.05 | (−0.23, 0.14) |
| placebo (95% CI) | | | | | | | | |
| P-value vs. placebo | | | 0.8932 | | | | 0.6211 | |
| ≥0.15 (400 mg BID only) | | | | | | | | |
| Number | 20 | | 17 | | | | | |
| Mean (SD) | −0.08 | (0.22) | −0.01 | (0.35) | | | | |
| LS Mean (SE) | −0.10 | (0.10) | −0.03 | (0.11) | | | | |
| LS Mean Diff vs. | | | 0.07 | (−0.15, 0.28) | | | | |
| placebo (95% CI) | | | | | | | | |
| P-value vs. placebo | | | 0.5498 | | | | | |
| Overall p-value for interaction | | | 0.3640 | | | | | |
| Baseline blood eosinophil | | | | | | | | |
| level 2 (10\hat{ }9/L) | | | | | | | | |
| (400 mg BID only) | | | | | | | | |
| <0.3 | | | | | | | | |
| Number | 14 | | 14 | | | | | |
| Mean (SD) | −0.01 | (0.25) | −0.10 | (0.42) | | | | |
| LS Mean (SE) | −0.08 | (0.11) | −0.15 | (0.12) | | | | |
| LS Mean Diff vs. | | | −0.07 | (−0.35, 0.20) | | | | |
| placebo (95% CI) | | | | | | | | |
| P-value vs. placebo | | | 0.6075 | | | | | |
| ≥0.3 | | | | | | | | |
| Number | 11 | | 11 | | | | | |
| Mean (SD) | −0.11 | (0.16) | 0.04 | (0.36) | | | | |
| LS Mean (SE) | −0.07 | (0.17) | 0.00 | (0.14) | | | | |
| LS Mean Diff vs. | | | 0.07 | (−0.21, 0.35) | | | | |
| placebo (95% CI) | | | | | | | | |
| P-value vs. placebo | | | 0.6310 | | | | | |
| Overall p-value | | | 0.2578 | | | | | |
| for interaction | | | | | | | | |
| Baseline blood eosinophil | | | | | | | | |
| level 1 (10\hat{ }9/L) | | | | | | | | |
| (400 mg TID only) | | | | | | | | |
| <0.15 | | | | | | | | |
| Number | | | | | 22 | | 22 | |
| Mean (SD) | | | | | −0.15 | (0.27) | −0.17 | (0.32) |
| LS Mean (SE) | | | | | −0.13 | (0.07) | −0.17 | (0.07) |
| LS Mean Diff vs. | | | | | | | −0.05 | (−0.23, 0.14) |
| placebo (95% CI) | | | | | | | | |
| P-value vs. placebo | | | | | | | 0.6211 | |
| ≥0.15-<0.3 | | | | | | | | |
| Number | | | | | 18 | | 13 | |
| Mean (SD) | | | | | −0.04 | (0.25) | −0.10 | (0.16) |
| LS Mean (SE) | | | | | −0.00 | (0.08) | −0.04 | (0.09) |
| LS Mean Diff vs. | | | | | | | −0.04 | (−0.24, 0.17) |
| placebo (95% CI) | | | | | | | | |
| P-value vs. placebo | | | | | | | 0.7282 | |
| ≥0.3 | | | | | | | | |
| Number | | | | | 14 | | 13 | |
| Mean (SD) | | | | | 0.04 | (0.42) | −0.16 | (0.27) |
| LS Mean (SE) | | | | | 0.04 | (0.12) | −0.16 | (0.11) |
| LS Mean Diff vs. | | | | | | | −0.20 | (−0.45, 0.05) |
| placebo (95% CI) | | | | | | | | |
| P-value vs. placebo | | | | | | | 0.1159 | |
| Overall p-value | | | | | | | 0.5643 | |
| for interaction | | | | | | | | |
| Baseline blood eosinophil | | | | | | | | |
| level 2 (10\hat{ }9/L) | | | | | | | | |
| (400 mg TID only) | | | | | | | | |
| <0.3 | | | | | | | | |
| Number | | | | | 40 | | 35 | |
| Mean (SD) | | | | | −0.10 | (0.27) | −0.15 | (0.27) |
| LS Mean (SE) | | | | | −0.08 | (0.05) | −0.13 | (0.05) |
| LS Mean Diff vs. | | | | | | | −0.05 | (−0.19, 0.09) |
| placebo (95% CI) | | | | | | | | |
| P-value vs. placebo | | | | | | | 0.4664 | |
| ≥0.3 | | | | | | | | |
| Number | | | | | 14 | | 13 | |
| Mean (SD) | | | | | 0.04 | (0.42) | −0.16 | (0.27) |
| LS Mean (SE) | | | | | 0.04 | (0.12) | −0.16 | (0.11) |
| LS Mean Diff vs. | | | | | | | −0.20 | (−0.45, 0.05) |
| placebo (95% CI) | | | | | | | | |
| P-value vs. placebo | | | | | | | 0.1159 | |
| Overall p-value | | | | | | | 0.3072 | |
| for interaction | | | | | | | | |
| Baseline FeNO | | | | | | | | |
| level 1 (ppb) | | | | | | | | |
| <25 | | | | | | | | |
| Number | 11 | | 9 | | 31 | | 32 | |
| Mean (SD) | −0.04 | (0.26) | −0.12 | (0.51) | −0.06 | (0.25) | −0.19 | (0.28) |
| LS Mean (SE) | −0.18 | (0.15) | −0.16 | (0.14) | −0.04 | (0.05) | −0.16 | (0.05) |
| LS Mean Diff vs. | | | 0.03 | (−0.32, 0.37) | | | −0.12 | (−0.26, 0.02) |
| placebo (95% CI) | | | | | | | | |
| P-value vs. placebo | | | 0.8792 | | | | 0.0953 | |
| ≥25 | | | | | | | | |
| Number | 14 | | 16 | | 19 | | 14 | |
| Mean (SD) | −0.07 | (0.19) | 0.01 | (0.32) | −0.06 | (0.41) | −0.09 | (0.25) |
| LS Mean (SE) | 0.01 | (0.11) | −0.00 | (0.11) | −0.04 | (0.10) | −0.09 | (0.11) |
| LS Mean Diff vs. | | | −0.01 | (−0.23, 0.22) | | | −0.04 | (−0.32, 0.23) |
| placebo (95% CI) | | | | | | | | |
| P-value vs. placebo | | | 0.9359 | | | | 0.7523 | |
| Overall p-value | | | 0.6767 | | | | 0.5841 | |
| for interaction | | | | | | | | |
| Baseline FeNO | | | | | | | | |
| level 2 (ppb) | | | | | | | | |
| <35 | | | | | | | | |
| Number | 14 | | 17 | | 38 | | 36 | |
| Mean (SD) | −0.03 | (0.25) | −0.10 | (0.43) | −0.09 | (0.27) | −0.18 | (0.27) |
| LS Mean (SE) | −0.15 | (0.13) | −0.19 | (0.11) | −0.06 | (0.05) | −0.14 | (0.05) |
| LS Mean Diff vs. | | | −0.04 | (−0.30, 0.21) | | | −0.09 | (−0.22, 0.04) |
| placebo (95% CI) | | | | | | | | |
| P-value vs. placebo | | | 0.7398 | | | | 0.1794 | |
| ≥35 | | | | | | | | |
| Number | 11 | | 8 | | 12 | | 10 | |
| Mean (SD) | −0.09 | (0.17) | 0.08 | (0.29) | 0.02 | (0.44) | −0.07 | (0.27) |
| LS Mean (SE) | −0.01 | (0.12) | 0.05 | (0.14) | 0.04 | (0.17) | −0.07 | (0.16) |
| LS Mean Diff vs. | | | 0.06 | (−0.26, 0.38) | | | −0.12 | (−0.49, 0.26) |
| placebo (95% CI) | | | | | | | | |

| | | |
|---|---|---|
| P-value vs. placebo | 0.7100 | 0.5388 |
| Overall p-value | 0.2631 | 0.8679 |
| for interaction | | |

LATAM: Latin America,
ROW: Rest of the world

TABLE 38
Subgroup analysis: change from baseline in pre-bronchodilator FEV1 (L) at EOT
(Week 12) in the baseline low EOS and low FeNO subgroup (mITT population).

| | 400 mg BID cohort | | | | 400 mg TID cohort | | | |
|---|---|---|---|---|---|---|---|---|
| | Placebo | | Rilzabrutinib | | Placebo | | Rilzabrutinib | |
| Pre-bronchodilator | BID | | 400 mg BID | | TID | | 400 mg TID | |
| FEV1 (L) | (N = 32) | | (N = 32) | | (N = 68) | | (N = 64) | |
| Baseline blood eosinophil | | | | | | | | |
| (10\hat{ }9/L) and FeNO (ppb) | | | | | | | | |
| Low EOS (<0.15) and | | | | | | | | |
| low FeNO | | | | | | | | |
| (<25) | | | | | | | | |
| Number | 3 | | 3 | | 16 | | 16 | |
| Mean (SD) | 0.02 | (0.17) | −0.17 | (0.85) | −0.08 | (0.23) | −0.24 | (0.35) |
| LS Mean (SE) | NC | (NC) | NC | (NC) | −0.06 | (0.07) | −0.20 | (0.07) |
| LS Mean Diff vs. | | | NC | (NC, NC) | | | −0.14 | (−0.33, 0.06) |
| placebo (95% CI) | | | | | | | | |
| P-value vs. placebo | | | NC | | | | 0.1770 | |
| All others | | | | | | | | |
| Number | 22 | | 22 | | 34 | | 30 | |
| Mean (SD) | −0.07 | (0.22) | −0.02 | (0.32) | −0.05 | (0.35) | −0.11 | (0.22) |
| LS Mean (SE) | −0.08 | (0.09) | −0.05 | (0.09) | −0.02 | (0.06) | −0.09 | (0.06) |
| LS Mean Diff vs. | | | 0.03 | (−0.16, 0.21) | | | −0.07 | (−0.23, 0.09) |
| placebo (95% CI) | | | | | | | | |

| | | |
|---|---|---|
| P-value vs. placebo | 0.7753 | 0.3909 |
| Overall p-value | 0.3586 | 0.6290 |
| for interaction | | |

FLATAM: Latin America,
ROW: Rest of the world

### Bronchodilator Therapy

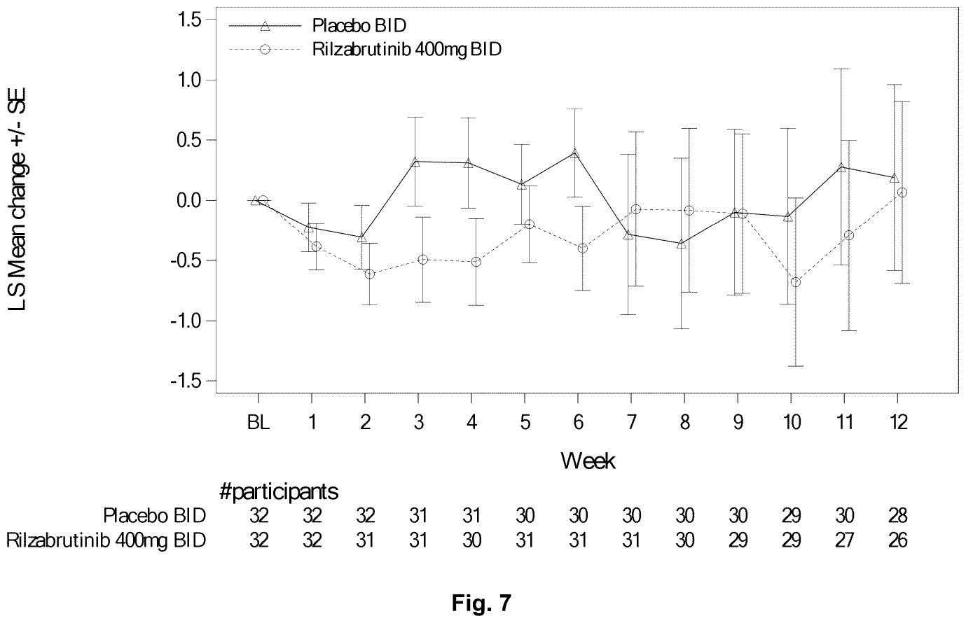

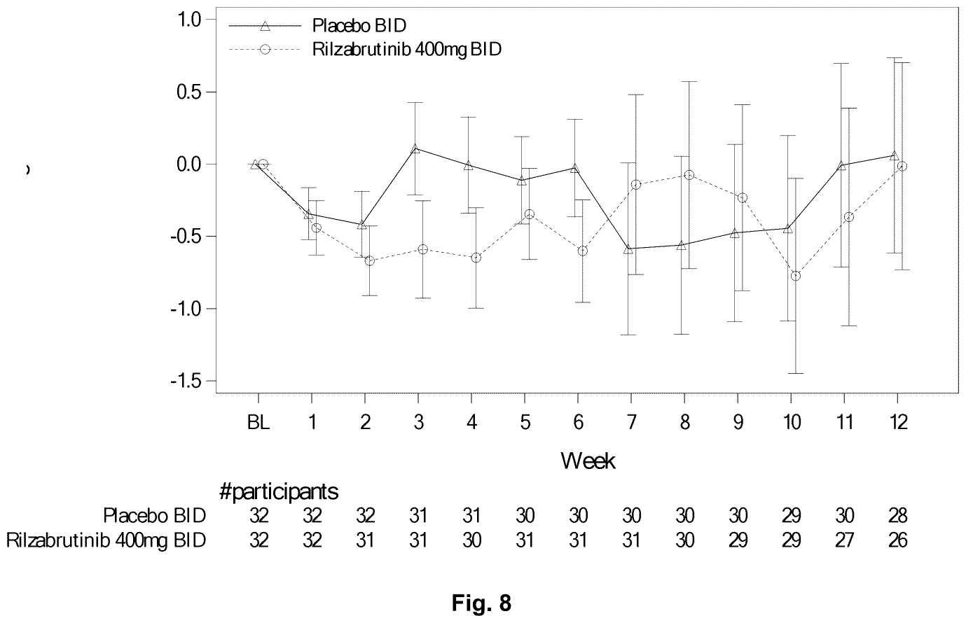

When used in combination with ICS/LABA, treatment with 400 mg BID rilzabrutinib led to a reduction in the number of inhalations/day relative to treatment with the placebo (FIGS. 7-8 and Tables 39-40A).

An analysis was performed on the number of participants with ≥2, ≥3, ≥4, and ≥5 additional reliever puffs of salbutamol/albuterol or levosalbutamol/levalbuterol compared with baseline on 2 consecutive days during the treatment period, to assess increase in reliever medication during 12-week treatment period. In both cohorts, number of participants with ≥2 to 5 additional reliever puffs compared to baseline was lower in the rilzabrutinib arms compared to the placebo arms (Table 40B).

TABLE 39
Change from baseline in inhalations/day of albuterol or levalbuterol
for symptom relief over time (mITT population).

| | | |
|---|---|---|
| | Placebo | Rilzabrutinib |
| Albuterol or levalbuterol | BID | 400 mg BID |
| for symptom relief | (N = 32) | (N = 32) |

| Baseline | | | | |
|---|---|---|---|---|
| Number | 32 | | 32 | |
| Mean (SD) | 2.52 | (3.02) | 1.57 | (1.98) |
| Median | 1.29 | | 0.62 | |
| Q1; Q3 | 0.00; | 4.71 | 0.00; | 2.93 |
| Min; Max | 0.0; | 12.3 | 0.0; | 7.4 |
| Week 1 | | | | |
| Number (observed/LOCF imputed) | 32 | (32/0) | 32 | (32/0) |
| Mean (SD) | 2.02 | (2.56) | 1.15 | (1.78) |
| Median | 0.59 | | 0.21 | |
| Q1; Q3 | 0.00; | 3.50 | 0.00; | 1.93 |
| Min; Max | 0.0; | 10.3 | 0.0; | 7.4 |
| Change from baseline | | | | |
| Number (observed/LOCF imputed) | 32 | (32/0) | 32 | (32/0) |
| Mean (SD) | −0.50 | (1.03) | −0.41 | (1.03) |
| Median | 0.00 | | 0.00 | |
| Q1; Q3 | −0.69; | 0.00 | −0.62; | 0.00 |
| Min; Max | −3.5; | 0.7 | −5.3; | 1.0 |
| LS Mean (SE) | −0.23 | (0.20) | −0.39 | (0.19) |
| LS Mean Diff vs. | | | −0.16 | (−0.64, 0.32) |
| placebo (95% CI) | | | | |
| P-value vs. placebo | | | 0.5052 | |
| Week 2 | | | | |
| Number (observed/LOCF imputed) | 32 | (31/1) | 31 | (31/0) |
| Mean (SD) | 1.91 | (2.63) | 0.94 | (1.76) |
| Median | 0.37 | | 0.14 | |
| Q1; Q3 | 0.00; | 3.21 | 0.00; | 0.80 |
| Min; Max | 0.0; | 9.5 | 0.0; | 7.4 |
| Change from baseline | | | | |
| Number (observed/LOCF imputed) | 32 | (31/1) | 31 | (31/0) |
| Mean (SD) | −0.61 | (1.34) | −0.68 | (1.24) |
| Median | 0.00 | | 0.00 | |
| Q1; Q3 | −1.50; | 0.00 | −1.14; | 0.00 |
| Min; Max | −4.0; | 2.9 | −5.3; | 0.3 |
| LS Mean (SE) | −0.31 | (0.27) | −0.61 | (0.26) |
| LS Mean Diff vs. | | | −0.31 | (−0.91, 0.30) |
| placebo (95% CI) | | | | |
| P-value vs. placebo | | | 0.3228 | |
| Week 3 | | | | |
| Number (observed/LOCF imputed) | 31 | (30/1) | 31 | (31/0) |
| Mean (SD) | 1.96 | (2.75) | 0.79 | (1.48) |
| Median | 0.29 | | 0.00 | |
| Q1; Q3 | 0.00; | 4.00 | 0.00; | 0.67 |
| Min; Max | 0.0; | 9.0 | 0.0; | 5.3 |
| Change from baseline | | | | |
| Number (observed/LOCF imputed) | 31 | (30/1) | 31 | (31/0) |
| Mean (SD) | −0.24 | (1.98) | −0.83 | (1.48) |
| Median | 0.00 | | 0.00 | |
| Q1; Q3 | −0.67; | 0.00 | −1.33; | 0.00 |
| Min; Max | −5.4; | 6.3 | −5.0; | 1.3 |
| LS Mean (SE) | 0.32 | (0.37) | −0.49 | (0.35) |
| LS Mean Diff vs. | | | −0.81 | (−1.64, 0.01) |
| placebo (95% CI) | | | | |
| P-value vs. placebo | | | 0.0539 | |
| Week 4 | | | | |
| Number (observed/LOCF imputed) | 31 | (29/2) | 30 | (29/1) |
| Mean (SD) | 1.92 | (2.71) | 0.86 | (1.72) |
| Median | 0.29 | | 0.00 | |
| Q1; Q3 | 0.00; | 3.43 | 0.00; | 0.50 |
| Min; Max | 0.0; | 9.0 | 0.0; | 6.0 |
| Change from baseline | | | | |
| Number (observed/LOCF imputed) | 31 | (29/2) | 30 | (29/1) |
| Mean (SD) | −0.28 | (2.14) | −0.81 | (1.41) |
| Median | 0.00 | | −0.29 | |
| Q1; Q3 | −1.29; | 0.00 | −1.71; | 0.00 |
| Min; Max | −5.4; | 6.3 | −5.5; | 2.0 |
| LS Mean (SE) | 0.31 | (0.37) | −0.51 | (0.36) |
| LS Mean Diff vs. | | | −0.82 | (−1.67, 0.03) |
| placebo (95% CI) | | | | |
| P-value vs. placebo | | | 0.0573 | |
| Week 5 | | | | |
| Number (observed/LOCF imputed) | 30 | (28/2) | 31 | (29/2) |
| Mean (SD) | 1.89 | (2.65) | 1.11 | (1.80) |
| Median | 0.25 | | 0.00 | |
| Q1; Q3 | 0.00; | 4.00 | 0.00; | 2.50 |
| Min; Max | 0.0; | 9.0 | 0.0; | 5.7 |
| Change from baseline | | | | |
| Number (observed/LOCF imputed) | 30 | (28/2) | 31 | (29/2) |
| Mean (SD) | −0.39 | (1.97) | −0.51 | (1.14) |
| Median | 0.00 | | 0.00 | |
| Q1; Q3 | −1.29; | 0.00 | −0.83; | 0.00 |
| Min; Max | −4.9; | 6.3 | −3.5; | 1.0 |
| LS Mean (SE) | 0.13 | (0.33) | −0.20 | (0.32) |
| LS Mean Diff vs. | | | −0.33 | (−1.08, 0.41) |
| placebo (95% CI) | | | | |
| P-value vs. placebo | | | 0.3824 | |
| Week 6 | | | | |
| Number (observed/LOCF imputed) | 30 | (28/2) | 31 | (29/2) |
| Mean (SD) | 2.13 | (2.77) | 1.00 | (1.92) |
| Median | 1.14 | | 0.00 | |
| Q1; Q3 | 0.00; | 3.14 | 0.00; | 0.50 |
| Min; Max | 0.0; | 9.0 | 0.0; | 7.3 |
| Change from baseline | | | | |
| Number (observed/LOCF imputed) | 30 | (28/2) | 31 | (29/2) |
| Mean (SD) | −0.15 | (1.94) | −0.61 | (1.44) |
| Median | 0.00 | | 0.00 | |
| Q1; Q3 | −0.83; | 0.00 | −1.00; | 0.05 |
| Min; Max | −4.9; | 6.3 | −5.0; | 1.3 |
| LS Mean (SE) | 0.39 | (0.36) | −0.40 | (0.35) |
| LS Mean Diff vs. | | | −0.79 | (−1.61, 0.03) |
| placebo (95% CI) | | | | |
| P-value vs. placebo | | | 0.0589 | |
| Week 7 | | | | |
| Number (observed/LOCF imputed) | 30 | (27/3) | 31 | (29/2) |
| Mean (SD) | 1.64 | (2.52) | 1.61 | (3.82) |
| Median | 0.57 | | 0.00 | |
| Q1; Q3 | 0.00; | 2.00 | 0.00; | 0.86 |
| Min; Max | 0.0; | 9.0 | 0.0; | 19.4 |
| Change from baseline | | | | |
| Number (observed/LOCF imputed) | 30 | (27/3) | 31 | (29/2) |
| Mean (SD) | −0.63 | (2.16) | −0.00 | (3.35) |
| Median | −0.05 | | 0.00 | |
| Q1; Q3 | −1.43; | 0.00 | −1.00; | 0.00 |
| Min; Max | −4.9; | 6.3 | −4.3; | 16.8 |
| LS Mean (SE) | −0.28 | (0.67) | −0.07 | (0.64) |
| LS Mean Diff vs. | | | 0.21 | (−1.27, 1.70) |
| placebo (95% CI) | | | | |
| P-value vs. placebo | | | 0.7812 | |
| Week 8 | | | | |
| Number (observed/LOCF imputed) | 30 | (27/3) | 30 | (27/3) |
| Mean (SD) | 1.59 | (2.52) | 1.66 | (3.98) |
| Median | 0.21 | | 0.00 | |
| Q1; Q3 | 0.00; | 3.00 | 0.00; | 1.14 |
| Min; Max | 0.0; | 9.0 | 0.0; | 19.4 |
| Change from baseline | | | | |
| Number (observed/LOCF imputed) | 30 | (27/3) | 30 | (27/3) |
| Mean (SD) | −0.68 | (2.34) | −0.01 | (3.76) |
| Median | 0.00 | | 0.00 | |
| Q1; Q3 | −1.86; | 0.00 | −1.00; | 0.00 |
| Min; Max | −5.9; | 6.3 | −5.5; | 16.8 |
| LS Mean (SE) | −0.36 | (0.71) | −0.08 | (0.68) |
| LS Mean Diff vs. | | | 0.28 | (−1.32, 1.87) |
| placebo (95% CI) | | | | |
| P-value vs. placebo | | | 0.7344 | |
| Week 9 | | | | |
| Number (observed/LOCF imputed) | 30 | (27/3) | 29 | (25/4) |
| Mean (SD) | 1.70 | (2.55) | 1.66 | (3.98) |
| Median | 0.29 | | 0.00 | |
| Q1; Q3 | 0.00; | 2.29 | 0.00; | 1.00 |
| Min; Max | 0.0; | 9.0 | 0.0; | 19.4 |
| Change from baseline | | | | |
| Number (observed/LOCF imputed) | 30 | (27/3) | 29 | (25/4) |
| Mean (SD) | −0.57 | (2.40) | −0.07 | (3.96) |
| Median | 0.00 | | −0.29 | |
| Q1; Q3 | −1.86; | 0.00 | −1.14; | 0.00 |
| Min; Max | −5.9; | 6.3 | −5.5; | 16.8 |
| LS Mean (SE) | −0.10 | (0.69) | −0.11 | (0.66) |
| LS Mean Diff vs. | | | −0.01 | (−1.58, 1.56) |
| placebo (95% CI) | | | | |
| P-value vs. placebo | | | 0.9891 | |
| Week 10 | | | | |
| Number (observed/LOCF imputed) | 29 | (25/4) | 29 | (25/4) |
| Mean (SD) | 1.90 | (2.72) | 1.49 | (3.98) |
| Median | 0.00 | | 0.00 | |
| Q1; Q3 | 0.00; | 3.29 | 0.00; | 0.57 |
| Min; Max | 0.0; | 9.0 | 0.0; | 19.4 |
| Change from baseline | | | | |
| Number (observed/LOCF imputed) | 29 | (25/4) | 29 | (25/4) |
| Mean (SD) | −0.25 | (2.97) | −0.23 | (3.88) |
| Median | 0.00 | | −0.29 | |
| Q1; Q3 | −1.86; | 0.33 | −1.86; | 0.00 |
| Min; Max | −6.9; | 6.3 | −5.5; | 16.8 |
| LS Mean (SE) | −0.13 | (0.73) | −0.68 | (0.70) |
| LS Mean Diff vs. | | | −0.55 | (−2.20, 1.11) |
| placebo (95% CI) | | | | |
| P-value vs. placebo | | | 0.5185 | |
| Week 11 | | | | |
| Number (observed/LOCF imputed) | 30 | (24/6) | 27 | (23/4) |
| Mean (SD) | 2.37 | (3.30) | 1.61 | (4.24) |
| Median | 0.33 | | 0.00 | |
| Q1; Q3 | 0.00; | 5.00 | 0.00; | 0.67 |
| Min; Max | 0.0; | 11.0 | 0.0; | 19.4 |
| Change from baseline | | | | |
| Number (observed/LOCF imputed) | 30 | (24/6) | 27 | (23/4) |
| Mean (SD) | 0.09 | (3.30) | −0.10 | (3.99) |
| Median | 0.00 | | −0.40 | |
| Q1; Q3 | −1.71; | 0.50 | −1.43; | 0.00 |
| Min; Max | −6.5; | 7.2 | −5.5; | 16.8 |
| LS Mean (SE) | 0.28 | (0.82) | −0.29 | (0.79) |
| LS Mean Diff vs. | | | −0.57 | (−2.39, 1.25) |
| placebo (95% CI) | | | | |
| P-value vs. placebo | | | 0.5406 | |
| Week 12 | | | | |
| Number (observed/LOCF imputed) | 28 | (19/9) | 26 | (21/5) |
| Mean (SD) | 2.57 | (3.34) | 1.97 | (4.32) |
| Median | 1.14 | | 0.00 | |
| Q1; Q3 | 0.00; | 4.60 | 0.00; | 2.00 |
| Min; Max | 0.0; | 11.0 | 0.0; | 19.4 |
| Change from baseline | | | | |
| Number (observed/LOCF imputed) | 28 | (19/9) | 26 | (21/5) |
| Mean (SD) | 0.16 | (3.31) | 0.26 | (3.85) |
| Median | 0.00 | | −0.19 | |
| Q1; Q3 | −2.41; | 1.00 | −1.00; | 0.00 |
| Min; Max | −5.3; | 7.2 | −4.3; | 16.8 |
| LS Mean (SE) | 0.19 | (0.77) | 0.07 | (0.76) |
| LS Mean Diff vs. | | | −0.12 | (−1.90, 1.66) |
| placebo (95% CI) | | | | |

| | |
|---|---|
| P-value vs. placebo | 0.8932 |

LATAM: Latin America,
ROW: Rest of the world

TABLE 40A
Change from baseline in inhalations/day of albuterol or levalbuterol
for symptom relief over time, reduced model (mITT population).

| | | |
|---|---|---|
| | Placebo | Rilzabrutinib |
| Albuterol or levalbuterol | BID | 400 mg BID |
| for symptom relief | (N = 32) | (N = 32) |

| Baseline | | | | |
|---|---|---|---|---|
| Number | 32 | | 32 | |
| Mean (SD) | 2.52 | (3.02) | 1.57 | (1.98) |
| Median | 1.29 | | 0.62 | |
| Q1; Q3 | 0.00; | 4.71 | 0.00; | 2.93 |
| Min; Max | 0.0; | 12.3 | 0.0; | 7.4 |
| Week 1 | | | | |
| Number (observed/LOCF imputed) | 32 | (32/0) | 32 | (32/0) |
| Mean (SD) | 2.02 | (2.56) | 1.15 | (1.78) |
| Median | 0.59 | | 0.21 | |
| Q1; Q3 | 0.00; | 3.50 | 0.00; | 1.93 |
| Min; Max | 0.0; | 10.3 | 0.0; | 7.4 |
| Change from baseline | | | | |
| Number (observed/LOCF imputed) | 32 | (32/0) | 32 | (32/0) |
| Mean (SD) | −0.50 | (1.03) | −0.41 | (1.03) |
| Median | 0.00 | | 0.00 | |
| Q1; Q3 | −0.69; | 0.00 | −0.62; | 0.00 |
| Min; Max | −3.5; | 0.7 | −5.3; | 1.0 |
| LS Mean (SE) | −0.34 | (0.18) | −0.44 | (0.19) |
| LS Mean Diff vs. | | | −0.10 | (−0.56, 0.36) |
| placebo (95% CI) | | | | |
| P-value vs. placebo | | | 0.6691 | |
| Week 2 | | | | |
| Number (observed/LOCF imputed) | 32 | (31/1) | 31 | (31/0) |
| Mean (SD) | 1.91 | (2.63) | 0.94 | (1.76) |
| Median | 0.37 | | 0.14 | |
| Q1; Q3 | 0.00; | 3.21 | 0.00; | 0.80 |
| Min; Max | 0.0; | 9.5 | 0.0; | 7.4 |
| Change from baseline | | | | |
| Number (observed/LOCF imputed) | 32 | (31/1) | 31 | (31/0) |
| Mean (SD) | −0.61 | (1.34) | −0.68 | (1.24) |
| Median | 0.00 | | 0.00 | |
| Q1; Q3 | −1.50; | 0.00 | −1.14; | 0.00 |
| Min; Max | −4.0; | 2.9 | −5.3; | 0.3 |
| LS Mean (SE) | −0.42 | (0.23) | −0.67 | (0.24) |
| LS Mean Diff vs. | | | −0.25 | (−0.82, 0.32) |
| placebo (95% CI) | | | | |
| P-value vs. placebo | | | 0.3848 | |
| Week 3 | | | | |
| Number (observed/LOCF imputed) | 31 | (30/1) | 31 | (31/0) |
| Mean (SD) | 1.96 | (2.75) | 0.79 | (1.48) |
| Median | 0.29 | | 0.00 | |
| Q1; Q3 | 0.00; | 4.00 | 0.00; | 0.67 |
| Min; Max | 0.0; | 9.0 | 0.0; | 5.3 |
| Change from baseline | | | | |
| Number (observed/LOCF imputed) | 31 | (30/1) | 31 | (31/0) |
| Mean (SD) | −0.24 | (1.98) | −0.83 | (1.48) |
| Median | 0.00 | | 0.00 | |
| Q1; Q3 | −0.67; | 0.00 | −1.33; | 0.00 |
| Min; Max | −5.4; | 6.3 | −5.0; | 1.3 |
| LS Mean (SE) | 0.11 | (0.32) | −0.59 | (0.34) |
| LS Mean Diff vs. | | | −0.70 | (−1.49, 0.09) |
| placebo (95% CI) | | | | |
| P-value vs. placebo | | | 0.0826 | |
| Week 4 | | | | |
| Number (observed/LOCF imputed) | 31 | (29/2) | 30 | (29/1) |
| Mean (SD) | 1.92 | (2.71) | 0.86 | (1.72) |
| Median | 0.29 | | 0.00 | |
| Q1; Q3 | 0.00; | 3.43 | 0.00; | 0.50 |
| Min; Max | 0.0; | 9.0 | 0.0; | 6.0 |
| Change from baseline | | | | |
| Number (observed/LOCF imputed) | 31 | (29/2) | 30 | (29/1) |
| Mean (SD) | −0.28 | (2.14) | −0.81 | (1.41) |
| Median | 0.00 | | −0.29 | |
| Q1; Q3 | −1.29; | 0.00 | −1.71; | 0.00 |
| Min; Max | −5.4; | 6.3 | −5.5; | 2.0 |
| LS Mean (SE) | −0.01 | (0.33) | −0.65 | (0.35) |
| LS Mean Diff vs. | | | −0.64 | (−1.46, 0.18) |
| placebo (95% CI) | | | | |
| P-value vs. placebo | | | 0.1245 | |
| Week 5 | | | | |
| Number (observed/LOCF imputed) | 30 | (28/2) | 31 | (29/2) |
| Mean (SD) | 1.89 | (2.65) | 1.11 | (1.80) |
| Median | 0.25 | | 0.00 | |
| Q1; Q3 | 0.00; | 4.00 | 0.00; | 2.50 |
| Min; Max | 0.0; | 9.0 | 0.0; | 5.7 |
| Change from baseline | | | | |
| Number (observed/LOCF imputed) | 30 | (28/2) | 31 | (29/2) |
| Mean (SD) | −0.39 | (1.97) | −0.51 | (1.14) |
| Median | 0.00 | | 0.00 | |
| Q1; Q3 | −1.29; | 0.00 | −0.83; | 0.00 |
| Min; Max | −4.9; | 6.3 | −3.5; | 1.0 |
| LS Mean (SE) | −0.11 | (0.30) | −0.35 | (0.32) |
| LS Mean Diff vs. | | | −0.23 | (−0.97, 0.50) |
| placebo (95% CI) | | | | |
| P-value vs. placebo | | | 0.5339 | |
| Week 6 | | | | |
| Number (observed/LOCF imputed) | 30 | (28/2) | 31 | (29/2) |
| Mean (SD) | 2.13 | (2.77) | 1.00 | (1.92) |
| Median | 1.14 | | 0.00 | |
| Q1; Q3 | 0.00; | 3.14 | 0.00; | 0.50 |
| Min; Max | 0.0; | 9.0 | 0.0; | 7.3 |
| Change from baseline | | | | |
| Number (observed/LOCF imputed) | 30 | (28/2) | 31 | (29/2) |
| Mean (SD) | −0.15 | (1.94) | −0.61 | (1.44) |
| Median | 0.00 | | 0.00 | |
| Q1; Q3 | −0.83; | 0.00 | −1.00; | 0.05 |
| Min; Max | −4.9; | 6.3 | −5.0; | 1.3 |
| LS Mean (SE) | −0.03 | (0.34) | −0.60 | (0.35) |
| LS Mean Diff vs. | | | −0.57 | (−1.40, 0.25) |
| placebo (95% CI) | | | | |
| P-value vs. placebo | | | 0.1706 | |
| Week 7 | | | | |
| Number (observed/LOCF imputed) | 30 | (27/3) | 31 | (29/2) |
| Mean (SD) | 1.64 | (2.52) | 1.61 | (3.82) |
| Median | 0.57 | | 0.00 | |
| Q1; Q3 | 0.00; | 2.00 | 0.00; | 0.86 |
| Min; Max | 0.0; | 9.0 | 0.0; | 19.4 |
| Change from baseline | | | | |
| Number (observed/LOCF imputed) | 30 | (27/3) | 31 | (29/2) |
| Mean (SD) | −0.63 | (2.16) | −0.00 | (3.35) |
| Median | −0.05 | | 0.00 | |
| Q1; Q3 | −1.43; | 0.00 | −1.00; | 0.00 |
| Min; Max | −4.9; | 6.3 | −4.3; | 16.8 |
| LS Mean (SE) | −0.59 | (0.59) | −0.14 | (0.62) |
| LS Mean Diff vs. | | | 0.45 | (−0.98, 1.87) |
| placebo (95% CI) | | | | |
| P-value vs. placebo | | | 0.5396 | |
| Week 8 | | | | |
| Number (observed/LOCF imputed) | 30 | (27/3) | 30 | (27/3) |
| Mean (SD) | 1.59 | (2.52) | 1.66 | (3.98) |
| Median | 0.21 | | 0.00 | |
| Q1; Q3 | 0.00; | 3.00 | 0.00; | 1.14 |
| Min; Max | 0.0; | 9.0 | 0.0; | 19.4 |
| Change from baseline | | | | |
| Number (observed/LOCF imputed) | 30 | (27/3) | 30 | (27/3) |
| Mean (SD) | −0.68 | (2.34) | −0.01 | (3.76) |
| Median | 0.00 | | 0.00 | |
| Q1; Q3 | −1.86; | 0.00 | −1.00; | 0.00 |
| Min; Max | −5.9; | 6.3 | −5.5; | 16.8 |
| LS Mean (SE) | −0.56 | (0.62) | −0.07 | (0.65) |
| LS Mean Diff vs. | | | 0.49 | (−1.03, 2.00) |
| placebo (95% CI) | | | | |
| P-value vs. placebo | | | 0.5291 | |
| Week 9 | | | | |
| Number (observed/LOCF imputed) | 30 | (27/3) | 29 | (25/4) |
| Mean (SD) | 1.70 | (2.55) | 1.66 | (3.98) |
| Median | 0.29 | | 0.00 | |
| Q1; Q3 | 0.00; | 2.29 | 0.00; | 1.00 |
| Min; Max | 0.0; | 9.0 | 0.0; | 19.4 |
| Change from baseline | | | | |
| Number (observed/LOCF imputed) | 30 | (27/3) | 29 | (25/4) |
| Mean (SD) | −0.57 | (2.40) | −0.07 | (3.96) |
| Median | 0.00 | | −0.29 | |
| Q1; Q3 | −1.86; | 0.00 | −1.14; | 0.00 |
| Min; Max | −5.9; | 6.3 | −5.5; | 16.8 |
| LS Mean (SE) | −0.48 | (0.61) | −0.23 | (0.64) |
| LS Mean Diff vs. | | | 0.24 | (−1.27, 1.76) |
| placebo (95% CI) | | | | |
| P-value vs. placebo | | | 0.7514 | |
| Week 10 | | | | |
| Number (observed/LOCF imputed) | 29 | (25/4) | 29 | (25/4) |
| Mean (SD) | 1.90 | (2.72) | 1.49 | (3.98) |
| Median | 0.00 | | 0.00 | |
| Q1; Q3 | 0.00; | 3.29 | 0.00; | 0.57 |
| Min; Max | 0.0; | 9.0 | 0.0; | 19.4 |
| Change from baseline | | | | |
| Number (observed/LOCF imputed) | 29 | (25/4) | 29 | (25/4) |
| Mean (SD) | −0.25 | (2.97) | −0.23 | (3.88) |
| Median | 0.00 | | −0.29 | |
| Q1; Q3 | −1.86; | 0.33 | −1.86; | 0.00 |
| Min; Max | −6.9; | 6.3 | −5.5; | 16.8 |
| LS Mean (SE) | −0.45 | (0.64) | −0.77 | (0.68) |
| LS Mean Diff vs. | | | −0.33 | (−1.91, 1.25) |
| placebo (95% CI) | | | | |
| P-value vs. placebo | | | 0.6832 | |
| Week 11 | | | | |
| Number (observed/LOCF imputed) | 30 | (24/6) | 27 | (23/4) |
| Mean (SD) | 2.37 | (3.30) | 1.61 | (4.24) |
| Median | 0.33 | | 0.00 | |
| Q1; Q3 | 0.00; | 5.00 | 0.00; | 0.67 |
| Min; Max | 0.0; | 11.0 | 0.0; | 19.4 |
| Change from baseline | | | | |
| Number (observed/LOCF imputed) | 30 | (24/6) | 27 | (23/4) |
| Mean (SD) | 0.09 | (3.30) | −0.10 | (3.99) |
| Median | 0.00 | | −0.40 | |
| Q1; Q3 | −1.71; | 0.50 | −1.43; | 0.00 |
| Min; Max | −6.5; | 7.2 | −5.5; | 16.8 |
| LS Mean (SE) | −0.01 | (0.71) | −0.36 | (0.75) |
| LS Mean Diff vs. | | | −0.36 | (−2.08, 1.37) |
| placebo (95% CI) | | | | |
| P-value vs. placebo | | | 0.6865 | |
| Week 12 | | | | |
| Number (observed/LOCF imputed) | 28 | (19/9) | 26 | (21/5) |
| Mean (SD) | 2.57 | (3.34) | 1.97 | (4.32) |
| Median | 1.14 | | 0.00 | |
| Q1; Q3 | 0.00; | 4.60 | 0.00; | 2.00 |
| Min; Max | 0.0; | 11.0 | 0.0; | 19.4 |
| Change from baseline | | | | |
| Number (observed/LOCF imputed) | 28 | (19/9) | 26 | (21/5) |
| Mean (SD) | 0.16 | (3.31) | 0.26 | (3.85) |
| Median | 0.00 | | −0.19 | |
| Q1; Q3 | −2.41; | 1.00 | −1.00; | 0.00 |
| Min; Max | −5.3; | 7.2 | −4.3; | 16.8 |
| LS Mean (SE) | 0.06 | (0.68) | −0.01 | (0.72) |
| LS Mean Diff vs. | | | −0.07 | (−1.75, 1.60) |
| placebo (95% CI) | | | | |

| | |
|---|---|
| P-value vs. placebo | 0.9303 |

LATAM: Latin America,
ROW: Rest of the world

TABLE 40B
Number of participants requiring ≥2, ≥3, ≥4, or ≥5 reliever
puffs of salbutamol/albuterol or levosalbutamol/levalbuterol

| | BID cohort | | TID cohort | |
|---|---|---|---|---|
| | | Rilzabrutinib | | Rilzabrutinib |
| | Placebo BID | 400 mg BID | Placebo TID | 400 mg TID |
| | (N = 32) | (N = 32) | (N = 68) | (N = 64) |
| Number of participants | 11 (34.4) | 6 (18.8) | 24 (35.3) | 11 (17.2) |
| with ≥2 additional | | | | |
| reliever puffs of | | | | |
| salbutamol/albuterol or | | | | |
| levosalbutamol/levalbuterol | | | | |
| compared with | | | | |
| baseline on 2 consecutive | | | | |
| days during the | | | | |
| treatment period | | | | |
| Number of participants | 7 (21.9) | 3 (9.4) | 16 (23.5) | 4 (6.3) |
| with ≥3 additional | | | | |
| reliever puffs of | | | | |
| salbutamol/albuterol or | | | | |
| levosalbutamol/levalbuterol | | | | |
| compared with | | | | |
| baseline on 2 consecutive | | | | |
| days during the | | | | |
| treatment period | | | | |
| Number of participants | 6 (18.8) | 2 (6.3) | 12 (17.6) | 3 (4.7) |
| with ≥4 additional | | | | |
| reliever puffs of | | | | |
| salbutamol/albuterol or | | | | |
| levosalbutamol/levalbuterol | | | | |
| compared with | | | | |
| baseline on 2 consecutive | | | | |
| days during the | | | | |
| treatment period, | | | | |
| Number of participants | 4 (12.5) | 2 (6.3) | 10 (14.7) | 1 (1.6) |
| with ≥5 additional | | | | |
| reliever puffs of | | | | |
| salbutamol/albuterol or | | | | |
| levosalbutamol/levalbuterol | | | | |
| compared with | | | | |
| baseline on 2 consecutive | | | | |
| days during the treatment | | | | |
| period | | | | |

### ACQ-5

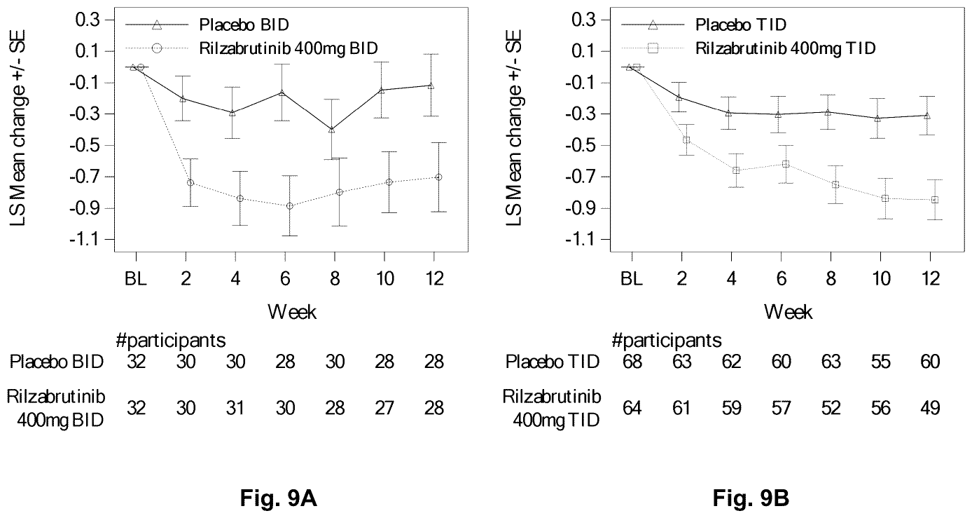

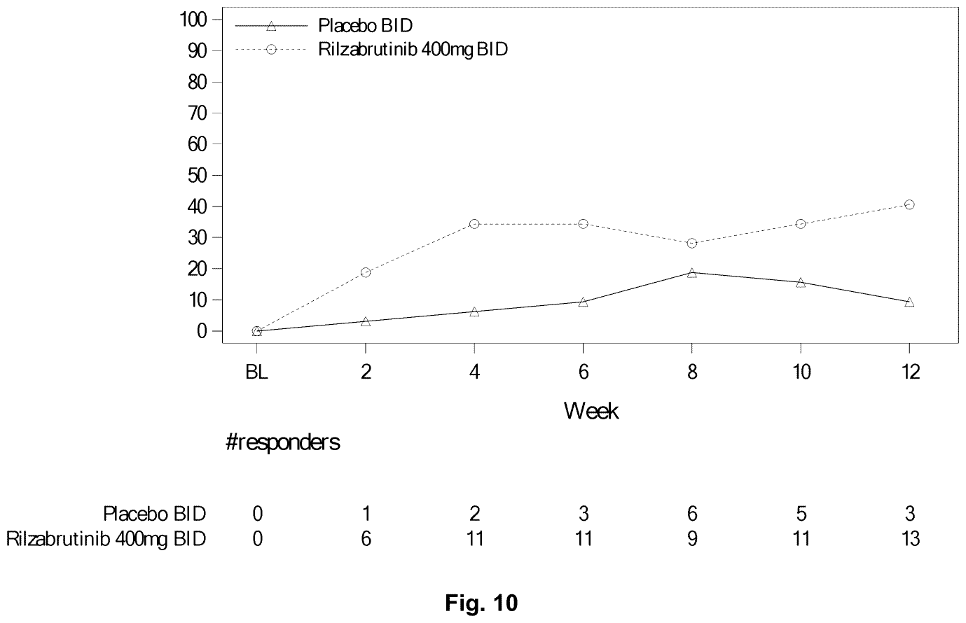

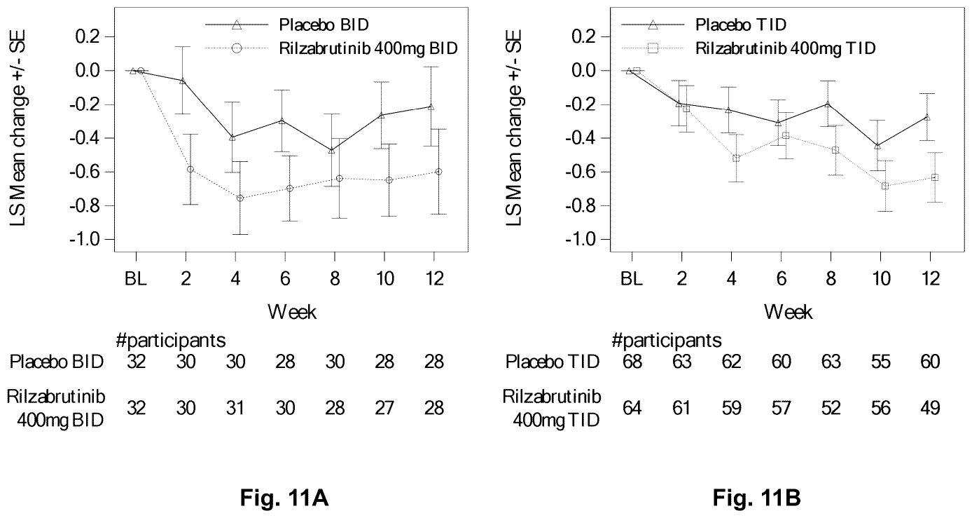

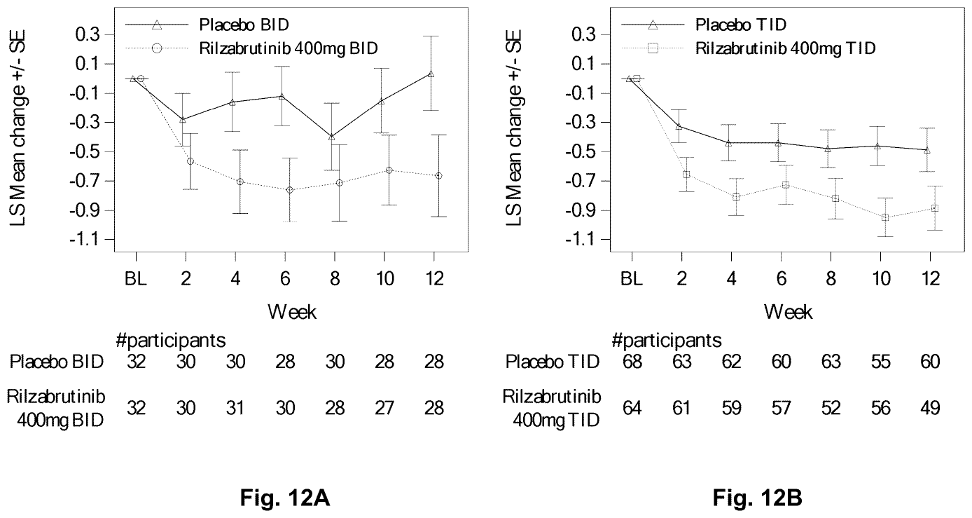

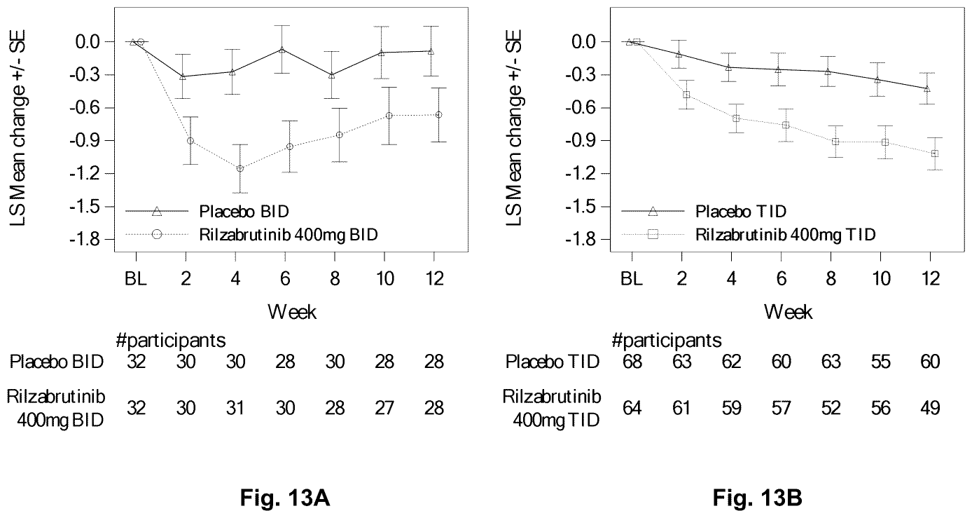

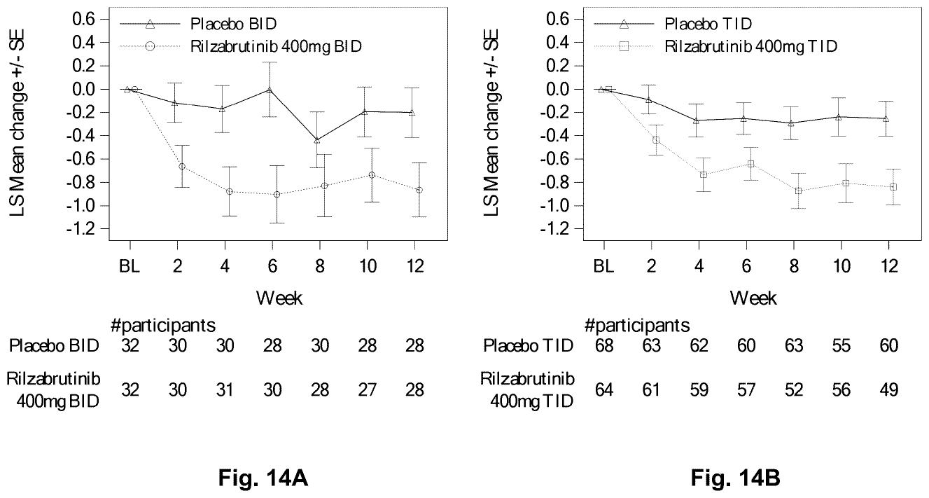

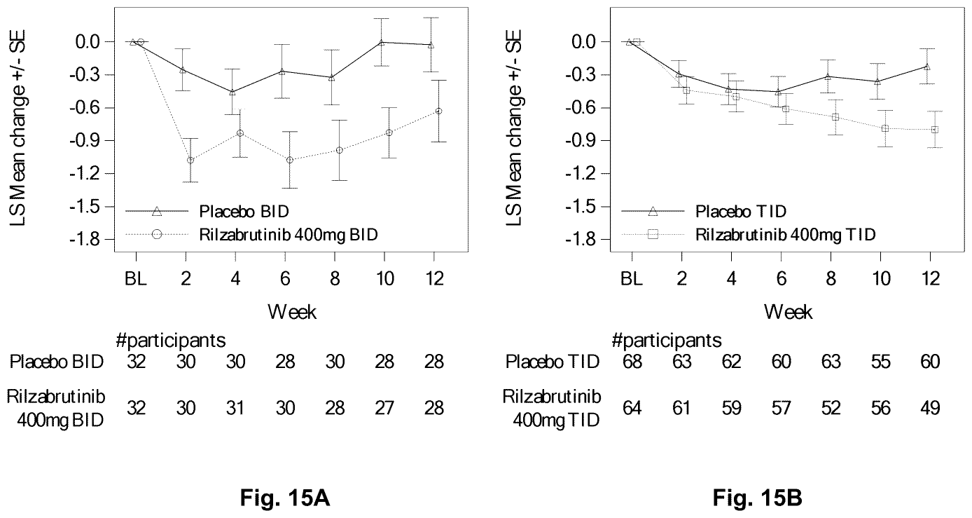

In both the 400 mg BID and 400 mg TID rilzabrutinib cohorts, patients experienced significant and clinically meaningful changes in ACQ-5 relative to patients who received the placebo (FIGS. 9-15) and Tables 41-50). The 400 mg BID rilzabrutinib cohort and placebo cohort exhibited LS mean (SE) values of −0.70 and −0.12 (p=0.0184), respectively, at Week 12 (Table 41). Meanwhile, the 400 mg TID rilzabrutinib cohort and placebo cohort exhibited LS mean (SE) values of −0.85 and −0.31 (p=0.0013), respectively, at Week 12 (Table 41). The overall ACQ-5 reduction observed in both of the rilzabrutinib cohorts therefore exceeded the minimal clinically important (MCID) difference of −0.5.

Consistent with this, a significant percentage of rilzabrutinib-treated patients also achieved ACQ-5 reductions in excess of −0.5. 67.9% of rilzabrutinib-treated patients in the 400 mg BID cohort showed ACQ-5 score reductions of at least −0.05 from baseline at Week 12. Meanwhile, only 35.7% of placebo-treated patients had equivalent reductions in ACQ-5 scores (p=0.0100) (Table 43). Meanwhile, 63.3% of patients in the 400 mg TID cohort showed ACQ-5 score reductions of at least −0.05 from baseline at Week 12, as compared to 40.0% of patients in the placebo group (p=0.0297) (Table 43). Additionally, at Week 12, 40.6% and 15.6% of rilzabrutinib-treated patients in the 400 mg BID and 400 mg TID cohorts, respectively, achieved an ACQ-5 below 0.75, while only 9.4% and 13.2% of placebo-treated patients in the 400 mg BID and 400 mg TID cohorts, respectively, achieved a similar reduction in score (p=0.0092 for the 400 mg BID cohort and 0.6286 for the 400 mg TID cohort) (Table 44).

Patients treated with rilzabrutinib also experienced a rapid decrease in ACQ-5. Significant (p<0.05) differences between rilzabrutinib- and placebo-treated patients in the two cohorts were observed as early as Week 2 and were largely sustained over the course of the treatment period. Moreover, in the 400 mg TID rilzabrutinib cohort, the difference between rilzabrutinib- and placebo-treated patients continued to increase throughout the treatment period.

Additionally, 34.4% of rilzabrutinib-treated patients in the 400 mg BID cohort achieved an ACQ-5 score below 0.75 as early as Week 4, as compared to 6.3% of placebo-treated patients in the 400 mg BID cohort (p=0.0133) (Table 44). Similarly, 17.2% of rilzabrutinib-treated patients in the 400 mg TID cohort achieved an ACQ-5 score below 0.75 by Week 4, as compared to 7.4% of placebo-treated patients in the 400 mg TID cohort (p=0.0919) (Table 44).

Rilzabrutinib was efficacious across patients with different baseline demographics, disease characteristics, with or without exacerbations in the previous year, ICS/LABA dose levels, biomarker levels, eosinophil counts, or FeNO (ppb) (Tables 46-50).

TABLE 41
Change from baseline in ACQ-5 over time (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| ACQ-5 | (N = 32) | (N = 32) | (N = 68) | (N = 64) |

Baseline

| Number | 32 | | 32 | | 68 | | 64 | |
|---|---|---|---|---|---|---|---|---|
| Mean (SD) | 2.19 | (0.40) | 2.04 | (0.37) | 2.18 | (0.40) | 2.24 | (0.48) |

| | | | | |
|---|---|---|---|---|
| Median | 2.20 | 2.00 | 2.20 | 2.20 |
| Q1; Q3 | 2.00; 2.40 | 1.80; 2.20 | 1.80; 2.40 | 2.00; 2.60 |
| Min; Max | 1.4; 3.2 | 1.4; 2.8 | 1.2; 3.2 | 1.4; 4.0 |

Week 2

| Number | 30 | (29/1) | 30 | (30/0) | 63 | (62/1) | 61 | (61/0) |
|---|---|---|---|---|---|---|---|---|
| (observed/LOCF | | | | | | | | |
| imputed) | | | | | | | | |
| Mean (SD) | 1.91 | (0.74) | 1.30 | (0.64) | 2.00 | (0.69) | 1.87 | (0.88) |

| | | | | |
|---|---|---|---|---|
| Median | 1.80 | 1.20 | 2.00 | 2.20 |
| Q1; Q3 | 1.60; 2.20 | 0.80; 1.80 | 1.60; 2.40 | 1.20; 2.60 |
| Min; Max | 0.4; 3.8 | 0.4; 2.6 | 0.4; 3.4 | 0.0; 3.4 |

Change from baseline

| Number | 30 | (29/1) | 30 | (30/0) | 63 | (62/1) | 61 | (61/0) |
|---|---|---|---|---|---|---|---|---|
| (observed/LOCF | | | | | | | | |
| imputed) | | | | | | | | |
| Mean (SD) | −0.25 | (0.78) | −0.74 | (0.62) | −0.17 | (0.62) | −0.37 | (0.79) |
| Median | −0.20 | | −1.00 | | 0.00 | | −0.20 | |
| Q1; Q3 | −0.80; 0.00 | | −1.20; −0.20 | | −0.60; 0.20 | | −0.80; 0.20 | |
| Min; Max | −1.8; 1.6 | | −1.8; 0.4 | | −1.8; 1.6 | | −2.0; 1.2 | |
| LS Mean (SE) | −0.20 | (0.14) | −0.74 | (0.15) | −0.19 | (0.09) | −0.47 | (0.10) |
| LS Mean Diff | | | −0.54 | (−0.89, −0.19) | | | −0.27 | (−0.53, −0.02) |

| vs. placebo | | |
|---|---|---|
| (95% CI) | | |
| P-value vs. | 0.0026 | 0.0335 |
| placebo | | |

Week 4

| Number | 30 | (28/2) | 31 | (30/1) | 62 | (61/1) | 59 | (59/0) |
|---|---|---|---|---|---|---|---|---|
| (observed/LOCF | | | | | | | | |
| imputed) | | | | | | | | |
| Mean (SD) | 1.77 | (0.82) | 1.10 | (0.78) | 1.93 | (0.84) | 1.66 | (0.86) |

| | | | | |
|---|---|---|---|---|
| Median | 1.80 | 1.00 | 2.00 | 1.80 |
| Q1; Q3 | 1.20; 2.20 | 0.40; 1.80 | 1.40; 2.60 | 1.00; 2.40 |
| Min; Max | 0.0; 3.4 | 0.0; 2.6 | 0.0; 4.0 | 0.0; 3.4 |

Change from baseline

| Number | 30 | (28/2) | 31 | (30/1) | 62 | (61/1) | 59 | (59/0) |
|---|---|---|---|---|---|---|---|---|
| (observed/LOCF | | | | | | | | |
| imputed) | | | | | | | | |
| Mean (SD) | −0.40 | (0.96) | −0.94 | (0.64) | −0.23 | (0.79) | −0.57 | (0.77) |
| Median | −0.30 | | −0.80 | | −0.20 | | −0.40 | |
| Q1; Q3 | −0.80; 0.20 | | −1.60; −0.40 | | −0.60; 0.20 | | −1.00; 0.00 | |
| Min; Max | −2.6; 1.6 | | −2.0; 0.4 | | −2.2; 2.0 | | −2.4; 1.0 | |
| LS Mean (SE) | −0.29 | (0.16) | −0.84 | (0.17) | −0.29 | (0.10) | −0.66 | (0.11) |
| LS Mean Diff | | | −0.55 | (−0.95, −0.14) | | | −0.37 | (−0.64, −0.09) |

| vs. placebo | | |
|---|---|---|
| (95% CI) | | |
| P-value vs. | 0.0085 | 0.0088 |
| placebo | | |

Week 6

| Number | 28 | (26/2) | 30 | (28/2) | 60 | (58/2) | 57 | (56/1) |
|---|---|---|---|---|---|---|---|---|
| (observed/LOCF | | | | | | | | |
| imputed) | | | | | | | | |
| Mean (SD) | 1.91 | (0.92) | 1.10 | (0.80) | 1.79 | (0.80) | 1.68 | (0.92) |

| | | | | |
|---|---|---|---|---|
| Median | 2.00 | 1.00 | 1.60 | 2.00 |
| Q1; Q3 | 1.20; 2.50 | 0.40; 1.60 | 1.20; 2.20 | 0.80; 2.40 |
| Min; Max | 0.4; 4.0 | 0.0; 3.2 | 0.0; 3.2 | 0.0; 3.2 |

Change from baseline

| Number | 28 | (26/2) | 30 | (28/2) | 60 | (58/2) | 57 | (56/1) |
|---|---|---|---|---|---|---|---|---|
| (observed/LOCF | | | | | | | | |
| imputed) | | | | | | | | |
| Mean (SD) | −0.26 | (0.99) | −0.97 | (0.74) | −0.38 | (0.75) | −0.57 | (0.83) |
| Median | −0.10 | | −1.20 | | −0.40 | | −0.60 | |
| Q1; Q3 | −1.00; 0.40 | | −1.60; −0.60 | | −1.00; 0.20 | | −1.00; 0.00 | |
| Min; Max | −2.4; 1.4 | | −1.8; 1.0 | | −2.0; 0.8 | | −2.8; 1.2 | |
| LS Mean (SE) | −0.16 | (0.18) | −0.89 | (0.19) | −0.30 | (0.12) | −0.62 | (0.12) |
| LS Mean Diff | | | −0.72 | (−1.16, −0.28) | | | −0.32 | (−0.62, −0.01) |

| vs. placebo | | |
|---|---|---|
| (95% CI) | | |
| P-value vs. | 0.0013 | 0.0412 |
| placebo | | |

Week 8

| Number | 30 | (27/3) | 28 | (24/4) | 63 | (60/3) | 52 | (49/3) |
|---|---|---|---|---|---|---|---|---|
| (observed/LOCF | | | | | | | | |
| imputed) | | | | | | | | |
| Mean (SD) | 1.61 | (0.97) | 1.12 | (0.90) | 1.95 | (0.99) | 1.54 | (0.77) |

| | | | | |
|---|---|---|---|---|
| Median | 1.80 | 0.80 | 1.80 | 1.80 |
| Q1; Q3 | 0.80; 2.20 | 0.60; 1.40 | 1.20; 2.40 | 1.00; 2.00 |
| Min; Max | 0.0; 4.0 | 0.0; 3.6 | 0.0; 5.2 | 0.0; 3.2 |

Change from baseline

| Number | 30 | (27/3) | 28 | (24/4) | 63 | (60/3) | 52 | (49/3) |
|---|---|---|---|---|---|---|---|---|
| (observed/LOCF | | | | | | | | |
| imputed) | | | | | | | | |
| Mean (SD) | −0.56 | (1.04) | −0.91 | (0.80) | −0.19 | (0.94) | −0.72 | (0.73) |
| Median | −0.40 | | −1.20 | | −0.40 | | −0.60 | |
| Q1; Q3 | −1.20; 0.00 | | −1.40; −0.80 | | −1.00; 0.40 | | −1.20; −0.20 | |
| Min; Max | −3.2; 1.4 | | −2.2; 1.0 | | −2.0; 3.0 | | −2.6; 1.0 | |
| LS Mean (SE) | −0.40 | (0.19) | −0.80 | (0.22) | −0.29 | (0.11) | −0.75 | (0.12) |
| LS Mean Diff | | | −0.40 | (−0.87, 0.07) | | | −0.46 | (−0.76, −0.17) |

| vs. placebo | | |
|---|---|---|
| (95% CI) | | |
| P-value vs. | 0.0968 | 0.0023 |
| placebo | | |

Week 10

| Number | 28 | (22/6) | 27 | (23/4) | 55 | (51/4) | 56 | (53/3) |
|---|---|---|---|---|---|---|---|---|
| (observed/LOCF | | | | | | | | |
| imputed) | | | | | | | | |
| Mean (SD) | 1.73 | (1.03) | 1.15 | (0.90) | 1.80 | (1.02) | 1.49 | (0.84) |

| | | | | |
|---|---|---|---|---|
| Median | 1.70 | 1.00 | 1.80 | 1.60 |
| Q1; Q3 | 0.90; 2.50 | 0.40; 1.60 | 1.00; 2.60 | 0.90; 2.00 |
| Min; Max | 0.0; 4.0 | 0.0; 3.2 | 0.0; 4.0 | 0.0; 3.2 |

Change from baseline

| Number | 28 | (22/6) | 27 | (23/4) | 55 | (51/4) | 56 | (53/3) |
|---|---|---|---|---|---|---|---|---|
| (observed/LOCF | | | | | | | | |
| imputed) | | | | | | | | |
| Mean (SD) | −0.42 | (1.11) | −0.92 | (0.89) | −0.33 | (0.90) | −0.75 | (0.81) |
| Median | −0.40 | | −1.00 | | −0.40 | | −0.70 | |
| Q1; Q3 | −1.20; 0.40 | | −1.60; −0.40 | | −1.00; 0.40 | | −1.40; −0.20 | |
| Min; Max | −2.6; 1.6 | | −2.4; 1.0 | | −2.2; 1.4 | | −2.6; 1.2 | |
| LS Mean (SE) | −0.15 | (0.18) | −0.73 | (0.19) | −0.33 | (0.13) | −0.84 | (0.13) |
| LS Mean Diff | | | −0.59 | (−1.03, −0.14) | | | −0.51 | (−0.84, −0.18) |

| vs. placebo | | |
|---|---|---|
| (95% CI) | | |
| P-value vs. | 0.0095 | 0.0022 |
| placebo | | |

Week 12

| Number | 28 | (19/9) | 28 | (23/5) | 60 | (50/10) | 49 | (46/3) |
|---|---|---|---|---|---|---|---|---|
| (observed/LOCF | | | | | | | | |
| imputed) | | | | | | | | |
| Mean (SD) | 1.83 | (0.98) | 1.16 | (0.94) | 1.93 | (1.12) | 1.46 | (0.75) |

| | | | | |
|---|---|---|---|---|
| Median | 2.00 | 1.00 | 1.80 | 1.60 |
| Q1; Q3 | 1.10; 2.20 | 0.40; 2.00 | 1.10; 2.50 | 1.00; 2.00 |
| Min; Max | 0.0; 4.0 | 0.0; 3.2 | 0.0; 5.4 | 0.0; 3.2 |

Change from baseline

| Number | 28 | (19/9) | 28 | (23/5) | 60 | (50/10) | 49 | (46/3) |
|---|---|---|---|---|---|---|---|---|
| (observed/LOCF | | | | | | | | |
| imputed) | | | | | | | | |
| Mean (SD) | −0.34 | (1.02) | −0.89 | (0.93) | −0.22 | (1.03) | −0.82 | (0.72) |
| Median | −0.10 | | −1.30 | | −0.40 | | −0.80 | |
| Q1; Q3 | −1.00; 0.10 | | −1.60; −0.20 | | −1.00; 0.40 | | −1.40; −0.20 | |
| Min; Max | −3.0; 1.6 | | −2.4; 1.0 | | −1.8; 3.2 | | −2.2; 0.4 | |
| LS Mean (SE) | −0.12 | (0.20) | −0.70 | (0.22) | −0.31 | (0.12) | −0.85 | (0.13) |

| | | | | |
|---|---|---|---|---|
| LS Mean Diff | −0.59 | (−1.07, −0.10) | −0.54 | (−0.86, −0.21) |

| vs. placebo | | |
|---|---|---|
| (95% CI) | | |
| P-value vs. | 0.0184 | 0.0013 |
| placebo | | |

LATAM: Latin America, ROW: Rest of the world

TABLE 42
Summary of ACQ-5 over time (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| ACQ-5 | (N = 32) | (N = 32) | (N = 68) | (N = 64) |

Mean score
Baseline

| Number | 32 | | 32 | | 68 | | 64 | |
|---|---|---|---|---|---|---|---|---|
| Mean (SD) | 2.19 | (0.40) | 2.04 | (0.37) | 2.18 | (0.40) | 2.24 | (0.48) |

| | | | | |
|---|---|---|---|---|
| Median | 2.20 | 2.00 | 2.20 | 2.20 |
| Q1; Q3 | 2.00; 2.40 | 1.80; 2.20 | 1.80; 2.40 | 2.00; 2.60 |
| Min; Max | 1.4; 3.2 | 1.4; 2.8 | 1.2; 3.2 | 1.4; 4.0 |

Week 2

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | 1.91 | (0.74) | 1.30 | (0.64) | 2.00 | (0.69) | 1.87 | (0.88) |

| | | | | |
|---|---|---|---|---|
| Median | 1.80 | 1.20 | 2.00 | 2.20 |
| Q1; Q3 | 1.60; 2.20 | 0.80; 1.80 | 1.60; 2.40 | 1.20; 2.60 |
| Min; Max | 0.4; 3.8 | 0.4; 2.6 | 0.4; 3.4 | 0.0; 3.4 |

Change from baseline

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | −0.25 | (0.78) | −0.74 | (0.62) | −0.17 | (0.62) | −0.37 | (0.79) |
| Median | −0.20 | | −1.00 | | 0.00 | | −0.20 | |
| Q1; Q3 | −0.80; 0.00 | | −1.20; −0.20 | | −0.60; 0.20 | | −0.80; 0.20 | |
| Min; Max | −1.8; 1.6 | | −1.8; 0.4 | | −1.8; 1.6 | | −2.0; 1.2 | |
| LS Mean (SE) | −0.20 | (0.14) | −0.74 | (0.15) | −0.19 | (0.09) | −0.47 | (0.10) |

| | | | | |
|---|---|---|---|---|
| LS Mean Diff vs. placebo | −0.54 | (−0.89, −0.19) | −0.27 | (−0.53, −0.02) |

(95% CI)

| | | |
|---|---|---|
| P-value vs. placebo | 0.0026 | 0.0335 |

Week 4

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | 1.77 | (0.82) | 1.10 | (0.78) | 1.93 | (0.84) | 1.66 | (0.86) |

| | | | | |
|---|---|---|---|---|
| Median | 1.80 | 1.00 | 2.00 | 1.80 |
| Q1; Q3 | 1.20; 2.20 | 0.40; 1.80 | 1.40; 2.60 | 1.00; 2.40 |
| Min; Max | 0.0; 3.4 | 0.0; 2.6 | 0.0; 4.0 | 0.0; 3.4 |

Change from baseline

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | −0.40 | (0.96) | −0.94 | (0.64) | −0.23 | (0.79) | −0.57 | (0.77) |
| Median | −0.30 | | −0.80 | | −0.20 | | −0.40 | |
| Q1; Q3 | −0.80; 0.20 | | −1.60; −0.40 | | −0.60; 0.20 | | −1.00; 0.00 | |
| Min; Max | −2.6; 1.6 | | −2.0; 0.4 | | −2.2; 2.0 | | −2.4; 1.0 | |
| LS Mean (SE) | −0.29 | (0.16) | −0.84 | (0.17) | −0.29 | (0.10) | −0.66 | (0.11) |

| | | | | |
|---|---|---|---|---|
| LS Mean Diff vs. placebo | −0.55 | (−0.95, −0.14) | −0.37 | (−0.64, −0.09) |

(95% CI)

| | | |
|---|---|---|
| P-value vs. placebo | 0.0085 | 0.0088 |

Week 6

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | 1.91 | (0.92) | 1.10 | (0.80) | 1.79 | (0.80) | 1.68 | (0.92) |

| | | | | |
|---|---|---|---|---|
| Median | 2.00 | 1.00 | 1.60 | 2.00 |
| Q1; Q3 | 1.20; 2.50 | 0.40; 1.60 | 1.20; 2.20 | 0.80; 2.40 |
| Min; Max | 0.4; 4.0 | 0.0; 3.2 | 0.0; 3.2 | 0.0; 3.2 |

Change from baseline

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | −0.26 | (0.99) | −0.97 | (0.74) | −0.38 | (0.75) | −0.57 | (0.83) |
| Median | −0.10 | | −1.20 | | −0.40 | | −0.60 | |
| Q1; Q3 | −1.00; 0.40 | | −1.60; −0.60 | | −1.00; 0.20 | | −1.00; 0.00 | |
| Min; Max | −2.4; 1.4 | | −1.8; 1.0 | | −2.0; 0.8 | | −2.8; 1.2 | |
| LS Mean (SE) | −0.16 | (0.18) | −0.89 | (0.19) | −0.30 | (0.12) | −0.62 | (0.12) |

| | | | | |
|---|---|---|---|---|
| LS Mean Diff vs. placebo | −0.72 | (−1.16, −0.28) | −0.32 | (−0.62, −0.01) |

(95% CI)

| | | |
|---|---|---|
| P-value vs. placebo | 0.0013 | 0.0412 |

Week 8

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | 1.61 | (0.97) | 1.12 | (0.90) | 1.95 | (0.99) | 1.54 | (0.77) |

| | | | | |
|---|---|---|---|---|
| Median | 1.80 | 0.80 | 1.80 | 1.80 |
| Q1; Q3 | 0.80; 2.20 | 0.60; 1.40 | 1.20; 2.40 | 1.00; 2.00 |
| Min; Max | 0.0; 4.0 | 0.0; 3.6 | 0.0; 5.2 | 0.0; 3.2 |

Change from baseline

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | −0.56 | (1.04) | −0.91 | (0.80) | −0.19 | (0.94) | −0.72 | (0.73) |
| Median | −0.40 | | −1.20 | | −0.40 | | −0.60 | |
| Q1; Q3 | −1.20; 0.00 | | −1.40; −0.80 | | −1.00; 0.40 | | −1.20; −0.20 | |
| Min; Max | −3.2; 1.4 | | −2.2; 1.0 | | −2.0; 3.0 | | −2.6; 1.0 | |
| LS Mean (SE) | −0.40 | (0.19) | −0.80 | (0.22) | −0.29 | (0.11) | −0.75 | (0.12) |

| | | | | |
|---|---|---|---|---|
| LS Mean Diff vs. placebo | −0.40 | (−0.87, 0.07) | −0.46 | (−0.76, −0.17) |

(95% CI)

| | | |
|---|---|---|
| P-value vs. placebo | 0.0968 | 0.0023 |

Week 10

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | 1.73 | (1.03) | 1.15 | (0.90) | 1.80 | (1.02) | 1.49 | (0.84) |

| | | | | |
|---|---|---|---|---|
| Median | 1.70 | 1.00 | 1.80 | 1.60 |
| Q1; Q3 | 0.90; 2.50 | 0.40; 1.60 | 1.00; 2.60 | 0.90; 2.00 |
| Min; Max | 0.0; 4.0 | 0.0; 3.2 | 0.0; 4.0 | 0.0; 3.2 |

Change from baseline

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | −0.42 | (1.11) | −0.92 | (0.89) | −0.33 | (0.90) | −0.75 | (0.81) |
| Median | −0.40 | | −1.00 | | −0.40 | | −0.70 | |
| Q1; Q3 | −1.20; 0.40 | | −1.60; −0.40 | | −1.00; 0.40 | | −1.40; −0.20 | |
| Min; Max | −2.6; 1.6 | | −2.4; 1.0 | | −2.2; 1.4 | | −2.6; 1.2 | |
| LS Mean (SE) | −0.15 | (0.18) | −0.73 | (0.19) | −0.33 | (0.13) | −0.84 | (0.13) |

| | | | | |
|---|---|---|---|---|
| LS Mean Diff vs. placebo | −0.59 | (−1.03, −0.14) | −0.51 | (−0.84, −0.18) |

(95% CI)

| | | |
|---|---|---|
| P-value vs. placebo | 0.0095 | 0.0022 |

Week 12

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | 1.83 | (0.98) | 1.16 | (0.94) | 1.93 | (1.12) | 1.46 | (0.75) |

| | | | | |
|---|---|---|---|---|
| Median | 2.00 | 1.00 | 1.80 | 1.60 |
| Q1; Q3 | 1.10; 2.20 | 0.40; 2.00 | 1.10; 2.50 | 1.00; 2.00 |
| Min; Max | 0.0; 4.0 | 0.0; 3.2 | 0.0; 5.4 | 0.0; 3.2 |

Change from baseline

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | −0.34 | (1.02) | −0.89 | (0.93) | −0.22 | (1.03) | −0.82 | (0.72) |
| Median | −0.10 | | −1.30 | | −0.40 | | −0.80 | |
| Q1; Q3 | −1.00; 0.10 | | −1.60; −0.20 | | −1.00; 0.40 | | −1.40; −0.20 | |
| Min; Max | −3.0; 1.6 | | −2.4; 1.0 | | −1.8; 3.2 | | −2.2; 0.4 | |
| LS Mean (SE) | −0.12 | (0.20) | −0.70 | (0.22) | −0.31 | (0.12) | −0.85 | (0.13) |

| | | | | |
|---|---|---|---|---|
| LS Mean Diff vs. placebo | −0.59 | (−1.07, −0.10) | −0.54 | (−0.86, −0.21) |

(95% CI)

| | | |
|---|---|---|
| P-value vs. placebo | 0.0184 | 0.0013 |

Frequency of awakening by asthma
Baseline

| Number | 32 | | 32 | | 68 | | 64 | |
|---|---|---|---|---|---|---|---|---|
| Mean (SD) | 1.91 | (0.64) | 1.63 | (0.66) | 1.91 | (0.69) | 1.89 | (0.72) |

| | | | | |
|---|---|---|---|---|
| Median | 2.00 | 2.00 | 2.00 | 2.00 |
| Q1; Q3 | 2.00; 2.00 | 1.00; 2.00 | 2.00; 2.00 | 2.00; 2.00 |
| Min; Max | 0.0; 3.0 | 0.0; 3.0 | 0.0; 3.0 | 0.0; 4.0 |

Week 2

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | 1.70 | (1.12) | 1.03 | (0.81) | 1.67 | (1.05) | 1.72 | (1.05) |

| | | | | |
|---|---|---|---|---|
| Median | 2.00 | 1.00 | 2.00 | 2.00 |
| Q1; Q3 | 1.00; 2.00 | 0.00; 2.00 | 1.00; 2.00 | 1.00; 2.00 |
| Min; Max | 0.0; 4.0 | 0.0; 2.0 | 0.0; 5.0 | 0.0; 4.0 |

Change from baseline

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | −0.17 | (1.15) | −0.57 | (0.82) | −0.24 | (1.07) | −0.18 | (1.09) |
| Median | 0.00 | | −0.50 | | 0.00 | | 0.00 | |
| Q1; Q3 | −1.00; 0.00 | | −1.00; 0.00 | | −1.00; 0.00 | | −1.00; 0.00 | |
| Min; Max | −3.0; 2.0 | | −2.0; 1.0 | | −3.0; 2.0 | | −2.0; 2.0 | |
| LS Mean (SE) | −0.06 | (0.20) | −0.58 | (0.21) | −0.19 | (0.14) | −0.23 | (0.14) |

| | | | | |
|---|---|---|---|---|
| LS Mean Diff vs. placebo | −0.53 | (−1.01, −0.04) | −0.03 | (−0.39, 0.32) |

(95% CI)

| | | |
|---|---|---|
| P-value vs. placebo | 0.0349 | 0.8553 |

Week 4

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | 1.40 | (1.04) | 0.90 | (0.98) | 1.65 | (1.03) | 1.39 | (0.93) |

| | | | | |
|---|---|---|---|---|
| Median | 2.00 | 1.00 | 2.00 | 2.00 |
| Q1; Q3 | 0.00; 2.00 | 0.00; 2.00 | 1.00; 2.00 | 1.00; 2.00 |
| Min; Max | 0.0; 3.0 | 0.0; 3.0 | 0.0; 4.0 | 0.0; 3.0 |

Change from baseline

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | −0.47 | (1.04) | −0.71 | (1.07) | −0.24 | (1.10) | −0.51 | (1.06) |
| Median | 0.00 | | −1.00 | | 0.00 | | 0.00 | |
| Q1; Q3 | −1.00; 0.00 | | −2.00; 0.00 | | −1.00; 0.00 | | −1.00; 0.00 | |
| Min; Max | −3.0; 1.0 | | −2.0; 2.0 | | −3.0; 3.0 | | −2.0; 2.0 | |
| LS Mean (SE) | −0.39 | (0.21) | −0.76 | (0.22) | −0.23 | (0.14) | −0.52 | (0.14) |

| | | | | |
|---|---|---|---|---|
| LS Mean Diff vs. placebo | −0.36 | (−0.88, 0.15) | −0.29 | (−0.64, 0.07) |

(95% CI)

| | | |
|---|---|---|
| P-value vs. placebo | 0.1702 | 0.1165 |

Week 6

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | 1.46 | (0.96) | 0.93 | (0.83) | 1.50 | (1.10) | 1.56 | (0.91) |

| | | | | |
|---|---|---|---|---|
| Median | 2.00 | 1.00 | 1.00 | 2.00 |
| Q1; Q3 | 1.00; 2.00 | 0.00; 1.00 | 1.00; 2.00 | 1.00; 2.00 |
| Min; Max | 0.0; 3.0 | 0.0; 3.0 | 0.0; 4.0 | 0.0; 4.0 |

Change from baseline

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | −0.39 | (0.96) | −0.67 | (0.88) | −0.40 | (1.06) | −0.33 | (0.87) |
| Median | 0.00 | | −1.00 | | 0.00 | | 0.00 | |
| Q1; Q3 | −1.00; 0.00 | | −1.00; 0.00 | | −1.00; 0.00 | | −1.00; 0.00 | |
| Min; Max | −3.0; 1.0 | | −2.0; 1.0 | | −3.0; 2.0 | | −2.0; 1.0 | |
| LS Mean (SE) | −0.30 | (0.18) | −0.70 | (0.19) | −0.31 | (0.14) | −0.39 | (0.14) |

| | | | | |
|---|---|---|---|---|
| LS Mean Diff vs. placebo | −0.40 | (−0.85, 0.05) | −0.08 | (−0.42, 0.27) |

(95% CI)

| | | |
|---|---|---|
| P-value vs. placebo | 0.0792 | 0.6678 |

Week 8

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | 1.23 | (0.97) | 0.93 | (1.05) | 1.70 | (1.16) | 1.46 | (0.87) |

| | | | | |
|---|---|---|---|---|
| Median | 1.00 | 1.00 | 2.00 | 1.50 |
| Q1; Q3 | 0.00; 2.00 | 0.00; 1.50 | 1.00; 3.00 | 1.00; 2.00 |
| Min; Max | 0.0; 3.0 | 0.0; 4.0 | 0.0; 5.0 | 0.0; 4.0 |

Change from baseline

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | −0.63 | (0.93) | −0.64 | (1.10) | −0.16 | (1.12) | −0.42 | (1.02) |
| Median | −0.50 | | −1.00 | | 0.00 | | 0.00 | |
| Q1; Q3 | −1.00; 0.00 | | −1.50; 0.00 | | −1.00; 1.00 | | −1.00; 0.00 | |
| Min; Max | −3.0; 1.0 | | −2.0; 2.0 | | −3.0; 3.0 | | −3.0; 2.0 | |
| LS Mean (SE) | −0.47 | (0.21) | −0.64 | (0.23) | −0.20 | (0.14) | −0.47 | (0.15) |

| | | | | |
|---|---|---|---|---|
| LS Mean Diff vs. placebo | −0.17 | (−0.69, 0.35) | −0.28 | (−0.64, 0.09) |

(95% CI)

| | | |
|---|---|---|
| P-value vs. placebo | 0.5311 | 0.1389 |

Week 10

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | 1.29 | (1.05) | 0.89 | (0.89) | 1.45 | (1.05) | 1.32 | (1.11) |

| | | | | |
|---|---|---|---|---|
| Median | 1.00 | 1.00 | 1.00 | 1.00 |
| Q1; Q3 | 0.00; 2.00 | 0.00; 1.00 | 1.00; 2.00 | 1.00; 2.00 |
| Min; Max | 0.0; 3.0 | 0.0; 3.0 | 0.0; 4.0 | 0.0; 5.0 |

Change from baseline

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | −0.57 | (1.10) | −0.67 | (1.07) | −0.42 | (1.08) | −0.55 | (1.14) |
| Median | 0.00 | | −1.00 | | 0.00 | | −1.00 | |
| Q1; Q3 | −1.00; 0.00 | | −1.00; 0.00 | | −1.00; 0.00 | | −1.00; 0.00 | |
| Min; Max | −3.0; 1.0 | | −3.0; 1.0 | | −3.0; 2.0 | | −2.0; 3.0 | |
| LS Mean (SE) | −0.26 | (0.20) | −0.65 | (0.21) | −0.44 | (0.15) | −0.68 | (0.15) |

| | | | | |
|---|---|---|---|---|
| LS Mean Diff vs. placebo | −0.38 | (−0.88, 0.11) | −0.24 | (−0.62, 0.14) |

(95% CI)

| | | |
|---|---|---|
| P-value vs. placebo | 0.1272 | 0.2171 |

Week 12

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | 1.39 | (1.10) | 0.89 | (1.07) | 1.63 | (1.16) | 1.31 | (0.89) |

| | | | | |
|---|---|---|---|---|
| Median | 1.00 | 1.00 | 2.00 | 1.00 |
| Q1; Q3 | 0.50; 2.00 | 0.00; 1.50 | 1.00; 2.00 | 1.00; 2.00 |
| Min; Max | 0.0; 4.0 | 0.0; 4.0 | 0.0; 5.0 | 0.0; 3.0 |

Change from baseline

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | −0.46 | (1.00) | −0.71 | (1.24) | −0.23 | (1.17) | −0.61 | (1.02) |
| Median | 0.00 | | −1.00 | | 0.00 | | −1.00 | |
| Q1; Q3 | −1.00; 0.00 | | −1.50; 0.00 | | −1.00; 0.50 | | −1.00; 0.00 | |
| Min; Max | −2.0; 2.0 | | −3.0; 2.0 | | −3.0; 3.0 | | −3.0; 1.0 | |
| LS Mean (SE) | −0.21 | (0.23) | −0.60 | (0.25) | −0.27 | (0.14) | −0.63 | (0.15) |

| | | | | |
|---|---|---|---|---|
| LS Mean Diff vs. placebo | −0.39 | (−0.95, 0.18) | −0.36 | (−0.73, 0.01) |

(95% CI)

| | | |
|---|---|---|
| P-value vs. placebo | 0.1778 | 0.0598 |

Asthma symptoms when woke up
Baseline

| Number | 32 | | 32 | | 68 | | 64 | |
|---|---|---|---|---|---|---|---|---|
| Mean (SD) | 2.25 | (0.62) | 2.16 | (0.51) | 2.34 | (0.61) | 2.52 | (0.67) |

| | | | | |
|---|---|---|---|---|
| Median | 2.00 | 2.00 | 2.00 | 3.00 |
| Q1; Q3 | 2.00; 3.00 | 2.00; 2.00 | 2.00; 3.00 | 2.00; 3.00 |
| Min; Max | 1.0; 4.0 | 1.0; 3.0 | 1.0; 4.0 | 1.0; 4.0 |

Week 2

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | 1.90 | (0.88) | 1.60 | (0.81) | 2.11 | (0.76) | 1.93 | (1.05) |

| | | | | |
|---|---|---|---|---|
| Median | 2.00 | 2.00 | 2.00 | 2.00 |
| Q1; Q3 | 1.00; 2.00 | 1.00; 2.00 | 2.00; 3.00 | 1.00; 3.00 |
| Min; Max | 0.0; 4.0 | 0.0; 3.0 | 0.0; 3.0 | 0.0; 3.0 |

Change from baseline

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | −0.33 | (0.92) | −0.57 | (1.01) | −0.21 | (0.79) | −0.57 | (1.09) |
| Median | 0.00 | | −1.00 | | 0.00 | | 0.00 | |
| Q1; Q3 | −1.00; 0.00 | | −1.00; 0.00 | | −1.00; 0.00 | | −1.00; 0.00 | |
| Min; Max | −2.0; 1.0 | | −3.0; 1.0 | | −2.0; 2.0 | | −3.0; 1.0 | |
| LS Mean (SE) | −0.28 | (0.18) | −0.57 | (0.19) | −0.33 | (0.11) | −0.66 | (0.12) |

| | | | | |
|---|---|---|---|---|
| LS Mean Diff vs. placebo | −0.28 | (−0.72, 0.15) | −0.33 | (−0.63, −0.03) |

(95% CI)

| | | |
|---|---|---|
| P-value vs. placebo | 0.2021 | 0.0333 |

Week 4

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | 1.77 | (1.14) | 1.13 | (0.92) | 2.08 | (0.89) | 1.83 | (1.10) |

| | | | | |
|---|---|---|---|---|
| Median | 2.00 | 1.00 | 2.00 | 2.00 |
| Q1; Q3 | 1.00; 3.00 | 0.00; 2.00 | 2.00; 3.00 | 1.00; 3.00 |
| Min; Max | 0.0; 4.0 | 0.0; 3.0 | 0.0; 4.0 | 0.0; 4.0 |

Change from baseline

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | −0.47 | (1.41) | −1.03 | (1.05) | −0.24 | (0.97) | −0.66 | (1.08) |
| Median | 0.00 | | −1.00 | | 0.00 | | 0.00 | |
| Q1; Q3 | −1.00; 0.00 | | −2.00; 0.00 | | −1.00; 0.00 | | −2.00; 0.00 | |
| Min; Max | −3.0; 3.0 | | −3.0; 1.0 | | −3.0; 2.0 | | −3.0; 1.0 | |
| LS Mean (SE) | −0.16 | (0.20) | −0.71 | (0.22) | −0.44 | (0.12) | −0.81 | (0.12) |

| | | | | |
|---|---|---|---|---|
| LS Mean Diff vs. placebo | −0.55 | (−1.05, −0.05) | −0.37 | (−0.69, −0.05) |

(95% CI)

| | | |
|---|---|---|
| P-value vs. placebo | 0.0324 | 0.0252 |

Week 6

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | 2.00 | (1.02) | 1.30 | (0.95) | 1.92 | (0.89) | 1.86 | (1.04) |

| | | | | |
|---|---|---|---|---|
| Median | 2.00 | 1.00 | 2.00 | 2.00 |
| Q1; Q3 | 1.00; 3.00 | 1.00; 2.00 | 1.00; 3.00 | 1.00; 3.00 |
| Min; Max | 0.0; 4.0 | 0.0; 3.0 | 0.0; 4.0 | 0.0; 3.0 |

Change from baseline

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | −0.25 | (1.14) | −0.87 | (1.04) | −0.45 | (0.91) | −0.61 | (0.98) |
| Median | 0.00 | | −1.00 | | 0.00 | | −1.00 | |
| Q1; Q3 | −1.00; 1.00 | | −2.00; 0.00 | | −1.00; 0.00 | | −1.00; 0.00 | |
| Min; Max | −2.0; 2.0 | | −3.0; 1.0 | | −3.0; 1.0 | | −3.0; 1.0 | |
| LS Mean (SE) | −0.12 | (0.20) | −0.76 | (0.22) | −0.44 | (0.13) | −0.73 | (0.13) |

| | | | | |
|---|---|---|---|---|
| LS Mean Diff vs. placebo | −0.64 | (−1.13, −0.15) | −0.29 | (−0.62, 0.05) |

(95% CI)

| | | |
|---|---|---|
| P-value vs. placebo | 0.0107 | 0.0947 |

Week 8

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | 1.60 | (1.10) | 1.25 | (1.11) | 2.00 | (1.03) | 1.73 | (0.95) |

| | | | | |
|---|---|---|---|---|
| Median | 2.00 | 1.00 | 2.00 | 2.00 |
| Q1; Q3 | 1.00; 2.00 | 1.00; 2.00 | 1.00; 3.00 | 1.00; 2.50 |
| Min; Max | 0.0; 4.0 | 0.0; 5.0 | 0.0; 5.0 | 0.0; 3.0 |

Change from baseline

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | −0.63 | (1.33) | −0.96 | (1.00) | −0.30 | (1.07) | −0.83 | (1.00) |
| Median | −1.00 | | −1.00 | | 0.00 | | −1.00 | |
| Q1; Q3 | −1.00; 0.00 | | −2.00; 0.00 | | −1.00; 0.00 | | −2.00; 0.00 | |
| Min; Max | −3.0; 2.0 | | −3.0; 2.0 | | −3.0; 3.0 | | −3.0; 1.0 | |
| LS Mean (SE) | −0.40 | (0.23) | −0.71 | (0.26) | −0.48 | (0.13) | −0.82 | (0.14) |

| | | | | |
|---|---|---|---|---|
| LS Mean Diff vs. placebo | −0.32 | (−0.87, 0.24) | −0.34 | (−0.69, 0.01) |

(95% CI)

| | | |
|---|---|---|
| P-value vs. placebo | 0.2646 | 0.0540 |

Week 10

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | 1.79 | (1.32) | 1.30 | (0.99) | 1.93 | (1.00) | 1.64 | (1.02) |

| | | | | |
|---|---|---|---|---|
| Median | 2.00 | 1.00 | 2.00 | 2.00 |
| Q1; Q3 | 1.00; 3.00 | 1.00; 2.00 | 1.00; 3.00 | 1.00; 2.00 |
| Min; Max | 0.0; 4.0 | 0.0; 3.0 | 0.0; 4.0 | 0.0; 3.0 |

Change from baseline

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | −0.43 | (1.37) | −0.89 | (0.97) | −0.35 | (0.87) | −0.86 | (1.02) |
| Median | 0.00 | | −1.00 | | 0.00 | | −1.00 | |
| Q1; Q3 | −1.50; 0.50 | | −2.00; 0.00 | | −1.00; 0.00 | | −2.00; 0.00 | |
| Min; Max | −3.0; 2.0 | | −2.0; 1.0 | | −2.0; 1.0 | | −3.0; 1.0 | |
| LS Mean (SE) | −0.15 | (0.22) | −0.63 | (0.24) | −0.46 | (0.13) | −0.95 | (0.13) |

| | | | | |
|---|---|---|---|---|
| LS Mean Diff vs. placebo | −0.47 | (−1.02, 0.07) | −0.49 | (−0.83, −0.15) |

(95% CI)

| | | |
|---|---|---|
| P-value vs. placebo | 0.0874 | 0.0050 |

Week 12

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | 2.00 | (1.15) | 1.25 | (1.08) | 2.00 | (1.25) | 1.67 | (0.97) |

| | | | | |
|---|---|---|---|---|
| Median | 2.00 | 1.00 | 2.00 | 2.00 |
| Q1; Q3 | 1.00; 3.00 | 0.00; 2.00 | 1.00; 3.00 | 1.00; 2.00 |
| Min; Max | 0.0; 4.0 | 0.0; 3.0 | 0.0; 6.0 | 0.0; 3.0 |

Change from baseline

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | −0.18 | (1.25) | −0.89 | (1.10) | −0.32 | (1.24) | −0.84 | (0.99) |
| Median | 0.00 | | −1.00 | | 0.00 | | −1.00 | |
| Q1; Q3 | −1.00; 1.00 | | −2.00; 0.00 | | −1.00; 0.00 | | −1.00; 0.00 | |
| Min; Max | −3.0; 2.0 | | −3.0; 1.0 | | −3.0; 4.0 | | −3.0; 1.0 | |
| LS Mean (SE) | 0.03 | (0.25) | −0.66 | (0.28) | −0.49 | (0.15) | −0.89 | (0.15) |

| | | | | |
|---|---|---|---|---|
| LS Mean Diff vs. placebo | −0.70 | (−1.28, −0.11) | −0.40 | (−0.79, −0.00) |

(95% CI)

| | | |
|---|---|---|
| P-value vs. placebo | 0.0192 | 0.0494 |

Limitation activities by asthma
Baseline

| Number | 32 | | 32 | | 68 | | 64 | |
|---|---|---|---|---|---|---|---|---|
| Mean (SD) | 2.34 | (0.87) | 2.06 | (0.56) | 2.13 | (0.64) | 2.17 | (0.52) |

| | | | | |
|---|---|---|---|---|
| Median | 2.00 | 2.00 | 2.00 | 2.00 |
| Q1; Q3 | 2.00; 3.00 | 2.00; 2.00 | 2.00; 3.00 | 2.00; 2.00 |
| Min; Max | 0.0; 4.0 | 1.0; 3.0 | 1.0; 3.0 | 1.0; 4.0 |

Week 2

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | 1.93 | (0.98) | 1.27 | (0.94) | 2.03 | (0.98) | 1.75 | (0.99) |

| | | | | |
|---|---|---|---|---|
| Median | 2.00 | 1.00 | 2.00 | 2.00 |
| Q1; Q3 | 1.00; 3.00 | 1.00; 2.00 | 2.00; 3.00 | 1.00; 3.00 |
| Min; Max | 0.0; 4.0 | 0.0; 3.0 | 0.0; 4.0 | 0.0; 3.0 |

Change from baseline

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | −0.37 | (1.16) | −0.80 | (1.03) | −0.13 | (0.89) | −0.39 | (1.04) |
| Median | 0.00 | | −1.00 | | 0.00 | | 0.00 | |
| Q1; Q3 | −1.00; 0.00 | | −2.00; 0.00 | | −1.00; 0.00 | | −1.00; 0.00 | |
| Min; Max | −3.0; 3.0 | | −2.0; 2.0 | | −2.0; 2.0 | | −3.0; 2.0 | |
| LS Mean (SE) | −0.31 | (0.20) | −0.90 | (0.22) | −0.11 | (0.13) | −0.48 | (0.13) |

| | | | | |
|---|---|---|---|---|
| LS Mean Diff vs. placebo | −0.59 | (−1.08, −0.09) | −0.37 | (−0.71, −0.03) |

(95% CI)

| | | |
|---|---|---|
| P-value vs. placebo | 0.0203 | 0.0323 |

Week 4

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | 1.93 | (1.01) | 0.94 | (1.00) | 1.95 | (1.00) | 1.56 | (0.95) |

| | | | | |
|---|---|---|---|---|
| Median | 2.00 | 1.00 | 2.00 | 2.00 |
| Q1; Q3 | 1.00; 3.00 | 0.00; 2.00 | 2.00; 2.00 | 1.00; 2.00 |
| Min; Max | 0.0; 4.0 | 0.0; 3.0 | 0.0; 4.0 | 0.0; 3.0 |

Change from baseline

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | −0.37 | (1.27) | −1.13 | (0.96) | −0.16 | (0.96) | −0.58 | (1.05) |
| Median | 0.00 | | −1.00 | | 0.00 | | 0.00 | |
| Q1; Q3 | −1.00; 0.00 | | −2.00; 0.00 | | −1.00; 1.00 | | −1.00; 0.00 | |
| Min; Max | −3.0; 2.0 | | −3.0; 1.0 | | −2.0; 2.0 | | −3.0; 1.0 | |
| LS Mean (SE) | −0.27 | (0.21) | −1.15 | (0.22) | −0.23 | (0.13) | −0.70 | (0.13) |

| | | | | |
|---|---|---|---|---|
| LS Mean Diff vs. placebo | −0.88 | (−1.40, −0.37) | −0.47 | (−0.80, −0.13) |

(95% CI)

| | | |
|---|---|---|
| P-value vs. placebo | 0.0007 | 0.0064 |

Week 6

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | 2.04 | (1.17) | 0.97 | (1.07) | 1.82 | (1.03) | 1.46 | (1.05) |

| | | | | |
|---|---|---|---|---|
| Median | 2.00 | 1.00 | 2.00 | 1.00 |
| Q1; Q3 | 1.00; 3.00 | 0.00; 2.00 | 1.00; 2.00 | 1.00; 2.00 |
| Min; Max | 0.0; 4.0 | 0.0; 3.0 | 0.0; 4.0 | 0.0; 4.0 |

Change from baseline

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | −0.29 | (1.08) | −1.13 | (1.04) | −0.32 | (1.08) | −0.72 | (1.15) |
| Median | 0.00 | | −1.00 | | 0.00 | | −1.00 | |
| Q1; Q3 | −1.00; 0.50 | | −2.00; 0.00 | | −1.00; 1.00 | | −2.00; 0.00 | |
| Min; Max | −3.0; 1.0 | | −3.0; 1.0 | | −3.0; 2.0 | | −3.0; 2.0 | |
| LS Mean (SE) | −0.07 | (0.22) | −0.95 | (0.23) | −0.25 | (0.15) | −0.76 | (0.15) |

| | | | | |
|---|---|---|---|---|
| LS Mean Diff vs. placebo | −0.89 | (−1.42, −0.35) | −0.51 | (−0.89, −0.13) |

(95% CI)

| | | |
|---|---|---|
| P-value vs. placebo | 0.0012 | 0.0090 |

Week 8

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | 1.73 | (1.17) | 1.00 | (0.98) | 1.94 | (1.15) | 1.31 | (0.88) |

| | | | | |
|---|---|---|---|---|
| Median | 2.00 | 1.00 | 2.00 | 1.00 |
| Q1; Q3 | 1.00; 3.00 | 0.00; 1.50 | 1.00; 2.00 | 1.00; 2.00 |
| Min; Max | 0.0; 4.0 | 0.0; 3.0 | 0.0; 5.0 | 0.0; 3.0 |

Change from baseline

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | −0.60 | (1.28) | −1.07 | (1.09) | −0.16 | (1.21) | −0.87 | (0.95) |
| Median | 0.00 | | −1.00 | | 0.00 | | −1.00 | |
| Q1; Q3 | −2.00; 0.00 | | −2.00; 0.00 | | −1.00; 1.00 | | −1.00; 0.00 | |
| Min; Max | −3.0; 1.0 | | −3.0; 1.0 | | −3.0; 4.0 | | −3.0; 1.0 | |
| LS Mean (SE) | −0.30 | (0.21) | −0.85 | (0.25) | −0.27 | (0.14) | −0.91 | (0.14) |

| | | | | |
|---|---|---|---|---|
| LS Mean Diff vs. placebo | −0.55 | (−1.08, −0.02) | −0.64 | (−1.00, −0.28) |

(95% CI)

| | | |
|---|---|---|
| P-value vs. placebo | 0.0432 | 0.0005 |

Week 10

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | 1.79 | (1.45) | 1.11 | (1.19) | 1.76 | (1.14) | 1.36 | (0.98) |

| | | | | |
|---|---|---|---|---|
| Median | 1.50 | 1.00 | 2.00 | 1.00 |
| Q1; Q3 | 1.00; 3.00 | 0.00; 2.00 | 1.00; 3.00 | 1.00; 2.00 |
| Min; Max | 0.0; 5.0 | 0.0; 3.0 | 0.0; 4.0 | 0.0; 3.0 |

Change from baseline

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | −0.54 | (1.48) | −0.96 | (1.16) | −0.35 | (1.11) | −0.80 | (1.05) |
| Median | 0.00 | | −1.00 | | 0.00 | | −1.00 | |
| Q1; Q3 | −2.00; 0.50 | | −2.00; 0.00 | | −1.00; 1.00 | | −1.00; 0.00 | |
| Min; Max | −3.0; 3.0 | | −2.0; 1.0 | | −2.0; 2.0 | | −3.0; 1.0 | |
| LS Mean (SE) | −0.10 | (0.24) | −0.67 | (0.26) | −0.34 | (0.15) | −0.91 | (0.15) |

| | | | | |
|---|---|---|---|---|
| LS Mean Diff vs. placebo | −0.58 | (−1.17, 0.02) | −0.57 | (−0.96, −0.19) |

(95% CI)

| | | |
|---|---|---|
| P-value vs. placebo | 0.0570 | 0.0035 |

Week 12

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | 1.93 | (1.27) | 1.18 | (1.12) | 1.78 | (1.12) | 1.20 | (0.96) |

| | | | | |
|---|---|---|---|---|
| Median | 2.00 | 1.00 | 2.00 | 1.00 |
| Q1; Q3 | 1.00; 3.00 | 0.00; 2.00 | 1.00; 3.00 | 0.00; 2.00 |
| Min; Max | 0.0; 5.0 | 0.0; 3.0 | 0.0; 5.0 | 0.0; 4.0 |

Change from baseline

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | −0.39 | (1.31) | −0.89 | (1.03) | −0.30 | (1.25) | −0.98 | (1.03) |
| Median | 0.00 | | −1.00 | | 0.00 | | −1.00 | |
| Q1; Q3 | −1.00; 0.50 | | −2.00; 0.00 | | −1.00; 1.00 | | −2.00; 0.00 | |
| Min; Max | −3.0; 3.0 | | −2.0; 1.0 | | −3.0; 4.0 | | −3.0; 2.0 | |
| LS Mean (SE) | −0.08 | (0.23) | −0.67 | (0.25) | −0.42 | (0.14) | −1.02 | (0.15) |

| | | | | |
|---|---|---|---|---|
| LS Mean Diff vs. placebo | −0.58 | (−1.13, −0.03) | −0.60 | (−0.97, −0.22) |

(95% CI)

| | | |
|---|---|---|
| P-value vs. placebo | 0.0391 | 0.0020 |

Shortness of breath experienced
Baseline

| Number | 32 | | 32 | | 68 | | 64 | |
|---|---|---|---|---|---|---|---|---|
| Mean (SD) | 2.50 | (0.62) | 2.31 | (0.69) | 2.37 | (0.62) | 2.41 | (0.71) |

| | | | | |
|---|---|---|---|---|
| Median | 2.00 | 2.00 | 2.00 | 2.00 |
| Q1; Q3 | 2.00; 3.00 | 2.00; 3.00 | 2.00; 3.00 | 2.00; 3.00 |
| Min; Max | 2.0; 4.0 | 1.0; 4.0 | 1.0; 4.0 | 0.0; 4.0 |

Week 2

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | 2.23 | (0.90) | 1.60 | (0.77) | 2.29 | (0.87) | 2.05 | (1.07) |

| | | | | |
|---|---|---|---|---|
| Median | 2.00 | 1.50 | 2.00 | 2.00 |
| Q1; Q3 | 2.00; 3.00 | 1.00; 2.00 | 2.00; 3.00 | 1.00; 3.00 |
| Min; Max | 0.0; 4.0 | 0.0; 3.0 | 0.0; 4.0 | 0.0; 4.0 |

Change from baseline

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | −0.23 | (0.94) | −0.70 | (0.84) | −0.06 | (0.90) | −0.38 | (1.11) |
| Median | 0.00 | | −1.00 | | 0.00 | | 0.00 | |
| Q1; Q3 | −1.00; 0.00 | | −1.00; 0.00 | | −1.00; 0.00 | | −1.00; 0.00 | |
| Min; Max | −2.0; 2.0 | | −2.0; 1.0 | | −3.0; 2.0 | | −3.0; 2.0 | |
| LS Mean (SE) | −0.12 | (0.17) | −0.66 | (0.18) | −0.09 | (0.13) | −0.44 | (0.13) |

| | | | | |
|---|---|---|---|---|
| LS Mean Diff vs. placebo | −0.55 | (−0.96, −0.13) | −0.35 | (−0.68, −0.01) |

| (95% CI) | | |
|---|---|---|
| P-value vs. placebo | 0.0099 | 0.0409 |

Week 4

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | 2.13 | (1.04) | 1.29 | (0.94) | 2.11 | (1.09) | 1.73 | (1.05) |

| | | | | |
|---|---|---|---|---|
| Median | 2.00 | 1.00 | 2.00 | 2.00 |
| Q1; Q3 | 2.00; 3.00 | 1.00; 2.00 | 2.00; 3.00 | 1.00; 3.00 |
| Min; Max | 0.0; 4.0 | 0.0; 3.0 | 0.0; 5.0 | 0.0; 3.0 |

Change from baseline

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | −0.33 | (1.21) | −1.03 | (0.87) | −0.24 | (1.07) | −0.66 | (1.06) |
| Median | 0.00 | | −1.00 | | 0.00 | | −1.00 | |
| Q1; Q3 | −1.00; 0.00 | | −2.00; 0.00 | | −1.00; 0.00 | | −1.00; 0.00 | |
| Min; Max | −3.0; 2.0 | | −3.0; 0.0 | | −3.0; 3.0 | | −3.0; 1.0 | |
| LS Mean (SE) | −0.17 | (0.20) | −0.88 | (0.21) | −0.27 | (0.14) | −0.73 | (0.15) |

| | | | | |
|---|---|---|---|---|
| LS Mean Diff vs. placebo | −0.71 | (−1.21, −0.21) | −0.47 | (−0.84, −0.09) |

| (95% CI) | | |
|---|---|---|
| P-value vs. placebo | 0.0053 | 0.0147 |

Week 6

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | 2.32 | (1.16) | 1.33 | (1.03) | 2.05 | (0.89) | 1.81 | (1.09) |

| | | | | |
|---|---|---|---|---|
| Median | 2.00 | 1.00 | 2.00 | 2.00 |
| Q1; Q3 | 1.50; 3.00 | 1.00; 2.00 | 2.00; 3.00 | 1.00; 3.00 |
| Min; Max | 0.0; 5.0 | 0.0; 4.0 | 0.0; 4.0 | 0.0; 4.0 |

Change from baseline

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | −0.14 | (1.27) | −1.03 | (1.03) | −0.30 | (0.83) | −0.61 | (1.18) |
| Median | 0.00 | | −1.00 | | 0.00 | | 0.00 | |
| Q1; Q3 | −1.00; 1.00 | | −2.00; −1.00 | | −1.00; 0.00 | | −1.00; 0.00 | |
| Min; Max | −3.0; 2.0 | | −3.0; 2.0 | | −3.0; 1.0 | | −4.0; 2.0 | |
| LS Mean (SE) | −0.00 | (0.24) | −0.90 | (0.25) | −0.25 | (0.14) | −0.64 | (0.14) |

| | | | | |
|---|---|---|---|---|
| LS Mean Diff vs. placebo | −0.90 | (−1.47, −0.33) | −0.39 | (−0.74, −0.04) |

| (95% CI) | | |
|---|---|---|
| P-value vs. placebo | 0.0019 | 0.0310 |

Week 8

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | 1.80 | (1.06) | 1.32 | (1.19) | 2.16 | (1.25) | 1.63 | (1.01) |

| | | | | |
|---|---|---|---|---|
| Median | 2.00 | 1.00 | 2.00 | 2.00 |
| Q1; Q3 | 1.00; 3.00 | 1.00; 2.00 | 1.00; 3.00 | 1.00; 2.00 |
| Min; Max | 0.0; 4.0 | 0.0; 5.0 | 0.0; 6.0 | 0.0; 3.0 |

Change from baseline

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | −0.67 | (1.21) | −0.93 | (1.25) | −0.21 | (1.12) | −0.77 | (1.08) |
| Median | 0.00 | | −1.00 | | 0.00 | | −1.00 | |
| Q1; Q3 | −1.00; 0.00 | | −2.00; −1.00 | | −1.00; 0.00 | | −1.00; 0.00 | |
| Min; Max | −4.0; 1.0 | | −3.0; 3.0 | | −2.0; 3.0 | | −3.0; 1.0 | |
| LS Mean (SE) | −0.44 | (0.24) | −0.83 | (0.27) | −0.29 | (0.14) | −0.87 | (0.15) |

| | | | | |
|---|---|---|---|---|
| LS Mean Diff vs. placebo | −0.39 | (−0.97, 0.19) | −0.58 | (−0.96, −0.20) |

| (95% CI) | | |
|---|---|---|
| P-value vs. placebo | 0.1848 | 0.0026 |

Week 10

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | 1.86 | (1.18) | 1.26 | (1.10) | 2.02 | (1.28) | 1.63 | (0.98) |

| | | | | |
|---|---|---|---|---|
| Median | 2.00 | 1.00 | 2.00 | 2.00 |
| Q1; Q3 | 1.00; 3.00 | 0.00; 2.00 | 1.00; 3.00 | 1.00; 2.00 |
| Min; Max | 0.0; 4.0 | 0.0; 4.0 | 0.0; 5.0 | 0.0; 3.0 |

Change from baseline

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | −0.57 | (1.37) | −1.15 | (1.17) | −0.31 | (1.22) | −0.79 | (1.16) |
| Median | 0.00 | | −1.00 | | 0.00 | | −1.00 | |
| Q1; Q3 | −2.00; 0.50 | | −2.00; 0.00 | | −1.00; 1.00 | | −1.00; 0.00 | |
| Min; Max | −4.0; 1.0 | | −3.0; 2.0 | | −3.0; 2.0 | | −3.0; 2.0 | |
| LS Mean (SE) | −0.19 | (0.21) | −0.74 | (0.23) | −0.24 | (0.16) | −0.81 | (0.17) |

| | | | | |
|---|---|---|---|---|
| LS Mean Diff vs. placebo | −0.54 | (−1.07, −0.01) | −0.57 | (−0.99, −0.15) |

| (95% CI) | | |
|---|---|---|
| P-value vs. placebo | 0.0442 | 0.0080 |

Week 12

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | 1.96 | (0.96) | 1.29 | (1.05) | 2.17 | (1.33) | 1.63 | (0.97) |

| | | | | |
|---|---|---|---|---|
| Median | 2.00 | 1.00 | 2.00 | 2.00 |
| Q1; Q3 | 1.00; 3.00 | 0.50; 2.00 | 1.00; 3.00 | 1.00; 2.00 |
| Min; Max | 0.0; 4.0 | 0.0; 4.0 | 0.0; 6.0 | 0.0; 3.0 |

Change from baseline

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | −0.50 | (1.17) | −1.07 | (1.18) | −0.22 | (1.22) | −0.86 | (1.02) |
| Median | 0.00 | | −1.00 | | 0.00 | | −1.00 | |
| Q1; Q3 | −1.00; 0.00 | | −2.00; −1.00 | | −1.00; 1.00 | | −2.00; 0.00 | |
| Min; Max | −4.0; 1.0 | | −3.0; 2.0 | | −3.0; 3.0 | | −3.0; 1.0 | |
| LS Mean (SE) | −0.20 | (0.21) | −0.87 | (0.23) | −0.25 | (0.15) | −0.84 | (0.15) |

| | | | | |
|---|---|---|---|---|
| LS Mean Diff vs. placebo | −0.66 | (−1.18, −0.15) | −0.59 | (−0.99, −0.19) |

| (95% CI) | | |
|---|---|---|
| P-value vs. placebo | 0.0116 | 0.0039 |

Wheeze
Baseline

| Number | 32 | | 32 | | 68 | | 64 | |
|---|---|---|---|---|---|---|---|---|
| Mean (SD) | 1.97 | (0.82) | 2.06 | (0.72) | 2.13 | (0.83) | 2.23 | (0.79) |

| | | | | |
|---|---|---|---|---|
| Median | 2.00 | 2.00 | 2.00 | 2.00 |
| Q1; Q3 | 1.00; 2.00 | 2.00; 2.00 | 2.00; 3.00 | 2.00; 3.00 |
| Min; Max | 1.0; 4.0 | 1.0; 4.0 | 0.0; 5.0 | 1.0; 4.0 |

Week 2

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | 1.80 | (1.19) | 1.00 | (0.87) | 1.90 | (0.93) | 1.89 | (1.05) |

| | | | | |
|---|---|---|---|---|
| Median | 2.00 | 1.00 | 2.00 | 2.00 |
| Q1; Q3 | 1.00; 2.00 | 0.00; 2.00 | 1.00; 3.00 | 1.00; 3.00 |
| Min; Max | 0.0; 6.0 | 0.0; 3.0 | 0.0; 4.0 | 0.0; 5.0 |

Change from baseline

| Number (observed/LOCF | 30 | (29/1) | 30 | (30/0) | 63 | (62/1) | 61 | (61/0) |
|---|---|---|---|---|---|---|---|---|
| imputed) | | | | | | | | |
| Mean (SD) | −0.17 | (1.05) | −1.07 | (0.94) | −0.21 | (1.09) | −0.34 | (1.00) |
| Median | 0.00 | | −1.00 | | 0.00 | | 0.00 | |
| Q1; Q3 | −1.00; 0.00 | | −2.00; 0.00 | | −1.00; 0.00 | | −1.00; 0.00 | |
| Min; Max | −2.0; 3.0 | | −3.0; 1.0 | | −4.0; 2.0 | | −2.0; 3.0 | |
| LS Mean (SE) | −0.25 | (0.19) | −1.08 | (0.20) | −0.29 | (0.12) | −0.44 | (0.12) |

| | | | | |
|---|---|---|---|---|
| LS Mean Diff vs. placebo | −0.83 | (−1.29, −0.36) | −0.15 | (−0.47, 0.17) |

| (95% CI) | | |
|---|---|---|
| P-value vs. placebo | 0.0005 | 0.3687 |

Week 4

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | 1.60 | (1.04) | 1.26 | (1.12) | 1.84 | (1.09) | 1.81 | (1.12) |

| | | | | |
|---|---|---|---|---|
| Median | 1.50 | 1.00 | 2.00 | 2.00 |
| Q1; Q3 | 1.00; 2.00 | 0.00; 2.00 | 1.00; 3.00 | 1.00; 3.00 |
| Min; Max | 0.0; 4.0 | 0.0; 5.0 | 0.0; 4.0 | 0.0; 5.0 |

Change from baseline

| Number (observed/LOCF | 30 | (28/2) | 31 | (30/1) | 62 | (61/1) | 59 | (59/0) |
|---|---|---|---|---|---|---|---|---|
| imputed) | | | | | | | | |
| Mean (SD) | −0.37 | (1.10) | −0.81 | (1.01) | −0.26 | (1.23) | −0.42 | (1.02) |
| Median | 0.00 | | −1.00 | | 0.00 | | 0.00 | |
| Q1; Q3 | −1.00; 0.00 | | −1.00; 0.00 | | −1.00; 0.00 | | −1.00; 0.00 | |
| Min; Max | −3.0; 2.0 | | −3.0; 2.0 | | −5.0; 2.0 | | −2.0; 3.0 | |
| LS Mean (SE) | −0.45 | (0.21) | −0.83 | (0.22) | −0.43 | (0.14) | −0.50 | (0.14) |

| | | | | |
|---|---|---|---|---|
| LS Mean Diff vs. placebo | −0.38 | (−0.89, 0.14) | −0.07 | (−0.44, 0.30) |

| (95% CI) | | |
|---|---|---|
| P-value vs. placebo | 0.1498 | 0.7130 |

Week 6

| Number (observed/LOCF | 28 | (26/2) | 30 | (28/2) | 60 | (58/2) | 57 | (56/1) |
|---|---|---|---|---|---|---|---|---|
| imputed) | | | | | | | | |
| Mean (SD) | 1.75 | (1.46) | 0.97 | (0.93) | 1.67 | (1.13) | 1.70 | (1.05) |
| Median | 2.00 | | 1.00 | | 2.00 | | 2.00 | |
| Q1; Q3 | 1.00; 2.00 | | 0.00; 2.00 | | 1.00; 2.00 | | 1.00; 2.00 | |
| Min; Max | 0.0; 6.0 | | 0.0; 3.0 | | 0.0; 4.0 | | 0.0; 4.0 | |
| Number (observed/LOCF | 28 | (26/2) | 30 | (28/2) | 60 | (58/2) | 57 | (56/1) |
| imputed) | | | | | | | | |
| Mean (SD) | −0.21 | (1.55) | −1.13 | (1.04) | −0.45 | (1.21) | −0.58 | (0.96) |
| Median | 0.00 | | −1.00 | | 0.00 | | −1.00 | |
| Q1; Q3 | −1.00; 0.00 | | −2.00; 0.00 | | −1.00; 0.00 | | −1.00; 0.00 | |
| Min; Max | −3.0; 3.0 | | −3.0; 1.0 | | −5.0; 2.0 | | −3.0; 2.0 | |
| LS Mean (SE) | −0.27 | (0.24) | −1.08 | (0.26) | −0.45 | (0.14) | −0.61 | (0.14) |

| | | | | |
|---|---|---|---|---|
| LS Mean Diff vs. placebo | −0.81 | (−1.40, −0.22) | −0.16 | (−0.52, 0.20) |

| (95% CI) | | |
|---|---|---|
| P-value vs. placebo | 0.0076 | 0.3907 |

Week 8

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | 1.70 | (1.44) | 1.11 | (1.07) | 1.94 | (1.32) | 1.58 | (0.98) |

| | | | | |
|---|---|---|---|---|
| Median | 2.00 | 1.00 | 2.00 | 2.00 |
| Q1; Q3 | 1.00; 2.00 | 0.00; 2.00 | 1.00; 3.00 | 1.00; 2.00 |
| Min; Max | 0.0; 5.0 | 0.0; 3.0 | 0.0; 5.0 | 0.0; 4.0 |

Change from baseline

| Number (observed/LOCF | 30 | (27/3) | 28 | (24/4) | 63 | (60/3) | 52 | (49/3) |
|---|---|---|---|---|---|---|---|---|
| imputed) | | | | | | | | |
| Mean (SD) | −0.27 | (1.44) | −0.96 | (1.07) | −0.13 | (1.41) | −0.69 | (0.98) |
| Median | 0.00 | | −1.00 | | 0.00 | | −1.00 | |
| Q1; Q3 | −1.00; 0.00 | | −2.00; 0.00 | | −1.00; 1.00 | | −1.00; 0.00 | |
| Min; Max | −4.0; 3.0 | | −3.0; 1.0 | | −5.0; 3.0 | | −3.0; 1.0 | |
| LS Mean (SE) | −0.32 | (0.25) | −0.99 | (0.27) | −0.31 | (0.15) | −0.69 | (0.16) |

| | | | | |
|---|---|---|---|---|
| LS Mean Diff vs. placebo | −0.67 | (−1.27, −0.06) | −0.37 | (−0.77, 0.03) |

| (95% CI) | | |
|---|---|---|
| P-value vs. placebo | 0.0315 | 0.0669 |

Week 10

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | 1.93 | (1.27) | 1.19 | (0.96) | 1.85 | (1.38) | 1.52 | (1.03) |

| | | | | |
|---|---|---|---|---|
| Median | 2.00 | 1.00 | 2.00 | 1.00 |
| Q1; Q3 | 1.00; 2.50 | 0.00; 2.00 | 1.00; 3.00 | 1.00; 2.00 |
| Min; Max | 0.0; 5.0 | 0.0; 3.0 | 0.0; 5.0 | 0.0; 4.0 |

Change from baseline

| Number (observed/LOCF | 28 | (22/6) | 27 | (23/4) | 55 | (51/4) | 56 | (53/3) |
|---|---|---|---|---|---|---|---|---|
| imputed) | | | | | | | | |
| Mean (SD) | 0.00 | (1.44) | −0.93 | (1.14) | −0.25 | (1.49) | −0.73 | (1.12) |
| Median | 0.00 | | −1.00 | | 0.00 | | −1.00 | |
| Q1; Q3 | −1.00; 1.00 | | −2.00; 0.00 | | −1.00; 1.00 | | −1.00; 0.00 | |
| Min; Max | −3.0; 2.0 | | −4.0; 1.0 | | −5.0; 3.0 | | −3.0; 2.0 | |
| LS Mean (SE) | −0.00 | (0.21) | −0.83 | (0.23) | −0.36 | (0.16) | −0.79 | (0.17) |

| | | | | |
|---|---|---|---|---|
| LS Mean Diff vs. placebo | −0.82 | (−1.35, −0.30) | −0.43 | (−0.85, −0.01) |

| (95% CI) | | |
|---|---|---|
| P-value vs. placebo | 0.0021 | 0.0450 |

Week 12

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | 1.86 | (1.35) | 1.21 | (1.10) | 2.05 | (1.53) | 1.49 | (0.84) |

| | | | | |
|---|---|---|---|---|
| Median | 2.00 | 1.00 | 2.00 | 2.00 |
| Q1; Q3 | 1.00; 2.00 | 0.00; 2.00 | 1.00; 3.50 | 1.00; 2.00 |
| Min; Max | 0.0; 5.0 | 0.0; 3.0 | 0.0; 5.0 | 0.0; 3.0 |

Change from baseline

| Number (observed/LOCF | 28 | (19/9) | 28 | (23/5) | 60 | (50/10) | 49 | (46/3) |
|---|---|---|---|---|---|---|---|---|
| imputed) | | | | | | | | |
| Mean (SD) | −0.14 | (1.58) | −0.86 | (1.11) | −0.05 | (1.41) | −0.80 | (1.00) |
| Median | 0.00 | | −1.00 | | 0.00 | | −1.00 | |
| Q1; Q3 | −1.00; 1.00 | | −2.00; 0.00 | | −1.00; 1.00 | | −1.00; 0.00 | |
| Min; Max | −4.0; 2.0 | | −2.0; 1.0 | | −3.0; 3.0 | | −3.0; 1.0 | |
| LS Mean (SE) | −0.03 | (0.25) | −0.63 | (0.28) | −0.22 | (0.16) | −0.80 | (0.17) |

| | | | | |
|---|---|---|---|---|
| LS Mean Diff vs. placebo | −0.60 | (−1.21, 0.00) | −0.58 | (−1.00, −0.15) |

(95% CI)

| | | |
|---|---|---|
| P-value vs. placebo | 0.0500 | 0.0078 |

ACQ-5: Asthma control questionnaire, 5-question version, LOAC: Loss of asthma control, LOCF: Last observation carried forward, LATAM: Latin America, EASTEU: Eastern Europe, ROW: Rest of the world

TABLE 43
Responder analysis for change from baseline
in ACQ-5 score at Week 12 (mITT population).

| 400 mg BID cohort | | 400 mg TID cohort | |
|---|---|---|---|
| | Rilzabrutinib | | Rilzabrutinib |
| Placebo BID | 400 mg BID | Placebo TID | 400 mg TID |
| (N = 32) | (N = 32) | (N = 68) | (N = 64) |

Week 12

| | | | | |
|---|---|---|---|---|
| Number (observed/LOCF | 28 | 28 | 60 | 49 |
| imputed) | (19/9) | (23/5) | (50/10) | (46/3) |
| Responder (observed/LOCF | 10 | 19 | 24 | 31 |
| imputed) | (35.7) (9/1) | (67.9) (17/2) | (40.0) (23/1) | (63.3) (31/0) |
| Non-responder | 18 | 9 | 36 | 18 |
| (observed/LOCF imputed) | (64.3) (10/8) | (32.1) (6/3) | (60.0) (27/9) | (36.7) (15/3) |
| Responder (including | 11.5 | 21.2 | 26.8 | 38.3 |
| multiply imputed) | (35.9) | (66.1) | (39.4) | (59.8) |
| OR vs. placebo (95% CI) | | 5.18 | | 2.38 |
| | | (1.48, 18.15) | | (1.09, 5.18) |
| P-value vs. placebo | | 0.0100 | | 0.0297 |

LATAM: Latin America, ROW: Rest of the world

TABLE 44
Responder analysis for ACQ-5 score less than 0.75 over time (mITT population).

| BID cohort | | TID cohort | |
|---|---|---|---|
| | Rilzabrutinib | | Rilzabrutinib |
| Placebo BID | 400 mg BID | Placebo TID | 400 mg TID |
| (N = 32) | (N = 32) | (N = 68) | (N = 64) |

Baseline

| | | | | | | | | |
|---|---|---|---|---|---|---|---|---|
| Number (observed/NR imputed) | 32 | (32/0) | 32 | (32/0) | 68 | (68/0) | 64 | (64/0) |

| Responder (observed) | 0 | 0 | 0 | 0 |
|---|---|---|---|---|
| Non-responder (observed/NR | 32 (100) | 32 (100) | 68 (100) | 64 (100) |
| imputed) | (32/0) | (32/0) | (68/0) | (64/0) |

Week 2

| | | | | | | | | |
|---|---|---|---|---|---|---|---|---|
| Number (observed/LOCF | 32 | (29/1/2) | 32 | (30/0/2) | 68 | (62/1/5) | 64 | (61/0/3) |

| imputed/NR imputed) | | | | |
|---|---|---|---|---|
| Responder (observed/LOCF | 1 (3.1) | 6 (18.8) | 4 (5.9) | 10 (15.6) |
| imputed) | (1/0) | (6/0) | (4/0) | (10/0) |
| Non-responder (observed/LOCF | 31 (96.9) | 26 (81.3) | 64 (94.1) | 54 (84.4) |
| imputed/NR imputed) | (28/1/2) | (24/0/2) | (58/1/5) | (51/0/3) |
| OR vs. placebo (95% CI) | | 5.66 (0.61, | | 3.39 (0.95, |
| | | 52.11) | | 12.05) |
| P-value vs. placebo | | 0.1262 | | 0.0590 |

Week 4

| | | | | | | | | |
|---|---|---|---|---|---|---|---|---|
| Number (observed/LOCF | 32 | (28/2/2) | 32 | (30/1/1) | 68 | (61/1/6) | 64 | (59/0/5) |

| imputed/NR imputed) | | | | |
|---|---|---|---|---|
| Responder (observed/LOCF | 2 (6.3) | 11 (34.4) | 5 (7.4) | 10 (15.6) |
| imputed) | (2/0) | (11/0) | (5/0) | (10/0) |
| Non-responder (observed/LOCF | 30 (93.8) | 21 (65.6) | 63 (92.6) | 54 (84.4) |
| imputed/NR imputed) | (26/2/2) | (19/1/1) | (56/1/6) | (49/0/5) |
| OR vs. placebo (95% CI) | | 8.34 (1.55, | | 2.78 (0.82, |
| | | 44.69) | | 9.38) |
| P-value vs. placebo | | 0.0133 | | 0.1003 |

Week 6

| | | | | | | | | |
|---|---|---|---|---|---|---|---|---|
| Number (observed/LOCF | 32 | (26/2/4) | 32 | (28/2/2) | 68 | (58/2/8) | 64 | (56/1/7) |

| imputed/NR imputed) | | | | |
|---|---|---|---|---|
| Responder (observed/LOCF | 3 (9.4) (3/0) | 11 (34.4) | 5 (7.4) (5/0) | 11 (17.2) (11/0) |
| imputed) | | (11/0) | | |
| Non-responder (observed/LOCF | 29 (90.6) | 21 (65.6) | 63 (92.6) | 53 (82.8) |
| imputed/NR imputed) | (23/2/4) | (17/2/2) | (53/2/8) | (45/1/7) |
| OR vs. placebo (95% CI) | | 4.74 (1.12, | | 3.22 (0.96, |
| | | 20.02) | | 10.79) |
| P-value vs. placebo | | 0.0344 | | 0.0575 |

Week 8

| | | | | | | | | |
|---|---|---|---|---|---|---|---|---|
| Number (observed/LOCF | 32 | (27/3/2) | 32 | (24/4/4) | 68 | (60/3/5) | 64 | (49/3/12) |

| imputed/NR imputed) | | | | |
|---|---|---|---|---|
| Responder (observed/LOCF | 6 (18.8) | 9 (28.1) | 4 (5.9) | 9 (14.1) |
| imputed) | (6/0) | (9/0) | (4/0) | (9/0) |
| Non-responder (observed/LOCF | 26 (81.3) | 23 (71.9) | 64 (94.1) | 55 (85.9) |
| imputed/NR imputed) | (21/3/2) | (15/4/4) | (56/3/5) | (40/3/12) |
| OR vs. placebo (95% CI) | | 1.51 (0.45, | | 3.03 (0.83, |
| | | 5.09) | | 11.04) |
| P-value vs. placebo | | 0.5033 | | 0.0919 |

Week 10

| | | | | | | | | |
|---|---|---|---|---|---|---|---|---|
| Number (observed/LOCF | 32 | (22/6/4) | 32 | (23/4/5) | 68 | (51/4/13) | 64 | (53/3/8) |

| imputed/NR imputed) | | | | |
|---|---|---|---|---|
| Responder (observed/LOCF | 5 (15.6) | 11 (34.4) | 7 (10.3) | 12 (18.8) |
| imputed) | (5/0) | (11/0) | (7/0) | (12/0) |
| Non-responder (observed/LOCF | 27 (84.4) | 21 (65.6) | 61 (89.7) | 52 (81.3) |
| imputed/NR imputed) | (17/6/4) | (12/4/5) | (44/4/13) | (41/3/8) |
| OR vs. placebo (95% CI) | | 3.58 (0.95, | | 2.45 (0.80, |
| | | 13.55) | | 7.47) |
| P-value vs. placebo | | 0.0599 | | 0.1146 |

Week 12

| | | | | | | | | |
|---|---|---|---|---|---|---|---|---|
| Number (observed/LOCF | 32 | (19/9/4) | 32 | (23/5/4) | 68 | (50/10/8) | 64 | (46/3/15) |

| imputed/NR imputed) | | | | |
|---|---|---|---|---|
| Responder (observed/LOCF | 3 (9.4) (3/0) | 13 (40.6) | 9 (13.2) (9/0) | 10 (15.6) (10/0) |
| imputed) | | (12/1) | | |
| Non-responder (observed/LOCF | 29 (90.6) | 19 (59.4) | 59 (86.8) | 54 (84.4) |
| imputed/NR imputed) | (16/9/4) | (11/4/4) | (41/10/8) | (36/3/15) |
| OR vs. placebo (95% CI) | | 6.79 (1.61, | | 1.29 (0.46, |
| | | 28.67) | | 3.64) |
| P-value vs. placebo | | 0.0092 | | 0.6286 |

LATAM: Latin America, ROW: Rest of the world, NR: non-responder

TABLE 45
Number and proportion of participants remained as ACQ-5 less than
0.75 responders since Week 4 over time (mITT population).

| | |
|---|---|
| | Rilzabrutinib |
| Placebo BID | 400 mg BID |
| (N = 32) | (N = 32) |

Week 4

| | | | | |
|---|---|---|---|---|
| Number (observed/LOCF | 32 | (28/2/2) | 32 | (30/1/1) |
| imputed/NR imputed) | | | | |

| | | |
|---|---|---|
| Responder (observed/ | 2 (6.3) | 11 (34.4) |
| LOCF imputed) | (2/0) | (11/0) |
| Non-responder (observed/ | 30 (93.8) | 21 (65.6) |
| LOCF imputed/NR imputed) | (26/2/2) | (19/1/1) |

Week 6

| | | | | |
|---|---|---|---|---|
| Number (observed/LOCF imputed) | 2 | (2/0) | 11 | (10/0) |

| | | |
|---|---|---|
| Responder (observed/ | 1 (50.0) | 7 (63.6) |
| LOCF imputed) | (1/0) | (7/0) |
| OR vs. placebo (95% CI) | | 0.81 (0.01, |
| | | 126.85) |
| P-value vs. placebo | | 0.9356 |

Week 8

| | | | | |
|---|---|---|---|---|
| Number (observed/LOCF | 2 | (2/0) | 11 | (10/1) |
| imputed) | | | | |

| | | |
|---|---|---|
| Responder (observed/ | 1 (50.0) | 5 (45.5) |
| LOCF imputed) | (1/0) | (5/0) |
| OR vs. placebo (95% CI) | | 0.26 (0.00, |
| | | 28.99) |
| P-value vs. placebo | | 0.5729 |

Week 10

| | | | | |
|---|---|---|---|---|
| Number (observed/LOCF | 2 | (2/0) | 11 | (8/1) |
| imputed) | | | | |

| | | |
|---|---|---|
| Responder (observed/ | 1 (50.0) | 4 (36.4) |
| LOCF imputed) | (1/0) | (4/0) |
| OR vs. placebo (95% CI) | | 0.12 (0.00, |
| | | 16.67) |
| P-value vs. placebo | | 0.3991 |

Week 12

| | | | | |
|---|---|---|---|---|
| Number (observed/LOCF | 2 | (1/1) | 11 | (8/2) |
| imputed) | | | | |

| | | |
|---|---|---|
| Responder (observed/ | 0 | 3 (27.3) |
| LOCF imputed) | | (2/1) |
| OR vs. placebo (95% CI) | | NC (NC, |
| | | NC) |
| P-value vs. placebo | | NC |

LATAM: Latin America, ROW: Rest of the world, NR: non-responder

TABLE 46
Subgroup analysis: change from baseline in ACQ-5 score at
EOT (Week 12) by demographics subgroups (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| ACQ-5 | (N = 32) | (N = 32) | (N = 68) | (N = 64) |

Age group 1 (years)
<45

| Number | 10 | | 9 | | 28 | | 23 | |
|---|---|---|---|---|---|---|---|---|
| Mean (SD) | −0.70 | (0.91) | −0.49 | (0.94) | −0.05 | (1.02) | −0.84 | (0.62) |
| LS Mean (SE) | −0.70 | (0.31) | −0.68 | (0.34) | −0.25 | (0.17) | −0.95 | (0.17) |
| LS Mean Diff vs. | | | 0.02 | (−0.76, 0.79) | | | −0.70 | (−1.15, −0.25) |
| placebo (95% CI) | | | | | | | | |

| | | |
|---|---|---|
| P-value vs. placebo | 0.9616 | 0.0024 |

≥45

| Number | 18 | | 19 | | 32 | | 26 | |
|---|---|---|---|---|---|---|---|---|
| Mean (SD) | −0.13 | (1.05) | −1.07 | (0.88) | −0.38 | (1.04) | −0.79 | (0.81) |
| LS Mean (SE) | 0.14 | (0.23) | −0.78 | (0.25) | −0.35 | (0.17) | −0.79 | (0.18) |
| LS Mean Diff vs. | | | −0.92 | (−1.50, −0.35) | | | −0.43 | (−0.90, 0.03) |
| placebo (95% CI) | | | | | | | | |

| | | |
|---|---|---|
| P-value vs. placebo | 0.0016 | 0.0643 |
| Overall p-value for interaction | 0.0568 | 0.5311 |

Gender
Male

| Number | 7 | | 12 | | 26 | | 22 | |
|---|---|---|---|---|---|---|---|---|
| Mean (SD) | −0.31 | (0.98) | −1.08 | (0.79) | −0.22 | (0.82) | −0.65 | (0.65) |
| LS Mean (SE) | −0.32 | (0.36) | −0.82 | (0.42) | −0.31 | (0.17) | −0.64 | (0.17) |
| LS Mean Diff vs. | | | −0.50 | (−1.67, 0.67) | | | −0.34 | (−0.78, 0.11) |
| placebo (95% CI) | | | | | | | | |

| | | |
|---|---|---|
| P-value vs. placebo | 0.4019 | 0.1396 |

Female

| Number | 21 | | 16 | | 34 | | 27 | |
|---|---|---|---|---|---|---|---|---|
| Mean (SD) | −0.34 | (1.06) | −0.74 | (1.02) | −0.22 | (1.18) | −0.96 | (0.76) |
| LS Mean (SE) | −0.02 | (0.27) | −0.58 | (0.27) | −0.31 | (0.17) | −0.96 | (0.18) |
| LS Mean Diff vs. | | | −0.56 | (−1.19, 0.06) | | | −0.65 | (−1.11, −0.19) |
| placebo (95% CI) | | | | | | | | |

| | | |
|---|---|---|
| P-value vs. placebo | 0.0747 | 0.0056 |
| Overall p-value for interaction | 0.9599 | 0.3680 |

Baseline weight group (kg)
<60

| Number | 4 | | 3 | | 10 | | 2 | |
|---|---|---|---|---|---|---|---|---|
| Mean (SD) | 0.35 | (0.47) | −1.13 | (0.83) | −0.54 | (1.12) | −1.90 | (0.42) |
| LS Mean (SE) | 0.32 | (0.47) | −1.71 | (0.99) | −0.18 | (0.41) | −1.72 | (0.80) |
| LS Mean Diff vs. | | | −2.03 | (−5.03, 0.98) | | | −1.54 | (−3.28, 0.21) |
| placebo (95% CI) | | | | | | | | |

| | | |
|---|---|---|
| P-value vs. placebo | 0.1010 | 0.0847 |

≥60-<90

| Number | 21 | | 20 | | 35 | | 29 | |
|---|---|---|---|---|---|---|---|---|
| Mean (SD) | −0.52 | (1.00) | −0.80 | (0.89) | −0.15 | (1.07) | −0.74 | (0.68) |
| LS Mean (SE) | −0.27 | (0.25) | −0.63 | (0.25) | −0.25 | (0.16) | −0.80 | (0.17) |
| LS Mean Diff vs. | | | −0.35 | (−0.93, 0.22) | | | −0.54 | (−0.97, −0.11) |
| placebo (95% CI) | | | | | | | | |

| | | |
|---|---|---|
| P-value vs. placebo | 0.2295 | 0.0130 |

≥90

| Number | 3 | | 5 | | 15 | | 18 | |
|---|---|---|---|---|---|---|---|---|
| Mean (SD) | 0.07 | (1.50) | −1.08 | (1.25) | −0.17 | (0.91) | −0.81 | (0.74) |
| LS Mean (SE) | −0.68 | (0.45) | −1.96 | (0.57) | −0.34 | (0.23) | −0.86 | (0.19) |
| LS Mean Diff vs. | | | −1.27 | (−2.46, −0.09) | | | −0.51 | (−1.08, 0.05) |
| placebo (95% CI) | | | | | | | | |

| | | |
|---|---|---|
| P-value vs. placebo | 0.0353 | 0.0737 |
| Overall p-value for interaction | 0.4298 | 0.4325 |

Baseline BMI group (kg/m²)
<25

| Number | 6 | | 8 | | 20 | | 8 | |
|---|---|---|---|---|---|---|---|---|
| Mean (SD) | 0.00 | (0.79) | −0.90 | (0.81) | −0.33 | (1.02) | −1.03 | (0.82) |
| LS Mean (SE) | 0.65 | (0.54) | 0.03 | (0.68) | −0.40 | (0.25) | −1.06 | (0.37) |
| LS Mean Diff vs. | | | −0.61 | (−1.75, 0.53) | | | −0.66 | (−1.45, 0.14) |
| placebo (95% CI) | | | | | | | | |

| | | |
|---|---|---|
| P-value vs. placebo | 0.2545 | 0.1043 |

≥25-<30

| Number | 15 | | 13 | | 23 | | 26 | |
|---|---|---|---|---|---|---|---|---|
| Mean (SD) | −0.53 | (1.16) | −0.69 | (0.94) | −0.22 | (0.98) | −0.86 | (0.73) |
| LS Mean (SE) | −0.40 | (0.29) | −0.67 | (0.33) | −0.42 | (0.18) | −1.01 | (0.17) |
| LS Mean Diff vs. | | | −0.27 | (−1.10, 0.56) | | | −0.59 | (−1.04, −0.14) |
| placebo (95% CI) | | | | | | | | |

| | | |
|---|---|---|
| P-value vs. placebo | 0.5253 | 0.0101 |

≥30

| Number | 7 | | 7 | | 17 | | 15 | |
|---|---|---|---|---|---|---|---|---|
| Mean (SD) | −0.20 | (0.92) | −1.23 | (1.06) | −0.11 | (1.16) | −0.63 | (0.65) |
| LS Mean (SE) | 0.50 | (0.37) | −0.67 | (0.37) | −0.13 | (0.24) | −0.66 | (0.23) |
| LS Mean Diff vs. | | | −1.17 | (−1.91, −0.42) | | | −0.53 | (−1.17, 0.11) |
| placebo (95% CI) | | | | | | | | |

| | | |
|---|---|---|
| P-value vs. placebo | 0.0021 | 0.1017 |
| Overall p-value for interaction | 0.3782 | 0.9246 |

Region
Eastern Europe

| Number | 4 | | 1 | | 43 | | 32 | |
|---|---|---|---|---|---|---|---|---|
| Mean (SD) | −0.05 | (1.17) | 1.00 | (NC) | −0.04 | (0.88) | −0.72 | (0.66) |
| LS Mean (SE) | −0.48 | (0.69) | 0.40 | (0.94) | −0.06 | (0.13) | −0.59 | (0.14) |
| LS Mean Diff vs. | | | 0.88 | (−1.58, 3.35) | | | −0.53 | (−0.90, −0.15) |
| placebo (95% CI) | | | | | | | | |

| | | |
|---|---|---|
| P-value vs. placebo | 0.4823 | 0.0059 |

Latin America

| Number | 22 | | 24 | | 15 | | 15 | |
|---|---|---|---|---|---|---|---|---|
| Mean (SD) | −0.54 | (0.91) | −1.02 | (0.85) | −0.93 | (0.81) | −0.87 | (0.77) |
| LS Mean (SE) | −0.54 | (0.21) | −1.12 | (0.20) | −0.91 | (0.26) | −1.04 | (0.25) |
| LS Mean Diff vs. | | | −0.58 | (−1.11, −0.05) | | | −0.14 | (−0.70, 0.43) |
| placebo (95% CI) | | | | | | | | |

| | | |
|---|---|---|
| P-value vs. placebo | 0.0315 | 0.6394 |

Western Countries

| Number | 1 | | 2 | | 1 | | 2 | |
|---|---|---|---|---|---|---|---|---|
| Mean (SD) | 1.60 | (NC) | −0.60 | (1.41) | 3.20 | (NC) | −2.00 | (0.00) |
| LS Mean (SE) | NC | (NC) | NC | (NC) | NC | (NC) | NC | (NC) |
| LS Mean Diff vs. | | | NC | (NC, NC) | | | NC | (NC, NC) |
| placebo (95% CI) | | | | | | | | |

| | | |
|---|---|---|
| P-value vs. placebo | NC | NC |

APAC

| Number | 1 | | 1 | | 1 | | 0 | |
|---|---|---|---|---|---|---|---|---|
| Mean (SD) | 1.00 | (NC) | −0.20 | (NC) | −0.80 | (NC) | NC | (NC) |
| LS Mean (SE) | NC | (NC) | NC | (NC) | NC | (NC) | NC | (NC) |
| LS Mean Diff vs. | | | NC | (NC, NC) | | | NC | (NC, NC) |
| placebo (95% CI) | | | | | | | | |

| P-value vs. placebo | NC | NC |
|---|---|---|
| Overall p-value for interaction | 0.6608 | 0.0007 |

LATAM: Latin America, ROW: Rest of the world

TABLE 47
Subgroup analysis: change from baseline in ACQ-5 score at EOT (Week
12) by disease and other characteristics subgroups (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| ACQ-5 | (N = 32) | (N = 32) | (N = 68) | (N = 64) |

Age at onset of asthma (years)
<12

| Number | 7 | | 8 | | 14 | | 12 | |
|---|---|---|---|---|---|---|---|---|
| Mean (SD) | −0.49 | (0.80) | −0.68 | (1.13) | 0.09 | (0.93) | −0.58 | (0.67) |
| LS Mean (SE) | −0.70 | (0.42) | −0.71 | (0.55) | −0.02 | (0.22) | −0.63 | (0.24) |
| LS Mean Diff vs. | | | −0.01 | (−1.21, 1.18) | | | −0.61 | (−1.24, 0.02) |
| placebo (95% CI) | | | | | | | | |

| | | |
|---|---|---|
| P-value vs. placebo | 0.9822 | 0.0595 |

≥12-<18

| Number | 2 | | 4 | | 5 | | 5 | |
|---|---|---|---|---|---|---|---|---|
| Mean (SD) | −0.40 | (0.57) | −1.30 | (0.20) | −0.76 | (1.18) | −1.04 | (0.65) |
| LS Mean (SE) | −0.14 | (0.88) | −0.96 | (0.54) | −1.10 | (0.84) | −1.08 | (0.64) |
| LS Mean Diff vs. | | | −0.82 | (−3.23, 1.59) | | | 0.01 | (−1.86, 1.89) |
| placebo (95% CI) | | | | | | | | |

| | | |
|---|---|---|
| P-value vs. placebo | 0.2812 | 0.9849 |

≥18-<40

| Number | 13 | | 13 | | 24 | | 20 | |
|---|---|---|---|---|---|---|---|---|
| Mean (SD) | −0.25 | (1.06) | −0.97 | (0.98) | −0.11 | (1.24) | −0.87 | (0.69) |
| LS Mean (SE) | 0.15 | (0.27) | −0.71 | (0.27) | −0.20 | (0.23) | −0.77 | (0.24) |
| LS Mean Diff vs. | | | −0.85 | (−1.52, −0.19) | | | −0.57 | (−1.15, 0.01) |
| placebo (95% CI) | | | | | | | | |

| | | |
|---|---|---|
| P-value vs. placebo | 0.0119 | 0.0537 |

≥40

| Number | 6 | | 3 | | 17 | | 12 | |
|---|---|---|---|---|---|---|---|---|
| Mean (SD) | −0.33 | (1.44) | −0.53 | (0.81) | −0.48 | (0.64) | −0.87 | (0.86) |
| LS Mean (SE) | −0.08 | (0.49) | −0.87 | (0.83) | −0.39 | (0.20) | −0.90 | (0.22) |
| LS Mean Diff vs. | | | −0.78 | (−2.44, 0.88) | | | −0.51 | (−1.06, 0.03) |
| placebo (95% CI) | | | | | | | | |

| | | |
|---|---|---|
| P-value vs. placebo | 0.3555 | 0.0660 |
| Overall p-value for interaction | 0.5580 | 0.8917 |

Age at onset of asthma (years)
<18

| Number | 9 | | 12 | | 19 | | 17 | |
|---|---|---|---|---|---|---|---|---|
| Mean (SD) | −0.47 | (0.72) | −0.88 | (0.96) | −0.14 | (1.04) | −0.72 | (0.68) |
| LS Mean (SE) | −0.67 | (0.34) | −0.91 | (0.39) | −0.22 | (0.20) | −0.66 | (0.21) |
| LS Mean Diff vs. | | | −0.24 | (−1.08, 0.59) | | | −0.44 | (−0.99, 0.12) |
| placebo (95% CI) | | | | | | | | |

| | | |
|---|---|---|
| P-value vs. placebo | 0.5658 | 0.1215 |

≥18

| Number | 19 | | 16 | | 41 | | 32 | |
|---|---|---|---|---|---|---|---|---|
| Mean (SD) | −0.27 | (1.15) | −0.89 | (0.94) | −0.26 | (1.04) | −0.87 | (0.75) |
| LS Mean (SE) | 0.05 | (0.23) | −0.70 | (0.26) | −0.30 | (0.15) | −0.84 | (0.17) |
| LS Mean Diff vs. | | | −0.76 | (−1.35, −0.16) | | | −0.54 | (−0.94, −0.14) |
| placebo (95% CI) | | | | | | | | |

| | | |
|---|---|---|
| P-value vs. placebo | 0.0136 | 0.0089 |
| Overall p-value for interaction | 0.4014 | 0.8634 |

Number of asthma exacerbations within 2 years before screening visit
1

| Number | 8 | | 10 | | 45 | | 31 | |
|---|---|---|---|---|---|---|---|---|
| Mean (SD) | −0.63 | (0.92) | −1.06 | (1.05) | −0.23 | (0.99) | −0.95 | (0.74) |
| LS Mean (SE) | −0.37 | (0.37) | −1.12 | (0.35) | −0.39 | (0.15) | −1.04 | (0.16) |
| LS Mean Diff vs. | | | −0.76 | (−1.61, 0.09) | | | −0.66 | (−1.07, −0.25) |
| placebo (95% CI) | | | | | | | | |

| | | |
|---|---|---|
| P-value vs. placebo | 0.0811 | 0.0016 |

2

| Number | 14 | | 10 | | 15 | | 15 | |
|---|---|---|---|---|---|---|---|---|
| Mean (SD) | −0.39 | (1.18) | −0.88 | (0.97) | −0.21 | (1.20) | −0.64 | (0.69) |
| LS Mean (SE) | 0.13 | (0.33) | −0.11 | (0.48) | −0.26 | (0.26) | −0.70 | (0.24) |
| LS Mean Diff vs. | | | −0.23 | (−1.17, 0.70) | | | −0.45 | (−1.07, 0.17) |
| placebo (95% CI) | | | | | | | | |

| | | |
|---|---|---|
| P-value vs. placebo | 0.6244 | 0.1584 |

>2

| Number | 6 | | 8 | | 0 | | 3 | |
|---|---|---|---|---|---|---|---|---|
| Mean (SD) | 0.17 | (0.61) | −0.68 | (0.78) | NC | (NC) | −0.33 | (0.31) |
| LS Mean (SE) | 0.18 | (0.30) | −0.53 | (0.25) | NC | (NC) | NC | (NC) |
| LS Mean Diff vs. | | | −0.70 | (−1.38, −0.03) | | | NC | (NC, NC) |
| placebo (95% CI) | | | | | | | | |

| P-value vs. placebo | 0.0417 | NC |
|---|---|---|
| Overall p-value for interaction | 0.8549 | 0.4193 |

Atopic medical conditions
Yes

| Number | 16 | | 19 | | 19 | | 16 | |
|---|---|---|---|---|---|---|---|---|
| Mean (SD) | −0.75 | (1.04) | −0.89 | (0.92) | −0.38 | (0.97) | −0.80 | (0.83) |
| LS Mean (SE) | −0.41 | (0.29) | −0.71 | (0.28) | −0.23 | (0.21) | −0.79 | (0.24) |
| LS Mean Diff vs. | | | −0.30 | (−0.92, 0.32) | | | −0.56 | (−1.15, 0.03) |
| placebo (95% CI) | | | | | | | | |

| | | |
|---|---|---|
| P-value vs. placebo | 0.3393 | 0.0640 |

No

| Number | 12 | | 9 | | 41 | | 33 | |
|---|---|---|---|---|---|---|---|---|
| Mean (SD) | 0.22 | (0.71) | −0.87 | (1.00) | −0.15 | (1.06) | −0.82 | (0.67) |
| LS Mean (SE) | 0.27 | (0.28) | −0.69 | (0.33) | −0.36 | (0.17) | −0.87 | (0.17) |
| LS Mean Diff vs. | | | −0.96 | (−1.71, −0.21) | | | −0.51 | (−0.92, −0.10) |
| placebo (95% CI) | | | | | | | | |

| | | |
|---|---|---|
| P-value vs. placebo | 0.0118 | 0.0149 |
| Overall p-value for interaction | 0.1288 | 0.8698 |

Background ICS dose level at randomization (400 mg TID only)
Medium

| Number | 32 | | 19 | |
|---|---|---|---|---|
| Mean (SD) | −0.11 | (0.96) | −0.72 | (0.72) |
| LS Mean (SE) | −0.29 | (0.19) | −0.76 | (0.20) |
| LS Mean Diff vs. | | | −0.46 | (−0.93, 0.00) |
| placebo (95% CI) | | | | |

| | |
|---|---|
| P-value vs. placebo | 0.0518 |

High

| Number | 28 | | 30 | |
|---|---|---|---|---|
| Mean (SD) | −0.36 | (1.11) | −0.88 | (0.73) |
| LS Mean (SE) | −0.32 | (0.18) | −0.92 | (0.18) |
| LS Mean Diff vs. | | | −0.60 | (−1.08, −0.11) |
| placebo (95% CI) | | | | |

| | |
|---|---|
| P-value vs. placebo | 0.0167 |
| Overall p-value for interaction | 0.6694 |

Smoking history
Former

| Number | 7 | | 4 | | 3 | | 3 | |
|---|---|---|---|---|---|---|---|---|
| Mean (SD) | −0.03 | (0.88) | −1.20 | (1.07) | 0.33 | (0.42) | 0.07 | (0.42) |
| LS Mean (SE) | 0.75 | (0.48) | −0.50 | (0.62) | 0.27 | (0.44) | −0.56 | (0.47) |
| LS Mean Diff vs. | | | −1.25 | (−2.44, −0.06) | | | −0.83 | (−1.97, 0.31) |
| placebo (95% CI) | | | | | | | | |

| | | |
|---|---|---|
| P-value vs. placebo | 0.0396 | 0.1522 |

Never

| Number | 21 | | 24 | | 57 | | 46 | |
|---|---|---|---|---|---|---|---|---|
| Mean (SD) | −0.44 | (1.07) | −0.83 | (0.92) | −0.25 | (1.05) | −0.87 | (0.70) |
| LS Mean (SE) | −0.25 | (0.23) | −0.69 | (0.23) | −0.40 | (0.13) | −0.92 | (0.13) |
| LS Mean Diff vs. | | | −0.44 | (−0.98, 0.10) | | | −0.52 | (−0.86, −0.18) |
| placebo (95% CI) | | | | | | | | |

| | | |
|---|---|---|
| P-value vs. placebo | 0.1102 | 0.0025 |
| Overall p-value for interaction | 0.2052 | 0.7507 |

Baseline pre-bronchodilator FEV1 (L) (400 mg BID only)
<Median (2.022)

| Number | 13 | | 15 | |
|---|---|---|---|---|
| Mean (SD) | −0.31 | (1.08) | −0.88 | (0.81) |
| LS Mean (SE) | 0.04 | (0.28) | −0.42 | (0.30) |
| LS Mean Diff vs. | | | −0.45 | (−1.09, 0.19) |
| placebo (95% CI) | | | | |

| | |
|---|---|
| P-value vs. placebo | 0.1647 |

≥Median (2.022)

| Number | 15 | | 12 | |
|---|---|---|---|---|
| Mean (SD) | −0.36 | (1.01) | −0.83 | (1.11) |
| LS Mean (SE) | −0.24 | (0.29) | −0.89 | (0.34) |
| LS Mean Diff vs. | | | −0.66 | (−1.46, 0.14) |
| placebo (95% CI) | | | | |

| | |
|---|---|
| P-value vs. placebo | 0.1073 |
| Overall p-value for interaction | 0.7510 |

Baseline pre-bronchodilator percent predicted FEV1 (%) (400 mg BID only)
<Median (68)

| Number | 12 | | 16 | |
|---|---|---|---|---|
| Mean (SD) | −0.32 | (1.13) | −0.98 | (0.85) |
| LS Mean (SE) | 0.27 | (0.32) | −0.23 | (0.32) |
| LS Mean Diff vs. | | | −0.50 | (−1.15, 0.16) |
| placebo (95% CI) | | | | |

| | |
|---|---|
| P-value vs. placebo | 0.1359 |

≥Median (68)

| Number | 16 | | 11 | |
|---|---|---|---|---|
| Mean (SD) | −0.35 | (0.97) | −0.69 | (1.07) |
| LS Mean (SE) | −0.26 | (0.25) | −0.79 | (0.33) |
| LS Mean Diff vs. | | | −0.53 | (−1.28, 0.22) |
| placebo (95% CI) | | | | |

| | |
|---|---|
| P-value vs. placebo | 0.1642 |
| Overall p-value for interaction | 0.9232 |

Baseline pre-bronchodilator FEV1 (L) (400 mg TID only)
<Median (2.1955)

| Number | 28 | | 19 | |
|---|---|---|---|---|
| Mean (SD) | −0.24 | (1.11) | −0.91 | (0.74) |
| LS Mean (SE) | −0.23 | (0.18) | −0.89 | (0.21) |
| LS Mean Diff vs. | | | −0.66 | (−1.18, −0.15) |
| placebo (95% CI) | | | | |

| | |
|---|---|
| P-value vs. placebo | 0.0120 |

≥Median (2.1955)

| Number | 31 | | 29 | |
|---|---|---|---|---|
| Mean (SD) | −0.21 | (0.99) | −0.76 | (0.72) |
| LS Mean (SE) | −0.39 | (0.17) | −0.83 | (0.16) |
| LS Mean Diff vs. | | | −0.44 | (−0.88, −0.01) |
| placebo (95% CI) | | | | |

| | |
|---|---|
| P-value vs. placebo | 0.0467 |
| Overall p-value for interaction | 0.4527 |

Baseline pre-bronchodilator percent predicted FEV1 group 1 (%) (400 mg TID only)
<Median (77)

| Number | 31 | | 21 | |
|---|---|---|---|---|
| Mean (SD) | −0.14 | (1.04) | −0.72 | (0.70) |
| LS Mean (SE) | −0.23 | (0.18) | −0.74 | (0.22) |
| LS Mean Diff vs. | | | −0.52 | (−1.01, −0.03) |
| placebo (95% CI) | | | | |

| | |
|---|---|
| P-value vs. placebo | 0.0393 |

≥Median (77)

| Number | 29 | | 27 | |
|---|---|---|---|---|
| Mean (SD) | −0.32 | (1.03) | −0.89 | (0.75) |
| LS Mean (SE) | −0.38 | (0.17) | −0.90 | (0.16) |
| LS Mean Diff vs. | | | −0.52 | (−0.98, −0.05) |
| placebo (95% CI) | | | | |

| | |
|---|---|
| P-value vs. placebo | 0.0286 |
| Overall p-value for interaction | 0.9797 |

Baseline pre-bronchodilator percent predicted FEV1 group 2 (%) (400 mg TID only)
<80

| Number | 36 | | 26 | |
|---|---|---|---|---|
| Mean (SD) | −0.13 | (1.03) | −0.75 | (0.73) |
| LS Mean (SE) | −0.21 | (0.17) | −0.75 | (0.19) |
| LS Mean Diff vs. | | | −0.54 | (−0.98, −0.10) |
| placebo (95% CI) | | | | |

| | |
|---|---|
| P-value vs. placebo | 0.0165 |

≥80

| Number | 24 | | 22 | |
|---|---|---|---|---|
| Mean (SD) | −0.36 | (1.04) | −0.89 | (0.73) |
| LS Mean (SE) | −0.38 | (0.18) | −0.95 | (0.19) |
| LS Mean Diff vs. | | | −0.57 | (−1.08, −0.06) |
| placebo (95% CI) | | | | |

| | |
|---|---|
| P-value vs. placebo | 0.0295 |
| Overall p-value for interaction | 0.9237 |

Baseline FEV1 reversibility (%)
<12

| Number | 17 | | 12 | | 43 | | 33 | |
|---|---|---|---|---|---|---|---|---|
| Mean (SD) | −0.14 | (0.82) | −0.65 | (0.88) | −0.33 | (1.11) | −0.79 | (0.75) |
| LS Mean (SE) | −0.15 | (0.23) | −0.61 | (0.28) | −0.41 | (0.15) | −0.85 | (0.17) |
| LS Mean Diff vs. | | | −0.46 | (−1.10, 0.18) | | | −0.45 | (−0.87, −0.03) |
| placebo (95% CI) | | | | | | | | |

| | | |
|---|---|---|
| P-value vs. placebo | 0.1609 | 0.0375 |

≥12

| Number | 9 | | 14 | | 10 | | 10 | |
|---|---|---|---|---|---|---|---|---|
| Mean (SD) | −0.31 | (1.06) | −1.03 | (1.01) | −0.20 | (0.72) | −0.94 | (0.68) |
| LS Mean (SE) | 0.12 | (0.33) | −0.29 | (0.35) | −0.22 | (0.30) | −0.85 | (0.32) |
| LS Mean Diff vs. | | | −0.41 | (−1.10, 0.28) | | | −0.63 | (−1.36, 0.10) |
| placebo (95% CI) | | | | | | | | |

| | | |
|---|---|---|
| P-value vs. placebo | 0.2413 | 0.0907 |
| Overall p-value for interaction | 0.9399 | 0.6991 |

Baseline ACQ-5 score
≤2

| Number | 13 | | 16 | | 25 | | 14 | |
|---|---|---|---|---|---|---|---|---|
| Mean (SD) | −0.15 | (0.83) | −0.76 | (0.96) | −0.29 | (0.80) | −0.91 | (0.89) |
| LS Mean (SE) | 0.12 | (0.28) | −0.60 | (0.27) | −0.27 | (0.18) | −0.81 | (0.21) |
| LS Mean Diff vs. | | | −0.72 | (−1.32, −0.12) | | | −0.54 | (−1.06, −0.02) |
| placebo (95% CI) | | | | | | | | |

| | | |
|---|---|---|
| P-value vs. placebo | 0.0197 | 0.0426 |

>2

| Number | 15 | | 12 | | 35 | | 35 | |
|---|---|---|---|---|---|---|---|---|
| Mean (SD) | −0.49 | (1.17) | −1.05 | (0.89) | −0.18 | (1.18) | −0.78 | (0.65) |
| LS Mean (SE) | −0.20 | (0.34) | −0.78 | (0.39) | −0.34 | (0.17) | −0.89 | (0.17) |
| LS Mean Diff vs. | | | −0.58 | (−1.39, 0.23) | | | −0.55 | (−0.98, −0.12) |
| placebo (95% CI) | | | | | | | | |

| | | |
|---|---|---|
| P-value vs. placebo | 0.1633 | 0.0118 |
| Overall p-value for interaction | 0.8615 | 0.9369 |

LATAM: Latin America, ROW: Rest of the world

TABLE 48
Subgroup analysis: change from baseline in ACQ-5 score at EOT
(Week 12) by baseline ICS/LABA dose level (mITT population).

| | | |
|---|---|---|
| | | Rilzabrutinib |
| | Placebo BID | 400 mg BID |
| ACQ-5 | (N = 32) | (N = 32) |

Background ICS/LABA dose level at randomization
Medium

| Number | 3 | | 3 | |
|---|---|---|---|---|
| Mean (SD) | 0.00 | (1.00) | −1.07 | (0.70) |
| LS Mean (SE) | 0.02 | (0.94) | −0.79 | (0.75) |
| LS Mean Diff vs. | | | −0.80 | (−2.49, 0.88) |
| placebo (95% CI) | | | | |

| | |
|---|---|
| P-value vs. placebo | 0.3496 |

High

| Number | 25 | | 25 | |
|---|---|---|---|---|
| Mean (SD) | −0.38 | (1.04) | −0.86 | (0.96) |
| LS Mean (SE) | −0.12 | (0.22) | −0.70 | (0.23) |
| LS Mean Diff vs. | | | −0.57 | (−1.12, −0.03) |
| placebo (95% CI) | | | | |

| | |
|---|---|
| P-value vs. placebo | 0.0372 |
| Overall p-value | 0.9013 |
| for interaction | |

LATAM: Latin America, ROW: Rest of the world

TABLE 49
Subgroup analysis: change from baseline in ACQ-5 score at EOT
(Week 12) by baseline biomarker subgroups (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo BID | 400 mg TID |
| ACQ-5 | (N = 32) | (N = 32) | (N = 68) | (N = 64) |

Baseline IgE level (IU/mL)
<100

| Number | 8 | | 7 | | 24 | | 18 | |
|---|---|---|---|---|---|---|---|---|
| Mean (SD) | −0.50 | (1.22) | −0.83 | (0.76) | −0.20 | (0.93) | −0.93 | (0.66) |
| LS Mean (SE) | −0.35 | (0.50) | −0.63 | (0.50) | −0.40 | (0.37) | −1.08 | (0.40) |

| | | | | |
|---|---|---|---|---|
| LS Mean Diff vs. | −0.28 | (−1.44, 0.88) | −0.67 | (−1.19, −0.16) |
| placebo (95% CI) | | | | |

| | | |
|---|---|---|
| P-value vs. placebo | 0.6362 | 0.0107 |

≥100

| Number | 20 | | 21 | | 36 | | 31 | |
|---|---|---|---|---|---|---|---|---|
| Mean (SD) | −0.27 | (0.96) | −0.90 | (1.00) | −0.24 | (1.11) | −0.75 | (0.76) |
| LS Mean (SE) | −0.24 | (0.51) | −0.83 | (0.48) | −0.18 | (0.22) | −0.64 | (0.24) |

| | | | | |
|---|---|---|---|---|
| LS Mean Diff vs. | −0.59 | (−1.14, −0.04) | −0.46 | (−0.89, −0.03) |
| placebo (95% CI) | | | | |

| | | |
|---|---|---|
| P-value vs. placebo | 0.0358 | 0.0366 |
| Overall p-value for interaction | 0.8951 | 0.5332 |

Baseline median IgE level (IU/mL) (400 mg BID only)
<Median (210.8)

| Number | 10 | | 18 | |
|---|---|---|---|---|
| Mean (SD) | −0.44 | (1.09) | −0.76 | (0.94) |
| LS Mean (SE) | −0.14 | (0.33) | −0.60 | (0.26) |

| | | |
|---|---|---|
| LS Mean Diff vs. | −0.46 | (−1.24, 0.32) |
| placebo (95% CI) | | |

| | |
|---|---|
| P-value vs. placebo | 0.2488 |

≥Median (210.8)

| Number | 18 | | 10 | |
|---|---|---|---|---|
| Mean (SD) | −0.28 | (1.01) | −1.12 | (0.90) |
| LS Mean (SE) | −0.08 | (0.24) | −0.87 | (0.36) |

| | | |
|---|---|---|
| LS Mean Diff vs. | −0.79 | (−1.49, −0.08) |
| placebo (95% CI) | | |

| | |
|---|---|
| P-value vs. placebo | 0.0282 |
| Overall p-value for interaction | 0.5416 |

Baseline median IgE level (IU/mL) (400 mg TID only)
<Median (121.85)

| Number | 30 | | 23 | |
|---|---|---|---|---|
| Mean (SD) | 0.01 | (1.11) | −0.95 | (0.63) |
| LS Mean (SE) | 0.02 | (0.23) | −0.79 | (0.23) |

| | | |
|---|---|---|
| LS Mean Diff vs. | −0.81 | (−1.30, −0.32) |
| placebo (95% CI) | | |

| | |
|---|---|
| P-value vs. placebo | 0.0011 |

≥Median (121.85)

| Number | 30 | | 26 | |
|---|---|---|---|---|
| Mean (SD) | −0.46 | (0.91) | −0.70 | (0.79) |
| LS Mean (SE) | −0.71 | (0.43) | −0.99 | (0.46) |

| | | |
|---|---|---|
| LS Mean Diff vs. | −0.27 | (−0.71, 0.16) |
| placebo (95% CI) | | |

| | |
|---|---|
| P-value vs. placebo | 0.2121 |
| Overall p-value for interaction | 0.0955 |

Baseline median IgA level (mg/L) (400 mg TID only)
<Median (2190)

| Number | 32 | | 23 | |
|---|---|---|---|---|
| Mean (SD) | −0.06 | (1.02) | −0.86 | (0.68) |
| LS Mean (SE) | −0.23 | (0.17) | −0.90 | (0.19) |

| | | |
|---|---|---|
| LS Mean Diff vs. | −0.67 | (−1.14, −0.19) |
| placebo (95% CI) | | |

| | |
|---|---|
| P-value vs. placebo | 0.0060 |

≥Median (2190)

| Number | 28 | | 26 | |
|---|---|---|---|---|
| Mean (SD) | −0.41 | (1.03) | −0.78 | (0.77) |
| LS Mean (SE) | −0.38 | (0.18) | −0.80 | (0.18) |

| | | |
|---|---|---|
| LS Mean Diff vs. | −0.41 | (−0.89, 0.07) |
| placebo (95% CI) | | |

| | |
|---|---|
| P-value vs. placebo | 0.0925 |
| Overall p-value for interaction | 0.4742 |

Baseline blood eosinophil level 1 (10\hat{ }9/L) (400 mg BID only)
<0.15

| Number | 6 | | 8 | |
|---|---|---|---|---|
| Mean (SD) | −0.80 | (1.40) | −0.50 | (0.99) |
| LS Mean (SE) | −0.34 | (0.65) | −0.57 | (0.45) |

| | | |
|---|---|---|
| LS Mean Diff vs. | −0.23 | (−2.01, 1.56) |
| placebo (95% CI) | | |

| | |
|---|---|
| P-value vs. placebo | 0.7785 |

≥0.15

| Number | 22 | | 20 | |
|---|---|---|---|---|
| Mean (SD) | −0.21 | (0.90) | −1.04 | (0.88) |
| LS Mean (SE) | −0.07 | (0.21) | −0.85 | (0.26) |

| | | |
|---|---|---|
| LS Mean Diff vs. | −0.78 | (−1.31, −0.24) |
| placebo (95% CI) | | |

| | |
|---|---|
| P-value vs. placebo | 0.0044 |
| Overall p-value for interaction | 0.1355 |

Baseline blood eosinophil level 2 (10\hat{ }9/L) (400 mg BID only)
<0.3

| Number | 17 | | 16 | |
|---|---|---|---|---|
| Mean (SD) | −0.52 | (1.21) | −1.04 | (0.92) |
| LS Mean (SE) | −0.13 | (0.24) | −0.81 | (0.27) |

| | | |
|---|---|---|
| LS Mean Diff vs. | −0.68 | (−1.33, −0.03) |
| placebo (95% CI) | | |

| | |
|---|---|
| P-value vs. placebo | 0.0399 |

≥0.3

| Number | 11 | | 12 | |
|---|---|---|---|---|
| Mean (SD) | −0.05 | (0.57) | −0.68 | (0.94) |
| LS Mean (SE) | −0.16 | (0.35) | −0.72 | (0.35) |

| | | |
|---|---|---|
| LS Mean Diff vs. | −0.55 | (−1.22, 0.12) |
| placebo (95% CI) | | |

| | |
|---|---|
| P-value vs. placebo | 0.1049 |
| Overall p-value for interaction | 0.8601 |

Baseline blood eosinophil level 1 (10\hat{ }9/L) (400 mg TID only)
<0.15

| Number | 27 | | 21 | |
|---|---|---|---|---|
| Mean (SD) | −0.38 | (0.84) | −0.85 | (0.80) |
| LS Mean (SE) | −0.49 | (0.16) | −0.84 | (0.17) |

| | | |
|---|---|---|
| LS Mean Diff vs. | −0.35 | (−0.79, 0.09) |
| placebo (95% CI) | | |

| | |
|---|---|
| P-value vs. placebo | 0.1217 |

≥0.15-<0.3

| Number | 18 | | 13 | |
|---|---|---|---|---|
| Mean (SD) | −0.02 | (1.32) | −0.78 | (0.66) |
| LS Mean (SE) | 0.03 | (0.27) | −0.81 | (0.29) |

| | | |
|---|---|---|
| LS Mean Diff vs. | −0.83 | (−1.55, −0.12) |
| placebo (95% CI) | | |

| | |
|---|---|
| P-value vs. placebo | 0.0229 |

≥0.3

| Number | 15 | | 15 | |
|---|---|---|---|---|
| Mean (SD) | −0.19 | (0.98) | −0.80 | (0.70) |
| LS Mean (SE) | −0.26 | (0.30) | −0.91 | (0.27) |

| | | |
|---|---|---|
| LS Mean Diff vs. | −0.65 | (−1.29, −0.01) |
| placebo (95% CI) | | |

| | |
|---|---|
| P-value vs. placebo | 0.0451 |
| Overall p-value for interaction | 0.5667 |

Baseline blood eosinophil level 2 (10\hat{ }9/L) (400 mg TID only)
<0.3

| Number | 45 | | 34 | |
|---|---|---|---|---|
| Mean (SD) | −0.24 | (1.06) | −0.82 | (0.74) |
| LS Mean (SE) | −0.34 | (0.15) | −0.86 | (0.15) |

| | | |
|---|---|---|
| LS Mean Diff vs. | −0.52 | (−0.92, −0.12) |
| placebo (95% CI) | | |

| | |
|---|---|
| P-value vs. placebo | 0.0104 |

≥0.3

| Number | 15 | | 15 | |
|---|---|---|---|---|
| Mean (SD) | −0.19 | (0.98) | −0.80 | (0.70) |
| LS Mean (SE) | −0.26 | (0.30) | −0.91 | (0.27) |

| | | |
|---|---|---|
| LS Mean Diff vs. | −0.65 | (−1.29, −0.01) |
| placebo (95% CI) | | |

| | |
|---|---|
| P-value vs. placebo | 0.0451 |
| Overall p-value for interaction | 0.7504 |

Baseline FeNO level 1 (ppb)
<25

| Number | 14 | | 11 | | 37 | | 30 | |
|---|---|---|---|---|---|---|---|---|
| Mean (SD) | −0.39 | (1.12) | −1.02 | (0.80) | −0.26 | (0.97) | −0.84 | (0.71) |
| LS Mean (SE) | −0.01 | (0.32) | −0.81 | (0.32) | −0.43 | (0.15) | −0.93 | (0.15) |

| | | | | |
|---|---|---|---|---|
| LS Mean Diff vs. | −0.80 | (−1.53, −0.06) | −0.51 | (−0.89, −0.12) |
| placebo (95% CI) | | | | |

| | | |
|---|---|---|
| P-value vs. placebo | 0.0344 | 0.0101 |

≥25

| Number | 14 | | 17 | | 19 | | 17 | |
|---|---|---|---|---|---|---|---|---|
| Mean (SD) | −0.29 | (0.95) | −0.80 | (1.02) | −0.31 | (1.18) | −0.84 | (0.78) |
| LS Mean (SE) | −0.15 | (0.26) | −0.62 | (0.30) | −0.29 | (0.27) | −0.82 | (0.26) |

| | | | | |
|---|---|---|---|---|
| LS Mean Diff vs. | −0.47 | (−1.15, 0.22) | −0.53 | (−1.19, 0.13) |
| placebo (95% CI) | | | | |

| | | |
|---|---|---|
| P-value vs. placebo | 0.1826 | 0.1127 |
| Overall p-value for interaction | 0.5887 | 0.9211 |

Baseline FeNO level 2 (ppb)
<35

| Number | 17 | | 20 | | 44 | | 35 | |
|---|---|---|---|---|---|---|---|---|
| Mean (SD) | −0.36 | (1.05) | −0.91 | (0.86) | −0.25 | (1.08) | −0.81 | (0.72) |
| LS Mean (SE) | 0.11 | (0.27) | −0.64 | (0.24) | −0.36 | (0.15) | −0.85 | (0.15) |

| | | | | |
|---|---|---|---|---|
| LS Mean Diff vs. | −0.75 | (−1.32, −0.19) | −0.49 | (−0.89, −0.10) |
| placebo (95% CI) | | | | |

| | | |
|---|---|---|
| P-value vs. placebo | 0.0089 | 0.0141 |

≥35

| Number | 11 | | 8 | | 12 | | 12 | |
|---|---|---|---|---|---|---|---|---|
| Mean (SD) | −0.29 | (1.02) | −0.83 | (1.15) | −0.37 | (0.90) | −0.92 | (0.77) |
| LS Mean (SE) | −0.26 | (0.32) | −0.67 | (0.46) | −0.38 | (0.36) | −0.89 | (0.32) |

| | | | | |
|---|---|---|---|---|
| LS Mean Diff vs. | −0.41 | (−1.47, 0.66) | −0.51 | (−1.24, 0.23) |
| placebo (95% CI) | | | | |

| | | |
|---|---|---|
| P-value vs. placebo | 0.4551 | 0.1744 |
| Overall p-value for interaction | 0.4819 | 0.8970 |

LATAM: Latin America, ROW: Rest of the world

TABLE 50
Subgroup analysis: change from baseline in ACQ-5 score at EOT (Week
12) in the baseline low EOS and low FeNO subgroup (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo BID | 400 mg BID |
| ACQ-5 | (N = 32) | (N = 32) | (N = 68) | (N = 68) |

Baseline blood eosinophil (10\hat{ }9/L) and FeNO (ppb)
Low EOS (<0.15) and low FeNO
(<25)

| Number | 4 | | 3 | | 21 | 14 | |
|---|---|---|---|---|---|---|---|
| Mean (SD) | −0.50 | (1.70) | −0.60 | (1.00) | −0.30 (0.86) | −1.04 | (0.80) |
| LS Mean (SE) | 0.83 | (1.42) | −1.61 | (1.12) | −0.41 (0.18) | −1.02 | (0.21) |

| | | | | |
|---|---|---|---|---|
| LS Mean Diff vs. | −2.44 | (−11.51, 6.64) | −0.61 | (−1.13, −0.09) |

| placebo (95% CI) | | |
|---|---|---|
| P-value vs. placebo | 0.3675 | 0.0214 |

All others

| Number | 24 | | 25 | | 35 | 33 | |
|---|---|---|---|---|---|---|---|
| Mean (SD) | −0.31 | (0.92) | −0.92 | (0.94) | −0.27 (1.14) | −0.75 | (0.68) |
| LS Mean (SE) | −0.09 | (0.20) | −0.68 | (0.23) | −0.26 (0.17) | −0.76 | (0.17) |

| | | | | |
|---|---|---|---|---|
| LS Mean Diff vs. | −0.59 | (−1.08, −0.11) | −0.50 | (−0.95, −0.06) |

| placebo (95% CI) | | |
|---|---|---|
| P-value vs. placebo | 0.0172 | 0.0267 |
| Overall p-value | 0.9709 | 0.8721 |
| for interaction | | |

LATAM: Latin America, ROW: Rest of the world

### AQLQ and AQLQ(S)

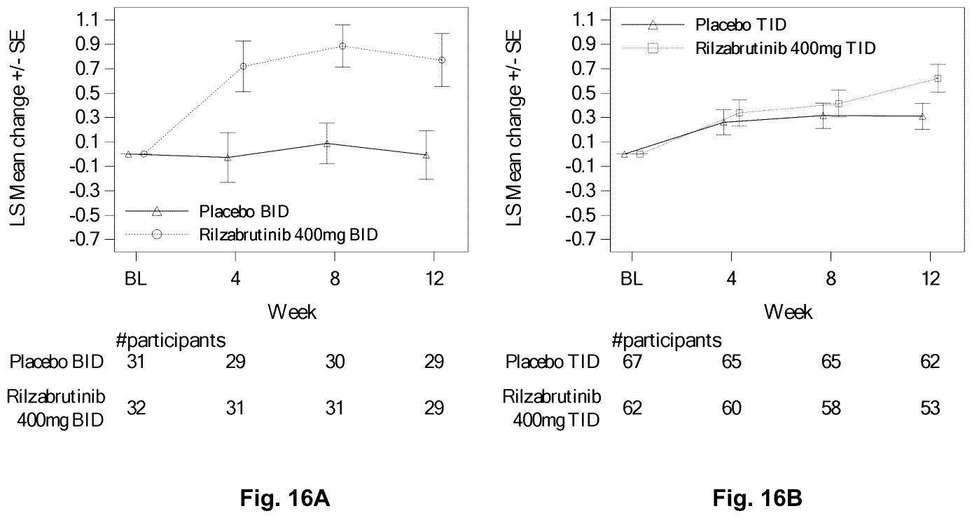

Rilzabrutinib treatment led to significant improvements in AQLQ and AQLQ(S) (FIG. 16 and Tables 51-57). Notably, 51.7% of participants in the 400 mg rilzabrutinib BID cohort and 54.7% of participants in the 400 mg rilzabrutinib TID cohort—but only 34.5% of participants in the placebo BID cohort and 40.3% of participants in the placebo TID cohort—achieved a response, defined as an improvement of at least 0.5 from baseline at Week 12 (p=0.0281 and p=0.0456, respectively). Clinically important results were observed as early as Week 4 and sustained through Week 12 (Tables 51 and 52).

TABLE 51
Responder analysis for change from baseline in
AQLQ global score at Week 12 (mITT population).

| 400 mg BID cohort | | 400 mg TID cohort | |
|---|---|---|---|
| | Rilzabrutinib | | Rilzabrutinib |
| Placebo BID | 400 mg BID | Placebo TID | 400 mg TID |
| (N = 32) | (N = 32) | (N = 68) | (N = 64) |

Week 12

| | | | | |
|---|---|---|---|---|
| Number (observed/LOCF | 29 | 29 | 62 | 53 |
| imputed) | (20/9) | (24/5) | (52/10) | (50/3) |
| Responder | 10 | 15 | 25 | 29 |
| (observed/LOCF imputed) | (34.5) (7/3) | (51.7) (14/1) | (40.3) (24/1) | (54.7) (28/1) |
| Non-responder | 19 | 14 | 37 | 24 |
| (observed/LOCF imputed) | (65.5) (13/6) | (48.3) (10/4) | (59.7) (28/9) | (45.3) (22/2) |
| Responder (including | 10.6 | 16.6 | 26.9 | 32.9 |
| multiply imputed) | (33.0) | (51.7) | (39.6) | (51.4) |
| OR vs. placebo (95% CI) | | 4.89 (1.19, | | 2.23 (1.02, |
| | | 20.17) | | 4.89) |
| P-value vs. placebo | | 0.0281 | | 0.0456 |

LATAM: Latin America, ROW: Rest of the world

TABLE 52
Change from baseline in AQLQ(S) over time (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| AQLQ(S) | (N = 32) | (N = 32) | (N = 68) | (N = 64) |

Global score
Baseline

| Number | 31 | | 32 | | 67 | | 62 | |
|---|---|---|---|---|---|---|---|---|
| Mean (SD) | 4.67 | (1.07) | 4.91 | (1.04) | 4.68 | (0.83) | 4.96 | (0.71) |

| | | | | |
|---|---|---|---|---|
| Median | 4.69 | 5.19 | 4.56 | 4.94 |
| Q1; Q3 | 4.13; 5.59 | 4.31; 5.64 | 4.00; 5.41 | 4.38; 5.44 |
| Min; Max | 2.1; 6.2 | 2.4; 6.4 | 2.8; 6.3 | 3.7; 6.6 |

Week 4

| Number (observed/LOCF | 30 | (28/2) | 31 | (30/1) | 65 | (64/1) | 61 | (61/0) |
|---|---|---|---|---|---|---|---|---|
| imputed) | | | | | | | | |
| Mean (SD) | 4.67 | (1.17) | 5.63 | (0.82) | 4.90 | (0.91) | 5.16 | (0.97) |

| | | | | |
|---|---|---|---|---|
| Median | 4.69 | 5.69 | 4.84 | 5.03 |
| Q1; Q3 | 4.03; 5.50 | 5.34; 6.28 | 4.22; 5.59 | 4.44; 6.03 |
| Min; Max | 1.9; 6.7 | 3.1; 6.7 | 2.2; 6.7 | 2.5; 6.9 |

Change from baseline

| Number (observed/LOCF | 29 | (27/2) | 31 | (30/1) | 65 | (64/1) | 60 | (60/0) |
|---|---|---|---|---|---|---|---|---|
| imputed) | | | | | | | | |
| Mean (SD) | −0.03 | (1.35) | 0.73 | (0.93) | 0.19 | (0.72) | 0.19 | (0.89) |
| Median | 0.00 | | 0.69 | | 0.28 | | 0.13 | |
| Q1; Q3 | −0.50; 0.63 | | 0.09; 1.09 | | −0.19; 0.59 | | −0.25; 0.67 | |
| Min; Max | −3.7; 2.2 | | −0.8; 3.2 | | −1.9; 1.7 | | −2.2; 2.7 | |
| LS Mean (SE) | −0.04 | (0.21) | 0.80 | (0.21) | 0.24 | (0.10) | 0.33 | (0.11) |

| LS Mean Diff vs. placebo | 0.84 (0.34, | 0.09 (−0.18, |
|---|---|---|
| (95% CI) | 1.34) | 0.36) |
| P-value vs. placebo | 0.0010 | 0.4929 |

Week 8

| Number (observed/LOCF | 31 | (28/3) | 31 | (27/4) | 65 | (62/3) | 59 | (56/3) |
|---|---|---|---|---|---|---|---|---|
| imputed) | | | | | | | | |
| Mean (SD) | 4.94 | (1.14) | 5.79 | (0.75) | 5.02 | (0.98) | 5.27 | (0.88) |

| | | | | |
|---|---|---|---|---|
| Median | 5.06 | 5.88 | 5.13 | 5.06 |
| Q1; Q3 | 3.88; 6.00 | 5.53; 6.28 | 4.06; 5.81 | 4.59; 6.16 |
| Min; Max | 2.8; 6.8 | 4.0; 7.0 | 2.6; 7.0 | 3.5; 6.9 |

Change from baseline

| Number (observed/LOCF | 30 | (27/3) | 31 | (27/4) | 65 | (62/3) | 58 | (55/3) |
|---|---|---|---|---|---|---|---|---|
| imputed) | | | | | | | | |
| Mean (SD) | 0.22 | (1.06) | 0.90 | (0.85) | 0.30 | (0.86) | 0.29 | (0.74) |
| Median | 0.33 | | 0.63 | | 0.34 | | 0.34 | |
| Q1; Q3 | −0.19; 0.88 | | 0.22; 1.31 | | −0.25; 1.00 | | −0.09; 0.75 | |
| Min; Max | −3.0; 1.9 | | −0.4; 2.8 | | −1.6; 2.6 | | −1.3; 2.0 | |
| LS Mean (SE) | 0.13 | (0.16) | 0.92 | (0.17) | 0.32 | (0.10) | 0.42 | (0.11) |

| LS Mean Diff vs. placebo | 0.79 (0.39, | 0.10 (−0.18, |
|---|---|---|
| (95% CI) | 1.20) | 0.37) |
| P-value vs. placebo | 0.0001 | 0.4993 |

Week 12

| Number (observed/LOCF | 30 | (21/9) | 29 | (24/5) | 62 | (52/10) | 54 | (51/3) |
|---|---|---|---|---|---|---|---|---|
| imputed) | | | | | | | | |
| Mean (SD) | 4.99 | (1.17) | 5.75 | (0.84) | 4.99 | (1.06) | 5.41 | (0.88) |

| | | | | |
|---|---|---|---|---|
| Median | 5.05 | 5.72 | 5.06 | 5.23 |
| Q1; Q3 | 3.88; 5.97 | 5.41; 6.34 | 4.13; 5.88 | 4.91; 6.13 |
| Min; Max | 2.9; 7.0 | 4.0; 7.0 | 1.4; 6.7 | 3.1; 7.0 |

Change from baseline

| Number (observed/LOCF | 29 | (20/9) | 29 | (24/5) | 62 | (52/10) | 53 | (50/3) |
|---|---|---|---|---|---|---|---|---|
| imputed) | | | | | | | | |
| Mean (SD) | 0.29 | (1.15) | 0.84 | (0.99) | 0.26 | (0.90) | 0.46 | (0.86) |
| Median | 0.22 | | 0.69 | | 0.30 | | 0.53 | |
| Q1; Q3 | −0.13; 1.25 | | 0.16; 1.38 | | −0.41; 0.78 | | 0.00; 1.00 | |
| Min; Max | −2.5; 2.3 | | −0.7; 3.1 | | −1.7; 3.1 | | −1.9; 2.6 | |
| LS Mean (SE) | 0.10 | (0.18) | 0.82 | (0.20) | 0.28 | (0.12) | 0.57 | (0.12) |

| LS Mean Diff vs. placebo | 0.72 (0.25, | 0.29 (−0.01, |
|---|---|---|
| (95% CI) | 1.18) | 0.60) |
| P-value vs. placebo | 0.0025 | 0.0620 |

Symptoms score
Baseline

| Number | 31 | | 32 | | 67 | | 62 | |
|---|---|---|---|---|---|---|---|---|
| Mean (SD) | 4.59 | (1.05) | 5.05 | (1.05) | 4.70 | (0.80) | 4.97 | (0.73) |

| | | | | |
|---|---|---|---|---|
| Median | 4.75 | 5.21 | 4.75 | 4.83 |
| Q1; Q3 | 4.25; 5.25 | 4.42; 5.83 | 4.08; 5.33 | 4.33; 5.42 |
| Min; Max | 1.7; 6.0 | 2.6; 6.6 | 3.0; 6.8 | 3.8; 6.6 |

Week 4

| Number (observed/LOCF | 30 | (28/2) | 31 | (30/1) | 65 | (64/1) | 61 | (61/0) |
|---|---|---|---|---|---|---|---|---|
| imputed) | | | | | | | | |
| Mean (SD) | 4.82 | (1.09) | 5.83 | (0.72) | 4.92 | (0.90) | 5.19 | (1.00) |

| | | | | |
|---|---|---|---|---|
| Median | 4.88 | 6.00 | 4.92 | 5.08 |
| Q1; Q3 | 4.00; 5.50 | 5.50; 6.33 | 4.42; 5.58 | 4.42; 6.00 |
| Min; Max | 1.8; 6.8 | 3.7; 6.7 | 2.2; 6.6 | 2.4; 7.0 |

Change from baseline

| Number (observed/LOCF | 29 | (27/2) | 31 | (30/1) | 65 | (64/1) | 60 | (60/0) |
|---|---|---|---|---|---|---|---|---|
| imputed) | | | | | | | | |
| Mean (SD) | 0.15 | (1.27) | 0.80 | (0.90) | 0.19 | (0.77) | 0.22 | (0.98) |
| Median | 0.08 | | 0.75 | | 0.25 | | 0.25 | |
| Q1; Q3 | −0.25; 0.75 | | 0.08; 1.25 | | −0.25; 0.67 | | −0.29; 0.67 | |
| Min; Max | −3.2; 2.4 | | −0.8; 3.3 | | −1.8; 1.7 | | −2.3; 2.8 | |
| LS Mean (SE) | 0.06 | (0.18) | 0.92 | (0.19) | 0.22 | (0.11) | 0.38 | (0.11) |

| LS Mean Diff vs. placebo | 0.86 (0.41, | 0.15 (−0.14, |
|---|---|---|
| (95% CI) | 1.32) | 0.44) |
| P-value vs. placebo | 0.0002 | 0.3018 |

Week 8

| Number (observed/LOCF | 31 | (28/3) | 31 | (27/4) | 65 | (62/3) | 59 | (56/3) |
|---|---|---|---|---|---|---|---|---|
| imputed) | | | | | | | | |
| Mean (SD) | 5.05 | (1.06) | 5.93 | (0.79) | 5.02 | (1.03) | 5.29 | (0.84) |

| | | | | |
|---|---|---|---|---|
| Median | 5.08 | 6.00 | 5.25 | 5.17 |
| Q1; Q3 | 4.25; 5.83 | 5.67; 6.50 | 4.17; 5.75 | 4.58; 6.08 |
| Min; Max | 2.3; 6.9 | 3.5; 6.9 | 2.6; 7.0 | 3.7; 7.0 |

Change from baseline

| Number (observed/LOCF | 30 | (27/3) | 31 | (27/4) | 65 | (62/3) | 58 | (55/3) |
|---|---|---|---|---|---|---|---|---|
| imputed) | | | | | | | | |
| Mean (SD) | 0.38 | (1.06) | 0.91 | (0.94) | 0.29 | (0.96) | 0.29 | (0.77) |
| Median | 0.13 | | 0.83 | | 0.17 | | 0.38 | |
| Q1; Q3 | −0.08; 0.83 | | 0.25; 1.25 | | −0.42; 1.08 | | −0.08; 0.75 | |
| Min; Max | −1.8; 2.6 | | −0.6; 3.0 | | −1.6; 2.3 | | −1.8; 2.0 | |
| LS Mean (SE) | 0.23 | (0.17) | 0.99 | (0.18) | 0.31 | (0.11) | 0.44 | (0.12) |

| LS Mean Diff vs. placebo | 0.76 (0.35, | 0.13 (−0.16, |
|---|---|---|
| (95% CI) | 1.18) | 0.43) |
| P-value vs. placebo | 0.0003 | 0.3792 |

Week 12

| Number (observed/LOCF | 30 | (21/9) | 29 | (24/5) | 62 | (52/10) | 54 | (51/3) |
|---|---|---|---|---|---|---|---|---|
| imputed) | | | | | | | | |
| Mean (SD) | 5.13 | (1.05) | 5.87 | (0.85) | 4.97 | (1.19) | 5.41 | (0.83) |

| | | | | |
|---|---|---|---|---|
| Median | 5.08 | 5.92 | 5.00 | 5.25 |
| Q1; Q3 | 4.67; 5.83 | 5.50; 6.50 | 4.42; 5.92 | 4.83; 6.08 |
| Min; Max | 3.0; 7.0 | 3.8; 7.0 | 1.0; 6.8 | 3.7; 7.0 |

Change from baseline

| Number (observed/LOCF | 29 | (20/9) | 29 | (24/5) | 62 | (52/10) | 53 | (50/3) |
|---|---|---|---|---|---|---|---|---|
| imputed) | | | | | | | | |
| Mean (SD) | 0.47 | (1.07) | 0.87 | (1.12) | 0.21 | (1.08) | 0.44 | (0.92) |
| Median | 0.33 | | 0.83 | | 0.08 | | 0.58 | |
| Q1; Q3 | −0.08; 1.17 | | 0.08; 1.58 | | −0.58; 0.92 | | 0.00; 0.92 | |
| Min; Max | −1.8; 2.6 | | −1.3; 3.3 | | −2.3; 3.2 | | −2.0; 2.8 | |
| LS Mean (SE) | 0.17 | (0.18) | 0.84 | (0.19) | 0.26 | (0.13) | 0.59 | (0.14) |

| LS Mean Diff vs. placebo | 0.67 (0.22, | 0.34 (−0.01, |
|---|---|---|
| (95% CI) | 1.11) | 0.68) |
| P-value vs. placebo | 0.0032 | 0.0542 |

Activity limitation score
Baseline

| Number | 31 | | 32 | | 67 | | 62 | |
|---|---|---|---|---|---|---|---|---|
| Mean (SD) | 4.74 | (1.13) | 4.78 | (1.12) | 4.70 | (0.83) | 4.99 | (0.78) |

| | | | | |
|---|---|---|---|---|
| Median | 4.73 | 5.00 | 4.64 | 4.91 |
| Q1; Q3 | 4.09; 5.73 | 4.09; 5.55 | 4.00; 5.36 | 4.45; 5.45 |
| Min; Max | 2.4; 6.6 | 1.9; 6.5 | 3.0; 6.6 | 3.4; 6.7 |

Week 4

| Number (observed/LOCF | 30 | (28/2) | 31 | (30/1) | 65 | (64/1) | 61 | (61/0) |
|---|---|---|---|---|---|---|---|---|
| imputed) | | | | | | | | |
| Mean (SD) | 4.70 | (1.24) | 5.48 | (0.88) | 4.93 | (0.97) | 5.15 | (0.95) |

| | | | | |
|---|---|---|---|---|
| Median | 4.68 | 5.55 | 5.00 | 5.18 |
| Q1; Q3 | 4.00; 5.45 | 5.09; 6.18 | 4.27; 5.64 | 4.45; 6.00 |
| Min; Max | 2.5; 7.0 | 3.2; 7.0 | 2.5; 6.7 | 2.6; 6.9 |

Change from baseline

| Number (observed/LOCF | 29 | (27/2) | 31 | (30/1) | 65 | (64/1) | 60 | (60/0) |
|---|---|---|---|---|---|---|---|---|
| imputed) | | | | | | | | |
| Mean (SD) | −0.07 | (1.21) | 0.73 | (0.98) | 0.20 | (0.80) | 0.16 | (0.89) |
| Median | 0.00 | | 0.55 | | 0.27 | | 0.09 | |
| Q1; Q3 | −0.64; 0.55 | | 0.00; 1.18 | | −0.18; 0.64 | | −0.41; 0.59 | |
| Min; Max | −3.2; 2.1 | | −1.1; 3.0 | | −2.2; 1.9 | | −2.0; 2.9 | |
| LS Mean (SE) | −0.03 | (0.20) | 0.72 | (0.21) | 0.26 | (0.10) | 0.34 | (0.11) |

| LS Mean Diff vs. placebo | 0.75 (0.26, | 0.08 (−0.20, |
|---|---|---|
| (95% CI) | 1.24) | 0.35) |
| P-value vs. placebo | 0.0029 | 0.5846 |

Week 8

| Number (observed/LOCF | 31 | (28/3) | 31 | (27/4) | 65 | (62/3) | 59 | (56/3) |
|---|---|---|---|---|---|---|---|---|
| imputed) | | | | | | | | |
| Mean (SD) | 4.96 | (1.22) | 5.72 | (0.81) | 5.05 | (0.94) | 5.28 | (0.90) |

| | | | | |
|---|---|---|---|---|
| Median | 5.09 | 5.91 | 5.09 | 5.27 |
| Q1; Q3 | 3.91; 6.27 | 5.09; 6.18 | 4.18; 5.82 | 4.64; 6.00 |
| Min; Max | 2.6; 6.9 | 3.9; 7.0 | 3.0; 7.0 | 3.5; 6.9 |

Change from baseline

| Number (observed/LOCF | 30 | (27/3) | 31 | (27/4) | 65 | (62/3) | 58 | (55/3) |
|---|---|---|---|---|---|---|---|---|
| imputed) | | | | | | | | |
| Mean (SD) | 0.17 | (0.97) | 0.96 | (0.92) | 0.30 | (0.86) | 0.29 | (0.83) |
| Median | 0.18 | | 0.82 | | 0.36 | | 0.36 | |
| Q1; Q3 | −0.18; 0.73 | | 0.18; 1.55 | | −0.18; 0.91 | | −0.27; 0.91 | |
| Min; Max | −3.1; 1.5 | | −0.4; 2.8 | | −1.8; 2.9 | | −1.4; 2.2 | |
| LS Mean (SE) | 0.09 | (0.17) | 0.89 | (0.17) | 0.32 | (0.11) | 0.42 | (0.11) |

| LS Mean Diff vs. placebo | 0.80 (0.39, | 0.10 (−0.18, |
|---|---|---|
| (95% CI) | 1.21) | 0.38) |
| P-value vs. placebo | 0.0001 | 0.4877 |

Week 12

| Number (observed/LOCF | 30 | (21/9) | 29 | (24/5) | 62 | (52/10) | 54 | (51/3) |
|---|---|---|---|---|---|---|---|---|
| imputed) | | | | | | | | |
| Mean (SD) | 4.90 | (1.31) | 5.66 | (0.96) | 5.03 | (1.00) | 5.45 | (0.86) |

| | | | | |
|---|---|---|---|---|
| Median | 4.95 | 5.82 | 4.95 | 5.36 |
| Q1; Q3 | 3.82; 6.00 | 5.18; 6.36 | 4.18; 5.82 | 4.91; 6.18 |
| Min; Max | 2.7; 7.0 | 3.8; 7.0 | 2.1; 6.6 | 3.1; 7.0 |

Change from baseline

| Number (observed/LOCF | 29 | (20/9) | 29 | (24/5) | 62 | (52/10) | 53 | (50/3) |
|---|---|---|---|---|---|---|---|---|
| imputed) | | | | | | | | |
| Mean (SD) | 0.14 | (1.14) | 0.85 | (0.96) | 0.29 | (0.82) | 0.50 | (0.86) |
| Median | 0.00 | | 0.45 | | 0.27 | | 0.55 | |
| Q1; Q3 | −0.18; 0.91 | | 0.18; 1.27 | | −0.27; 0.91 | | −0.18; 1.09 | |
| Min; Max | −3.0; 2.3 | | −0.7; 2.9 | | −1.8; 2.8 | | −1.3; 2.8 | |
| LS Mean (SE) | −0.01 | (0.20) | 0.77 | (0.22) | 0.31 | (0.11) | 0.62 | (0.11) |

| LS Mean Diff vs. placebo | 0.78 (0.28, | 0.31 (0.02, |
|---|---|---|
| (95% CI) | 1.28) | 0.60) |
| P-value vs. placebo | 0.0023 | 0.0339 |

Emotional function score
Baseline

| Number | 31 | | 32 | | 67 | | 62 | |
|---|---|---|---|---|---|---|---|---|
| Mean (SD) | 4.70 | (1.71) | 5.21 | (1.26) | 4.87 | (1.14) | 5.15 | (1.14) |

| | | | | |
|---|---|---|---|---|
| Median | 5.40 | 5.30 | 4.80 | 5.20 |
| Q1; Q3 | 3.80; 5.80 | 4.10; 6.50 | 4.20; 5.60 | 4.40; 6.00 |
| Min; Max | 1.2; 7.0 | 2.0; 7.0 | 1.8; 6.8 | 1.2; 7.0 |

Week 4

| Number (observed/LOCF | 30 | (28/2) | 31 | (30/1) | 65 | (64/1) | 61 | (61/0) |
|---|---|---|---|---|---|---|---|---|
| imputed) | | | | | | | | |
| Mean (SD) | 4.51 | (1.71) | 5.85 | (1.36) | 5.02 | (1.12) | 5.37 | (1.23) |

| | | | | |
|---|---|---|---|---|
| Median | 4.70 | 6.40 | 5.00 | 5.40 |
| Q1; Q3 | 3.60; 5.80 | 5.00; 7.00 | 4.40; 5.80 | 4.80; 6.40 |
| Min; Max | 1.0; 7.0 | 1.6; 7.0 | 2.0; 7.0 | 1.2; 7.0 |

Change from baseline

| Number (observed/LOCF | 29 | (27/2) | 31 | (30/1) | 65 | (64/1) | 60 | (60/0) |
|---|---|---|---|---|---|---|---|---|
| imputed) | | | | | | | | |
| Mean (SD) | −0.23 | (1.80) | 0.61 | (1.24) | 0.10 | (0.99) | 0.22 | (1.08) |
| Median | 0.20 | | 0.40 | | 0.20 | | 0.20 | |
| Q1; Q3 | −0.60; 0.60 | | −0.40; 1.60 | | −0.40; 0.80 | | −0.40; 0.80 | |
| Min; Max | −5.2; 2.8 | | −2.4; 3.0 | | −2.4; 3.0 | | −2.6; 2.8 | |
| LS Mean (SE) | −0.25 | (0.29) | 0.77 | (0.31) | 0.14 | (0.13) | 0.35 | (0.13) |

| LS Mean Diff vs. placebo | 1.02 (0.30, | 0.21 (−0.12, |
|---|---|---|
| (95% CI) | 1.74) | 0.54) |
| P-value vs. placebo | 0.0058 | 0.2202 |

Week 8

| Number (observed/LOCF | 31 | (28/3) | 31 | (27/4) | 65 | (62/3) | 59 | (56/3) |
|---|---|---|---|---|---|---|---|---|
| imputed) | | | | | | | | |
| Mean (SD) | 4.92 | (1.63) | 6.12 | (1.01) | 5.25 | (1.21) | 5.52 | (1.11) |

| | | | | |
|---|---|---|---|---|
| Median | 5.20 | 6.20 | 5.60 | 5.40 |
| Q1; Q3 | 3.40; 6.20 | 5.80; 7.00 | 4.40; 6.20 | 4.80; 6.60 |
| Min; Max | 1.0; 7.0 | 2.8; 7.0 | 2.0; 7.0 | 1.2; 7.0 |

Change from baseline

| Number (observed/LOCF | 30 | (27/3) | 31 | (27/4) | 65 | (62/3) | 58 | (55/3) |
|---|---|---|---|---|---|---|---|---|
| imputed) | | | | | | | | |
| Mean (SD) | 0.15 | (1.34) | 0.87 | (1.17) | 0.33 | (1.09) | 0.37 | (0.87) |
| Median | 0.20 | | 0.60 | | 0.20 | | 0.40 | |
| Q1; Q3 | −0.40; 1.00 | | 0.00; 1.40 | | −0.60; 1.20 | | 0.00; 0.80 | |
| Min; Max | −3.8; 2.2 | | −1.2; 4.2 | | −1.8; 3.0 | | −1.8; 2.6 | |
| LS Mean (SE) | 0.08 | (0.23) | 1.06 | (0.24) | 0.38 | (0.12) | 0.51 | (0.13) |

| LS Mean Diff vs. placebo | 0.98 (0.42, | 0.13 (−0.20, |
|---|---|---|
| (95% CI) | 1.54) | 0.45) |
| P-value vs. placebo | 0.0006 | 0.4378 |

Week 12

| Number (observed/LOCF | 30 | (21/9) | 29 | (24/5) | 62 | (52/10) | 54 | (51/3) |
|---|---|---|---|---|---|---|---|---|
| imputed) | | | | | | | | |
| Mean (SD) | 5.14 | (1.55) | 5.96 | (1.16) | 5.12 | (1.42) | 5.57 | (1.28) |

| | | | | |
|---|---|---|---|---|
| Median | 5.40 | 6.20 | 5.10 | 5.60 |
| Q1; Q3 | 4.00; 6.20 | 5.20; 7.00 | 4.40; 6.40 | 5.20; 6.60 |
| Min; Max | 1.0; 7.0 | 3.2; 7.0 | 1.0; 7.0 | 1.2; 7.0 |

Change from baseline

| Number (observed/LOCF | 29 | (20/9) | 29 | (24/5) | 62 | (52/10) | 53 | (50/3) |
|---|---|---|---|---|---|---|---|---|
| imputed) | | | | | | | | |
| Mean (SD) | 0.37 | (1.62) | 0.72 | (1.14) | 0.19 | (1.20) | 0.43 | (1.04) |
| Median | 0.40 | | 0.40 | | 0.20 | | 0.40 | |
| Q1; Q3 | 0.00; 1.20 | | 0.00; 1.40 | | −0.40; 1.00 | | 0.00; 1.00 | |
| Min; Max | −3.8; 4.8 | | −0.8; 3.4 | | −3.8; 3.4 | | −2.4; 2.8 | |
| LS Mean (SE) | 0.23 | (0.24) | 0.97 | (0.27) | 0.21 | (0.15) | 0.53 | (0.16) |

| LS Mean Diff vs. placebo | 0.74 (0.12, | 0.32 (−0.08, |
|---|---|---|
| (95% CI) | 1.36) | 0.72) |
| P-value vs. placebo | 0.0195 | 0.1134 |

Environmental stimuli score
Baseline

| Number | 31 | | 32 | | 67 | | 62 | |
|---|---|---|---|---|---|---|---|---|
| Mean (SD) | 4.62 | (1.46) | 4.43 | (1.64) | 4.31 | (1.16) | 4.64 | (1.06) |

| | | | | |
|---|---|---|---|---|
| Median | 4.75 | 4.75 | 4.25 | 4.75 |
| Q1; Q3 | 3.75; 5.75 | 3.63; 5.75 | 3.50; 5.25 | 4.00; 5.25 |
| Min; Max | 1.8; 7.0 | 1.0; 7.0 | 1.0; 7.0 | 1.0; 7.0 |

Week 4

| Number (observed/LOCF | 30 | (28/2) | 31 | (30/1) | 65 | (64/1) | 61 | (61/0) |
|---|---|---|---|---|---|---|---|---|
| imputed) | | | | | | | | |
| Mean (SD) | 4.37 | (1.62) | 5.13 | (1.26) | 4.62 | (1.25) | 4.78 | (1.20) |

| | | | | |
|---|---|---|---|---|
| Median | 4.88 | 5.25 | 4.75 | 4.75 |
| Q1; Q3 | 3.00; 5.75 | 4.25; 6.00 | 3.75; 5.50 | 4.00; 5.75 |
| Min; Max | 1.3; 6.5 | 2.0; 7.0 | 1.0; 7.0 | 1.8; 7.0 |

Change from baseline

| Number (observed/LOCF | 29 | (27/2) | 31 | (30/1) | 65 | (64/1) | 60 | (60/0) |
|---|---|---|---|---|---|---|---|---|
| imputed) | | | | | | | | |
| Mean (SD) | −0.22 | (2.04) | 0.67 | (1.52) | 0.28 | (0.97) | 0.12 | (1.09) |
| Median | 0.00 | | 0.50 | | 0.00 | | 0.25 | |
| Q1; Q3 | −0.50; 1.25 | | −0.75; 1.75 | | −0.25; 0.75 | | −0.50; 0.63 | |
| Min; Max | −5.8; 2.5 | | −2.3; 4.8 | | −2.5; 2.8 | | −2.5; 2.3 | |
| LS Mean (SE) | −0.15 | (0.30) | 0.60 | (0.31) | 0.33 | (0.12) | 0.30 | (0.13) |

| LS Mean Diff vs. placebo | 0.76 (0.02, | −0.03 (−0.37, |
|---|---|---|
| (95% CI) | 1.49) | 0.30) |
| P-value vs. placebo | 0.0432 | 0.8468 |

Week 8

| Number (observed/LOCF | 31 | (28/3) | 31 | (27/4) | 65 | (62/3) | 59 | (56/3) |
|---|---|---|---|---|---|---|---|---|
| imputed) | | | | | | | | |
| Mean (SD) | 4.60 | (1.53) | 5.19 | (1.24) | 4.66 | (1.34) | 4.90 | (1.29) |

| | | | | |
|---|---|---|---|---|
| Median | 5.25 | 5.25 | 5.00 | 4.75 |
| Q1; Q3 | 3.75; 5.75 | 4.50; 6.00 | 3.75; 5.75 | 4.00; 6.00 |
| Min; Max | 1.3; 6.8 | 2.0; 7.0 | 1.0; 7.0 | 2.0; 7.0 |

Change from baseline

| Number (observed/LOCF | 30 | (27/3) | 31 | (27/4) | 65 | (62/3) | 58 | (55/3) |
|---|---|---|---|---|---|---|---|---|
| imputed) | | | | | | | | |
| Mean (SD) | 0.01 | (1.65) | 0.73 | (1.57) | 0.33 | (1.21) | 0.24 | (1.15) |
| Median | 0.25 | | 0.50 | | 0.00 | | 0.25 | |
| Q1; Q3 | −0.50; 1.25 | | −0.25; 1.25 | | −0.25; 1.00 | | −0.50; 1.00 | |
| Min; Max | −5.8; 2.0 | | −1.8; 4.8 | | −2.8; 3.8 | | −2.8; 2.8 | |
| LS Mean (SE) | −0.02 | (0.27) | 0.66 | (0.28) | 0.29 | (0.15) | 0.36 | (0.16) |

| LS Mean Diff vs. placebo | 0.68 (0.03, | 0.06 (−0.34, |
|---|---|---|
| (95% CI) | 1.33) | 0.47) |
| P-value vs. placebo | 0.0394 | 0.7524 |

Week 12

| Number (observed/LOCF | 30 | (21/9) | 29 | (24/5) | 62 | (52/10) | 54 | (51/3) |
|---|---|---|---|---|---|---|---|---|
| imputed) | | | | | | | | |
| Mean (SD) | 4.66 | (1.66) | 5.34 | (1.20) | 4.76 | (1.35) | 5.08 | (1.27) |

| | | | | |
|---|---|---|---|---|
| Median | 4.88 | 5.25 | 5.00 | 5.00 |
| Q1; Q3 | 3.75; 6.00 | 5.00; 6.25 | 3.75; 5.75 | 4.50; 5.75 |
| Min; Max | 1.0; 7.0 | 2.0; 7.0 | 1.0; 7.0 | 1.0; 7.0 |

Change from baseline

| Number (observed/LOCF | 29 | (20/9) | 29 | (24/5) | 62 | (52/10) | 53 | (50/3) |
|---|---|---|---|---|---|---|---|---|
| imputed) | | | | | | | | |
| Mean (SD) | 0.09 | (1.71) | 0.87 | (1.65) | 0.40 | (1.10) | 0.46 | (1.18) |
| Median | 0.25 | | 0.50 | | 0.25 | | 0.50 | |
| Q1; Q3 | −0.50; 1.25 | | 0.00; 1.75 | | −0.25; 1.00 | | 0.00; 1.25 | |
| Min; Max | −5.5; 2.3 | | −2.0; 5.3 | | −2.0; 3.0 | | −3.3; 2.5 | |
| LS Mean (SE) | −0.08 | (0.28) | 0.70 | (0.31) | 0.37 | (0.15) | 0.54 | (0.16) |

| LS Mean Diff vs. placebo | 0.78 (0.06, | 0.17 (−0.23, |
|---|---|---|
| (95% CI) | 1.49) | 0.57) |
| P-value vs. placebo | 0.0331 | 0.4016 |

TABLE 53
Subgroup analysis: change from baseline in AQLQ global score
at EOT (Week 12) by demographics subgroups (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo TID | 400 mg TID |
| AQLQ global score | (N = 32) | (N = 32) | (N = 68) | (N = 64) |

Age group 1 (years)
<45

| Number | 10 | | 10 | | 29 | | 23 |
|---|---|---|---|---|---|---|---|
| Mean (SD) | 0.80 | (0.76) | 0.62 | (0.82) | 0.12 | (1.03) | 0.53 (0.64) |
| LS Mean (SE) | 0.66 | (0.23) | 0.86 | (0.26) | 0.26 | (0.16) | 0.69 (0.17) |

| LS Mean Diff vs. | 0.20 (−0.36, | 0.43 (0.00, |
|---|---|---|
| placebo (95% CI) | 0.76) | 0.85) |
| P-value vs. placebo | 0.4823 | 0.0494 |

≥45

| Number | 19 | | 19 | | 33 | | 30 |
|---|---|---|---|---|---|---|---|
| Mean (SD) | 0.03 | (1.25) | 0.95 | (1.07) | 0.37 | (0.76) | 0.41 (1.01) |
| LS Mean (SE) | −0.16 | (0.22) | 0.89 | (0.25) | 0.32 | (0.16) | 0.47 (0.18) |

| LS Mean Diff vs. | 1.04 (0.47, | 0.15 (−0.29, |
|---|---|---|
| placebo (95% CI) | 1.62) | 0.59) |
| P-value vs. placebo | 0.0004 | 0.5139 |
| Overall p-value for interaction | 0.0607 | 0.5248 |

Gender
Male

| Number | 7 | | 13 | | 27 | | 23 |
|---|---|---|---|---|---|---|---|
| Mean (SD) | 0.31 | (0.58) | 0.97 | (0.88) | 0.17 | (0.82) | 0.40 (0.92) |
| LS Mean (SE) | 0.32 | (0.28) | 0.73 | (0.30) | 0.26 | (0.17) | 0.41 (0.18) |

| LS Mean Diff vs. | 0.41 (−0.46, | 0.15 (−0.31, |
|---|---|---|
| placebo (95% CI) | 1.28) | 0.60) |
| P-value vs. placebo | 0.3547 | 0.5238 |

Female

| Number | 22 | | 16 | | 35 | | 30 |
|---|---|---|---|---|---|---|---|
| Mean (SD) | 0.29 | (1.30) | 0.74 | (1.09) | 0.32 | (0.96) | 0.51 (0.83) |
| LS Mean (SE) | 0.02 | (0.27) | 0.74 | (0.27) | 0.33 | (0.16) | 0.66 (0.17) |

| LS Mean Diff vs. | 0.73 (0.11, | 0.32 (−0.11, |
|---|---|---|
| placebo (95% CI) | 1.35) | 0.75) |
| P-value vs. placebo | 0.0220 | 0.1394 |
| Overall p-value for interaction | 0.6030 | 0.5902 |

Baseline weight group (kg)
<60

| Number | 4 | | 3 | | 10 | | 2 |
|---|---|---|---|---|---|---|---|
| Mean (SD) | −0.12 | (1.31) | 0.95 | (1.45) | 0.58 | (0.79) | 0.78 (0.40) |
| LS Mean (SE) | −0.81 | (0.11) | 0.82 | (0.16) | 0.67 | (0.29) | 0.59 (0.63) |

| LS Mean Diff vs. | 1.63 (0.98, | −0.08 (−1.53, |
|---|---|---|
| placebo (95% CI) | 2.28) | 1.37) |
| P-value vs. placebo | 0.0084 | 0.9112 |

≥60-<90

| Number | 22 | | 20 | | 37 | | 32 |
|---|---|---|---|---|---|---|---|
| Mean (SD) | 0.26 | (1.17) | 0.87 | (0.91) | 0.23 | (0.98) | 0.56 (0.86) |
| LS Mean (SE) | 0.06 | (0.22) | 0.77 | (0.24) | 0.25 | (0.16) | 0.66 (0.17) |

| LS Mean Diff vs. | 0.70 (0.17, | 0.41 (−0.02, |
|---|---|---|
| placebo (95% CI) | 1.23) | 0.84) |
| P-value vs. placebo | 0.0092 | 0.0612 |

≥90

| Number | 3 | | 6 | | 15 | | 19 |
|---|---|---|---|---|---|---|---|
| Mean (SD) | 1.09 | (0.62) | 0.70 | (1.21) | 0.11 | (0.73) | 0.26 (0.89) |
| LS Mean (SE) | 1.06 | (0.70) | 0.80 | (1.03) | 0.15 | (0.21) | 0.32 (0.18) |

| LS Mean Diff vs. | −0.26 (−3.62, | 0.17 (−0.34, |
|---|---|---|
| placebo (95% CI) | 3.10) | 0.68) |
| P-value vs. placebo | 0.8398 | 0.5090 |
| Overall p-value for interaction | 0.4319 | 0.7466 |

Baseline BMI group (kg/m²)
<25

| Number | 6 | | 8 | | 20 | | 8 |
|---|---|---|---|---|---|---|---|
| Mean (SD) | 0.09 | (1.08) | 1.12 | (0.99) | 0.24 | (0.88) | 0.65 (0.74) |
| LS Mean (SE) | −0.45 | (0.56) | 0.55 | (0.62) | 0.42 | (0.19) | 0.98 (0.29) |

| LS Mean Diff vs. | 1.00 (−0.23, | 0.56 (−0.07, |
|---|---|---|
| placebo (95% CI) | 2.22) | 1.18) |
| P-value vs. placebo | 0.0991 | 0.0813 |

≥25-<30

| Number | 15 | | 13 | | 25 | | 26 |
|---|---|---|---|---|---|---|---|
| Mean (SD) | 0.13 | (1.24) | 0.61 | (0.73) | 0.25 | (1.05) | 0.75 (0.71) |
| LS Mean (SE) | 0.02 | (0.25) | 0.69 | (0.29) | 0.48 | (0.16) | 0.96 (0.16) |

| LS Mean Diff vs. | 0.67 (−0.05, | 0.47 (0.07, |
|---|---|---|
| placebo (95% CI) | 1.39) | 0.87) |
| P-value vs. placebo | 0.0679 | 0.0210 |

≥30

| Number | 8 | | 8 | | 17 | | 19 |
|---|---|---|---|---|---|---|---|
| Mean (SD) | 0.75 | (1.03) | 0.93 | (0.45) | 0.28 | (0.70) | 0.00 (0.94) |
| LS Mean (SE) | 0.56 | (0.46) | 1.30 | (1.36) | 0.20 | (0.21) | 0.19 (0.20) |

| LS Mean Diff vs. | 0.73 (−0.21, | −0.01 (−0.58, |
|---|---|---|
| placebo (95% CI) | 1.68) | 0.56) |
| P-value vs. placebo | 0.1287 | 0.9701 |
| Overall p-value for interaction | 0.9298 | 0.3404 |

Region
Eastern Europe

| Number | 4 | | 2 | | 44 | | 35 |
|---|---|---|---|---|---|---|---|
| Mean (SD) | −0.04 | (0.34) | −0.19 | (0.66) | 0.08 | (0.91) | 0.46 (0.68) |
| LS Mean (SE) | −0.03 | (0.30) | 0.04 | (0.53) | 0.06 | (0.11) | 0.48 (0.12) |

| LS Mean Diff vs. | 0.08 (−1.12, | 0.43 (0.10, |
|---|---|---|
| placebo (95% CI) | 1.28) | 0.76) |
| P-value vs. placebo | 0.9007 | 0.0111 |

Latin America

| Number | 22 | | 24 | | 16 | | 16 |
|---|---|---|---|---|---|---|---|
| Mean (SD) | 0.35 | (1.29) | 0.98 | (0.96) | 0.83 | (0.51) | 0.44 (1.22) |
| LS Mean (SE) | 0.30 | (0.20) | 1.16 | (0.20) | 0.74 | (0.28) | 0.52 (0.28) |

| LS Mean Diff vs. | 0.87 (0.36, | −0.22 (−0.83, |
|---|---|---|
| placebo (95% CI) | 1.38) | 0.39) |
| P-value vs. placebo | 0.0009 | 0.4745 |

Western Countries

| Number | 1 | | 2 | | 1 | | 2 |
|---|---|---|---|---|---|---|---|
| Mean (SD) | 1.25 | (NC) | 0.44 | (1.64) | −1.44 | (NC) | 0.78 (0.75) |
| LS Mean (SE) | NC | (NC) | NC | (NC) | NC | (NC) | NC (NC) |

| | | |
|---|---|---|
| LS Mean Diff vs. | NC (NC, | NC (NC, |
| placebo (95% CI) | NC) | NC) |
| P-value vs. placebo | NC | NC |

APAC

| Number | 2 | | 1 | | 1 | | 0 |
|---|---|---|---|---|---|---|---|
| Mean (SD) | −0.11 | (0.60) | 0.22 | (NC) | 0.72 | (NC) | NC (NC) |
| LS Mean (SE) | NC | (NC) | NC | (NC) | NC | (NC) | NC (NC) |

| | | |
|---|---|---|
| LS Mean Diff vs. | NC (NC, | NC (NC, |
| placebo (95% CI) | NC) | NC) |
| P-value vs. placebo | NC | NC |
| Overall p-value for interaction | 0.4777 | 0.0019 |

LATAM: Latin America, ROW: Rest of the world

TABLE 54
Subgroup analysis: change from baseline in AQLQ global score at EOT (Week
12) by disease and other characteristics subgroups (mITT population).

| | 400 mg BID cohort | | 400 mg TID cohort | |
|---|---|---|---|---|
| | | Rilzabrutinib | | Rilzabrutinib |
| | Placebo BID | 400 mg BID | Placebo BID | 400 mg TID |
| AQLQ global score | (N = 32) | (N = 32) | (N = 68) | (N = 64) |

Age at onset of asthma (years)
<12

| Number | 7 | | 8 | | 14 | | 12 | |
|---|---|---|---|---|---|---|---|---|
| Mean (SD) | 0.58 | (0.91) | 0.89 | (0.71) | −0.04 | (0.87) | 0.51 | (0.94) |
| LS Mean (SE) | 0.61 | (0.29) | 0.95 | (0.38) | 0.03 | (0.23) | 0.49 | (0.26) |

| LS Mean Diff vs. | 0.34 (−0.43, | 0.46 (−0.19, |
|---|---|---|
| placebo (95% CI) | 1.11) | 1.12) |
| P-value vs. placebo | 0.3884 | 0.1645 |

≥12-<18

| Number | 2 | | 5 | | 5 | | 5 | |
|---|---|---|---|---|---|---|---|---|
| Mean (SD) | 0.44 | (0.57) | 0.91 | (1.28) | 0.34 | (0.67) | 0.53 | (1.61) |
| LS Mean (SE) | −3.05 | (5.06) | −0.56 | (2.39) | 0.77 | (0.98) | 0.40 | (0.73) |

| LS Mean Diff vs. | 2.49 (−9.16, | −0.37 (−2.70, |
|---|---|---|
| placebo (95% CI) | 14.14) | 1.96) |
| P-value vs. placebo | 0.4551 | 0.7001 |

≥18-<40

| Number | 14 | | 13 | | 26 | | 22 | |
|---|---|---|---|---|---|---|---|---|
| Mean (SD) | 0.51 | (1.05) | 0.86 | (1.12) | 0.13 | (0.95) | 0.34 | (0.74) |
| LS Mean (SE) | 0.20 | (0.30) | 0.77 | (0.31) | 0.23 | (0.18) | 0.48 | (0.19) |

| LS Mean Diff vs. | 0.57 (−0.18, | 0.25 (−0.23, |
|---|---|---|
| placebo (95% CI) | 1.32) | 0.72) |
| P-value vs. placebo | 0.1339 | 0.3043 |

≥40

| Number | 6 | | 3 | | 17 | | 14 | |
|---|---|---|---|---|---|---|---|---|
| Mean (SD) | −0.59 | (1.53) | 0.48 | (0.97) | 0.67 | (0.81) | 0.60 | (0.71) |
| LS Mean (SE) | −0.70 | (0.46) | 0.23 | (0.73) | 0.41 | (0.22) | 0.71 | (0.25) |

| LS Mean Diff vs. | 0.93 (−0.59, | 0.29 (−0.34, |
|---|---|---|
| placebo (95% CI) | 2.46) | 0.92) |
| P-value vs. placebo | 0.2299 | 0.3596 |
| Overall p-value for interaction | 0.8090 | 0.8443 |

Age at onset of asthma (years)
<18

| Number | 9 | | 13 | | 19 | | 17 | |
|---|---|---|---|---|---|---|---|---|
| Mean (SD) | 0.55 | (0.81) | 0.90 | (0.92) | 0.06 | (0.82) | 0.52 | (1.12) |
| LS Mean (SE) | 0.60 | (0.23) | 1.03 | (0.23) | 0.15 | (0.22) | 0.45 | (0.23) |

| LS Mean Diff vs. | 0.43 (−0.08, | 0.31 (−0.27, |
|---|---|---|
| placebo (95% CI) | 0.95) | 0.89) |
| P-value vs. placebo | 0.0978 | 0.2994 |

≥18

| Number | 20 | | 16 | | 43 | | 36 | |
|---|---|---|---|---|---|---|---|---|
| Mean (SD) | 0.18 | (1.28) | 0.79 | (1.07) | 0.34 | (0.93) | 0.44 | (0.73) |
| LS Mean (SE) | −0.07 | (0.25) | 0.68 | (0.29) | 0.34 | (0.14) | 0.57 | (0.15) |

| LS Mean Diff vs. | 0.75 (0.08, | 0.24 (−0.14, |
|---|---|---|
| placebo (95% CI) | 1.43) | 0.61) |
| P-value vs. placebo | 0.0286 | 0.2154 |
| Overall p-value for interaction | 0.5227 | 0.7855 |

Number of asthma exacerbations within 2 years before screening visit
1

| Number | 9 | | 11 | | 47 | | 31 | |
|---|---|---|---|---|---|---|---|---|
| Mean (SD) | 0.84 | (0.91) | 1.05 | (0.99) | 0.30 | (0.86) | 0.57 | (0.88) |
| LS Mean (SE) | 0.42 | (0.34) | 1.16 | (0.37) | 0.39 | (0.13) | 0.68 | (0.16) |

| LS Mean Diff vs. | 0.75 (−0.16, | 0.29 (−0.08, |
|---|---|---|
| placebo (95% CI) | 1.65) | 0.66) |
| P-value vs. placebo | 0.1070 | 0.1248 |

2

| Number | 13 | | 10 | | 15 | | 19 | |
|---|---|---|---|---|---|---|---|---|
| Mean (SD) | 0.35 | (0.98) | 0.76 | (0.86) | 0.12 | (1.01) | 0.34 | (0.89) |
| LS Mean (SE) | 0.15 | (0.27) | 0.76 | (0.37) | 0.14 | (0.24) | 0.41 | (0.22) |

| LS Mean Diff vs. | 0.61 (−0.11, | 0.27 (−0.33, |
|---|---|---|
| placebo (95% CI) | 1.33) | 0.87) |
| P-value vs. placebo | 0.0972 | 0.3775 |

>2

| Number | 7 | | 8 | | 0 | | 3 | |
|---|---|---|---|---|---|---|---|---|
| Mean (SD) | −0.51 | (1.41) | 0.66 | (1.20) | NC | (NC) | 0.14 | (0.53) |
| LS Mean (SE) | −0.64 | (0.36) | 0.54 | (0.33) | NC | (NC) | NC | (NC) |

| LS Mean Diff vs. | 1.18 (0.30, | NC (NC, |
|---|---|---|
| placebo (95% CI) | 2.07) | NC) |
| P-value vs. placebo | 0.0089 | NC |
| Overall p-value for interaction | 0.4656 | 0.2099 |

Atopic medical conditions
Yes

| Number | 16 | | 20 | | 20 | | 18 | |
|---|---|---|---|---|---|---|---|---|
| Mean (SD) | 0.28 | (1.35) | 0.81 | (0.98) | 0.52 | (0.92) | 0.48 | (0.84) |
| LS Mean (SE) | 0.14 | (0.28) | 0.66 | (0.26) | 0.43 | (0.21) | 0.56 | (0.23) |

| LS Mean Diff vs. | 0.52 (−0.09, | 0.13 (−0.45, |
|---|---|---|
| placebo (95% CI) | 1.13) | 0.71) |
| P-value vs. placebo | 0.0945 | 0.6600 |

No

| Number | 13 | | 9 | | 42 | | 35 | |
|---|---|---|---|---|---|---|---|---|
| Mean (SD) | 0.31 | (0.91) | 0.91 | (1.06) | 0.13 | (0.87) | 0.46 | (0.89) |
| LS Mean (SE) | −0.10 | (0.27) | 0.99 | (0.32) | 0.27 | (0.16) | 0.64 | (0.16) |

| LS Mean Diff vs. | 1.08 (0.29, | 0.37 (0.00, |
|---|---|---|
| placebo (95% CI) | 1.87) | 0.73) |
| P-value vs. placebo | 0.0074 | 0.0477 |
| Overall p-value for interaction | 0.2340 | 0.6262 |

Background ICS dose level at randomization (400 mg TID only)
Medium

| Number | 34 | | 22 | |
|---|---|---|---|---|
| Mean (SD) | 0.15 | (0.85) | 0.41 | (0.73) |
| LS Mean (SE) | 0.23 | (0.17) | 0.53 | (0.19) |

| LS Mean Diff vs. | 0.31 (−0.11, |
|---|---|
| placebo (95% CI) | 0.72) |
| P-value vs. placebo | 0.1446 |

High

| Number | 28 | | 31 | |
|---|---|---|---|---|
| Mean (SD) | 0.38 | (0.95) | 0.50 | (0.96) |
| LS Mean (SE) | 0.33 | (0.18) | 0.59 | (0.19) |

| LS Mean Diff vs. | 0.26 (−0.22, |
|---|---|
| placebo (95% CI) | 0.74) |
| P-value vs. placebo | 0.2872 |
| Overall p-value for interaction | 0.8865 |

Smoking history
Former

| Number | 7 | | 4 | | 4 | | 4 | |
|---|---|---|---|---|---|---|---|---|
| Mean (SD) | 0.43 | (1.52) | 0.73 | (1.45) | 0.23 | (0.26) | −0.55 | (1.82) |
| LS Mean (SE) | −0.16 | (0.46) | 1.74 | (0.68) | −0.09 | (0.79) | −0.11 | (0.84) |

| LS Mean Diff vs. | 1.89 (0.62, | −0.02 (−1.90, |
|---|---|---|
| placebo (95% CI) | 3.16) | 1.86) |
| P-value vs. placebo | 0.0035 | 0.9823 |

Never

| Number | 22 | | 25 | | 58 | | 49 | |
|---|---|---|---|---|---|---|---|---|
| Mean (SD) | 0.25 | (1.05) | 0.86 | (0.94) | 0.26 | (0.93) | 0.55 | (0.71) |
| LS Mean (SE) | 0.09 | (0.21) | 0.74 | (0.21) | 0.34 | (0.11) | 0.68 | (0.12) |

| LS Mean Diff vs. | 0.65 (0.15, | 0.34 (0.04, |
|---|---|---|
| placebo (95% CI) | 1.15) | 0.63) |
| P-value vs. placebo | 0.0106 | 0.0266 |
| Overall p-value for interaction | 0.5532 | 0.5469 |

Baseline pre-bronchodilator FEV1 (L) (400 mg BID only)
<Median (2.022)

| Number | 13 | | 15 | |
|---|---|---|---|---|
| Mean (SD) | 0.04 | (1.27) | 0.89 | (0.98) |
| LS Mean (SE) | −0.30 | (0.26) | 0.59 | (0.28) |

| LS Mean Diff vs. | 0.89 (0.28, |
|---|---|
| placebo (95% CI) | 1.51) |
| P-value vs. placebo | 0.0046 |

≥Median (2.022)

| Number | 16 | | 13 | |
|---|---|---|---|---|
| Mean (SD) | 0.50 | (1.04) | 0.74 | (1.06) |
| LS Mean (SE) | 0.40 | (0.26) | 0.94 | (0.29) |

| LS Mean Diff vs. | 0.54 (−0.14, |
|---|---|
| placebo (95% CI) | 1.21) |
| P-value vs. placebo | 0.1187 |
| Overall p-value for interaction | 0.3692 |

Baseline pre-bronchodilator percent predicted FEV1 (%) (400 mg BID only)
<Median (68)

| Number | 11 | | 16 | |
|---|---|---|---|---|
| Mean (SD) | 0.24 | (1.11) | 1.00 | (1.01) |
| LS Mean (SE) | −0.22 | (0.30) | 0.69 | (0.30) |

| LS Mean Diff vs. | 0.92 (0.29, |
|---|---|
| placebo (95% CI) | 1.55) |
| P-value vs. placebo | 0.0044 |

≥Median (68)

| Number | 18 | | 12 | |
|---|---|---|---|---|
| Mean (SD) | 0.32 | (1.21) | 0.58 | (0.98) |
| LS Mean (SE) | 0.20 | (0.25) | 0.67 | (0.31) |

| LS Mean Diff vs. | 0.47 (−0.26, |
|---|---|
| placebo (95% CI) | 1.20) |
| P-value vs. placebo | 0.2058 |
| Overall p-value for interaction | 0.3643 |

Baseline pre-bronchodilator FEV1 (L) (400 mg TID only)
<Median (2.1955)

| Number | 30 | | 23 | |
|---|---|---|---|---|
| Mean (SD) | 0.26 | (0.84) | 0.48 | (0.93) |
| LS Mean (SE) | 0.25 | (0.17) | 0.53 | (0.20) |

| LS Mean Diff vs. | 0.28 (−0.19, |
|---|---|
| placebo (95% CI) | 0.76) |
| P-value vs. placebo | 0.2414 |

≥Median (2.1955)

| Number | 31 | | 29 | |
|---|---|---|---|---|
| Mean (SD) | 0.22 | (0.96) | 0.47 | (0.84) |
| LS Mean (SE) | 0.32 | (0.16) | 0.59 | (0.16) |

| LS Mean Diff vs. | 0.27 (−0.15, |
|---|---|
| placebo (95% CI) | 0.69) |
| P-value vs. placebo | 0.2026 |
| Overall p-value for interaction | 0.7803 |

Baseline pre-bronchodilator percent predicted FEV1 group 1 (%) (400 mg TID only)
<Median (77)

| Number | 32 | | 23 | |
|---|---|---|---|---|
| Mean (SD) | 0.05 | (0.87) | 0.49 | (0.92) |
| LS Mean (SE) | 0.13 | (0.18) | 0.55 | (0.21) |

| LS Mean Diff vs. | 0.42 (−0.05, |
|---|---|
| placebo (95% CI) | 0.88) |
| P-value vs. placebo | 0.0787 |

≥Median (77)

| Number | 30 | | 29 | |
|---|---|---|---|---|
| Mean (SD) | 0.48 | (0.89) | 0.47 | (0.84) |
| LS Mean (SE) | 0.43 | (0.16) | 0.60 | (0.16) |

| LS Mean Diff vs. | 0.16 (−0.26, |
|---|---|
| placebo (95% CI) | 0.58) |
| P-value vs. placebo | 0.4566 |
| Overall p-value for interaction | 0.2958 |

Baseline pre-bronchodilator percent predicted FEV1 group 2 (%) (400 mg TID only)
<80

| Number | 37 | | 30 | |
|---|---|---|---|---|
| Mean (SD) | 0.10 | (0.82) | 0.41 | (0.94) |
| LS Mean (SE) | 0.16 | (0.16) | 0.50 | (0.18) |

| LS Mean Diff vs. | 0.35 (−0.06, |
|---|---|
| placebo (95% CI) | 0.76) |
| P-value vs. placebo | 0.0977 |

≥80

| Number | 25 | | 22 | |
|---|---|---|---|---|
| Mean (SD) | 0.49 | (0.97) | 0.57 | (0.77) |
| LS Mean (SE) | 0.43 | (0.17) | 0.70 | (0.18) |

| LS Mean Diff vs. | 0.27 (−0.20, |
|---|---|
| placebo (95% CI) | 0.74) |
| P-value vs. placebo | 0.2629 |
| Overall p-value for interaction | 0.5797 |

Baseline FEV1 reversibility (%)
<12

| Number | 19 | | 13 | | 45 | | 36 | |
|---|---|---|---|---|---|---|---|---|
| Mean (SD) | 0.11 | (1.26) | 0.61 | (0.72) | 0.33 | (0.86) | 0.42 | (0.85) |
| LS Mean (SE) | −0.03 | (0.23) | 0.71 | (0.28) | 0.36 | (0.13) | 0.48 | (0.15) |

| LS Mean Diff vs. | 0.73 (0.07, | 0.12 (−0.24, |
|---|---|---|
| placebo (95% CI) | 1.40) | 0.48) |
| P-value vs. placebo | 0.0303 | 0.5068 |

≥12

| Number | 9 | | 14 | | 10 | | 10 | |
|---|---|---|---|---|---|---|---|---|
| Mean (SD) | 0.57 | (0.88) | 0.97 | (1.23) | 0.39 | (0.79) | 0.56 | (0.72) |
| LS Mean (SE) | 0.47 | (0.33) | 0.76 | (0.33) | 0.30 | (0.32) | 0.63 | (0.41) |

| LS Mean Diff vs. | 0.29 (−0.39, | 0.32 (−0.63, |
|---|---|---|
| placebo (95% CI) | 0.97) | 1.28) |
| P-value vs. placebo | 0.4038 | 0.5035 |
| Overall p-value for interaction | 0.3793 | 0.3913 |

Baseline ACQ-5 score
≤2

| Number | 14 | | 17 | | 26 | | 18 | |
|---|---|---|---|---|---|---|---|---|
| Mean (SD) | 0.47 | (0.92) | 0.50 | (0.80) | 0.46 | (0.94) | 0.53 | (1.02) |
| LS Mean (SE) | 0.17 | (0.27) | 0.60 | (0.24) | 0.35 | (0.19) | 0.64 | (0.22) |

| LS Mean Diff vs. | 0.43 (−0.16, | 0.29 (−0.26, |
|---|---|---|
| placebo (95% CI) | 1.02) | 0.85) |
| P-value vs. placebo | 0.1568 | 0.2966 |

>2

| Number | 15 | | 12 | | 36 | | 35 | |
|---|---|---|---|---|---|---|---|---|
| Mean (SD) | 0.13 | (1.35) | 1.32 | (1.06) | 0.11 | (0.85) | 0.43 | (0.78) |
| LS Mean (SE) | 0.10 | (0.33) | 1.05 | (0.40) | 0.22 | (0.14) | 0.60 | (0.15) |

| LS Mean Diff vs. | 0.95 (0.11, | 0.38 (0.01, |
|---|---|---|
| placebo (95% CI) | 1.79) | 0.75) |
| P-value vs. placebo | 0.0267 | 0.0447 |
| Overall p-value for interaction | 0.3060 | 0.6263 |

LATAM: Latin America, ROW: Rest of the world

TABLE 55
Subgroup analysis: change from baseline in
AQLQ global score at EOT (Week 12) by baseline
ICS/LABA dose level (mITT population).

| | | |
|---|---|---|
| | | Rilzabrutinib |
| | Placebo BID | 400 mg BID |
| AQLQ global score | (N = 32) | (N = 32) |

Background ICS/LABA dose level at randomization
Medium

| Number | 3 | | 4 | |
|---|---|---|---|---|
| Mean (SD) | 0.02 | (0.48) | 1.07 | (0.57) |
| LS Mean (SE) | −0.08 | (0.50) | 0.84 | (0.50) |

| | | |
|---|---|---|
| LS Mean Diff vs. | 0.93 | (0.72, 1.13) |
| placebo (95% CI) | | |

| | |
|---|---|
| P-value vs. placebo | 0.0007 |

High

| Number | 26 | | 25 | |
|---|---|---|---|---|
| Mean (SD) | 0.32 | (1.21) | 0.80 | (1.04) |
| LS Mean (SE) | 0.10 | (0.20) | 0.83 | (0.22) |

| | | |
|---|---|---|
| LS Mean Diff vs. | 0.73 | (0.21, 1.24) |
| placebo (95% CI) | | |

| | |
|---|---|
| P-value vs. placebo | 0.0057 |
| Overall p-value | 0.8629 |
| for interaction | |

LATAM: Latin America, ROW: Rest of the world

TABLE 56
Subgroup analysis: change from baseline in AQLQ global score at
EOT (Week 12) by baseline biomarker subgroups (mITT population).

| | 400 mg BID cohort | | 400 mg TID cohort | |
|---|---|---|---|---|
| | | Rilzabrutinib | | Rilzabrutinib |
| | (N = 32) | 400 mg BID | Placebo TID | 400 mg TID |
| AQLQ global score | Placebo BID | (N = 32) | (N = 68) | (N = 64) |

Baseline IgE level (IU/mL)
<100

| Number | 10 | | 7 | 24 | 18 |
|---|---|---|---|---|---|
| Mean (SD) | 0.00 | (1.71) | 1.01 (1.21) | 0.13 (0.98) | 0.55 (0.59) |
| LS Mean (SE) | −0.25 | (0.58) | 0.64 (0.61) | 0.23 (0.30) | 0.69 (0.36) |

| | | |
|---|---|---|
| LS Mean Diff vs. | 0.90 (−0.36, | 0.46 (0.01, |
| placebo (95% CI) | 2.16) | 0.91) |
| P-value vs. placebo | 0.1622 | 0.0448 |

≥100

| Number | 19 | | 22 | 38 | 35 |
|---|---|---|---|---|---|
| Mean (SD) | 0.45 | (0.74) | 0.78 (0.94) | 0.34 (0.85) | 0.42 (0.98) |
| LS Mean (SE) | 0.15 | (0.41) | 0.74 (0.39) | 0.32 (0.22) | 0.49 (0.23) |

| | | |
|---|---|---|
| LS Mean Diff vs. | 0.59 (0.14, | 0.16 (−0.25, |
| placebo (95% CI) | 1.04) | 0.58) |
| P-value vs. placebo | 0.0095 | 0.4392 |
| Overall p-value for interaction | 0.6080 | 0.4953 |

Baseline median IgE level (IU/mL) (400 mg BID only)
<Median (210.8)

| Number | 12 | | 18 |
|---|---|---|---|
| Mean (SD) | −0.03 | (1.56) | 0.73 (0.89) |
| LS Mean (SE) | −0.34 | (0.34) | 0.68 (0.27) |

| | |
|---|---|
| LS Mean Diff vs. | 1.03 (0.21, |
| placebo (95% CI) | 1.84) |
| P-value vs. placebo | 0.0141 |

≥Median (210.8)

| Number | 17 | | 11 |
|---|---|---|---|
| Mean (SD) | 0.52 | (0.72) | 1.01 (1.15) |
| LS Mean (SE) | 0.26 | (0.20) | 0.94 (0.26) |

| | |
|---|---|
| LS Mean Diff vs. | 0.67 (0.12, |
| placebo (95% CI) | 1.22) |
| P-value vs. placebo | 0.0161 |
| Overall p-value for interaction | 0.7770 |

Baseline median IgE level (IU/mL) (400 mg TID only)
<Median (121.85)

| | | |
|---|---|---|
| Number | 31 | 25 |
| Mean (SD) | 0.03 (1.00) | 0.64 (0.61) |
| LS Mean (SE) | 0.10 (0.19) | 0.71 (0.20) |
| LS Mean Diff vs. | | 0.60 (0.19, |
| placebo (95% CI) | | 1.02) |
| P-value vs. placebo | | 0.0044 |

≥Median (121.85)

| | | |
|---|---|---|
| Number | 31 | 28 |
| Mean (SD) | 0.48 (0.73) | 0.31 (1.03) |
| LS Mean (SE) | 0.76 (0.45) | 0.74 (0.48) |
| LS Mean Diff vs. | | −0.02 (−0.48, |
| placebo (95% CI) | | 0.44) |
| P-value vs. placebo | | 0.9314 |
| Overall p-value for interaction | | 0.0564 |

Baseline median IgA level (mg/L) (400 mg TID only)
<Median (2190)

| | | |
|---|---|---|
| Number | 33 | 26 |
| Mean (SD) | 0.14 (0.93) | 0.69 (0.74) |
| LS Mean (SE) | 0.27 (0.16) | 0.77 (0.17) |
| LS Mean Diff vs. | | 0.50 (0.09, |
| placebo (95% CI) | | 0.92) |
| P-value vs. placebo | | 0.0162 |

≥Median (2190)

| | | |
|---|---|---|
| Number | 29 | 27 |
| Mean (SD) | 0.39 (0.85) | 0.25 (0.94) |
| LS Mean (SE) | 0.33 (0.17) | 0.41 (0.18) |
| LS Mean Diff vs. | | 0.08 (−0.39, |
| placebo (95% CI) | | 0.55) |
| P-value vs. placebo | | 0.7400 |
| Overall p-value for interaction | | 0.1785 |

Baseline blood eosinophil level 1 (10\hat{ }9/L) (400 mg BID only)
<0.15

| Number | 5 | | 8 |
|---|---|---|---|
| Mean (SD) | −0.01 | (1.38) | 0.82 (1.24) |
| LS Mean (SE) | 0.07 | (0.59) | 0.69 (0.40) |

| | |
|---|---|
| LS Mean Diff vs. | 0.62 (−0.95, |
| placebo (95% CI) | 2.19) |
| P-value vs. placebo | 0.3897 |

≥0.15

| Number | 24 | | 21 |
|---|---|---|---|
| Mean (SD) | 0.36 | (1.12) | 0.85 (0.91) |
| LS Mean (SE) | 0.14 | (0.20) | 0.83 (0.26) |

| | |
|---|---|
| LS Mean Diff vs. | 0.69 (0.15, |
| placebo (95% CI) | 1.23) |
| P-value vs. placebo | 0.0128 |
| Overall p-value for interaction | 0.9910 |

Baseline blood eosinophil level 2 (10\hat{ }9/L) (400 mg BID only)
<0.3

| Number | 17 | | 16 |
|---|---|---|---|
| Mean (SD) | 0.33 | (1.18) | 0.90 (1.18) |
| LS Mean (SE) | 0.10 | (0.26) | 0.89 (0.29) |

| | |
|---|---|
| LS Mean Diff vs. | 0.79 (0.09, |
| placebo (95% CI) | 1.49) |
| P-value vs. placebo | 0.0267 |

≥0.3

| Number | 12 | | 13 |
|---|---|---|---|
| Mean (SD) | 0.23 | (1.16) | 0.77 (0.73) |
| LS Mean (SE) | −0.11 | (0.32) | 0.56 (0.30) |

| | |
|---|---|
| LS Mean Diff vs. | 0.67 (0.10, |
| placebo (95% CI) | 1.25) |
| P-value vs. placebo | 0.0221 |
| Overall p-value for interaction | 0.9184 |

Baseline blood eosinophil level 1 (10\hat{ }9/L) (400 mg TID only)
<0.15

| | | |
|---|---|---|
| Number | 28 | 21 |
| Mean (SD) | 0.40 (0.76) | 0.41 (0.58) |
| LS Mean (SE) | 0.47 (0.13) | 0.54 (0.15) |
| LS Mean Diff vs. | | 0.07 (−0.29, |
| placebo (95% CI) | | 0.44) |
| P-value vs. placebo | | 0.6928 |

≥0.15-<0.3

| | | |
|---|---|---|
| Number | 19 | 15 |
| Mean (SD) | 0.16 (1.13) | 0.43 (0.93) |
| LS Mean (SE) | 0.07 (0.27) | 0.39 (0.29) |
| LS Mean Diff vs. | | 0.32 (−0.36, |
| placebo (95% CI) | | 1.01) |
| P-value vs. placebo | | 0.3579 |

≥0.3

| | | |
|---|---|---|
| Number | 15 | 17 |
| Mean (SD) | 0.12 (0.83) | 0.57 (1.11) |
| LS Mean (SE) | 0.02 (0.31) | 0.72 (0.28) |
| LS Mean Diff vs. | | 0.70 (0.04, |
| placebo (95% CI) | | 1.36) |
| P-value vs. placebo | | 0.0378 |
| Overall p-value for interaction | | 0.3666 |

Baseline blood eosinophil level 2 (10\hat{ }9/L) (400 mg TID only)
<0.3

| | | |
|---|---|---|
| Number | 47 | 36 |
| Mean (SD) | 0.30 (0.92) | 0.42 (0.73) |
| LS Mean (SE) | 0.33 (0.13) | 0.50 (0.14) |
| LS Mean Diff vs. | | 0.17 (−0.18, |
| placebo (95% CI) | | 0.52) |
| P-value vs. placebo | | 0.3450 |

≥0.3

| | | |
|---|---|---|
| Number | 15 | 17 |
| Mean (SD) | 0.12 (0.83) | 0.57 (1.11) |
| LS Mean (SE) | 0.02 (0.31) | 0.72 (0.28) |
| LS Mean Diff vs. | | 0.70 (0.04, |
| placebo (95% CI) | | 1.36) |
| P-value vs. placebo | | 0.0378 |
| Overall p-value for interaction | | 0.2659 |

Baseline FeNO level 1 (ppb)
<25

| Number | 13 | | 12 | 39 | 32 |
|---|---|---|---|---|---|
| Mean (SD) | 0.41 | (1.26) | 0.92 (0.93) | 0.33 (0.91) | 0.40 (0.88) |
| LS Mean (SE) | 0.18 | (0.28) | 1.16 (0.28) | 0.38 (0.14) | 0.53 (0.16) |

| | | |
|---|---|---|
| LS Mean Diff vs. | 0.98 (0.31, | 0.15 (−0.24, |
| placebo (95% CI) | 1.66) | 0.54) |
| P-value vs. placebo | 0.0044 | 0.4375 |

≥25

| Number | 16 | | 17 | 19 | 19 |
|---|---|---|---|---|---|
| Mean (SD) | 0.20 | (1.10) | 0.78 (1.05) | 0.29 (0.80) | 0.61 (0.86) |
| LS Mean (SE) | 0.08 | (0.25) | 0.59 (0.29) | 0.26 (0.22) | 0.70 (0.22) |

| | | |
|---|---|---|
| LS Mean Diff vs. | 0.51 (−0.15, | 0.44 (−0.12, |
| placebo (95% CI) | 1.17) | 1.00) |
| P-value vs. placebo | 0.1276 | 0.1229 |
| Overall p-value for interaction | 0.4272 | 0.2955 |

Baseline FeNO level 2 (ppb)
<35

| Number | 17 | | 21 | 46 | 37 |
|---|---|---|---|---|---|
| Mean (SD) | 0.40 | (1.25) | 0.74 (0.99) | 0.28 (0.91) | 0.41 (0.84) |
| LS Mean (SE) | 0.01 | (0.25) | 0.85 (0.23) | 0.30 (0.13) | 0.48 (0.15) |

| | | |
|---|---|---|
| LS Mean Diff vs. | 0.84 (0.28, | 0.18 (−0.19, |
| placebo (95% CI) | 1.40) | 0.54) |
| P-value vs. placebo | 0.0032 | 0.3422 |

≥35

| Number | 12 | | 8 | 12 | 14 |
|---|---|---|---|---|---|
| Mean (SD) | 0.15 | (1.03) | 1.09 (1.01) | 0.47 (0.70) | 0.65 (0.97) |
| LS Mean (SE) | 0.08 | (0.30) | 0.85 (0.40) | 0.23 (0.34) | 0.68 (0.30) |

| | | |
|---|---|---|
| LS Mean Diff vs. | 0.77 (−0.16, | 0.45 (−0.24, |
| placebo (95% CI) | 1.70) | 1.14) |
| P-value vs. placebo | 0.1046 | 0.2020 |
| Overall p-value for interaction | 0.8943 | 0.5672 |

AQLQ(S): Asthma quality of life questionnaire with standardized activities, LOAC: Loss of asthma control, LOCF: Last observation carried forward, LATAM: Latin America, ROW: Rest of the world

TABLE 57
Subgroup analysis: change from baseline in AQLQ global score at EOT (Week
12) in the baseline low EOS and low FeNO subgroup (mITT population).

| | 400 mg BID | | 400 mg TID | |
|---|---|---|---|---|
| | | Rilzabrutinib | | Rilzabrutinib |
| | Placebo BID | 400 mg BID | Placebo BID | 400 mg TID |
| AQLQ global score | (N = 32) | (N = 32) | (N = 68) | (N = 64) |

Baseline blood eosinophil (10\hat{ }9/L) and FeNO (ppb)
Low EOS (<0.15) and low FeNO (<25)

| Number | 3 | | 3 | 22 | 14 |
|---|---|---|---|---|---|
| Mean (SD) | −0.49 | (1.63) | 0.71 (1.25) | 0.41 (0.72) | 0.41 (0.64) |
| LS Mean (SE) | −1.16 | (0.91) | 1.28 (0.65) | 0.45 (0.15) | 0.55 (0.19) |

| | | |
|---|---|---|
| LS Mean Diff vs. | 2.45 (−13.85, | 0.10 (−0.36, |
| placebo (95% CI) | 18.75) | 0.56) |
| P-value vs. placebo | 0.3072 | 0.6810 |

All others

| Number | 26 | | 26 | 36 | 37 |
|---|---|---|---|---|---|
| Mean (SD) | 0.38 | (1.09) | 0.85 (0.99) | 0.26 (0.95) | 0.51 (0.95) |
| LS Mean (SE) | 0.16 | (0.19) | 0.77 (0.22) | 0.21 (0.17) | 0.56 (0.16) |

| | | |
|---|---|---|
| LS Mean Diff vs. | 0.61 (0.13, | 0.35 (−0.07, |
| placebo (95% CI) | 1.09) | 0.77) |
| P-value vs. placebo | 0.0135 | 0.1044 |
| Overall p-value for interaction | 0.1818 | 0.4365 |

LATAM: Latin America, ROW: Rest of the world

### Safety

Rilzabrutinib was generally safe, with no evidence of new safety concerns (Table 58). Participants receiving 400 mg BID or 400 mg TID rilzabrutinib experienced fewer TEAEs than patients receiving the placebo, further attesting to the safety of rilzabrutinib.

TABLE 58
Overview of adverse event profile: treatment-emergent adverse
events (safety population).

| | 400 mg BID cohort | | 400 mg TID cohort | | | |
|---|---|---|---|---|---|---|
| | Placebo | Rilzabrutinib | Placebo | Rilzabrutinib | Combined | |
| | BID | 400 mg BID | TID | 400 mg TID | Placebo | Rilzabrutinib |
| n (%) | (N = 32) | (N = 32) | (N = 68) | (N = 64) | (N = 100) | (N = 96) |
| Participants | 20 (62.5) | 14 (43.8) | 18 (26.5) | 26 (40.6) | 38 (38.0) | 40 (41.7) |
| with any TEAE | | | | | | |
| Participants | 0 | 0 | 0 | 1 (1.6) | 0 | 1 (1.0) |
| with any severe | | | | | | |
| TEAE | | | | | | |
| Participants | 0 | 2 (6.3) | 0 | 0 | 0 | 2 (2.1) |
| with any | | | | | | |
| treatment | | | | | | |
| emergent SAE | | | | | | |
| Participants | 0 | 0 | 0 | 0 | 0 | 0 |
| with any TEAE | | | | | | |
| leading to death | | | | | | |
| Participants | 2 (6.3) | 4 (12.5) | 3 (4.4) | 7 (10.9) | 5 (5.0) | 11 (11.5) |
| with any TEAE | | | | | | |
| leading to | | | | | | |
| permanent study | | | | | | |
| intervention | | | | | | |
| discontinuation | | | | | | |
| Participants | 2 (6.3) | 1 (3.1) | 4 (5.9) | 3 (4.7) | 6 (6.0) | 4 (4.2) |
| with any | | | | | | |
| treatment | | | | | | |
| emergent AESI | | | | | | |
| Participants | 1 (3.1) | 5 (15.6) | 7 (10.3) | 10 (15.6) | 8 (8.0) | 15 (15.6) |
| with any TEAE | | | | | | |
| related to IMP | | | | | | |

TEAE: Treatment-emergent adverse event,
SAE: Serious adverse event,
AESI: Adverse event of special interest,
IMP: Investigational medicinal product
n (%) = number and percentage of participants with at least one TEAE

Change from Baseline at EOT and Change from Baseline at Each Week for Asthma Daytime Symptom Diary (ADSD) and Asthma Nighttime Symptom Diary (ANSD) Scores

In both cohorts, patients treated with rilzabrutinib showed an improvement in ADSD and ANSD symptom score as indicated by a reduction from baseline in ADSD and ANSD symptom score at Week 12 relative to patients treated with the placebo. The improvement has been observed across all ADSD and ANSD components, including difficulty breathing, wheezing, shortness of breath, chest tightness, chest pain, and cough.

Patients in the rilzabrutinib 400 mg BID arm exhibited a reduction in daytime and nighttime asthma symptom score as measured by change from baseline at Week 12 (LS mean ADSD: −0.27, LS mean ANSD: −0.35) that was greater than that observed in patients in the placebo BID arm (LS mean ADSD: 0. 12, ANSD: 0.33); the LS mean difference versus placebo was −0.39 [95% CI: −1.03, 0.26, nominal p-value=0.2377] for ADSD symptom score and −0.68 [95% CI: −1.33, −0.03, nominal p-value=0.0390] for ANSD symptom score. The improvement has been observed across all ADSD and ANSD components.

Patients in the rilzabrutinib 400 mg TID arm also exhibited a reduction in daytime and nighttime asthma symptom score as measured by change from baseline at Week 12 (LS mean ADSD: −0.43, LS mean ANSD: −0.48) that was greater than that observed in patients in the placebo TID arm (LS mean ADSD: 0.08, ANSD: 0.06), the LS mean difference versus placebo was −0.50 [95% CI: −1.15, 0.14, nominal p-value=0.1276] for ADSD symptom score and −0.54 [95% CI: −1.13, 0.06, nominal p-value=0.0758] for ANSD symptom score. The improvement has been observed across all ADSD and ANSD components.

### EMBODIMENTS

Non-limiting embodiments of the disclosure include:
1. A method of treating asthma in a human patient in need thereof, comprising administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof.
2. The method of Embodiment 1, wherein the human patient has mild asthma.
3. The method of Embodiment 1, wherein the human patient has moderate-to-severe asthma.
4. The method of Embodiment 1 or 3, wherein the human patient has moderate asthma.
5. The method of Embodiment 1 or 3, wherein the human patient has severe asthma.
6. The method of any one of Embodiments 1-5, wherein the human patient has asthma that is not well controlled.
7. The method of any one of the preceding Embodiments, wherein the human patient has asthma that is not well controlled for one or more symptoms.
8. The method of Embodiment 7, wherein the one or more symptoms is selected from frequency of awakening by asthma, asthma symptoms when woke up, limitation of activities by asthma, shortness of breath, and wheeze.
9. The method of any one of the preceding Embodiments, wherein the human patient is receiving treatment with step 3, 4, or 5 of the Global Initiative for Asthma (GINA).
10. The method of any one of the preceding Embodiments, wherein the human patient has asthma that is not well controlled by treatment with an inhaled corticosteroid (ICS) and/or a long-acting β2 adrenergic agonist (LABA).
11. The method of any one of the preceding Embodiments, wherein the human patient is withdrawn from ICS/LABA therapy.
12. The method of any one of the preceding Embodiments, wherein the human patient does not experience a ≥30% reduction from baseline in morning peak expiratory flow (PEF) on at least 2 consecutive days.
13. The method of any one of the preceding Embodiments, wherein the human patient does not experience a ≥30% reduction from baseline in morning PEF.
14. The method of any one of the preceding Embodiments, wherein the human patient does not require ≥6 additional reliever puffs of salbutamol/albuterol or levosalbutamol/levalbuterol within a 24-hour period for two consecutive 24-hour periods.
15. The method of any one of the preceding Embodiments, wherein the human patient does not require ≥6 additional reliever puffs of salbutamol/albuterol or levosalbutamol/levalbuterol within a 24-hour period.
16. The method of any one of the preceding Embodiments, wherein the human patient does not require ≥5 additional reliever puffs of salbutamol/albuterol or levosalbutamol/levalbuterol within a 24-hour period.
17. The method of any one of the preceding Embodiments, wherein the human patient does not require ≥4 additional reliever puffs of salbutamol/albuterol or levosalbutamol/levalbuterol within a 24-hour period.
18. The method of any one of the preceding Embodiments, wherein the human patient does not require ≥3 additional reliever puffs of salbutamol/albuterol or levosalbutamol/levalbuterol within a 24-hour period.
19. The method of any one of the preceding Embodiments, wherein the human patient does not require ≥2 additional reliever puffs of salbutamol/albuterol or levosalbutamol/levalbuterol within a 24-hour period.
20. The method of any one of the preceding Embodiments, wherein the human patient does not require an increase in ICS≥4 times the last prescribed ICS dose.
21. The method of any one of the preceding Embodiments, wherein the human patient does not require an increase in ICS≥50% of the last prescribed ICS dose.
22. The method of any one of the preceding Embodiments, wherein the human patient does not require the use of systemic steroid treatment, optionally wherein the systemic steroid treatment comprises oral or parenteral treatment.
23. The method of any one of the preceding Embodiments, wherein the human patient does not require hospitalization or an emergency room visit for asthma exacerbation.
24. The method of any one of the preceding Embodiments, wherein the human patient achieves an increase in pre-bronchodilator FEV1 relative to baseline pre-bronchodilator FEV1.
25. The method of any one of the preceding Embodiments, wherein the human patient achieves an increase in the percent predicted pre-bronchodilator FEV1 prior to baseline pre-bronchodilator FEV1.
26. The method of any one of the preceding Embodiments, wherein the human patient achieves a reduction in Asthma Control Questionnaire-5 (ACQ-5) score relative to a baseline ACQ-5 score.
27. The method of any one of the preceding Embodiments, wherein the human patient achieves a reduction in ACQ-5 of ≥0.5 relative to baseline ACQ-5.
28. The method of any one of the preceding Embodiments, wherein the human patient achieves an ACQ-5 score of ≤0.75.
29. The method of any one of the preceding Embodiments, wherein the human patient achieves an increase in Asthma Quality of Life Questionnaire with Standardized Activities (AQLQ(S)) relative to baseline AQLQ(S).
30. The method of any one of the preceding Embodiments, wherein the human patient achieves a reduction in asthma daytime symptom diary (ADSD) relative to baseline ADSD.
31. The method of any one of the preceding Embodiments, wherein the human patient achieves a reduction in asthma nighttime symptom diary (ANSD) relative to baseline ANSD.
32. The method of any one of Embodiments 1-10 or 12-31, wherein the human patient achieves a reduction in inhalations/day of albuterol or levalbuterol for symptom relief relative to baseline inhalations/day.
33. The method of any one of Embodiments 1-10 or 12-32, wherein the human patient achieves a reduction in inhalations/day of albuterol or levalbuterol for symptom relief relative to baseline inhalations/day, further wherein the patient receives concomitant treatment with ICS and/or LABA.
34. The method of any one of the preceding Embodiments, wherein the human patient achieves a reduction in Patient Global Impression of Severity (PGIS) relative to baseline PGIS.
35. The method of any one of the preceding Embodiments, wherein the human patient achieves an improvement in Patient Global Impression of Change (PGIC).
36. The method of any one of the preceding Embodiments, wherein the human patient has had asthma for at least 12 months.
37. The method of any one of the preceding Embodiments, wherein the human patient is being treated with a moderate to high dose of ICS therapy in combination with LABA.
38. The method of Embodiment 37, wherein the ICS is fluticasone propionate and the human patient is being treated with ≥250 μg of fluticasone propionate BID or comparable ICS daily dosage to a maximum of 2000 μg/day of fluticasone propionate or clinically comparable ICS.
39. The method of Embodiment 37 or 38, wherein the human patient has received treatment with the ICS for at least 3 months.
40. The method of any one of the preceding Embodiments, wherein the human patient has a baseline reversibility of at least 12% and 200 mL in FEV1 15 to 30 minutes after administration of 200-400 μg of albuterol/salbutamol or levalbuterol/levosalbutamol.
41. The method of any one of the preceding Embodiments, wherein the human patient has a history reversibility of at least 12% and 200 mL in FEV1 15 to 30 minutes after administration of 200-400 μg of albuterol/salbutamol or levalbuterol/levosalbutamol within the 5 years prior to initiation rilzabrutinib treatment.
42. The method of any one of the preceding Embodiments, wherein the human patient has a history of positive response to methacholine challenge within the 5 years prior to initiation rilzabrutinib treatment.
43. The method of any one of the preceding Embodiments, wherein the human patient has a history of one or more of the following within the 2 years prior to initiation of rilzabrutinib treatment:
   a. treatment with a systemic steroid for worsening asthma, optionally wherein the systemic steroid treatment comprises oral or parenteral treatment; and
   b. hospitalization or an emergency room visit for asthma exacerbation.
44. The method of any one of the preceding Embodiments, wherein the treatment period is at least 12 weeks.
45. The method of any one of the preceding Embodiments, wherein the at least one compound consists of at least one compound chosen from the (E) isomer of rilzabrutinib and pharmaceutically acceptable salts thereof.
46. The method of any one of Embodiments 1-44, wherein the at least one compound consists of at least one compound chosen from the (Z) isomer of rilzabrutinib and pharmaceutically acceptable salts thereof.
47. The method of any one of Embodiments 1-44, wherein the at least one compound consists of a mixture of (E) and (Z) isomers of rilzabrutinib or a pharmaceutically acceptable salt of the foregoing.
48. The method of any one of Embodiments 1-47, comprising administering rilzabrutinib to the human patient twice a day.
49. The method of any one of Embodiments 1-48, comprising administering to the human patient 400 mg of rilzabrutinib twice a day.
50. The method of any one of Embodiments 1-47, comprising administering rilzabrutinib to the human patient rilzabrutinib three times a day.
51. The method of any one of Embodiments 1-47 or 50, comprising administering to the human patient 400 mg of rilzabrutinib three times a day.
52. The method of any one of Embodiments 1-45 or 48-51 wherein the at least one compound is the (E) isomer of rilzabrutinib.
53. The method of any one of Embodiments 1-44, 46, or 48-51, wherein the at least one compound is the (Z) isomer of rilzabrutinib.
54. The method of any one of Embodiments 1-44 or 47-51, wherein the at least one compound consists of a mixture of (E) and (Z) isomers of rilzabrutinib.
55. The method of any one of the preceding Embodiments, wherein the at least one compound is orally administered to the human patient.
56. The method of any one of the preceding Embodiments, wherein the at least one compound is administered to the human patient in the form of at least one tablet.
57. The method of any one of the preceding Embodiments, wherein the at least one compound is administered with water.
58. The method of any one of Embodiments 1-44 and 55-57, wherein the at least one compound is rilzabrutinib.
59. A method of treating asthma in a human patient in need thereof, comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, wherein the human patient in need thereof has asthma that is not well controlled by treatment with an inhaled corticosteroid (ICS) and/or a long-acting β2 adrenergic agonist (LABA).
60. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof.
61. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof, wherein the human patient in need thereof has asthma that is not well controlled by treatment with an inhaled corticosteroid (ICS) and/or a long-acting β2 adrenergic agonist (LABA).
62. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof.
63. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA).
64. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof.
65. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof, wherein the human patient in need thereof has asthma that is not well controlled by treatment with an inhaled corticosteroid (ICS) and/or a long-acting β2 adrenergic agonist (LABA).
66. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof.
67. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA)

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

What is claimed is:

1. A method of treating asthma in a human patient in need thereof, comprising administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof.

2. The method of claim 1, wherein the human patient has mild asthma.

3. The method of claim 1, wherein the human patient has moderate-to-severe asthma.

4. The method of claim 1 or 3, wherein the human patient has moderate asthma.

5. The method of claim 1 or 3, wherein the human patient has severe asthma.

6. The method of any one of claims 1-5, wherein the human patient has asthma that is not well controlled.

7. The method of any one of the preceding claims, wherein the human patient has asthma that is not well controlled for one or more symptoms.

8. The method of claim 7, wherein the one or more symptoms is selected from frequency of awakening by asthma, asthma symptoms when woke up, limitation of activities by asthma, shortness of breath, and wheeze.

9. The method of any one of the preceding claims, wherein the human patient is receiving treatment with step 3, 4, or 5 of the Global Initiative for Asthma (GINA).

10. The method of any one of the preceding claims, wherein the human patient has asthma that is not well controlled by treatment with an inhaled corticosteroid (ICS) and/or a long-acting β2 adrenergic agonist (LABA).

11. The method of any one of the preceding claims, wherein the human patient is withdrawn from ICS/LABA therapy.

12. The method of any one of the preceding claims, wherein the human patient does not experience a ≥30% reduction from baseline in morning peak expiratory flow (PEF) on at least 2 consecutive days.

13. The method of any one of the preceding claims, wherein the human patient does not experience a ≥30% reduction from baseline in morning PEF.

14. The method of any one of the preceding claims, wherein the human patient does not require ≥6 additional reliever puffs of salbutamol/albuterol or levosalbutamol/levalbuterol within a 24-hour period for two consecutive 24-hour periods.

15. The method of any one of the preceding claims, wherein the human patient does not require ≥6 additional reliever puffs of salbutamol/albuterol or levosalbutamol/levalbuterol within a 24-hour period.

16. The method of any one of the preceding claims, wherein the human patient does not require an increase in ICS≥4 times the last prescribed ICS dose.

17. The method of any one of the preceding claims, wherein the human patient does not require an increase in ICS≥50% of the last prescribed ICS dose.

18. The method of any one of the preceding claims, wherein the human patient does not require the use of systemic steroid treatment, optionally wherein the systemic steroid treatment comprises oral or parenteral treatment.

19. The method of any one of the preceding claims, wherein the human patient does not require hospitalization or an emergency room visit for asthma exacerbation.

20. The method of any one of the preceding claims, wherein the human patient achieves an increase in pre-bronchodilator FEV1 relative to baseline pre-bronchodilator FEV1.

21. The method of any one of the preceding claims, wherein the human patient achieves an increase in the percent predicted pre-bronchodilator FEV1 prior to baseline pre-bronchodilator FEV1.

22. The method of any one of the preceding claims, wherein the human patient achieves a reduction in Asthma Control Questionnaire-5 (ACQ-5) score relative to a baseline ACQ-5 score.

23. The method of any one of the preceding claims, wherein the human patient achieves a reduction in ACQ-5 of ≥0.5 relative to baseline ACQ-5.

24. The method of any one of the preceding claims, wherein the human patient achieves an ACQ-5 score of ≤0.75.

25. The method of any one of the preceding claims, wherein the human patient achieves an increase in Asthma Quality of Life Questionnaire with Standardized Activities (AQLQ(S)) relative to baseline AQLQ(S).

26. The method of any one of the preceding claims, wherein the human patient achieves a reduction in asthma daytime symptom diary (ADSD) relative to baseline ADSD.

27. The method of any one of the preceding claims, wherein the human patient achieves a reduction in asthma nighttime symptom diary (ANSD) relative to baseline ANSD.

28. The method of any one of claims 1-10 or 12-27, wherein the human patient achieves a reduction in inhalations/day of albuterol or levalbuterol for symptom relief relative to baseline inhalations/day.

29. The method of any one of claims 1-10 or 12-28, wherein the human patient achieves a reduction in inhalations/day of albuterol or levalbuterol for symptom relief relative to baseline inhalations/day, further wherein the patient receives concomitant treatment with ICS and/or LABA.

30. The method of any one of the preceding claims, wherein the human patient achieves a reduction in Patient Global Impression of Severity (PGIS) relative to baseline PGIS.

31. The method of any one of the preceding claims, wherein the human patient achieves an improvement in Patient Global Impression of Change (PGIC).

32. The method of any one of the preceding claims, wherein the human patient has had asthma for at least 12 months.

33. The method of any one of the preceding claims, wherein the human patient is being treated with a moderate to high dose of ICS therapy in combination with LABA.

34. The method of claim 33, wherein the ICS is fluticasone propionate and the human patient is being treated with ≥250 μg of fluticasone propionate BID or comparable ICS daily dosage to a maximum of 2000 μg/day of fluticasone propionate or clinically comparable ICS.

35. The method of claim 33 or 34, wherein the human patient has received treatment with the ICS for at least 3 months.

36. The method of any one of the preceding claims, wherein the human patient has a baseline reversibility of at least 12% and 200 mL in FEV1 15 to 30 minutes after administration of 200-400 μg of albuterol/salbutamol or levalbuterol/levosalbutamol.

37. The method of any one of the preceding claims, wherein the human patient has a history reversibility of at least 12% and 200 mL in FEV1 15 to 30 minutes after administration of 200-400 μg of albuterol/salbutamol or levalbuterol/levosalbutamol within the 5 years prior to initiation rilzabrutinib treatment.

38. The method of any one of the preceding claims, wherein the human patient has a history of positive response to methacholine challenge within the 5 years prior to initiation rilzabrutinib treatment.

39. The method of any one of the preceding claims, wherein the human patient has a history of one or more of the following within the 2 years prior to initiation of rilzabrutinib treatment:
a. treatment with a systemic steroid for worsening asthma, optionally wherein the systemic steroid treatment comprises oral or parenteral treatment; and
b. hospitalization or an emergency room visit for asthma exacerbation.

40. The method of any one of the preceding claims, wherein the treatment period is at least 12 weeks.

41. The method of any one of the preceding claims, wherein the at least one compound consists of at least one compound chosen from the (E) isomer of rilzabrutinib and pharmaceutically acceptable salts thereof.

42. The method of any one of claims 1-40, wherein the at least one compound consists of at least one compound chosen from the (Z) isomer of rilzabrutinib and pharmaceutically acceptable salts thereof.

43. The method of any one of claims 1-40, wherein the at least one compound consists of a mixture of (E) and (Z) isomers of rilzabrutinib or a pharmaceutically acceptable salt of the foregoing.

44. The method of any one of claims 1-43, comprising administering rilzabrutinib to the human patient twice a day.

45. The method of any one of claims 1-44, comprising administering to the human patient 400 mg of rilzabrutinib twice a day.

46. The method of any one of claims 1-43, comprising administering rilzabrutinib to the human patient three times a day.

47. The method of any one of claims 1-43 or 46, comprising administering to the human patient 400 mg of rilzabrutinib three times a day.

48. The method of any one of claims 1-41 or 44-47 wherein the at least one compound is the (E) isomer of rilzabrutinib.

49. The method of any one of claims 1-40, 42, or 44-47, wherein the at least one compound is the (Z) isomer of rilzabrutinib.

50. The method of any one of claims 1-40 or 43-47, wherein the at least one compound consists of a mixture of (E) and (Z) isomers of rilzabrutinib.

51. The method of any one of the preceding claims, wherein the at least one compound is orally administered to the human patient.

52. The method of any one of the preceding claims, wherein the at least one compound is administered to the human patient in the form of at least one tablet.

53. The method of any one of the preceding claims, wherein the at least one compound is administered with water.

54. The method of any one of claims 1-40 and 51-53, wherein the at least one compound is rilzabrutinib.

55. A method of treating asthma in a human patient in need thereof, comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, wherein the human patient in need thereof has asthma that is not well controlled by treatment with an inhaled corticosteroid (ICS) and/or a long-acting β2 adrenergic agonist (LABA).

56. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof.

57. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for treating asthma in a human patient in need thereof, wherein the human patient in need thereof has asthma that is not well controlled by treatment with an inhaled corticosteroid (ICS) and/or a long-acting β2 adrenergic agonist (LABA).

58. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof.

59. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA).

60. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof.

61. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in treating asthma in a human patient in need thereof, wherein the human patient in need thereof has asthma that is not well controlled by treatment with an inhaled corticosteroid (ICS) and/or a long-acting β2 adrenergic agonist (LABA).

62. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof.

63. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof for use in the manufacture of a medicament for treating asthma in a human patient in need thereof, wherein the human patient in need thereof has asthma that is not well controlled by treatment with an inhaled corticosteroid (ICS) and/or a long-acting β2 adrenergic agonist (LABA).
